(19) United States
(12) Patent Application Publication
(10) Pub. No.: US 2007/0054268 A1
(43) Pub. Date: Mar. 8, 2007

(54) Methods of diagnosis and prognosis of ovarian cancer

(76) Inventors: Robert Sutherland, New South Wales (AU); Susan Henshall, New South Wales (AU); Philippa O'Brien, New South Wales (AU)

Correspondence Address: COZEN O'CONNOR, P.C., 1900 MARKET STREET, PHILADELPHIA, PA, 19103-3508, US

(21) Appl. No.: 10/526,979

(22) PCT Filed: Sep. 5, 2003
(86) PCT No.: PCT/AU03/01166

§ 371 Date: May 17, 2006

(30) Foreign Application Priority Data
Sep. 5, 2002 (AU) 20022951346

(51) Int. Cl. C12Q 1/68; G01N 33/574

(52) U.S. Cl. 435/6; 435/7.23

(57) ABSTRACT

The present invention provides novel genes and proteins for diagnosing ovarian cancer and/or a likelihood for survival, or recurrence of disease, wherein the expression of the genes and proteins is up-regulated or down-regulated or associated with the occurrence or recurrence of a specific scanner sub-type. The ovarian cancer-associated genes and proteins of the invention are specifically exemplified by the genes and proteins set forth in Tables 1 to 3 and the Sequence Listing.

### FIELD OF THE INVENTION

The present invention relates to the identification of nucleic acid and protein expression profiles and nucleic acids, products, and antibodies thereto that are involved in ovarian cancer; and to the use of such expression profiles and compositions in the diagnosis, prognosis and therapy of ovarian cancer. More particularly, this invention relates to novel genes that are expressed at elevated or reduced levels in malignant tissues and uses therefor in the diagnosis of cancer or malignant tumors in human subjects. This Invention also relates to the use of nucleic acid or antibody probes to specifically detect ovarian cancer cells, such as, for example, in the ovarian surface epithelium, wherein over-expression or reduced expression of nucleic acids hybridizing to the probes is highly associated with the occurrence and/or recurrence of an ovarian tumor, and/or the likelihood of patient survival. The diagnostic and prognostic test of the present invention is particularly useful for the early detection of ovarian cancer or metastases thereof, or other cancers, and for monitoring the progress of disease, such as, for example, during remission or following surgery or chemotherapy. The present invention is also directed to methods of therapy wherein the activity of a protein encoded by a diagnostic/prognostic gene described herein is modulated.

### BACKGROUND OF THE INVENTION

1. General

As used herein the term “derived from” shall be taken to indicate that a specified integer are obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

The embodiments of the invention described herein with respect to any single embodiment shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally equivalent products, compositions and methods are clearly within the scope of the invention, as described herein.

The present invention is performed without undue experimentation using, unless otherwise indicated, conventional techniques of molecular biology, microbiology, virology, recombining DNA technology, peptide synthesis in solution, solid phase peptide synthesis, and immunology. Such procedures are described, for example, in the following texts that are incorporated herein by reference:
1. Sambrook, Fritsch & Maniatis, Molecular Cloning: A Laboratory Manual, Cold Spring Harbor Laboratories, New York, Second Edition (1989), whole of Vols I, II, and II;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Perbal, B., A Practical Guide to Molecular Cloning (1984);
6. Wunsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
7. Handbook of Experimental Immunology, Vols. I-IV (D. M. Weir and C. C. Blackwell, eds., 1986, Blackwell Scientific Publications).

This specification contains nucleotide and amino acid sequence information prepared using Patentin Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

2. Description of the Related Art

Cancer is a multi-factorial disease and major cause of morbidity in humans and other animals, and deaths resulting from cancer in humans are increasing and expected to surpass deaths from heart disease in future. Carcinomas of the lung, prostate, breast, colon, pancreas, and ovary are major contributing factors to total cancer death in humans. For example, prostate cancer is the fourth most prevalent cancer and the second leading cause of cancer death in males. Similarly, cancer of the ovary is the second most common cancer of the female reproductive organs and the fourth most common cause of cancer death among females. With few exceptions, metastatic disease from carcinoma is fatal. Even if patients survive their primary cancers, recurrence or metastases are common.

It is widely recognized that simple and rapid tests for solid cancers or tumors have considerable clinical potential. Not only can such tests be used for the early diagnosis of cancer but they also allow the detection of tumor recurrence following surgery and chemotherapy. A number of cancer-specific blood tests have been developed which depend upon the detection of tumor-specific antigens in the circulation (Catalona, W. J., et al., 1991, “Measurement of prostate-specific antigen in serum as a screening test for prostate cancer”, N. Engl. J. Med. 324, 1156-1161; Barrenetxea, G., et al., 1998, “Use of serum tumor markers for the diagnosis and follow-up of breast cancer”, Oncology, 55, 447-449; Cairns, P., and Sidreansky, D., 1999, “Molecular methods for the diagnosis of cancer”. Biochim. Biophys. Acta. 1423, C 11-C 18).

Ovarian cancer is the fourth most frequent cause of cancer death in females and in the United States, and accounts for approximately 13,000 deaths annually. Furthermore, ovarian cancer remains the number one killer of women with gynaecological malignant hyperplasia and the incidence is rising in industrialized countries. The etiology of the neoplastic transformation remains unknown although there is epidemiological evidence for an association with disordered endocrine function. The incidence of ovarian carcinoma is higher in nulliparous females and in those with early menopause.

Most ovarian cancers are thought to arise from the ovarian surface of epithelium (OSE). Epithelial ovarian cancer is seldom encountered in women less than 35 years of age. Its incidence increases sharply with advancing age and peaks at ages 75 to 80, with the median age being 60 years. The single most important known risk factor is a strong familial history of breast or ovarian cancer. To date, little is known about the structure and function of the OSE cells. It is known that the OSE is highly dynamic tissue that undergoes morphogenic changes, and has proliferative properties sufficient to cover the ovulatory site following ovulation. Morphological and histochemical studies suggest that the OSE has secretory, endocytotic and transport functions which are hormonally-controlled (Blaustein and Lee, Oncol. 8, 34-43, 1979; Nicosia and Johnson, Int J. Gynecol. Pathol., 3, 249-260, 1983; Papadaki and Beilby, J. Cell Sci. 8, 445-464, 1971; Anderson et al., J. Morphol., 150, 135-164, 1976).

Ovarian cancers are not readily detectable by diagnostic techniques (Siemens et al., J. Cell. Physiol., 134: 347-356, 1988). In fact, the diagnosis of carcinoma of the ovary is generally only possible when the disease has progressed to a late stage of development. Approximately 75% of women diagnosed with ovarian cancer are already at an advanced stage (III and IV) of the disease at their initial diagnosis. During the past 20 years, neither diagnosis nor five year survival rates have greatly improved for these patients. This is substantially due to the high percentage of high-stage initial detection of the disease. There is therefore a need to develop new markers that improve early diagnosis and thereby reduce the percentage of high-stage initial diagnoses.

A number of proteinaceous ovarian tumor markers were evaluated several years ago, however these were found to be non-specific, and determined to be of low value as markers for primary ovarian cancer (Kudlacek et al., Gyn. One. 35, 323-329, 1989; Rustin et al., J. Clin. One., 7, 1667-1671, 1989; Sevelda et al., Am. J. Obstet. Gynecol., 161, 1213-1216, 1989; Omar et al., Tumor Biol., 10, 316-323, 1989). Several monoclonal antibodies were also shown to react with ovarian tumor associated antigens, however they were not specific for ovarian cancer and merely recognize determinants associated with high molecular weight mucin-like glycoproteins (Kenemans et al., Eur. J. Obstet Gynecol. Repod. Biol, 29, 207-218, 1989; McDuffy, Ann. Clin. Biochem., 26, 379-387, 1989). More recently, oncogenes associated with ovarian cancers have been identified, including HER-21neu (c-erbB-2) which is over-expressed in one-third of ovarian cancers (U.S. Pat. No. 6,075,122 by Cheever et al, issued Jun. 13, 2000), the fms oncogene, and abnormalities in the p53 gene, which are seen in about half of ovarian cancers.

Whilst previously identified markers for carcinomas of the ovary have facilitated efforts to diagnose and treat these serious diseases, there is a clear need for the identification of additional markers and therapeutic targets. The identification of tumor markers that are amenable to the early-stage detection of localized tumors is critical for more effective management of carcinomas of the ovary.

### SUMMARY OF THE INVENTION

In work leading up to the present invention, the inventors sought to identify nucleic acid markers that were diagnostic of ovarian cancers generally, or diagnostic of specific ovarian cancers such as, for example, serous ovarian cancer (SOC), mucinous ovarian cancer (MOC), non-invasive (borderline ovarian cancer or low malignant potential ovarian cancer), mixed phenotype ovarian cancer, endometrioid ovarian cancer (EnOC) and clear cell ovarian cancer (ClCA), papillary serous ovarian cancer, Brenner cell or undifferentiated adenocarcinoma, by virtue of their modulated expression in cancer tissues derived from a patient cohort compared to their expression in healthy or non-cancerous cells and tissues. Additionally, the inventors sought to determine whether any correlation exists between the expression of any particular gene in a subject having ovarian cancer and the survival, or likelihood for survival, of the subject during the medium to long term (i.e. in the period between about 1-2 years from primary diagnosis, or longer). The inventors also sought to to determine whether any correlation exists between the expression of any particular gene in a subject following treatment for ovarian cancer and the recurrence, or likelihood for recurrence, of ovarian cancer in the subject during the medium to long term (i.e. in the period between about 1-2 years from primary diagnosis, or longer).

As exemplified herein, the inventors identified a number of genes whose expression is altered (up-regulated or down-regulated) in individuals with ovarian cancer compared to healthy individuals, eg., subjects who do not have ovarian cancer. The particular genes are identified in Tables 1 and 2. Preferably, the genes are selected from the group of candidate genes set forth in Table 3.

The list of genes and proteins exemplified herein by Table 1 were identified by a statistical analysis as outlined in the examples which gave a P-value, eg., by comparison of expression to the expression of that gene in normal ovaries.

Accordingly, one aspect of the present invention provides a method of detecting an ovarian cancer-associated transcript in a biological sample, the method comprising contacting the biological sample with a polynucleotide that selectively hybridizes to a sequence at least 80% identical to a sequence as shown in Table 1 or 2 or 3. Preferably the percentage identity to a sequence disclosed in any one of Tables 1-3 is at least about 85% or 90% or 95%, and still more preferably at least about 98% or 99%.

In a preferred embodiment, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(ii) a sequence that hybridizes under at least low stringency hybridization conditions to at least about 20 contiguous nucleotides from a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(iii) a sequence that is at least about 80% identical to a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(iv) a sequence that encodes an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 47, 49, 51, 54, 56, 58, 60, 62, 64, 66, 68, 70, 72, 74, 76, 78, 80, 82 and 84; and
(v) a sequence that is complementary to any one of the sequences set forth in (i) or (ii) or (iii) or (iv).

In a preferred embodiment, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(ii) a sequence that hybridizes under at least low stringency hybridization conditions to at least about 20 contiguous nucleotides from a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(iii) a sequence that is at least about 80% identical to a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(iv) a sequence that encodes an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 47, 49, 51, 54, 56, 58, 60, 62, 64, 66, 68, 70, 72, 74, 76, 78, 80, 82 and 84; and
(v) a sequence that is complementary to any one of the sequences set forth in (i) or (ii) or (iii) or (iv).

Even more preferably, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from a sequence selected from the group consisting of SEQ ID NOs: 1, 5, 7, 9, 11, 13, 15, 17, 21, 23, 25, 27, 29, 31, 33, 35, 37, 39, 41, 45, 46, 48, 52, 53, 55, 57, 59, 61, 63, 65, 67, 69, 71, 73, 75, 77, 79, 81 and 83;
(ii) a sequence that hybridizes under at least low stringency hybridization conditions to at least about 20 contiguous nucleotides from a sequence selected from the group consisting of SEQ ID NOs: 1, 5, 7, 9, 11, 13, 15, 17, 21, 23, 25, 27, 29, 31, 33, 35, 37, 39, 41, 45, 46, 48, 52, 53, 55, 57, 59, 61, 63, 65, 67, 69, 71, 73, 75, 77, 79, 81 and 83;
(iii) a sequence that is at least about 80% identical to a sequence selected from the group consisting of SEQ ID NOs: 1, 5, 7, 9, 11, 13, 15, 17, 21, 23, 25, 27, 29, 31, 33, 35, 37, 39, 41, 45, 46, 48, 52, 53, 55, 57, 59, 61, 63, 65, 67, 69, 71, 73, 75, 77, 79, 81 and 83;
(iv) a sequence that encodes an amino acid sequence selected from the group consisting of SEQ ID NOs: 2, 6, 8, 10, 12, 14, 16, 18, 22, 24, 26, 28, 30, 32, 34, 36, 38, 40, 42, 47, 49, 54, 56, 58, 60, 62, 64, 66, 68, 70, 72, 74, 76, 78, 80, 82 and 84; and
(v) a sequence that is complementary to (i) or (ii) or (iii) or (iv).

As used herein, the term “modified level” includes an enhanced, increased or elevated level of an integer being assayed, or alternatively, a reduced or decreased level of an integer being assayed.

In one embodiment an elevated, enhanced or increased level of expression of the nucleic acid is detected. In accordance with this embodiment, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having ovarian cancer indicates that the subject being tested has an ovarian ovarian cancer, and wherein said nucleic acid probe comprises a sequence selected from the group consisting of:
(i) a sequence comprising at least about 20 contiguous nucleotides from a nucleic acid set forth in Table 1 or 2 other than a nucleic acid having an Accession Number selected from the group consisting of NM_{—}022117, NM_{—}005460, NM_{—}002387, AI745249 and AI694200;
(ii) a sequence that hybridizes under at least low stringency hybridization conditions to at least about 20 contiguous nucleotides from a nucleic acid set forth in Table 1 or 2 other than a nucleic acid having an Accession Number selected from the group consisting of NM_{—}022117, NM_{—}005460, NM_{—}002387, AI745249 and AI694200;
(iii) a sequence that is at least about 80% identical to (i) or (ii);
(iv) a sequence that encodes a polypeptide encoded by a nucleic acid set forth in Table 1 or 2 other than a nucleic acid having an Accession Number selected from the group consisting of NM_{—}022117, NM_{—}005460, NM_{—}002387, AI745249 and AI694200; and
(v) a sequence that is complementary to any one of the sequences set forth in (i) or (ii) or (iii) or (iv).

In a preferred embodiment, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from a sequence selected from the group consisting of SEQ ID NOs: 7, 9, 11, 13, 15, 17, 21, 23, 25, 27, 29, 31, 33, 35, 37, 39, 41, 45, 46, 48, 52, 53, 55, 57, 59, 61, 63, 65, 67, 69, 71, 73, 75, 77, 79, 81 and 83;
(ii) a sequence that hybridizes under at least low stringency hybridization conditions to at least about 20 contiguous nucleotides from a sequence selected from the group consisting of SEQ ID NOs: 7, 9, 11, 13, 15, 17, 21, 23, 25, 27, 29, 31, 33, 35, 37, 39, 41, 45, 46, 48, 52, 53, 55, 57, 59, 61, 63, 65, 67, 69, 71, 73, 75, 77, 79, 81 and 83;
(iii) a sequence that is at least about 80% identical to a sequence selected from the group consisting of SEQ ID NOs: 7, 9, 11, 13, 15, 17, 21, 23, 25, 27, 29, 31, 33, 35, 37, 39, 41, 45, 46, 48, 52, 53, 55, 57, 59, 61, 63, 65, 67, 69, 71, 73, 75, 77, 79, 81 and 83;
(iv) a sequence that encodes an amino acid sequence selected from the group consisting of SEQ ID NOs: 8, 10, 12, 14, 16, 18, 22, 24, 26, 28, 30, 32, 34, 36, 38, 40, 42, 47, 49, 54, 56, 58, 60, 62, 64, 66, 68, 70, 72, 74, 76, 78, 80, 82 and 84; and
(v) a sequence that is complementary to any one of the sequences set forth in (i) or (ii) or (iii) or (iv).

In an alternative preferred embodiment, a reduced level of a diagnostic marker is indicative of ovarian cancer. In accordance with this embodiment, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a reduced level of hybridization of the probe for the subject being tested compared to the hybridization obtained for a control subject not having ovarian cancer indicates that the subject being tested has an ovarian ovarian cancer, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of NM_{—}022117, NM_{—}005460, NM_{—}002387, AI745249 and AI694200;
(ii) a sequence that hybridizes under at least low stringency hybridization conditions to at least about 20 contiguous nucleotides from a nucleic acid set forth in Table 1 and having an Accession Number selected from the group consisting of NM_{—}022117, NM_{—}005460, NM_{—}002387, AI745249 and AI694200;
(iii) a sequence that is at least about 80% identical to (i) or (ii);
(iv) a sequence that encodes a polypeptide encoded by a nucleic acid set forth in Table 1 and having an Accession Number selected from the group consisting of NM_{—}022117, NM_{—}005460, NM_{—}002387, AI745249 and AI694200; and
(v) a sequence that is complementary to any one of the sequences set forth in (i) or (ii) or (iii) or (iv).

In a preferred embodiment, the present invention provides a method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a reduced level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from a sequence selected from the group consisting of SEQ ID NOs: 1, 3, and 5;
(ii) a sequence that hybridizes under at least low stringency hybridization conditions to at least about 20 contiguous nucleotides from a sequence selected from the group consisting of SEQ ID NOs: 1, 3, and 5;
(iii) a sequence that is at least about 80% identical to a sequence selected from the group consisting of SEQ ID NOs: 1, 3, and 5;
(iv) a sequence that encodes an amino acid sequence selected from the group consisting of SEQ ID NOs: 2, 4, and 6; and
(v) a sequence that is complementary to any one of the sequences set forth in (i) or (ii) or (iii) or (iv).

Preferably, the ovarian cancer that is diagnosed according to the present invention is an epithelial ovarian cancer, such as, for example, serous ovarian cancer, non-invasive ovarian cancer, mixed phenotype ovarian cancer, mucinous ovarian cancer, endometrioid ovarian cancer, clear cell ovarian cancer, papillary serous ovarian cancer, Brenner cell or undifferentiated adenocarcinoma. As will be apparent from the preferred embodiments described below, certain of the genes represented in Table 1, Table 2 and Table 3 are expressed at modified levels in subjects having serous or mucinous ovarian cancers. Data presented in FIGS. 1-4 also exemplify novel diagnostics and prognostics for serous ovarian cancer, mucinous ovarian cancer, endometrioid ovarian cancer and clear cell ovarian cancer.

As exemplified herein by Table 2, the present inventors have identified those genes having an elevated or reduced average ratio of expression of specific genes between ovarian cancer patients vs non-ovarian cancer patients, wherein a high ratio in Table 2 indicates an enhanced expression in an ovarian cancer patients and wherein a negative ratio indicates that a reduced expression in an ovarian cancer patient.

In an alternative preferred embodiment, the present invention provides a method of diagnosing a serous ovarian cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having ovarian cancer indicates that the subject being tested has a serous ovarian cancer, and wherein said nucleic acid probe comprises a sequence selected from the group consisting of:
(i) a sequence comprising at least about 20 contiguous nucleotides from a nucleic acid set forth in Table 2 or as set forth in Table 1 and having an Accession Number selected from the group consisting of: U62801, D49441, X51630, and AB018305;
(ii) a sequence that hybridizes under at least low stringency hybridization conditions to at least about 20 contiguous nucleotides from a nucleic acid set forth in Table 2 or as set forth in Table 1 and having an Accession Number selected from the group consisting of: U62801, D49441, X51630, And AB018305;
(iii) a sequence that is at least about 80% identical to (i) or (ii);
(iv) a sequence that encodes a polypeptide encoded by a nucleic acid set forth in Table 2 or as set forth in Table 1 and having an Accession Number selected from the group consisting of: U62801, D49441, X51630, And AB018305; and
(v) a sequence that is complementary to any one of the sequences set forth in (i) or (ii) or (iii) or (iv).

In a further alternative preferred embodiment, the present invention provides a method of diagnosing a mucinous ovarian cancer in a human or animal subject being tested said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a mucinous ovarian cancer, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA132961 and AF111856;
(ii) a sequence that hybridizes under at least low stringency hybridization conditions to at least about 20 contiguous nucleotides from a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA132961 and AF111856;
(iii) a sequence that is at least about 80% identical to (i) or (ii);
(iv) a sequence that encodes a polypeptide encoded by a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA132961 and AF1111856; and
(v) a sequence that is complementary to any one of the sequences set forth in (i) or (ii) or (iii) or (iv).

In a preferred embodiment, the present invention provides a method of diagnosing a mucinous ovarian cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from SEQ ID NO: 57 or 59 or 61;
(ii) a sequence that hybridizes under at least low stringency hybridization conditions to at least about 20 contiguous nucleotides from SEQ ID NO: 57 or 59 or 61;
(iii) a sequence that is at least about 80% identical to SEQ ID NO: 57 or 59 or 61;
(iv) a sequence that encodes the amino acid sequence set forth in SEQ ID NO: 58 or 60 or 62; and
(v) a sequence that is complementary to any one of the sequences set forth in (i) or (ii) or (iii) or (iv).

Those skilled in the art will be aware that as a carcinoma progresses, metastases occur in organs and tissues outside the site of the primary tumor. For example, in the case of ovarian cancer, metastases commonly appear in a tissue selected from the group consisting of omentum, abdominal fluid, lymph nodes, lung, liver, brain, and bone. Accordingly, the term “ovarian cancer” as used herein shall be taken to include an early or developed tumor of the ovary, such as, for example, any one or more of a number of cancers of epithelial origin, such as serous, mucinous, endometrioid, clear cell, papillary serous, Brenner cell or undifferentiated adenocarcinoma, non-invasive ovarian cancer such as borderline ovarian cancer or low-malignant potential ovarian cancer, or a mixed phenotype ovarian cancer, and optionally, any metastases outside the ovary that occurs in a subject having a primary tumor of the ovary.

As used herein, the term “diagnosis”, and variants thereof, such as, but not limited to “diagnose”, “diagnosed” or “diagnosing” shall not be limited to a primary diagnosis of a clinical state, however should be taken to include any primary diagnosis or prognosis of a clinical state. For example, the “diagnostic assay” formats described herein are equally relevant to assessing the remission of a patient, or monitoring disease recurrence, or tumor recurrence, such as following surgery or chemotherapy, or determining the appearance of metastases of a primary tumor. All such uses of the assays described herein are encompassed by the present invention.

Both classical hybridization and amplification formats, and combinations thereof, are encompassed by the invention. In one embodiment, the hybridization comprises performing a nucleic acid hybridization reaction between a labeled probe and a second nucleic acid in the biological sample from the subject being tested, and detecting the label. In another embodiment, the hybridization comprising performing a nucleic acid amplification reaction eg., polymerase chain reaction (PCR), wherein the probe consists of a nucleic acid primer and nucleic acid copies of the nucleic acid in the biological sample are amplified. As will be known to the skilled artisan, amplification may proceed classical nucleic acid hybridization detection systems, to enhance specificity of detection, particularly in the case of less abundant mRNA species in the sample.

In a preferred embodiment, the polynucleotide is immobilised on a solid surface.

The present invention clearly encompasses nucleic acid-based methods and protein-based methods for diagnosing cancer in humans and other mammals.

Accordingly, in a related embodiment, the present invention provides a method of detecting an ovarian cancer-associated polypeptide in a biological sample the method comprising contacting the biological sample with an antibody that binds specifically to an ovarian cancer-associated polypeptide in the biological sample, the polypeptide being encoded by a polynucleotide that selectively hybridizes to a sequence at least 80% identical to a sequence as shown in Tables 1-3.

Preferably the percentage identity to a sequence disclosed in any one of Tables 1-3 is at least about 85% or 90% or 95%, and still more preferably at least about 98% or 99%.

In a preferred embodiment, the present invention provides a method of diagnosing an ovarian cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a sequence having at least about 80% identity to a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 47, 49, 51, 54, 56, 58, 60, 62, 64, 66, 68, 70, 72, 74, 76, 78, 80, 82 and 84.

In a preferred embodiment, the present invention provides a method of diagnosing an ovarian cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a sequence having at least about 80% identity to a sequence selected from the group consisting of SEQ ID NOs: 2, 6, 8, 10, 12, 14, 16, 18, 22, 24, 26, 28, 30, 32, 34, 36, 38, 40, 42, 47, 49, 54, 56, 58, 60, 62, 64, 66, 68, 70, 72, 74, 76, 78, 80, 82 and 84.

In one embodiment an elevated, enhanced or increased level of expression of the antigen-antibody complex is detected. In accordance with this embodiment, the present invention provides a method of diagnosing an ovarian cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a polypeptide encoded by a nucleic acid set forth in Table 1 or 2 other than a nucleic acid having an Accession Number selected from the group consisting of NM_{—}022117, NM_{—}005460, NM_{—}002387, AI745249 and AI694200.

In a preferred embodiment, the present invention provides a method of diagnosing an ovarian cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a sequence having at least about 80% identity to a sequence selected from the group consisting of SEQ ID NOs: 8, 10, 12, 14, 16, 18, 20, 22, 24, 26, 28, 30, 32, 34, 36, 38, 40, 42, 44, 47, 49, 51, 54, 56, 58, 60, 62, 64, 66, 68, 70, 72, 74, 76, 78, 80, 82 and 84.

In an alternative preferred embodiment, a reduced level of a diagnostic marker is indicative of ovarian cancer. In accordance with this embodiment, the present invention provides a method of diagnosing an ovarian cancer in a human or animal subject being tested said method comprising contacting a biological sample from said subject being tested with an antibody for a time and under conditions sufficient for an antigen-antibody complex to form and then detecting the complex wherein a reduced level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a polypeptide encoded by a nucleic acid set forth in Table 1 and having an Accession Number selected from the group consisting of NM_{—}022117, NM_{—}005460, NM_{—}002387, AI745249 and AI694200.

In a preferred embodiment, the present invention provides a method of diagnosing an ovarian cancer in a human or animal subject being tested said method comprising contacting a biological sample from said subject being tested with an antibody for a time and under conditions sufficient for an antigen-antibody complex to form and then detecting the complex wherein a reduced level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a sequence having at least about 80% identity to a sequence selected from the group consisting of SEQ ID NOs: 2, 4, and 6.

Preferably, the ovarian cancer that is diagnosed according to the present invention is an epithelial ovarian cancer, such as, for example, serous ovarian cancer or mucinous ovarian cancer.

In an alternative preferred embodiment, the present invention provides a method of diagnosing a serous ovarian cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having ovarian cancer indicates that the subject being tested has a serous ovarian cancer, and wherein said antibody binds to a polypeptide comprising an amino acid sequence comprising at least about 10 contiguous amino acid residues of a polypeptide encoded by a nucleic acid set forth in Table 2 or as set forth in Table 1 and having an Accession Number selected from the group consisting of: U62801, D49441, X51630, And AB018305.

In a further alternative preferred embodiment, the present invention provides a method of diagnosing a mucinous ovarian cancer in a human or animal subject being tested said method comprising contacting a biological sample from said subject being tested with an antibody for a time and under conditions sufficient for an antigen-antibody complex to form and then detecting the complex wherein a reduced level of the antigen-antibody complex for the subject being tested compared to the amount of the antigen-antibody complex formed for a control subject not having ovarian cancer indicates that the subject being tested has a mucinous ovarian cancer, and wherein said antibody binds to a polypeptide comprising an amino acid sequence comprising at least about 10 contiguous amino acid residues of a polypeptide encoded by a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA132961 and AF111856.

In a preferred embodiment, the present invention provides a method of diagnosing a mucinous ovarian cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has a mucinous ovarian cancer, and wherein said antibody binds to a polypeptide comprising an amino acid sequence comprising at least about 10 contiguous amino acid residues of a sequence having at least about 80% identity to SEQ ID NO: 58 or 60 or 62.

In a further related embodiment, the present invention provides a method of detecting an ovarian cancer-associated antibody in a biological sample the method comprising contacting the biological sample with a polypeptide encoded by a polynucleotide that selectively hybridizes to a sequence at least 80% identical to a sequence as shown in Tables 1-3, wherein the polypeptide specifically binds to the ovarian cancer-associated antibody.

Preferably, in the above methods, the biological sample is contacted with a plurality of the polynucleotides, polypeptides or antibodies referred to above.

In a particularly preferred embodiment, the present invention provides an antibody-based mulptiplex assay for determining the likelihood of survival of a subject from an ovarian cancer. In one embodiment, the invention provides a method of determining the likelihood of survival of a subject suffering from a serous ovarian cancer, said method comprising contacting a biological sample from said subject being tested with at least two antibodies for a time and under conditions sufficient for antigen-antibody complexes to form and then detecting the complexes wherein an enhanced level of the antigen-antibody complexes for the subject being tested compared to the amount of the antigen-antibody complexes formed for a control subject not having ovarian cancer indicates that the subject being tested has a poor probability of survival, and wherein one antibody binds to an sFRP polypeptide comprising the amino acid sequence set forth in SEQ ID NO: 72 and wherein one antibody binds to a SOCS3 polypeptide comprising the amino acid sequence set forth in SEQ ID NO: 74.

The present invention is not to be limited by the source or nature of the biological sample. In one embodiment, the biological sample is from a patient undergoing a therapeutic regimen to treat ovarian cancer. In an alternative preferred embodiment, the biological sample is from a patient suspected of having ovarian cancer.

In addition to providing up-regulated and down-regulated genes, the list of genes and proteins exemplified herein by Table 1 were identified by a statistical analysis as outlined in the examples which gave a P-value, eg., by comparison of expression to clinicopathological parameters for disease recurrence or patent survival. Accordingly, the present invention is particularly useful for prognostic applications, in particular for assessing the medium-to-long term survival of a subject having an ovarian cancer, or alternatively or in addition, for assessing the likelihood of disease recurrence.

Accordingly, a further aspect of the present invention provides a method of monitoring the efficacy of a therapeutic treatment of ovarian cancer, the method comprising:
(i) providing a biological sample from a patient undergoing the therapeutic treatment; and
(ii) determining the level of a ovarian cancer-associated transcript in the biological sample by contacting the biological sample with a polynucleotide that selectively hybridizes to a sequence having at least about 80% identity to a sequence as shown in any one of Tables 1-3, thereby monitoring the efficacy of the therapy.

Preferably the method further comprises comparing the level of the ovarian cancer-associated transcript to a level of the ovarian cancer-associated transcript in a biological sample from the patient prior to, or earlier in, the therapeutic treatment.

In a related embodiment, the present invention provides a method of monitoring the efficacy of a therapeutic treatment of ovarian cancer, the method comprising:
(i) providing a biological sample from a patient undergoing the therapeutic treatment; and
(ii) determining the level of a ovarian cancer-associated antibody in the biological sample by contacting the biological sample with a polypeptide encoded by a polynucleotide that selectively hybridizes to a sequence at least 80% identical to a sequence as shown in Tables 1-3, wherein the polypeptide specifically binds to the ovarian cancer-associated antibody, thereby monitoring the efficacy of the therapy.

Preferably the method further comprises comparing the level of the ovarian cancer-associated antibody to a level of the ovarian cancer-associated antibody in a biological sample from the patient prior to, or earlier in, the therapeutic treatment.

In a further related embodiment, the present invention provides a method of monitoring the efficacy of a therapeutic treatment of ovarian cancer, the method comprising:
(i) providing a biological sample from a patient undergoing the therapeutic treatment; and
(ii) determining the level of a ovarian cancer-associated polypeptide in the biological sample by contacting the biological sample with an antibody, wherein the antibody specifically binds to a polypeptide encoded by a polynucleotide that selectively hybridizes to a sequence at least 80% identical to a sequence as shown in Tables 1-3, thereby monitoring the efficacy of the therapy.

Preferably the method further comprises comparing the level of the ovarian cancer-associated polypeptide to a level of the ovarian cancer-associated polypeptide in a biological sample from the patient prior to, or earlier in, the therapeutic treatment.

It will also be apparent from the following preferred embodiments, that the expression of certain genes listed in Table 1 and Table 3 is statistically correlated with survival and death of patients having ovarian cancer, wherein a low P value indicates an enhanced likelihood that a patient having altered expression of the gene will die from the cancer.

Accordingly, in one embodiment, the present invention provides a method of determining the likelihood of survival of a subject suffering from an ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a poor probability of survival, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI1796870, X02761, AW968613, AW972565, AF045229, AW953853, U52426, F06700, AI1798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955;
(ii) a sequence that hybridizes under at least low stringency hybridization conditions to at least about 20 contiguous nucleotides from a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955;
(iii) a sequence that is at least about 80% identical to (i) or (ii);
(iv) a sequence that encodes a polypeptide encoded by a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955; and
(v) a sequence that is complementary to any one of the sequences set forth in (i) or (ii) or (iii) or (iv).

In a preferred embodiment, the present invention provides a method of determining the likelihood of survival of a subject suffering from an ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a poor probability of survival, and wherein said nucleic acid probe comprises a sequence selected from the group consisting of:
(i) a sequence comprising at least about 20 contiguous nucleotides from a sequence selected from the group consisting of SEQ ID NOs: 63, 65, 67, 69, 71, 73, 75, 77, 79, 81 and 83;
(ii) a sequence that hybridizes under at least low stringency hybridization conditions to at least about 20 contiguous nucleotides from a sequence selected from the group consisting of SEQ ID NOs: 63, 65, 67, 69, 71, 73, 75, 77, 79, 81 and 83;
(iii) a sequence that is at least about 80% identical to a sequence selected from the group consisting of SEQ ID NOs: 63, 65, 67, 69, 71, 73, 75, 77, 79, 81 and 83;
(iv) a sequence that encodes an amino acid sequence selected from the group consisting of SEQ ID NOs: 64, 66, 68, 70, 72, 74, 76, 78, 80, 82 and 84; and
(v) a sequence that is complementary to (i) or (ii) or (iii) or (iv).

In an alternative preferred embodiment, the present invention provides a method of determining the likelihood of survival of a subject suffering from an ovarian cancer, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has has a poor probability of survival, and wherein said antibody binds to a polypeptide comprising an amino acid sequence comprising at least about 10 contiguous amino acid residues of a sequence encoded by a nucleic acid set forth in Table 1 and having an Accession Number selected from the group consisting of: NM_{—}003014, AA046217, NM 015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955.

In an alternative preferred embodiment, the present invention provides a method of determining the likelihood of survival of a subject suffering from an ovarian cancer, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has has a poor probability of survival, and wherein said antibody binds to a polypeptide comprising an amino acid sequence comprising at least about 10 contiguous amino acid residues of a sequence having at least about 80% identity to a sequence selected from the group consisting of SEQ ID NOs: 64, 66, 68, 70, 72, 74, 76, 78, 80, 82 and 84.

In a particularly preferred embodiment, the present invention provides a marker for determining the likelihood of a subject surviving from serous cancer. In accordance with this embodiment of the invention, there is provided a method of determining the likelihood of survival of a subject suffering from a serous ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a poor probability of survival, and wherein said nucleic acid probe comprises a sequence selected from the group consisting of:
(i) a sequence comprising at least about 20 contiguous nucleotides from a nucleic acid comprising the nucleotide sequence set forth in SEQ ID NO: 71 or 73;
(ii) a sequence that hybridizes under at least low stringency hybridization conditions to at least about 20 contiguous nucleotides from a nucleic acid comprising the nucleotide sequence set forth in SEQ ID NO: 71 or 73;
(iii) a sequence that is at least about 80% identical to (i) or (ii) and encoding an sFRP protein or a SOCS3 protein;
(iv) a sequence that encodes a polypeptide comprising the amino acid sequence set forth in SEQ ID NO: 72 or 74; and
(v) a sequence that is complementary to any one of the sequences set forth in (i) or (ii) or (iii) or (iv).

In an alternative preferred embodiment, the present invention provides a method of determining the likelihood of survival of a subject suffering from a serous ovarian cancer, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has a poor probability of survival, and wherein said antibody binds to an sFRP polypeptide comprising the amino acid sequence set forth in SEQ ID NO: 72 or a SOCS3 polypeptide comprising the amino acid sequence set forth in SEQ ID NO: 74 or.

It will also be apparent from the following preferred embodiments, that the expression of certain genes listed in Table 1 and Table 3 is statistically correlated with recurrence of ovarian cancer, wherein a low P value indicates an enhanced likelihood that a patent having altered expression of the gene will experience recurrence of the disease.

In yet another preferred embodiment, the present invention provides a method of determining the likelihood that a subject will suffer from a recurrence of an ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a high probability of recurrence, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM_{—}000947, AJ250562, AL040183, BE207573, BE564162, BE439580, AW067800, AA569756, AW138190, AF126245, L10343, NM_{—}002514, AI863735, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N51357, N80486, NM_{—}000954, NM_{—}005756, NM_{—}016652, R26584, R31178, W05391, W25005, W45393, W68815, X65965, X76732 and Z45051,
(ii) a sequence that hybridizes under at least low stringency hybridization conditions to at least about 20 contiguous nucleotides from a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM 000947, AJ250562, AL040183, BE207573, BE564162, BE439580, AW067800, AA569756, AW138190, AF126245, L10343, NM_{—}002514, AI186373, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N513571 N80486, NM_{—}000954, NM_{—}005756, NM_{—}016652, R26584, R31178, W05391, W25005, W45393, W68815, X65965, X76732 and Z45051;
(iii) a sequence that is at least about 80% identical to (i) or (ii);
(iv) a sequence that encodes a polypeptide encoded by a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM_{—}000947, AJ250562, AL040183, BE207573, BE564162, BE439580, AW067800, AA569756, AW138190, AF126245, L10343, NM_{—}002514, AI863735, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N51357, N80486, NM_{—}000954, NM_{—}005756, NM_{—}016652, R26584, R31178, W05391, W25005, W45393, W68815, X65965, X76732 and Z45051; and
(v) a sequence that is complementary to any one of the sequences set forth in (i) or (ii) or (iii) or (iv).

In an alternative preferred embodiment, the present invention provides a method of determining the likelihood that a subject will suffer from a recurrence of an ovarian cancer, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has a high probability of recurrence, and wherein said antibody binds to a polypeptide comprising an amino acid sequence comprising at least about 10 contiguous amino acid residues of a sequence encoded by a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM_{—}000947, AJ250562, AL040183, BE207573, BE564162, BE439580, AW067800, M569756, AW138190, AF126245, L10343, NM_{—}002514, AI863735, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N51357, N80486, NM 000954, NM_{—}005756, NM_{—}016652, R26584, R31178, W05391, W25005, W45393, W68815, X65965, X76732 and Z45051.

The recurrence of ovarian cancer is a clinical recurrence as determined by the presence of one or more clinical symptoms of an ovarian cancer, such as, for example, a metastases, or alternatively, as determined in a biochemical test, immunological test or serological test such as, for example, a cross-reactivity in a biological sample to a CA125 antibody.

Preferably, the recurrence is capable of being detected at least about 2 years from treatment, more preferably about 2-3 years from treatment, and even more preferably about 4 or 5 or 10 years from treatment.

Preferably, in the above diagnostic and/or prognostic methods, the biological sample is contacted with a plurality of the nucleic acids and/or polypeptides and/or antibodies referred to above. In a particularly preferred embodiment, mulpiplex assays are performed to detect enhanced expression at least of sFRP4 and SOC3 at the protein level (eg., using antigen-based or antibody-based assays) or at the mRNA level (eg., by detecting elevated levels of mRNA transcripts).

A further embodiment of the present invention provides a method of diagnosing epithelial ovarian cancer by detecting aberrant methylation of a promoter that regulates expression of a tumor suppressor gene eg., MCC. In particular, the present invention contemplates the detection of hypermethylation of the promoter of a tumor suppressor gene. Without being bound by any theory or mode of action, such hypermethylation leads to gene inactivation, thereby reducing expression for the tumor suppressor gene and permitting oncogenesis. In one preferred embodiment, the present invention provides a method of diagnosing an ovarian cancer in a human or animal subject being tested said method comprising determining aberrant methylation in a promoter sequence that regulates expression of a tumor suppressor gene in a biological sample from said subject compared to the methylation of the promoter in nucleic acid obtained for a control subject not having ovarian cancer wherein said aberrant methylation indicates that the subject being tested has an ovarian ovarian cancer.

In a further aspect, the present invention provides a method for identifying a compound that modulates an ovarian cancer-associated polypeptide, the method comprising:
(i) contacting the compound with a ovarian cancer-associated polypeptide, the polypeptide encoded by a polynucleotide that selectively hybridizes to a sequence at least 80% identical to a sequence as shown in Tables 1-3; and
(ii) determining the functional effect of the compound upon the polypeptide.

The functional effect may, for example, be a physical effect or a chemical effect. In one embodiment, the functional effect is determined by measuring ligand binding to the polypeptide. In a particular embodiment, the polypeptide is expressed in a eukaryotic host cell or cell membrane. Preferably the polypeptide is recombinant.

In another aspect, the present invention provides a method of inhibiting proliferation of a ovarian tumour cell, which method comprises contacting said cell with a compound identified using the method supra for identifying a compound that modulates an ovarian cancer-associated polypeptide.

In a further aspect, the present invention provides a method of inhibiting proliferation of a ovarian cancer-associated cell to treat ovarian cancer in a patient, the method comprising the step of administering to the patient a therapeutically effective amount of a compound identified using the method supra for identifying a compound that modulates an ovarian cancer-associated polypeptide.

In a further aspect, the present invention provides a drug screening assay comprising:
(i) administering a test compound to a mammal having ovarian cancer or a cell isolated therefrom;
(ii) comparing the level of gene expression of a polynucleotide that selectively hybridizes to a sequence at least 80% identical to a sequence as shown in Tables 1-3 in a treated cell or mammal with the level of gene expression of the polynucleotide in a control cell or mammal, wherein a test compound that modulates the level of expression of the polynucleotide is a candidate for the treatment of ovarian cancer.

Typically, the control is a mammal with ovarian cancer or a cell therefrom that has not been treated with the test compound. Alternatively, the control is a normal cell or mammal.

The present invention also provides a method for treating a mammal having ovarian cancer comprising administering a compound identified the drug screening method supra.

In a further aspect, the present invention provides a pharmaceutical composition for use in treating a mammal having ovarian cancer, the composition comprising a compound identified the screening method supra for identifying a compound that modulates an ovarian cancer-associated polypeptide, or alternatively, using the drug screening method supra, and a physiologically acceptable carrier or diluent.

In a further aspect, the present invention provides an assay device, preferably for use in the diagnosis or prognosis of ovarian cancer, said device comprising a plurality of polynucleotides immobilized to a solid phase, wherein each of said polynucleotides consists of a gene as listed in any one of Tables 1-3. Preferably, the solid phase is a substantially planar chip.

In a related embodiment, the present invention provides an assay device, preferably for use in the diagnosis or prognosis of ovarian cancer, said device comprising a plurality of different antibodies immobilized to a solid phase, wherein each of said antibodies binds to a polypeptide listed in Tables 1-3. Preferably, the solid phase is a substantially planar chip.

Preferably, the assay device supra is used in a method of diagnosis or prognosis as described herein.

Alternatively, the assay device is used to identify modulatory compounds of the expression of one or more genes/proteins listed in any one of Tables 1-3.

In a further aspect, the present invention provides a non-human transgenic animal which is transgenic by virtue of comprising a gene set forth in any one of Tables 1-3 and, in particular, to the use of any such transgenic animal in the performance of a diagnostic or prognostic method of the invention as transgenic “knock-out” animals that have disrupted expression of a gene as set forth in any one of Tables 1-3.

In a further aspect, the present invention provides an isolated polynucleotide selected from the group consisting of;
(a) polynucleotides comprising a nucleotide sequence as shown in Tables 1-3, or the complement thereof;
(b) polynucleotides comprising a nucleotide sequence capable of selectively hybridizing to a nucleotide sequence as shown in Tables 1-3;
(c) polynucleotides comprising a nucleotide sequence capable of selectively hybridizing to the complement of a nucleotide sequence as shown in Tables 1-3; and
(d) polynucleotides comprising a polynucleotide sequence which is degenerate as a result of the genetic code to the polynucleotides defined in (a), (b) or (c).

Preferred polynucleotides comprise a polynucleotide sequence as shown in Tables 1-3 or a sequence having at least 80% homology thereto.

Preferably, the isolated polynucleotide is used for the diagnosis or prognosis of ovarian cancer, more preferably by a method as described herein. In a particularly preferred embodiment, the present invention provides for the use of a polynucleotide as set forth in any one of Tables 1-3 in the diagnosis or prognosis of ovarian cancer or for the preparation of a medicament for the treatment of ovarian cancer.

The present invention also provides a nucleic acid vector comprising a polynucleotide of the Invention. In one embodiment, the polynucleotide is operably linked to a regulatory control sequence capable of directing expression of the polynucleotide in a host cell. In a particularly preferred embodiment, the present invention provides for the use of a vector comprising a polynucleotide as set forth in any one of Tables 1-3 In the diagnosis or prognosis of ovarian cancer or for the preparation of a medicament for the treatment of ovarian cancer.

The present invention further provides a host cell comprising a vector as described in the preceding paragraph. In a particularly preferred embodiment, the present invention provides for the use of a host cell comprising an introduced polynucleotide as set forth in any one of Tables 1-3 in the diagnosis or prognosis of ovarian cancer or for the preparation of a medicament for the treatment of ovarian cancer.

In a further aspect, the present invention provides an isolated polypeptide which is encoded by a gene set forth in any one of Tables 1-3. The present invention also provides an isolated polypeptide encoded by a polynucleotide that selectively hybridizes to a sequence at least 80% identical to a sequence as shown in Tables 1-3. In a particularly preferred embodiment, the present invention provides for the use of an isolated polypeptide as set forth in any one of Tables 1-3 in the diagnosis or prognosis of ovarian cancer or for the preparation of a medicament for the treatment of ovarian cancer.

In a further aspect the present invention provides an antibody that binds specifically a polypeptide listed in Tables 1-3. In a particularly preferred embodiment, the present invention provides for the use of an antibody that binds to an isolated polypeptide as set forth in any one of Tables 1-3 in the diagnosis or prognosis of ovarian cancer or for the preparation of a medicament for the treatment of ovarian cancer.

### BRIEF DESCRIPTION OF THE DRAWINGS

FIG. 1 is a photographic representation showing expression of genes as identified by immunohistochemical staining of fixed normal (i.e. non-cancerous or healthy) tissues (panel A) or ovarian cancer tissue (panel B). The inset in panel A shows inclusion cysts. The expression levels of the following genes listed in Table 1 or Table 3 were determined: Claudin-3 (SEQ ID NO: 15); EP-CAM (Accession No. NM_{—}002354); and SOX17 (SEQ ID NO: 17). Positive controls CA125, MUC-1 and E-Cadherin were also included for comparison.

FIG. 2 is a graphical representation showing the correlation between expression of different genes in serous ovarian cancer (SOC), mucinous ovarian cancer (MOC), endometroid ovarian cancer (EnOC) and clear cell ovarian cancer (CICA). Genes indicated on the x-axis in each case are as in the legend to FIG. 1.

FIG. 3 is a copy of a photographic representation showing immunohistochemical staining of ovary tissue from a normal healthy subject (normal ovary), a subject diagnosed with mucinous ovarian cancer (MOC) and a subject diagnosed with serous ovarian cancer (SOC), following staining with probes that are specific for L1-Cadherin (top row), meprin alpha (middle row) or galectin-4 (lower row). Magnification is indicated as 20-40×.

FIG. 4a is a copy of a photographic representation showing immunohistochemical staining of samples from a normal healthy subject (normal) or primary serous ovarian tumor (SOC), following staining with probes that are specific for sFRP4 (top row), or SOCS3 (lower row). Magnification is indicated as 20×.

FIG. 4b is a copy of a graphical representation showing a Kaplan-Meier survival curve correlating sFRP4 expression to patient survival over the medium term (i.e., from about 12 months to about 48 months) to long term (more than about 48 months), indicating that high expression of sFRP4 is associated with poor survival in patients (n=127) having SOC (p=0.0056).

### DETAILED DESCRIPTION OF THE PREFERRED EMBODIMENTS

Ovarian Cancer-Associated Sequences

Ovarian cancer-associated sequences can include both nucleic acid (i.e., “ovarian cancer-associated genes”) and protein (i.e., “ovarian cancer-associated proteins”).

As used herein, the term “ovarian cancer-associated protein” shall be taken to mean any protein that has an expression pattern correlated to an ovarian cancer, the recurrence of an ovarian cancer or the survival of a subject suffering from ovarian cancer.

Similarly, the term “ovarian cancer-associated gene” shall be taken to mean any nucleic acid encoding an ovarian cancer-associated protein or nucleic acid having an expression profile that is correlated to an ovarian cancer, the recurrence of an ovarian cancer or the survival of a subject suffering from ovarian cancer.

As will be appreciated by those in the art and is more fully outlined below, ovarian cancer-associated genes are useful in a variety of applications, including diagnostic applications, which will detect naturally occurring nucleic acids, as well as screening applications; e.g., biochips comprising nucleic acid probes or PCR microtitre plates with selected probes to the ovarian cancer sequences are generated.

For identifying ovarian cancer-associated sequences, the ovarian cancer screen typically includes comparing genes identified in different tissues, e.g., normal and cancerous tissues, or tumour tissue samples from patients who have metastatic disease vs. non metastatic tissue. Other suitable tissue comparisons include comparing ovarian cancer samples with metastatic cancer samples from other cancers, such as lung, breast, gastrointestinal cancers, ovarian, etc. Samples of different stages of ovarian cancer, e.g., survivor tissue, drug resistant states, and tissue undergoing metastasis, are applied to biochips comprising nucleic acid probes. The samples are first microdissected, if applicable, and treated as is known in the art for the preparation of mRNA. Suitable biochips are commercially available, e.g. from Affymetrix. Gene expression profiles as described herein are generated and the data analyzed.

In one embodiment, the genes showing changes in expression as between normal and disease states are compared to genes expressed in other normal tissues, preferably normal ovarian, but also Including, and not limited to lung, heart, brain, liver, breast, kidney, muscle, colon, small intestine, large intestine, spleen, bone and placenta. In a preferred embodiment, those genes identified during the ovarian cancer screen that are expressed in any significant amount in other tissues are removed from the profile, although in some embodiments, this is not necessary. That is, when screening for drugs, it is usually preferable that the target be disease specific, to minimise possible side effects.

In a preferred embodiment, ovarian cancer-associated sequences are those that are up-regulated in ovarian cancer; that is, the expression of these genes is modified (up-regulated or down-regulated) in ovarian cancer tissue as compared to non-cancerous tissue (see Table 1).

“Up-regulation” as used herein means at least about a two-fold change, preferably at least about a three fold change, with at least about five-fold or higher being preferred. All Unigene cluster identification numbers and accession numbers herein are for the GenBank sequence database and the sequences of the accession numbers are hereby expressly incorporated by reference. Sequences are also available in other databases, e.g., European Molecular Biology Laboratory (EMBL) and DNA Database of Japan (DDBJ).

“Down-regulation” as used herein often means at least about a 1.5-fold change more preferably a two-fold change, preferably at least about a three fold change, with at least about five-fold or higher being most preferred.

Particularly preferred sequences are those referred to in Tables 1 or 3 that have a P value of less than 0.05, more preferably a P value of less than about 0.01.

Similarly, preferred sequences are those referred to in Table 2 as having an absolute ratio of expression between ovarian patients and normal patients of at least about ±5.0, more preferably at least about ±6.0 even more preferrably at least about ±7.0 or at least about ±8.0 or at least about ±9.0 or at least about ±0.0.

Detection of Ovarian Cancer Sequences for Diagnostic/Prognostic Applications

In one aspect, the RNA expression levels of genes are determined for different cellular states in the ovarian cancer phenotype. Expression levels of genes in normal tissue (i.e., not undergoing ovarian cancer) and in ovarian cancer tissue (and in some cases, for varying severities of ovarian cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis are performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus ovarian cancer tissue. Genes are turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression are quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection. As outlined above, preferably the change in expression (i.e., upregulation or downregulation) is at least about 50%, more preferably at least about 100%, more preferably at least about 150%, more preferably at least about 200%, with from 300 to at least 1000% being especially preferred.

Evaluation are at the gene transcript, or the protein level. The amount of gene expression are monitored using nucleic acid probes to the DNA or RNA equivalent of the gene transcript, and the quantification of gene expression levels, or, alternatively, the final gene product itself (protein) are monitored, e.g., with antibodies to the ovarian cancer-associated protein and standard immunoassays (ELISAS, etc.) or other techniques, including mass spectroscopy assays, 2D gel electrophoresis assays, etc. Proteins corresponding to ovarian cancer genes, i.e., those identified as being important in a ovarian cancer phenotype, are evaluated in a ovarian cancer diagnostic test.

In a preferred embodiment, gene expression monitoring is performed on a plurality of genes. Multiple protein expression monitoring are performed as well. Similarly, these assays are performed on an individual basis as well.

In this embodiment, the ovarian cancer nucleic acid probes are attached to biochips as outlined herein for the detection and quantification of ovarian cancer sequences in a particular cell. The assays are further described below in the example. PCR techniques are used to provide greater sensitivity.

In a preferred embodiment nucleic acids encoding the ovarian cancer-associated protein are detected. Although DNA or RNA encoding the ovarian cancer-associated protein are detected, of particular interest are methods wherein an mRNA encoding a ovarian cancer-associated protein is detected. Probes to detect mRNA are a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a ovarian cancer-associated protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The ovarian cancer-associated proteins, antibodies, nucleic acids, modified proteins and cells containing ovarian cancer sequences are used in diagnostic assays. This are performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, ovarian cancer-associated proteins, including intracellular, transmembrane or secreted proteins, find use as markers of ovarian cancer. Detection of these proteins in putative ovarian cancer tissue allows for detection or diagnosis of ovarian cancer. In one embodiment, antibodies are used to detect ovarian cancer-associated proteins. A preferred method separates proteins from a sample by electrophoresis on a gel (typically a denaturing and reducing protein gel, but are another type of gel, including isoelectric focusing gels and the like). Following separation of proteins, the ovarian cancer-associated protein is detected, e.g., by immunoblotting with antibodies raised against the ovarian cancer-associated protein. Methods of immunoblotting are well known to those of ordinary skill in the art.

In another preferred method, antibodies to the ovarian cancer-associated protein find use in in situ imaging techniques, e.g., in histology (e.g., Methods in Cell Biology: Antibodies in Cell Biology, volume 37 (Asai, ed. 1993)). In this method cells are contacted with from one to many antibodies to the ovarian cancer-associated protein(s). Following washing to remove non-specific antibody binding, the presence of the antibody or antibodies is detected. In one embodiment the antibody is detected by incubating with a secondary antibody that contains a detectable label. In another method the primary antibody to the ovarian cancer-associated proteins) contains a detectable label, e.g. an enzyme marker that can act on a substrate. In another preferred embodiment each one of multiple primary antibodies contains a distinct and detectable label. This method finds particular use in simultaneous screening for a plurality of ovarian cancer-associated proteins. As will be appreciated by one of ordinary skill in the art, many other histological imaging techniques are also provided by the invention.

In a preferred embodiment the label is detected in a fluorometer which has the ability to detect and distinguish emissions of different wavelengths. In addition, a fluorescence activated cell sorter (FACS) are used in the method. In another preferred embodiment, antibodies find use in diagnosing ovarian cancer from blood, serum, plasma, stool, and other samples. Such samples, therefore, are useful as samples to be probed or tested for, the presence of ovarian cancer-associated proteins. Antibodies are used to detect a ovarian cancer-associated protein by previously described immunoassay techniques including ELISA, immunoblotting (western blotting), immunoprecipitation, BIACORE technology and the like. Conversely, the presence of antibodies may indicate an immune response against an endogenous ovarian cancer-associated protein.

In a preferred embodiment, in situ hybridization of labeled ovarian cancer nucleic acid probes to tissue arrays is done. For example, arrays of tissue samples, including ovarian cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or are predictive of outcomes.

In a preferred embodiment, the ovarian cancer-associated proteins, antibodies, nucleic acids, modified proteins and cells containing ovarian cancer sequences are used in prognosis assays. As above, gene expression profiles are generated that correlate to ovarian cancer, in terms of long term prognosis. Again, this are done on either a protein or gene level, with the use of genes being preferred. As above, ovarian cancer probes are attached to biochips for the detection and quantification of ovarian cancer sequences in a tissue or patient. The assays proceed as outlined above for diagnosis. PCR method may provide more sensitive and accurate quantification.

Characteristics of Ovarian Cancer-Associated Proteins and Genes Encoding Same

Ovarian cancer-associated proteins of the present invention are classified as secreted proteins, transmembrane proteins or intracellular proteins. In one embodiment, the ovarian cancer-associated protein is an intracellular protein. Intracellular proteins are found in the cytoplasm and/or in the nucleus. Intracellular proteins are involved in all aspects of cellular function and replication (including, e.g., signaling pathways); aberrant expression of such proteins often results in unregulated or disregulated cellular processes (see, e.g., Molecular Biology of the Cell (Alberts, ed., 3rd ed., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterising proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs are identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden (see, e.g., Bateman et al., 2000, Nuc. Acids Res. 28: 263-266; Sonnhammer et al., 1997, Proteins 28: 405-420; Bateman et al., 1999, Nuc. Acids Res. 27:260-262; and Sonnhammer et al., 1998, Nuc. Acids Res. 26: 320-322.

In another embodiment, the ovarian cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that are followed by charged amino acids. Therefore, upon analysis of the amino acid sequence of a particular protein, the localization and number of transmembrane domains within the protein are predicted (see, e.g. PSORT web site http://psort.nibb.ac.jp/). Important transmembrane protein receptors include, but are not limited to the insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor,

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which are peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands are tethered to the cell, e.g., via a glycosylphosphatidylinositol (GPI) anchor, or may themselves be transmembrane proteins. Extracellular domains also associate with the extracellular matrix and contribute to the maintenance of the cell structure.

Ovarian cancer-associated proteins that are transmembrane are particularly preferred in the present invention as they are readily accessible targets for immunotherapeutics, as are described herein. In addition, as outlined below, transmembrane proteins are also useful in imaging modalities. Antibodies are used to label such readily accessible proteins in situ. Alternatively, antibodies can also label intracellular proteins, in which case samples are typically permeablized to provide access to intracellular proteins.

It will also be appreciated by those in the art that a transmembrane protein are made soluble by removing transmembrane sequences, e.g., through recombinant methods. Furthermore, transmembrane proteins that have been made soluble are made to be secreted through recombinant means by adding an appropriate signal sequence.

In another embodiment, the ovarian cancer-associated proteins are secreted proteins; the secretion of which are either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Ovarian cancer-associated proteins that are secreted proteins are particularly preferred in the present invention as they serve as good targets for diagnostic markers, e.g., for blood, plasma, serum, or stool tests.

Mammalian Subjects

The present invention provides nucleic acid and protein sequences that are differentially expressed in ovarian cancer, herein termed “ovarian cancer sequences.” As outlined below, ovarian cancer sequences include those that are up-regulated (i.e., expressed at a higher level) in ovarian cancer, as well as those that are down-regulated (i.e., expressed at a lower level). In a preferred embodiment, the ovarian cancer sequences are from humans; however, as will be appreciated by those in the art, ovarian cancer sequences from other organisms are useful in animal models of disease and drug evaluation; thus, other ovarian cancer sequences are provided, from vertebrates, including mammals, including rodents (rats, mice, hamsters, guinea pigs, etc.), primates, farm animals (including sheep, goats, pigs, cows, horses, etc.) and pets, e.g., (dogs, cats, etc.).

Assay Control Samples

It will be apparent from the preceding discussion that many of the diagnostic methods provided by the present invention involve a degree of quantification to determine, on the one hand, the over-expression or reduced-expression of a diagnostic/prognostic marker in tissue that is suspected of comprising a cancer cell. Such quantification can be readily provided by the inclusion of appropriate control samples in the assays described below, derived from healthy or normal individuals. Alternatively, if internal controls are not included in each assay conducted, the control may be derived from an established data set that has been generated from healthy or normal individuals.

In the present context, the term “healthy individual” shall be taken to mean an individual who is known not to suffer from ovarian cancer, such knowledge being derived from clinical data on the individual, including, but not limited to, a different cancer assay to that described herein. As the present invention is particularly useful for the early detection of ovarian cancer, it is preferred that the healthy individual is asymptomatic with respect to the early symptoms associated with ovarian cancer. Although early detection using well-known procedures is difficult, reduced urinary frequency, rectal pressure, and abdominal bloating and swelling, are associated with the disease in its early stages, and, as a consequence, healthy individuals should not have any of these clinical symptoms. Clearly, subjects suffering from later symptoms associated with ovarian cancer, such as, for example, metastases in the omentum, abdominal fluid, lymph nodes, lung, liver, brain, or bone, and subjects suffering from spinal cord compression, elevated calcium level, chronic pain, or pleural effusion, should also be avoided from the “healthy individual” data set.

The term “normal individual” shall be taken to mean an individual having a normal level of expression of a cancer-associate gene or cancer-associated protein in a particular sample derived from said individual. As will be known to those skilled in the art, data obtained from a sufficiently large sample of the population will normalize, allowing the generation of a data set for determining the average level of a particular parameter. Accordingly, the level of expression of a cancer-associate gene or cancer-associated protein can be determined for any population of individuals, and for any sample derived from said individual, for subsequent comparison to levels determined for a sample being assayed. Where such normalized data sets are relied upon, internal controls are preferably included in each assay conducted to control for variation.

In one embodiment, the present invention provides a method for detecting a cancer cell in a subject, said method comprising:
(i) determining the level of mRNA encoding a cancer-associated protein expressed in a test sample from said subject; and
(ii) comparing the level of mRNA determined at (i) to the level of mRNA encoding a cancer-associated protein expressed in a comparable sample from a healthy or normal individual,
   wherein a level of mRNA at (i) that is modified in the test sample relative to the comparable sample from the normal or healthy individual is indicative of the presence of a cancer cell in said subject.

Alternatively, or in addition, the control may comprise a cancer-associated sequence that is known to be expressed at a particular level in an ovarian cancer, eg., CA125, MUC-1 or E-Cadherin, amongast others.

Biological Samples

Preferred biological samples in which the assays of the invention are performed include bodily fluids, ovarian tissue and cells, and those tissues known to comprise cancer cells arising from a metastasis of an ovarian cancer, such as, for example, in carcinomas of the lung, prostate, breast, colon, pancreas, placenta, or omentum, and in cells of brain anaplastic oligodendrogliomas.

Bodily fluids shall be taken to include whole blood, serum, peripheral blood mononuclear cells (PBMC), or buffy coat fraction.

In the present context, the term “cancer cell” includes any biological specimen or sample comprising a cancer cell irrespective of its degree of isolation or purity, such as, for example, tissues, organs, cell lines, bodily fluids, or histology specimens that comprise a cell in the early stages of transformation or having been transformed.

As the present invention is particularly useful for the early detection and prognosis of cancer ofe rthe medium to long term, the definition of “cancer cell” is not to be limited by the stage of a cancer in the subject from which said cancer cell is derived (ie. whether or not the patient is in remission or undergoing disease recurrence or whether or not the cancer is a primary tumor or the consequence of metastases). Nor is the term “cancer cell” to be limited by the stage of the cell cycle of said cancer cell.

Preferably, the sample comprises ovarian tissue, prostate tissue, kidney tissue, uterine tissue, placenta, a cervical specimen, omentum, rectal tissue, brain tissue, bone tissue, lung tissue, lymphatic tissue, urine, semen, blood, abdominal fluid, or serum, or a cell preparation or nucleic acid preparation derived therefrom. More preferably, the sample comprises serum or abdominal fluid, or a tissue selected from the group consisting of: ovary, lymph, lung, liver, brain, placenta, brain, omentum, and prostate. Even more preferably, the sample comprises serum or abdominal fluid, ovary (eg. OSE), or lymph node tissue. The sample can be prepared on a solid matrix for histological analyses, or alternatively, in a suitable solution such as, for example, an extraction buffer or suspension buffer, and the present invention clearly extends to the testing of biological solutions thus prepared.

Polynucleotide Probes and Amplification Primers

Polynucleotide probes are derived from or comprise the nucleic acid sequences whose nucleotide sequences are provided by reference to the public database accession numbers given in Tables 1-3 (referred to herein as the nucleotide sequences shown in Tables 1-3), and sequences homologues thereto as well as variants, derivatives and fragments thereof.

Whilst the probes may comprise double-stranded or single-stranded nucleic acid, single-stranded probes are preferred because they do not require melting prior to use in hybridizations. On the other hand, longer probes are also preferred because they can be used at higher hybridization stringency than shorter probes and may produce lower background hybridization than shorter probes.

So far as shorter probes are concerned, single-stranded, chemically-synthesized oligonucleotide probes are particularly preferred by the present invention. To reduce the noise associated with the use of such probes during hybridization, the nucleotide sequence of the probe is carefully selected to maximize the Tm at which hybridizations can be performed, reduce non-specific hybridization, and to reduce self-hybridization. Such considerations may be particularly important for applications involving high throughput screening using microarray technology. In general, this means that the nucleotide sequence of an oligonucleotide probe is selected such that it is unique to the target RNA or protein-encoding sequence, has a low propensity to form secondary structure, low self-complementary, and is not highly A/T-rich.

The only requirement for the probes is that they cross-hybridize to nucleic acid encoding the target diagnostic protein or the complementary nucleotide sequence thereto and are sufficiently unique in sequence to generate high signal:noise ratios under specified hybridization conditions. As will be known to those skilled in the art, long nucleic acid probes are preferred because they tend to generate higher signal:noise ratios than shorter probes and/or the duplexes formed between longer molecules have higher melting temperatures (i.e. Tm values) than duplexes Involving short probes. Accordingly, full-length DNA or RNA probes are contemplated by the present invention, as are specific probes comprising the sequence of the 3′-untranslated region or complementary thereto.

In a particularly preferred embodiment, the nucleotide sequence of an oligonucleotide probe has no detectable nucleotide sequence identity to a nucleotide sequence in a BLAST search (Altschul et al., J. Mol. Biol. 215, 403-410, 1990) or other database search, other than a sequence selected from the group consisting of: (a) a sequence encoding a polypeptide listed in any one of Tables 1-3; (b) the 5′-untranslated region of a sequence encoding a polypeptide listed in any one of Tables 1-3; (c) a 3′-untranslated region of a sequence encoding a polypeptide listed in any one of Tables 1-3; and (d) an exon region of a sequence encoding a polypeptide listed in any one of Tables 1-3.

Additionally, the self-complementarity of a nucleotide sequence can be determined by aligning the sequence with its reverse complement, wherein detectable regions of identity are indicative of potential self-complementarity. As will be known to those skilled in the art, such sequences may not necessarily form secondary structures during hybridization reaction, and, as a consequence, successfully identify a target nucleotide sequence. It is also known to those skilled in the art that, even where a sequence does form secondary structures during hybridization reactions, reaction conditions can be modified to reduce the adverse consequences of such structure formation. Accordingly, a potential for self-complementarity should not necessarily exclude a particular candidate oligonucleotide from selection. In cases where it is difficult to determine nucleotide sequences having no potential self-complementarity, the uniqueness of the sequence should outweigh a consideration of its potential for secondary structure formation.

Recommended pre-requisites for selecting oligonucleotide probes, particularly with respect to probes suitable for microarray technology, are described in detail by Lockhart et al., “Expression monitoring by hybridization to high-density oligonucleotide arrays”, Nature Biotech. 14, 1675-1680, 1996.

The nucleic acid probe may comprise a nucleotide sequence that is within the coding strand of a gene listed in any one of Tables 1-3. Such “sense” probes are useful for detecting RNA by amplification procedures, such as, for example, polymerase chain reaction (PCR), and more preferably, quantitative PCR or reverse transcription polymerase chain reaction (RT-PCR). Alternatively, “sense” probes may be expressed to produce polypeptides or immunologically active derivatives thereof that are useful for detecting the expressed protein in samples.

The nucleotide sequences referred to in Tables 1-3 and homologues thereof, typically encode polypeptides. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotides may comprise DNA or RNA. They are single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein are modified by any method available in the art. Such modifications are carried out in order to enhance the in vivo activity or life span of the diagnostic/prognostic polynucleotides.

The terms “variant” or “derivative” in relation to the nucleotide sequences of the present invention include any substitution of, variation of, modification of, replacement of, deletion of or addition of one (or more) nucleic acid from or to the sequence provided that the resultant nucleotide sequence codes for a polypeptide having biological activity, preferably having substantially the same activity as the polypeptide sequences presented in the sequence listings.

With respect to sequence homology, preferably there is at least 75%, more preferably at least 85%, more preferably at least 90% homology to a sequence shown in Tables 1-3 herein over a region of at least 20, preferably at least 25 or 30, for instance at least 40, 60, 100, 500, 1000 or more contiguous nucleotides. More preferably there is at least 95%, more preferably at least 98%, homology. In one embodiment, homologues are naturally occurring sequences, such as orthologues, tissue-specific isoforms and allelic variants.

Homology comparisons are conducted by eye, or more usually, with the aid of readily available sequence comparison programs. These commercially available computer programs can calculate % homology between two or more sequences.

Percentage (%) homology are calculated over contiguous sequences, i.e. one sequence is aligned with the other sequence and each nucleotide in one sequence directly compared with the corresponding nucleotide in the other sequence, one base at a time. This is called an “ungapped” alignment. Typically, such ungapped alignments are performed only over a relatively short number of bases (for example less than 50 contiguous nucleotides).

Although this is a very simple and consistent method, it fails to take into consideration that, for example, in an otherwise identical pair of sequences, one insertion or deletion will cause the following nucleotides to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

In determining whether or not two amino acid sequences fall within the stated defined percentage identity limits, those skilled in the art will be aware that it is necessary to conduct a side-by-side comparison of amino acid sequences. In such comparisons or alignments, differences will arise in the positioning of non-identical amino acid residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using the GAP program of the Computer Genetics Group, Inc., University Research Park, Madison, Wis., United States of America (Devereaux et al, Nucl. Acids Res. 12, 387-395, 1984), which utilizes the algorithm of Needleman and Wunsch J. Mol. Biol. 48, 443-453, 1970, or alternatively, the CLUSTAL W algorithm of Thompson et al., Nucl. Acids Res. 22, 4673-4680, 1994, for multiple alignments, to maximize the number of identical/similar amino acids and to minimize the number and/or length of sequence gaps in the alignment.

A suitable computer program for carrying out such an alignment is the GCG Wisconsin Bestfit package (University of Wisconsin, U.S.A.; Devereux et al., 1984, Nucleic Acids Research 12:387). The default scoring matrix has a match value of 10 for each identical nucleotide and −9 for each mismatch. The default gap creation penalty is −50 and the default gap extension penalty is −3 for each nucleotide.

Examples of other software than can perform sequence comparisons include, but are not limited to, the BLAST package (see Ausubel et al., 1999 ibid—Chapter 18), FASTA (Atschul et al, 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Once the software has produced an optimal alignment, it is possible to calculate % homology, preferably % sequence identity. The software typically does this as part of the sequence comparison and generates a numerical result.

A preferred sequence comparison program is the GCG Wisconsin Bestfit program described above.

The present invention also encompasses the use of nucleotide sequences that are capable of hybridizing selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridizing to the nucleotide sequences presented herein, or to their complement, will be generally at least 70%, preferably at least 80 or 90% and more preferably at least 95% or 98% homologous to the corresponding nucleotide sequences referred to in Tables 1-3 over a region of at least 20, preferably at least 25 or 30, for instance at least 40, 60, 100, 500, 1000 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction are measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

For the purposes of defining the level of stringency, a high stringency hybridization is achieved using a hybridization buffer and/or a wash solution comprising the following:
(i) a salt concentration that is equivalent to 0.1×SSC-0.2×SSC buffer or lower salt concentration;
(ii) a detergent concentration equivalent to 0.1% (w/v) SDS or higher; and
(iii) an incubation temperature of 55° C. or higher.

Conditions for specifically hybridizing nucleic acid, and conditions for washing to remove non-specific hybridizing nucleic acid, are well understood by those skilled in the art. For the purposes of further clarification only, reference to the parameters affecting hybridization between nucleic acid molecules is found in Ausubel et al. (Current Protocols in Molecular Biology, Wiley Interscience, ISBN 047150338, 1992), which is herein incorporated by reference.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization are used to identify or detect identical polynucleotide sequences while an intermediate (or low) stringency hybridization are used to identify or detect similar or related polynucleotide sequences.

In a preferred aspect, the present invention covers nucleotide sequences that can hybridize to the nucleotide sequence of the present invention under stringent conditions (e.g. 65° C. and 0.1×SSC {1×SSC=0.15 M NaCl, 0.015 M Na₃Citrate pH 7.0}).

Where the diagnostic/prognostic polynucleotide is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences of the present invention but are useful in perfoming the diagnostic and/or prognostic assays of the invention by virtue of their ability to selectively hybridize to the target gene transcript, or to encode an immunologically cross-reactive protein to the target protein, are obtained in a number of ways, such as, for example by probing DNA libraries made from a range of individuals, for example individuals from different populations. In particular, given that that changes in the expression of diagnostic/prognostic cancer-associated genes correlate with ovarian cancer, characterisation of variant sequences in individuals suffering from ovarian cancer is used to identify variations in the sequences of ovarian-cancer associated genes (and proteins) that are predictive of and/or causative of ovarian cancer.

Accordingly the present invention provides methods of identifying sequence variants that are associated with ovarian cancer which methods comprise determining all or part of the nucleotide sequence of a gene referred to in Tables 1-3, derived from an individual suffering from ovarian cancer and comparing the sequence to that of the corresponding wild-type gene.

In addition, other viral/bacterial, or cellular homologues particularly cellular homologues found in mammalian cells (e.g. rat, mouse, bovine and primate cells), are obtained and such homologues and fragments thereof in general will be capable of selectively hybridizing to the sequences shown in the sequence listing herein. Such sequences are obtained by probing cDNA libraries made from or genomic DNA libraries from other animal species, and probing such libraries with probes comprising all or part of the sequences referred to in Tables 1-3 under conditions of medium to high stringency. Similar considerations apply to obtaining species homologues and allelic variants of the nucleotide sequences referred to in Tables 1-3.

Variants and strain/species homologues may also be obtained using degenerate PCR which will use primers designed to target sequences within the variants and homologues encoding conserved amino acid sequences within the sequences of the present invention. Conserved sequences are predicted, for example, by aligning the amino acid sequences from several variants/homologues. Sequence alignments are performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides are obtained by site-directed mutagenesis of characterised sequences, such as the sequences referred to in Tables 1-3. This are useful where for example silent codon changes are required to sequences to optimise codon preferences for a particular host cell in which the polynucleotide sequences are being expressed. Other sequence changes are desired in order to introduce restriction enzyme recognition sites, or to alter the property or function of the polypeptides encoded by the polynucleotides.

Polynucleotides comprising a diagnostic/prognostic cancer-associated gene are used to produce a primer by standard derivatization means, e.g. a PCR primer, a primer for an alternative amplification reaction, a probe e.g. labelled with a detectable label by conventional means using radioactive or nonradioactive labels, or the polynucleotides are cloned into vectors. Such primers, probes and other fragments will be at least 15, preferably at least 20, for example at least 25, 30 or 40 nucleotides in length. Preferred fragments are less than 5000, 2000, 1000, 500 or 200 nucleotides in length.

Polynucleotides such as a DNA polynucleotides and probes according to the invention are produced by recombinant or synthetic means, including cloning by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers are designed to contain suitable restriction enzyme recognition sites so that the amplified DNA are cloned into a suitable cloning vector

Polynucleotide probes or primers preferably carry a detectable label. Suitable labels include radioisotopes such as ³²P or ³⁵S, enzyme labels, or other protein labels such as biotin. Such labels are added to polynucleotides or primers and are detected using by techniques known in the art.

Polynucleotide probes or primers labeled or unlabeled are also used by a person skilled in the art in nucleic acid-based tests for detecting or sequencing diagnostic/prognostic cancer-associated gene.

Such tests for detecting generally comprise bringing a biological sample containing DNA or RNA into contact with a probe comprising a polynucleotide probe or primer under at least low stringency hybridization conditions and detecting any duplex formed between the probe/primer and nucleic acid in the sample. Such detection are achieved using techniques such as PCR or by immobilising the probe on a solid support, removing nucleic acid in the sample which is not hybridized to the probe, and then detecting nucleic acid which has hybridized to the probe. Alternatively, the sample nucleic acid are immobilised on a solid support, and the amount of probe bound to such a support are detected. Suitable assay methods of this and other formats are found in for example WO89/03891 and WO90/13667.

Tests for sequencing nucleotides include bringing a biological sample containing target DNA or RNA into contact with a probe comprising a polynucleotide probe or primer under at least low stringency hybridization conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing nucleotides in a biological sample are used as part of the methods of the invention for detecting ovarian cancer-associated transcripts and monitoring the efficacy of treatment of patients suffering from ovarian cancer as described in more detail herein.

The probes/primers may conveniently be packaged in the form of a test kit in a suitable container. In such kits the probe are bound to a solid support where the assay format for which the kit is designed requires such binding. The kit may also contain suitable reagents for treating the sample to be probed, hybridizing the probe to nucleic acid in the sample, control reagents, instructions, and the like.

Preferably, a kit of the invention comprises primers/probes suitable for selectively detecting a plurality of sequences, more preferably for selectively detecting a plurality of sequences that are listed in one or more of Tables 1-3 as having a P value of less than 0.05, more preferably a P value of less than 0.01. Similarly, a kit of the invention preferably comprises primers suitable for selectively detecting a plurality of sequences referred to in Table 1 or 2 or 3.

Nucleic Acid-Based Assay Formats

As discussed in detail below, the status of expression of a cancer-associated gene in patient samples may be analyzed by a variety protocols that are well known in the art including in situ hybridization, northern blotting techniques, RT-PCR analysis (such as, for example, performed on laser capture microdissected samples), and microarray technology, such as, for example, using tissue microarrays probed with nucleic acid probes, or nucleic acid microarrays (ie. RNA microarrays or amplified DNA microarrays) microarrays probed with nucleic acid probes. All such assay formats are encompassed by the present invention.

For high throughput screening of large numbers of samples, such as, for example, public health screening of subjects, particularly human subjects, having a higher risk of developing cancer, microarray technology is a preferred assay format.

In accordance with such high throughput formats, techniques for producing immobilised arrays of DNA molecules have been described in the art. Generally, most prior art methods describe how to synthesise single-stranded nucleic acid molecule arrays, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which are used to produced the immobilised DNA arrays. U.S. Pat. No. 5,837,832 also provides references for earlier techniques that may also be used.

Thus DNA are synthesised in situ on the surface of the substrate. However, DNA may also be printed directly onto the substrate using for example robotic devices equipped with either pins or piezo electric devices.

The plurality of polynucleotide sequences are typically immobilised onto or in discrete regions of a solid substrate. The substrate are porous to allow immobilisation within the substrate or substantially non-porous, in which case the library sequences are typically immobilised on the surface of the substrate. The solid substrate are made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes are mounted on a more robust solid surface such as glass. The surfaces may optionally be coated with a layer of metal, such as gold, platinum or other transition metal. A particular example of a suitable solid substrate is the commercially available BIACore™ chip (Pharmacia Biosensors).

Preferably, the solid substrate is generally a material having a rigid or semi-rigid surface. In preferred embodiments, at least one surface of the substrate will be substantially flat, although in some embodiments it are desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 cm⁻².

The solid substrate is conveniently divided up into sections. This are achieved by techniques such as photoetching, or by the application of hydrophobic inks, for example teflon-based inks (Cel-line, USA).

Discrete positions, in which each different member of the array is located may have any convenient shape, e.g., circular, rectangular, elliptical, wedge-shaped, etc.

Attachment of the polynucleotide sequences to the substrate are by covalent or non-covalent means. The plurality of polynucleotide sequences are attached to the substrate via a layer of molecules to which the sequences bind. For example, the sequences are labelled with biotin and the substrate coated with avidin and/or streptavidin. A convenient feature of using biotinylated sequences is that the efficiency of coupling to the solid substrate are determined easily. Since the library sequences may bind only poorly to some solid substrates, it is often necessary to provide a chemical interface between the solid substrate (such as in the case of glass) and the sequences. Examples of suitable chemical interfaces include hexaethylene glycol. Another example is the use of polylysine coated glass, the polylysine then being chemically modified using standard procedures to introduce an affinity ligand. Other methods for attaching molecules to the surfaces of solid substrate by the use of coupling agents are known in the art, see for example WO98/49557.

The complete DNA array is typically read at the same time by charged coupled device (CCD) camera or confocal imaging system. Alternatively, the DNA array are placed for detection in a suitable apparatus that can move in an x-y direction, such as a plate reader. In this way, the change in characteristics for each discrete position are measured automatically by computer controlled movement of the array to place each discrete element in turn in line with the detection means.

The detection means are capable of interrogating each position in the library array optically or electrically. Examples of suitable detection means include CCD cameras or confocal imaging systems.

In a preferred embodiment, the level of expression of the cancer-associated gene in the test sample is determined by hybridizing a probe/primer to RNA in the test sample under at least low stringency hybridization conditions and detecting the hybridization using a detection means.

Similarly, the level of mRNA in the comparable sample from the healthy or normal individual is preferably determined by hybridizing a probe/primer to RNA in said comparable sample under at least low stringency hybridization conditions and detecting the hybridization using a detection means.

For the purposes of defining the level of stringency to be used in these diagnostic assays, a low stringency is defined herein as being a hybridization and/or a wash carried out in 6×SSC buffer, 0.1% (w/v) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample RNA, or the type of hybridization probe used.

In general, the sample or the probe is immobilized on a solid matrix or surface (e.g., nitrocellulose). For high throughput screening, the sample or probe will generally comprise an array of nucleic acids on glass or other solid matrix, such as, for example, as described in WO 96/17958. Techniques for producing high density arrays are described, for example, by Fodor et al., Science 767-773, 1991, and in U.S. Pat. No. 5,143,854. Typical protocols for other assay formats can be found, for example in Current Protocols In Molecular Biology, Unit 2 (Northern Blotting), Unit 4 (Southern Blotting), and Unit 18 (PCR Analysis), Frederick M. Ausubul et al. (ed)., 1995.

The detection means according to this aspect of the invention may be any nucleic acid-based detection means such as, for example, nucleic acid hybridization or amplification reaction (eg. PCR), a nucleic acid sequence-based amplification (NASBA) system, inverse polymerase chain reaction (iPCR), in situ polymerase chain reaction, or reverse transcription polymerase chain reaction (RT-PCR), amongst others.

The probe can be labelled with a reporter molecule capable of producing an identifiable signal (e.g., a radioisotope such as ³²P or ³⁵S, or a fluorescent or biotinylated molecule). According to this embodiment, those skilled in the art will be aware that the detection of said reporter molecule provides for identification of the probe and that, following the hybridization reaction, the detection of the corresponding nucleotide sequences in the sample is facilitated. Additional probes can be used to confirm the assay results obtained using a single probe.

Wherein the detection means is an amplification reaction such as, for example, a polymerase chain reaction or a nucleic acid sequence-based amplification (NASBA) system or a variant thereof, one or more nucleic acid probes molecules of at least about contiguous nucleotides in length is hybridized to mRNA encoding a cancer-associated protein, or alternatively, hybridized to cDNA or cRNA produced from said mRNA, and nucleic acid copies of the template are enzymically-amplified.

Those skilled in the art will be aware that there must be a sufficiently high percentage of nucleotide sequence identity between the probes and the RNA sequences in the sample template molecule for hybridization to occur. As stated previously, the stringency conditions can be selected to promote hybridization.

In one format, PCR provides for the hybridization of non-complementary probes to different strands of a double-stranded nucleic acid template molecule (ie. a DNA/RNA, RNA/RNA or DNA/DNA template), such that the hybridized probes are positioned to facilitate the 5′- to 3′ synthesis of nucleic acid in the intervening region, under the control of a thermostable DNA polymerase enzyme. In accordance with this embodiment, one sense probe and one antisense probe as described herein would be used to amplify DNA from the hybrid RNA/DNA template or cDNA.

In the present context, the cDNA would generally be produced by reverse transcription of mRNA present in the sample being tested (ie. RT-PCR). RT-PCR is particularly useful when it is desirable to determine expression of a cancer-associated gene. It is also known to those skilled in the art to use mRNA/DNA hybrid molecules as a template for such amplification reactions, and, as a consequence, first strand cDNA synthesis is all that is required to be performed prior to the amplification reaction.

Variations of the embodiments described herein are described in detail by McPherson et al., PCR: A Practical Approach. (series eds, D. Rickwood and B. D. Hames), IRL Press Limited, Oxford. pp 1-253, 1991.

The amplification reaction detection means described supra can be further coupled to a classical hybridization reaction detection means to further enhance sensitivity and specificity of the inventive method, such as by hybridizing the amplified DNA with a probe which is different from any of the probes used in the amplification reaction.

Similarly, the hybridization reaction detection means described supra can be further coupled to a second hybridization step employing a probe which is different from the probe used in the first hybridization reaction.

The comparison to be performed in accordance with the present invention may be a visual comparison of the signal generated by the probe, or alternatively, a comparison of data integrated from the signal, such as, for example, data that have been corrected or normalized to allow for variation between samples. Such comparisons can be readily performed by those skilled in the art.

Polypeptides

Cancer-associated polypeptides are encoded by cancer-associated genes. It will be understood that such polypeptides include those polypeptide and fragments thereof that are homologous to the polypeptides encoded by the nucleotide sequences referred to in Tables 1-3, which are obtained from any source, for example related viral/bacterial proteins, cellular homologues and synthetic peptides, as well as variants or derivatives thereof.

Thus, the present invention encompasses the use of variants, homologues or derivatives of the cancer-associated proteins described in the accompanying Tables. In one embodiment, homologues are naturally occurring sequences, such as orthologues, tissue-specific isoforms and allelic variants.

In the context of the present invention, a homologous sequence is taken to include an amino acid sequence which is at least 60, 70, 80 or 90% identical, preferably at least 95 or 98% identical at the amino acid level over at least 20, 40, 60 or 80 amino acids with a sequence encoded by a nucleotide sequence referred to in any one of Tables 1-3. In particular, homology should typically be considered with respect to those regions of the sequence known to be essential for specific biological functions rather than non-essential neighbouring sequences.

Although amino acid homology can also be considered in terms of similarity (i.e. amino acid residues having similar chemical properties/functions), in the context of the present invention it is preferred to express homology in terms of sequence identity.

Homology comparisons are carried out as described above for nucleotide sequences with the appropriate modifications for amino acid sequences. For example when using the GCG Wisconsin Bestfit package (see below) the default gap penalty for amino acid sequences is −12 for a gap and −4 for each extension.

It should also be noted that where computer algorithms are used to align amino acid sequences, although the final % homology are measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence preferably has biological activity, preferably having at least 25 to 50% of the activity as the polypeptides referred to in the sequence listings, more preferably at least substantially the same activity. Particular details of biological activity for each polypeptide are given in Tables 1-3.

Thus, the polypeptides referred to in Tables 1-3 and homologues thereof, are modified for use in the present invention. Typically, modifications are made that maintain the activity of the sequence. Thus, in one embodiment, amino acid substitutions are made, for example from 1, 2 or 3 to 10, 20 or 30 substitutions provided that the modified sequence retains at least about 25 to 50% of, or substantially the same activity. However, in an alternative preferred embodiment, modifications to the amino acid sequences of a cancer-associated protein are made intentionally to reduce the biological activity of the polypeptide. For example truncated polypeptides that remain capable of binding to target molecules but lack functional effector domains are useful as inhibitors of the biological activity of the full length molecule.

In general, preferably less than 20%, 10% or 5% of the amino acid residues of a variant or derivative are altered as compared with the corresponding region of the polypeptides referred to in Tables 1-3.

Amino acid substitutions may include the use of non-naturally occurring analogues, for example to Increase blood plasma half-life of a therapeutically administered polypeptide (see below for further details on the production of peptide derivatives for use in therapy).

Conservative substitutions are made, for example according to the Table below. Amino acids In the same block in the second column and preferably In the same line in the third column are substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Cancer-associated proteins also include fragments of the above mentioned full length polypeptides and variants thereof, including fragments of the sequences referred to in Tables 1-3 and homologues thereof. Preferred fragments include those which include an epitope. Suitable fragments will be at least about 6 or 8, e.g. at least 10, 12, 15 or 20 amino acids in length. They may also be less than 200, 100 or 50 amino acids in length. Polypeptide fragments may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered.

Cancer-associated proteins are preferably in a substantially isolated form. It will be understood that the protein are mixed with carriers or diluents which will not interfere with the intended purpose of the protein and still be regarded as substantially isolated. A cancer-associated protein of the invention may also be in a substantially purified form, in which case it will generally comprise the protein in a preparation in which more than 90%, e.g. 95%, 98% or 99% pure as determined by SDS/PAGE or other art-recognized means for assessing protein purity.

Protein Production

For producing full-length polypeptides or immunologically active derivatives thereof by recombinant means, a protein-encoding region comprising at least about 15 contiguous nucleotides of the protein-encoding region of a nucleic acid referred to in any one of Tables 1-3 is placed in operable connection with a promoter or other regulatory sequence capable of regulating expression in a cell-free system or cellular system.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. Furthermore, the regulatory elements comprising a promoter are usually positioned within 2 kb of the start site of transcription of the gene. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning. A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, Calif.), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 293T cells) include, but are not limited to, the pcDNA Vector suite supplied by Invitrogen, in particular pcDNA 3.1 myc-His-tag comprising the CMV promoter and encoding a C-terminal 6xHis and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of a protein in 293T cells, wherein the expressed peptide or protein can be purified free of conspecific proteins, using standard affinity techniques that employ a Nickel column to bind the protein via the His tag.

A wide range of additional host/vector systems suitable for expressing polypeptides or immunological derivatives thereof are available publicly, and described, for example, in Sambrook et al (In: Molecular cloning. A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

For producing mutants, nucleotide insertion derivatives of the protein-encoding region are produced by making 5′ and 3′ terminal fusions, or by making intra-sequence insertions of single or multiple nucleotides or nucleotide analogues. Insertion nucleotide sequence variants are produced by introducing one or more nucleotides or nucleotide analogues into a predetermined site in the nucleotide sequence of said sequence, although random insertion is also possible with suitable screening of the resulting product being performed. Deletion variants are produced by removing one or more nucleotides from the nucleotide sequence. Substitutional nucleotide variants are produced by substituting at least one nucleotide in the sequence with a different nucleotide or a nucleotide analogue in its place, with the immunologically active derivative encoded therefor having an identical amino acid sequence, or only a limited number of amino acid modifications that do not alter its antigenicity compared to the base peptide or its ability to bind antibodies prepared against the base peptide. Such mutant derivatives will preferably have at least 80% identity With the base amino acid sequence from which they are derived.

Preferred immunologically active derivatives of a full-length polypeptide encoded by a gene referred to in any one of Tables 1-3 will comprise at least about 5-10 contiguous amino acids of the full-length amino acid sequence, more preferably at least about 10-20 contiguous amino acids in length, and even more preferably 20-30 contiguous amino acids in length.

For the purposes of producing derivatives using standard peptide synthesis techniques, such as, for example, Fmoc chemistry, a length not exceeding about 30-50 amino acids in length is preferred, as longer peptides are difficult to produce at high efficiency. Longer peptide fragments are readily achieved using recombinant DNA techniques wherein the peptide is expressed in a cell-free or cellular expression system comprising nucleic acid encoding the desired peptide fragment.

It will be apparent to the skilled artisan that any sufficiently antigenic region of at least about 5-10 amino acid residues can be used to prepare antibodies that bind generally to the polypeptides listed in Tables 1-3.

An expressed protein or synthetic peptide is preferably produced as a recombinant fusion protein, such as for example, to aid in extraction and purification. To produce a fusion polypeptide, the open reading frames are covalently linked in the same reading frame, such as, for example, using standard cloning procedures as described by Ausubel et al. (Current Protocols in Molecular Biology, Wiley Interscience, ISBN 047150338, 1992), and expressed under control of a promoter. Examples of fusion protein partners include glutathione-5-transferase (GST), FLAG, hexahistidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the immune function of the target protein.

In a particularly preferred embodiment, polypeptides are produced substantially free of conspecific proteins. Such purity can be assessed by standard procedures, such as, for example, SDS/polyacrylamide gel electrophoresis, 2-dimensional gene electrophoresis, chromatography, amino acid composition analysis, or amino acid sequence analysis.

To produce isolated polypeptides or fragments, eg., for antibody production, standard protein purification techniques may be employed. For example, gel filtration, ion exchange chromatography, reverse phase chromatography, or affinity chromatography, or a combination of any one or more said procedures, may be used. High pressure and low pressure procedures can also be employed, such as, for example, FPLC, or HPLC. To isolate the full-length proteins or peptide fragments comprising more than about 50-100 amino acids in length, it is particularly preferred to express the polypeptide in a suitable cellular expression system in combination with a suitable affinity tag, such as a 6xHis tag, and to purify the polypeptide using an affinity step that bonds it via the tag (supra). Optionally, the tag may then be cleaved from the expressed polypeptide.

Alternatively, for short immunologically active derivatives of a full-length polypeptide, preferably those peptide fragments comprising less than about 50 amino acids in length, chemical synthesis techniques are conveniently used. As will be known to those skilled in the art, such techniques may also produce contaminating peptides that are shorter than the desired peptide, in which case the desired peptide is conveniently purified using reverse phase and/or ion exchange chromatography procedures at high pressure (ie. HPLC or FPLC).

Antibodies

The invention also provides monoclonal or polyclonal antibodies that bind specifically to polypeptides of the invention or fragments thereof. Thus, the present invention further provides a process for the production of monoclonal or polyclonal antibodies to polypeptides of the invention.

The phrase “binds specifically” to a polypeptide means that the binding of the antibody to the protein or peptide is determinative of the presence of the protein, in a heterogeneous population of proteins and other biologics. Thus, under designated immunoassay conditions, the specified antibodies bind to a particular protein at least two times the background and more typically more than 10 to 100 times background. Typically, antibodies of the invention bind to a protein of interest with a Kd of at least about 0.1 mM, more usually at least about 1 μM, preferably at least about 0.1 μM, and most preferably at least, 0.01 μM.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

In accordance with this aspect of the invention, the antibodies may be produced for the purposes of immunizing the subject, in which case high titer or neutralizing antibodies that bind to a B cell epitope will be especially preferred. Suitable subjects for immunization will, of course, depend upon the immunizing antigen or antigenic B cell epitope. It is contemplated that the present invention will be broadly applicable to the immunization of a wide range of animals, such as, for example, farm animals (e.g. horses, cattle, sheep, pigs, goats, chickens, ducks, turkeys, and the like), laboratory animals (e.g. rats, mice, guinea pigs, rabbits), domestic animals (cats, dogs, birds and the like), feral or wild exotic animals (e.g. possums, cats, pigs, buffalo, wild dogs and the like) and humans.

Alternatively, the antibodies may be for commercial or diagnostic purposes, in which case the subject to whom the diagnostic/prognostic protein or immunogenic fragment or epitope thereof is administered will most likely be a laboratory or farm animal. A wide range of animal species are used for the production of antisera. Typically the animal used for production of antisera is a rabbit, a mouse, rat, hamster, guinea pig, goat, sheep, pig, dog, horse, or chicken. Because of the relatively large blood volume of rabbits, a rabbit is a preferred choice for production of polyclonal antibodies. However, as will be known to those skilled tin the art, larger amounts of immunogen are required to obtain high antibodies from large animals as opposed to smaller animals such as mice. In such cases, it will be desirable to isolate the antibody from the immunized animal.

Preferably, the antibody is a high titer antibody. By “high titer” means a sufficiently high titer to be suitable for use in diagnostic or therapeutic applications. As will be known in the art, there is some variation in what might be considered “high titer”. For most applications a titer of at least about 10³-10⁴ is preferred. More preferably, the antibody titer will be in the range from about 1 to about 10⁵, even more preferably in the range from about 10⁵ to about 10⁶.

More preferably, in the case of B cell epitopes from pathogens, viruses or bacteria, the antibody is a neutralizing antibody (i.e. it is capable of neutralizing the infectivity of the organism fro which the B cell epitope is derived).

To generate antibodies, the diagnostic/prognostic protein or immunogenic fragment or epitope thereof, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers. Means for preparing and characterizing antibodies are well known in the art. (See, e.g., ANTIBODIES: A LABORATORY MANUAL, Cold Spring Harbor Laboratory, 1988, incorporated herein by reference).

The efficacy of the diagnostic/prognostic protein or immunogenic fragment or epitope thereof in producing an antibody is established by injecting an animal, for example, a mouse, rat, rabbit, guinea pig, dog, horse, cow, goat or pig, with a formulation comprising the diagnostic/prognostic protein or immunogenic fragment or epitope thereof, and then monitoring the immune response to the B cell epitope, as described in the Examples. Both primary and secondary immune responses are monitored. The antibody titer is determined using any conventional immunoassay, such as, for example, ELISA, or radio immunoassay.

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

For the production of monoclonal antibodies (Mabs) any one of a number of well-known techniques may be used, such as, for example, the procedure exemplified in U.S. Pat. No. 4,196,265, incorporated herein by reference.

For example, a suitable animal will be immunized with an effective amount of the diagnostic/prognostic protein or immunogenic fragment or epitope thereof under conditions sufficient to stimulate antibody producing cells. Rodents such as mice and rats are preferred animals, however, the use of rabbit, sheep, or frog cells is also possible. The use of rats may provide certain advantages, but mice are preferred, with the BALB/c mouse being most preferred as the most routinely used animal and one that generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer removed. Spleen lymphocytes are obtained by homogenizing the spleen with a syringe. Typically, a spleen from an immunized mouse contains approximately 5×10⁷ to 2×10⁸ lymphocytes.

The B cells from the immunized animal are then fused with cells of an immortal myeloma cell, generally derived from the same species as the animal that was immunized with the diagnostic/prognostic protein or immunogenic fragment or epitope thereof. Myeloma cell lines suited for use in hybridoma-producing fusion procedures preferably are non-antibody-producing, have high fusion efficiency and enzyme deficiencies that render them incapable of growing in certain selective media which support the growth of only the desired fused cells, or hybridomas. Any one of a number of myeloma cells may be used and these are known to those of skill in the art (e.g. murine P3-X63/Ag8, X63-Ag8.653, NS1/1.Ag 41, Sp210-Ag14, FO, NSO/U, MPC11, MPC11-X45-GTG 1.7 and S194/5XX0; or rat R210.RCY3, Y3-Ag 1.2.3, IR983F and 4B210; and U-266, GM1500-GRG2, LICR-LON-HMy2 and UC729-6). A preferred murine myeloma cell is the NS-1 myeloma cell line (also termed P3-NS-1-Ag4-1), which is readily available from the NIGMS Human Genetic Mutant Cell Repository under Accession No. GM3573. Alternatively, a murine myeloma SP2/0 non-producer cell line that is 8-azaguanine-resistant is used.

To generate hybrids of antibody-producing spleen or lymph node cells and myeloma cells, somatic cells are mixed with myeloma cells in a proportion between about 20:1 to about 1:1, respectively, in the presence of an agent or agents (chemical or electrical) that promote the fusion of cell membranes. Fusion methods using Sendai virus have been described by Kohler and Milstein, Nature 256, 495-497, 1975; and Kohler and Milstein, Eur. J. Immunol. 6, 511-519, 1976. Methods using polyethylene glycol (PEG), such as 37% (v/v) PEG, are described in detail by Gefter et al., Somatic Cell Genet 3, 231-236, 1977. The use of electrically induced fusion methods is also appropriate.

Hybrids are amplified by culture in a selective medium comprising an agent that blocks the de novo synthesis of nucleotides in the tissue culture media. Exemplary and preferred agents are aminopterin, methotrexate and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT, because only those hybridomas capable of operating nucleotide salvage pathways are able to survive in HAT medium, whereas myeloma cells are defective in key enzymes of the salvage pathway, (e.g., hypoxanthine phosphoribosyl transferase or HPRT), and they cannot survive. B cells can operate this salvage pathway, but they have a limited life span in culture and generally die within about two weeks. Accordingly, the only cells that can survive in the selective media are those hybrids formed from myeloma and B cells.

The amplified hybridomas are subjected to a functional selection for antibody specificity and/or titer, such as, for example, by immunoassay (e.g. radioimmunoassay, enzyme immunoassay, cytotoxicity assay, plaque assay, dot immunobinding assay, and the like).

The selected hybridomas are serially diluted and cloned into individual antibody-producing cell lines, which clones can then be propagated indefinitely to provide MAbs. The cell lines may be exploited for MAb production in two basic ways. A sample of the hybridoma is injected, usually in the peritoneal cavity, into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they are readily obtained in high concentrations. MAbs produced by either means may be further purified, if desired, using filtration, centrifugation and various chromatographic methods such as HPLC or affinity chromatography.

Monoclonal antibodies of the present invention also include anti-idiotypic antibodies produced by methods well-known in the art. Monoclonal antibodies according to the present invention also may be monoclonal heteroconjugates, (i.e., hybrids of two or more antibody molecules). In another embodiment, monoclonal antibodies according to the invention are chimeric monoclonal antibodies. In one approach, the chimeric monoclonal antibody is engineered by cloning recombinant DNA containing the promoter, leader, and variable-region sequences from a mouse anti-PSA producing cell and the constant-region exons from a human antibody gene. The antibody encoded by such a recombinant gene is a mouse-human chimera. Its antibody specificity is determined by the variable region derived from mouse sequences. Its isotype, which is determined by the constant region, is derived from human DNA.

In another embodiment, the monoclonal antibody according to the present invention is a “humanized” monoclonal antibody, produced by any one of a number of techniques well-known in the art. That is, mouse complementary determining regions (“CDRs”) are transferred from heavy and light V-chains of the mouse Ig into a human V-domain, followed by the replacement of some human residues in the framework regions of their murine counterparts. “Humanized” monoclonal antibodies in accordance with this invention are especially suitable for use in vivo in diagnostic and therapeutic methods.

As stated above, the monoclonal antibodies and fragments thereof according to this invention are multiplied according to in vitro and in vivo methods well-known in the art. Multiplication in vitro is carried out in suitable culture media such as Dulbecco's modified Eagle medium or RPMI 1640 medium, optionally replenished by a mammalian serum such as fetal calf serum or trace elements and growth-sustaining supplements, e.g., feeder cells, such as normal mouse peritoneal exudate cells, spleen cells, bone marrow macrophages or the like. In vitro production provides relatively pure antibody preparations and allows scale-up to give large amounts of the desired antibodies. Techniques for large scale hybridoma cultivation under tissue culture conditions are known in the art and include homogenous suspension culture, (e.g., in an airlift reactor or in a continuous stirrer reactor or immobilized or entrapped cell culture).

Large amounts of the monoclonal antibody of the present invention also may be obtained by multiplying hybridoma cells in vivo. Cell clones are injected into mammals which are histocompatible with the parent cells, (e.g., syngeneic mice, to cause growth of antibody-producing tumors. Optionally, the animals are primed with a hydrocarbon, especially oils such as Pristane (tetramethylpentadecane) prior to injection.

In accordance with the present invention, fragments of the monoclonal antibody of the invention are obtained from monoclonal antibodies produced as described above, by methods which include digestion with enzymes such as pepsin or papain and/or cleavage of disulfide bonds by chemical reduction. Alternatively, monoclonal antibody fragments encompassed by the present invention are synthesized using an automated peptide synthesizer, or they may be produced manually using techniques well known in the art.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents, or by reaction with an Isothiocyanate. Conjugates with metal chelates are similarly produced. Other moieties to which antibodies may be conjugated include radionuclides such as, for example, ³H, ¹²⁵I, ³²P, ³⁵S, ¹⁴C, ⁵¹Cr, ³⁶Cl, ⁵⁷Co, ⁵⁸Co, ⁵⁹Fe, ⁷⁵Se, and ¹⁵²Eu.

Radioactively labeled monoclonal antibodies of the present invention are produced according to well-known methods in the art. For instance, monoclonal antibodies are iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium⁹⁹ by ligand exchange process, for example, by reducing pertechnetate with stannous solution, chelating the reduced technetium onto a Sephadex column and applying the antibody to this column or by direct labeling techniques, (e.g., by incubating pertechnate, a reducing agent such as SNCl₂, a buffer solution such as sodium-potassium phthalate solution, and the antibody).

Any immunoassay may be used to monitor antibody production by the diagnostic/prognostic protein or immunogenic fragment or epitope thereof. Immunoassays, in their most, simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

Most preferably, the assay will be capable of generating quantitative results.

For example, antibodies are tested in simple competition assays. A known antibody preparation that binds to the B cell epitope and the test antibody are incubated with an antigen composition comprising the B cell epitope, preferably in the context of the native antigen. “Antigen composition” as used herein means any composition that contains some version of the B cell epitope in an accessible form. Antigen-coated wells of an ELISA plate are particularly preferred. In one embodiment, one would pre-mix the known antibodies with varying amounts of the test antibodies (e.g., 1:1, 1:10 and 1:100) for a period of time prior to applying to the antigen composition. If one of the known antibodies is labeled, direct detection of the label bound to the antigen is possible; comparison to an unmixed sample assay will determine competition by the test antibody and, hence, cross-reactivity.

Alternatively, using secondary antibodies specific for either the known or test antibody, one will be able to determine competition.

An antibody that binds to the antigen composition will be able to effectively compete for binding of the known antibody and thus will significantly reduce binding of the latter. The reactivity of the known antibodies in the absence of any test antibody is the control. A significant reduction in reactivity in the presence of a test antibody is indicative of a test antibody that binds to the B cell epitope (i.e., it cross-reacts with the known antibody).

In one exemplary ELISA, the antibodies against the diagnostic/prognostic protein or immunogenic fragment or B cell epitope are immobilized onto a selected surface exhibiting protein affinity, such as a well in a polystyrene microtiter plate. Then, a composition containing a peptide comprising the B cell epitope is added to the wells. After binding and washing to remove non-specifically bound immune complexes, the bound epitope may be detected. Detection is generally achieved by the addition of a second antibody that is known to bind to the B cell epitope and is linked to a detectable label. This type of ELISA is a simple “sandwich ELISA”. Detection may also be achieved by the addition of said second antibody, followed by the addition of a third antibody that has binding affinity for the second antibody, with the third antibody being linked to a detectable label.

Antibodies of the invention may be bound to a solid support and/or packaged into kits in a suitable container along with suitable reagents, controls, instructions and the like.

Immunoassay Formats

In one embodiment, a cancer-associated protein or an immunogenic fragment or epitope thereof is detected in a patient sample, wherein the level of the protein or immunogenic fragment or epitope in the sample is indicative of ovarian cancer or disease recurrence or an indicator of poor survival. Preferably, the method comprises contacting a biological sample derived from the subject with an antibody capable of binding to a cancer-associated protein or an immunogenic fragment or epitope thereof, and detecting the formation of an antigen-antibody complex.

In another embodiment, an antibody against a cancer-associated protein or epitope thereof is detected in a patient sample, wherein the level of the antibody in the sample is indicative of ovarian cancer or disease recurrence or an indicator of poor survival.

Preferably, the method comprises contacting a biological sample derived from the subject with a cancer-associated protein or an antigenic fragment eg., a B cell epitope or other immunogenic fragment thereof, and detecting the formation of an antigen-antibody complex.

The diagnostic assays of the invention are useful for determining the progression of ovarian cancer or a metastasis thereof in a subject. In accordance with these prognostic applications of the invention, the level of a cancer-associated protein or an immunogenic fragment or epitope thereof in a biological sample is correlated with the disease state eg., as determined by clinical symptoms or biochemical tests (eg., CA125 levels).

Accordingly, a further embodiment of the invention provides a method for detecting a cancer cell in a subject, said method comprising:
(i) determining the level of a cancer-associate protein in a test sample from said subject; and
(ii) comparing the level determined at (i) to the level of said cancer-associated protein in a comparable sample from a healthy or normal individual,
   wherein a level of said cancer-associate protein at (i) that is modified in the test sample relative to the comparable sample from the normal or healthy individual is indicative of the presence of a cancer cell in said subject.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte.

Preferred detection systems contemplated herein include any known assay for detecting proteins or antibodies in a biological sample isolated from a human subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are particularly contemplated.

Immunoassay formats are particularly preferred, eg., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

Preferably, the assay is a semi-quantitative assay or quantitative assay.

Standard solid phase ELISA formats are particularly useful in determining the concentration of a protein or antibody from a variety of patient samples.

In one form such as an assay involves immobilising a biological sample comprising antibodies against the cancer-associated protein or epitope, or alternatively an ovarian cancer-associated protein or an immunogenic fragment thereof, onto a solid matrix, such as, for example a polystyrene or polycarbonate microwell or dipstick, a membrane, or a glass support (e.g. a glass slide).

In the case of an antigen-based assay, an antibody that specifically binds an ovarian cancer-associated protein is brought into direct contact with the immobilised biological sample, and forms a direct bond with any of its target protein present in said sample. For an antibody-based assay, an immobilized ovarian cancer-associated protein or an immunogenic fragment or epitope thereof is contacted with the sample. The added antibody or protein in solution is generally labelled with a detectable reporter molecule, such as for example, a fluorescent label (e.g. FITC or Texas Red) or an enzyme (e.g. horseradish peroxidase (HRP)), alkaline phosphatase (AP) or β-galactosidase.

Alternatively, or in addition, a second labelled antibody can be used that binds to the first antibody or to the isolated/recombinant antigen. Following washing to remove any unbound antibody or antigen, as appropriate, the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds an ovarian cancer-associated protein on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein can then be detected using a labelled antibody. For example if the protein is captured from a human sample, an anti-human antibody is used to detect the captured protein. Alternatively, a third labelled antibody can be used that binds the second (detecting) antibody.

It will be apparent to the skilled person that the assay formats described herein are amenable to high throughput formats, such as, for example automation of screening processes, or a microarray format as described in Mendoza et al, Biotechniques 27(4): 778-788, 1999. Furthermore, variations of the above described assay will be apparent to those skilled in the art, such as, for example, a competitive ELISA.

Alternatively, the presence of antibodies against the cancer-associate protein, or alternatively an oarian cancer-associated protein or an immunogenic fragment thereof, is detected using a radioimmunoassay (RIA). The basic principle of the assay is the use of a radiolabelled antibody or antigen to detect antibody antigen interactions. For example, an antibody that specifically binds to an ovarian cancer-associated protein can be bound to a solid support and a biological sample brought into direct contact with said antibody. To detect the bound antigen, an isolated and/or recombinant form of the antigen is radiolabelled is brought into contact with the same antibody. Following washing the amount of bound radioactivity is detected. As any antigen in the biological sample inhibits binding of the radiolabelled antigen the amount of radioactivity detected is inversely proportional to the amount of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

Western blotting is also useful for detecting an ovarian cancer-associated protein or an immunogenic fragment thereof. In such an assay protein from a biological sample is separated using sodium dodecyl sulphate (SDS) polyacrylamide gel electrophoresis (SDS-PAGE) using techniques well known in the art and described in, for example, Scopes (In: Protein Purification: Principles and Practice, Third Edition, Springer Verlag, 1994). Separated proteins are then transferred to a solid support, such as, for example, a membrane or more specifically PVDF membrane, using methods well known in the art, for example, electrotransfer. This membrane may then be blocked and probed with a labelled antibody or ligand that specifically binds an ovarian cancer-associated protein. Alternatively, a labelled secondary, or even tertiary, antibody or ligand can be used to detect the binding of a specific primary antibody.

High-throughput methods for detecting the presence or absence of antibodies, or alternatively ovarian cancer-associated protein or an immunogenic fragment thereof are particularly preferred.

In one embodiment, MALDI-TOF is used for the rapid identification of a protein. Accordingly, there is no need to detect the proteins of interest using an antibody or ligand that specifically binds to the protein of interest. Rather, proteins from a biological sample are separated using gel electrophoresis using methods well known in the art and those proteins at approximately the correct molecular weight and/or isoelectric point are analysed using MALDI-TOF to determine the presence or absence of a protein of interest.

Alternatively, MALDI or ESI or a combination of approaches is used to determine the concentration of a particular protein in a biological sample, such as, for example sputum. Such proteins are preferably well characterised previously with regard to parameters such as molecular weight and isoelectric point.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a patient (for example sputum that has been solubilised using the methods described herein) contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several epitopes in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. In order to bind a protein to a solid support it is often necessary to treat the solid support so as to create chemically reactive groups on the surface, such as, for example, with an aldehyde-containing silane reagent.

Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip is preferably generated such that several proteins, ligands or antibodies are arrayed on said chip. This format permits the simultaneous screening for the presence of several proteins in a sample.

Alternatively, a protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a sample to be analysed using a protein chip is attached to a reporter molecule, such as, for example, a fluorescent molecule, a radioactive molecule, an enzyme, or an antibody that is detectable using methods well known in the art. Accordingly, by contacting a protein chip with a labelled sample and subsequent washing to remove any unbound proteins the presence of a bound protein is detected using methods well known in the art, such as, for example using a DNA microarray reader.

Alternatively, biomolecular interaction analysis-mass spectrometry (BIA-MS) is used to rapidly detect and characterise a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in U.S. Patent Application 20020139751.

As will be apparent to the skilled artisan, protein chips are particularly amenable to multiplexing of detection reagents. Accordingly, several antibodies or ligands each able to specifically bind a different peptide or protein may be bound to different regions of said protein chip. Analysis of a biological sample using said chip then permits the detecting of multiple proteins of interest, or multiple B cell epitopes of the ovarian cancer-associated protein. Multiplexing of diagnostic and prognostic markers is particularly contemplated in the present invention.

In a further embodiment, the samples are analysed using ICAT, essentially as described in US Patent Application No. 20020076739. This system relies upon the labelling of a protein sample from one source (i.e. a healthy individual) with a reagent and the labelling of a protein sample from another source (i.e. a tuberculosis patient) with a second reagent that is chemically identical to the first reagent, but differs in mass due to isotope composition. It is preferable that the first and second reagents also comprise a biotin molecule. Equal concentrations of the two samples are then mixed, and peptides recovered by avidin affinity chromatography. Samples are then analysed using mass spectrometry. Any difference in peak heights between the heavy and light peptide ions directly correlates with a difference in protein abundance in a biological sample. The identity of such proteins may then be determined using a method well known in the art, such as, for example MALDI-TOF, or ESI.

As will be apparent to those skilled in the art a diagnostic or prognostic assay described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial biological sample, or two or more biological samples isolated at the same point in time.

Accordingly, a multiplexed assay may comprise an assay that detects several antibodies and/or epitopes in the same reaction and simultaneously, or alternatively, it may detect other one or more antigens/antibodies in addition to one or more antibodies and/or epitopes. As will be apparent to the skilled artisan, if such an assay is antibody or ligand based, both of these antibodies must function under the same conditions.

Diagnostic Assay Kits

A further aspect of the present invention provides a kit for detecting M. tuberculosis infection in a biological sample. In one embodiment, the kit comprises:
(i) one or more isolated antibodies that bind to an ovarian cancer-associated protein or an immunogenic fragment or epitope thereof; and
(ii) means for detecting the formation of an antigen-antibody complex.

In an alternative embodiment, the kit comprises:
(i) an isolated or recombinant ovarian cancer-associated protein or an immunogenic fragment or epitope thereof; and
(ii) means for detecting the formation of an antigen-antibody complex.

Optionally, the kit further comprises means for the detection of the binding of an antibody, fragment thereof or a ligand to an ovarian cancer-associated protein. Such means include a reporter molecule such as, for example, an enzyme (such as horseradish peroxidase or alkaline phosphatase), a substrate, a cofactor, an inhibitor, a dye, a radionucleotide, a luminescent group, a fluorescent group, biotin or a colloidal particle, such as colloidal gold or selenium. Preferably such a reporter molecule is directly linked to the antibody or ligand.

In yet another embodiment, a kit may additionally comprise a reference sample. Such a reference sample.

In another embodiment, a reference sample comprises a peptide that is detected by an antibody or a ligand. Preferably, the peptide is of known concentration. Such a peptide is of particular use as a standard. Accordingly various known concentrations of such a peptide may be detected using a prognostic or diagnostic assay described herein.

In yet another embodiment, a kit comprises means for protein isolation (Scopes (In: Protein Purification: Principles and Practice, Third Edition, Springer Verlag, 1994).

Bioinformatics

The ability to identify genes that are over or under expressed in ovarian cancer can additionally provide high-resolution, high-sensitivity datasets which are used in the areas of diagnostics, therapeutics, drug development, pharmacogenetics, protein structure, biosensor development, and other related areas. For example, the expression profiles are used in diagnostic or prognostic evaluation of patients with ovarian cancer. Or as another example, subcellular toxicological information are generated to better direct drug structure and activity correlation (see Anderson, Pharmaceutical Proteomics: Targets, Mechanism, and Function, paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database are in substantially any form in which data are maintained and transmitted, but is preferably an electronic database. The electronic database of the invention are maintained on any electronic device allowing for the storage of and access to the database, such as a personal computer, but is preferably distributed on a wide area network, such as the World Wide Web.

The focus of the present section on databases that include peptide sequence data is for clarity of illustration only. It, will be apparent to those of skill in the art that similar databases are assembled for any assay data acquired using an assay of the invention.

The compositions and methods for identifying and/or quantitating the relative and/or absolute abundance of a variety of molecular and macromolecular species from a biological sample undergoing ovarian cancer, i.e., the identification of ovarian cancer-associated sequences described herein, provide an abundance of information, which are correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which are viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioiraformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods, and Protocols (Misener & Krawetz, eds, 2000); Bioinformatics: Sequence, Structure, and Databanks: A Practical Approach (Higgins & Taylor, eds., 2000); Brown, Bioinfor7natics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for prostate cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which are on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerised comparison between a peptide or nucleic acid sequence assay record stored in or retrieved from a computer storage device or database and at least one other sequence. The comparison can include a sequence analysis or comparison algorithm or computer program embodiment thereof (e.g., BLAST, FASTA, TFASTA, GAP, BESTFIT see above) and/or the comparison are of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program are transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor are a conventional computer (e.g., Intel Pentium, PowerPC, Alpha, PA-8000, SPARC, MIPS 4400, MIPS 10000, VAX, etc.); a program are a commercial or public domain molecular biology software package (e.g., UWGCG Sequence Analysis Software, Darwin); a data file are an optical or magnetic disk, a data server, a memory device (e.g., DRAM, SRAM, SGRAM, SDRAM, EPROM, bubble memory, flash memory, etc.); an I/O device are a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which are stored in the computer; (3) a comparison target, such as' a query target; and (4) a program for alignment and comparison, typically with rank-ordering of comparison results on the basis of computed similarity values.

Transgenic Animals Expressing Ovarian Cancer-Associated Proteins and “Knock-Out” Animals

The present invention also contemplates transgenic animals which are transgenic by virtue of comprising a polynucleotide of the invention, i.e. animals transformed with a cancer-associated gene of the invention. Suitable animals are generally from the phylum chordata. Chordates includes vertebrate groups such as mammals, birds, reptiles and amphibians. Particular examples of mammals include non-human primates, cats, dogs, ungulates such as cows, goats, pigs, sheep and horses and rodents such as mice, rats, gerbils and hamsters. Transgenic animals within the meaning of the present invention are non-human animals and the production of transgenic humans is specifically excluded.

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Advances in technologies for embryo micromanipulation now permit introduction of heterologous DNA into, for example, fertilized mammalian ova. For instance, totipotent or pluripotent stem cells are transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals. Those techniques as well known. See reviews of standard laboratory procedures for microinjection of heterologous DNAs into mammalian fertilized ova, including Hogan et al., Manipulating the Mouse Embryo, (Cold Spring Harbor Press 1986); Krimpenfort et al., Bio/Technology 9:844 (1991); Palmiter et al., Cell, 41: 343 (1985); Kraemer et al., Genetic manipulation of the Mammalian Embryo, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated herein by reference

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology as described in Schnieke, A. E. et al., 1997, Science, 278: 2130 and Cibelli, J. B. et al., 1998, Science, 280: 1256. Using this method, fibroblasts from donor animals are stably transfected with a plasmid incorporating the coding sequences for a binding domain or binding partner of interest under the control of regulatory. Stable transfectants are then fused to enucleated oocytes, cultured and transferred into female recipients.

Analysis of animals which may contain transgenic sequences would typically be performed by either PCR or Southern blot analysis following standard methods.

By way of a specific example for the construction of transgenic mammals, such as cows, nucleotide constructs comprising a sequence encoding a binding domain fused to GFP are microinjected using, for example, the technique described in U.S. Pat. No. 4,873,191, into oocytes which are obtained from ovaries freshly removed from the mammal. The oocytes are aspirated from the follicles and allowed to settle before fertilization with thawed frozen sperm capacitated with heparin and prefractionated by Percoll gradient to isolate the motile fraction.

The fertilized oocytes are centrifuged, for example, for eight minutes at 15,000 g to visualize the pronuclei for injection and then cultured from the zygote to morula or blastocyst stage in oviduct tissue-conditioned medium. This medium is prepared by using luminal tissues scraped from oviducts and diluted In culture medium. The zygotes must be placed in the culture medium Within two hours following microinjection.

Oestrous is then synchronized in the intended recipient mammals, such as cattle, by administering coprostanol. Oestrous is produced within two days and the embryos are transferred to the recipients 5-7 days after estrous. Successful transfer are evaluated in the offspring by Southern blot.

Alternatively, the desired constructs are introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny are obtained using conventional cross-breeding. This technique is described, for example, in WO91/10741.

In another embodiment, transgenic animals of the present invention are transgenic “knock-out” animals where a specific gene corresponding to a polynucleotide referred to in Tables 1-3 has been rendered non-functional by homologous recombination. The generation of “knock-out” animals is similar to the production of other transgenic animals except that the polynucleotide constructs are designed to integrate into the endogenous genes and disrupt the function of the endogenous sequences. The generation of “knock-out” animals is known in the art, including the design of suitable constructs that will recombine at the appropriate site in the genome.

In one embodiment, the heterologous sequence which it is desired to recombine into the genome of a target animal comprises a functional sequence but under the control of an inducible promoter so that expression of the gene are regulated by administration of an endogenous molecule. This are advantageous where disruption of the gene is embryonic-lethal.

“Knock-out” animals are used as animal models for the study of gene function.

Therapeutic Peptides

In accordance with this embodiment, ovarian cancer-associated proteins of the present invention are administered therapeutically to patients for a time and under conditions sufficient to ameliorate the growth of a tumor in the subject or to prevent tumor recurrence.

It is preferred to use peptides that do not consisting solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify peptides for therapeutic application. One approach is to link the peptides or proteins to a variety of polymers, such as polyethylene glycol (PEG) and polypropylene glycol (PPG)—see for example U.S. Pat. Nos. 5,091,176, 5,214,131 and U.S. Pat. No. 5,264,209.

Replacement of naturally-occurring amino acids with a variety of uncoded or modified amino acids such as D-amino acids and N-methyl amino acids may also be used to modify peptides

Another approach is to use bifunctional crosslinkers, such as N-succinimidyl 3-(2 pyridyidithio)propionate, succinimidyl 6-[3-(2 pyridyidithio)propionamido]hexanoate, and sulfosuccinimidyl 6-[3-(2 pyridyldithio)propionamido]hexanoate (see U.S. Pat. No. 5,580,853).

It are desirable to use derivatives of the ovarian cancer-associated proteins of the invention which are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide are stabilized by a covalent modification, such as cyclization or by incorporation of gamma-lactam or other types of bridges. For example, side chains are cyclized to the backbone so as create a L-gamma-lactam moiety on each side of the interaction site. See, generally, Hruby et al., “Applications of Synthetic Peptides,” in Synthetic Peptides: A User's Guide: 259-345 (W. H. Freeman & Co. 1992). Cyclization also are achieved, for example, by formation of cystine bridges, coupling of amino and carboxy terminal groups of respective terminal amino acids, or coupling of the amino group of a Lys residue or a related homolog with a carboxy group of Asp, Glu or a related homolog. Coupling of the .alpha-amino group of a polypeptide with the epsilon-amino group of a lysine residue, using iodoacetic anhydride, are also undertaken. See Wood and Wetzel, 1992, Int'l J. Peptide Protein Res. 39: 533-39.

Another approach described in U.S. Pat. No. 5,891,418 is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids are chemically altered to include a coordinating group, such as for example oxime, hydrazino, sulfhydryl, phosphate, cyano, pyridino, piperidino, or morpholino. The peptide construct are either linear or cyclic, however a linear construct is typically preferred. One example of a small linear peptide is Gly-Gly-Gly-Gly which has four nitrogens (an N₄ complexation system) in the back bone that can complex to a metal ion with a coordination number of four.

A further technique for improving the properties of therapeutic peptides is to use non-peptide peptidomimetics. A wide variety of useful techniques are used to elucidating the precise structure of a peptide. These techniques include amino acid sequencing, x-ray crystallography, mass spectroscopy, nuclear magnetic resonance spectroscopy, computer-assisted molecular modeling, peptide mapping, and combinations thereof. Structural analysis of a peptide generally provides a large body of data which comprise the amino acid sequence of the peptide as well as the three-dimensional positioning of its atomic components. From this information, non-peptide peptidomimetics are designed that have the required chemical functionalities for therapeutic activity but are more stable, for example less susceptible to biological degradation. An example of this approach is provided in U.S. Pat. No. 5,811,512.

Techniques for chemically synthesising therapeutic peptides of the invention are described in the above references and also reviewed by Borgia and Fields, 2000, TibTech 18: 243-251 and described in detail in the references contained therein.

Assays for Therapeutic Compounds

The ovarian cancer proteins, nucleic acids, and antibodies as described herein are used in drug screening assays to identify candidate compounds for use in treating ovarian cancer. The ovarian cancer-associated proteins, antibodies, nucleic acids, modified proteins and cells containing ovarian cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., 1998, Science 279: 84-88); Heid, 1996, Genome Res 6: 986-94).

In a preferred embodiment, the ovarian cancer-associated proteins, antibodies, nucleic acids, modified proteins and cells containing the native or modified ovarian cancer-associated proteins are used in screening assays. That is, the present invention provides methods for screening for compounds/agents which modulate the ovarian cancer phenotype or an identified physiological function of a ovarian cancer-associated protein. As above, this are done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays are executed. In a preferred embodiment, assays are run on an individual gene or protein level. That is, having identified a particular gene as up regulated in ovarian cancer, test compounds are screened for the ability to modulate gene expression or for binding to the ovarian cancer-associated protein. “Modulation” thus includes both an increase and a decrease in gene expression. The preferred amount of modulation will depend on the original change of the gene expression in normal versus tissue undergoing ovarian cancer, with changes of at least 10%, preferably 50%, more preferably 100-300%, and in some embodiments 300-1000% or greater. Thus, if a gene exhibits a 4-fold increase in ovarian cancer tissue compared to normal tissue, a decrease of about four-fold is often desired; similarly, a 10-fold decrease in ovarian cancer tissue compared to normal tissue often provides a target value of a 10-fold increase in expression to be induced by the test compound.

The amount of gene expression are monitored using nucleic acid probes and the quantification of gene expression levels, or, alternatively, the gene product itself are monitored, e.g., through the use of antibodies to the ovarian cancer-associated protein and standard immunoassays. Proteomics and separation techniques may also allow quantification of expression.

In a preferred embodiment, gene expression or protein monitoring of a number of entities, i.e., an expression profile, is monitored simultaneously. Such profiles will typically involve a plurality of those entities described herein.

In this embodiment, the ovarian cancer nucleic acid probes are attached to biochips as outlined herein for the detection and quantification of ovarian cancer sequences in a particular cell. Alternatively, PCR are used. Thus, a series are used with dispensed primers in desired wells. A PCR reaction can then be performed and analyzed for each well.

Expression monitoring are performed to identify compounds that modify the expression of one or more ovarian cancer-associated sequences, e.g., a polynucleotide sequence set out in Tables 1-3. In a preferred embodiment, a test modulator is added to the cells prior to analysis. Moreover, screens are also provided to identify agents that modulate ovarian cancer, modulate ovarian cancer-associated proteins, bind to a ovarian cancer-associated protein, or interfere with the binding of a ovarian cancer-associated protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the ovarian cancer phenotype or the expression of a ovarian cancer sequence, e.g., a nucleic acid or protein sequence. In preferred embodiments, modulators alter expression profiles, or expression profile nucleic acids or proteins provided herein. In one embodiment, the modulator suppresses a ovarian cancer phenotype, e.g. to a normal tissue fingerprint. In another embodiment, a modulator induced a ovarian cancer phenotype. Generally, a plurality of assay mixtures are run in parallel with different agent concentrations to obtain a differential response to the various concentrations. Typically, one of these concentrations serves as a negative control, i.e., at zero concentration or below the level of detection.

Drug candidates encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

In one aspect, a modulator will neutralize the effect of a ovarian cancer-associated protein. By “neutralize” is meant that activity of a protein is inhibited or blocked and the consequent effect on the cell.

In certain embodiments, combinatorial libraries of potential modulators will be screened for an ability to bind to a ovarian cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to Identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds are synthesized through such combinatorial mixing of chemical building blocks (Gallop et al., 1994, J. Med. Chem. 37(9):1233-1251).

Preparation and screening of combinatorial chemical libraries is well known to those of skill in the art. Such combinatorial chemical libraries include, but are not limited to, peptide libraries, peptoids, encoded peptides, random bio-oligomers, nonpeptidal peptidomimetics, analogous organic syntheses of small compound libraries, nucleic acid libraries, peptide nucleic acid libraries, antibody libraries, carbohydrate libraries and small organic molecule libraries.

The assays to identify modulators are amenable to high throughput screening. Preferred assays thus detect enhancement or inhibition of ovarian cancer gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all samisle and reagent pipetting, liquid dispensing, timed incubations, and final readings of the microplate in detectors) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, are used. In this way libraries of proteins are made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides are digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process are designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of ovarian cancer can also be nucleic acids, as defined below. As described above generally for proteins, nucleic acid modulating agents are naturally occurring nucleic acids, random nucleic acids, or “biased” random nucleic acids. For example, digests of procaryotic or eucaryotic genomes are used as is outlined above for proteins.

In certain embodiments, the activity of a ovarian cancer-associated protein is down-regulated, or entirely inhibited, by the use of antisense polynucleotide, i.e., a nucleic acid complementary to, and which can preferably hybridize specifically to, a coding mRNA nucleic acid sequence, e.g., a ovarian cancer-associated protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the ovarian cancer-associated protein mRNA. See, e.g., Isis Pharmaceuticals, Carlsbad, Calif.; Sequitor, Inc., Natick, Mass.

Such antisense polynucleotides can readily be synthesized using recombinant means, or are synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for ovarian cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein & Cohen (Cancer Res. 48:2659 (1988 and van der Krol et ai. (BioTechniques 6:958 (1988)).

In addition to antisense polynucleotides, ribozymes are used to target and inhibit transcription of ovarian cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different 5 ribozymes).

Methods of preparing ribozymes are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

Polynucleotide modulators of ovarian cancer are introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of ovarian cancer are introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

As noted above, gene expression monitoring is conveniently used to test candidate modulators (e.g., protein, nucleic acid or small molecule). After the candidate agent has been added and the cells allowed to incubate for some period of time, the sample containing a target sequence to be analyzed is added to the biochip. If required, the target sequence is prepared using known techniques. For example, the sample are treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cyS.

In a preferred embodiment, the target sequence is labeled with, e.g., a fluorescent, a chemiluminescent, a chemical, or a radioactive signal, to provide a means of detecting the target sequence's specific binding to a probe. The label also are an enzyme, such as, alkaline phosphatase or horseradish peroxidase, which when provided with an appropriate substrate produces a product that are detected. Alternatively, the label are a labeled compound or small molecule, such as an enzyme inhibitor, that binds but is not catalyzed or altered by the enzyme. The label also are a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays are direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby Incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions are used in the present invention, including high, moderate and low stringency conditions as outlined above. The assays are generally run under stringency conditions which allows formation of the label probe hybridization complex only in the presence of target. Stringency are controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it are desirable to perform certain steps at higher stringency conditions to reduce non-specific binding.

The reactions outlined herein are accomplished in a variety of ways. Components of the reaction are added simultaneously, or sequentially, in different orders, with preferred embodiments outlined below. In addition, the reaction may include a variety of other reagents. These include salts, buffers, neutral proteins, e.g. albumin, detergents, etc. which are used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the ovarian cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens are performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens are done for genes that are induced in response to a candidate agent. After identifying a modulator based upon its ability to suppress a ovarian cancer expression pattern leading to a normal expression pattern, or to modulate a single ovarian cancer gene expression profile so as to mimic the expression of the gene from normal tissue, a screen as described above are performed to identify genes that are specifically modulated in response to the agent. Comparing expression profiles between normal tissue and agent treated ovarian cancer tissue reveals genes that are not expressed in normal tissue or ovarian cancer tissue, but are expressed in agent treated tissue. These agent-specific sequences are identified and used by methods described herein for ovarian cancer genes or proteins. In particular these sequences and the proteins they encode find use in marking or identifying agent treated cells. In addition, antibodies are raised against the agent induced proteins and used to target novel therapeutics to the treated ovarian cancer tissue sample.

Thus, in one embodiment, a test compound is administered to a population of ovarian cancer cells, that have an associated ovarian cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) are put into a viral construct such as an adenoviral or retroviral construct, and added to the cell, such that expression of the peptide agent is accomplished. Regulatable gene administration systems can also be used.

Once the test compound has been administered to the cells, the cells are washed if desired and are allowed to incubate under preferably physiological conditions for some period of time. The cells are then harvested and a new gene expression profile is generated, as outlined herein.

Thus, e.g., ovarian cancer tissue are screened for agents that modulate, e.g., induce or suppress the ovarian cancer phenotype. A change in at least one gene, preferably many, of the expression profile indicates that the agent has an effect on ovarian cancer activity. By defining such a signature for the ovarian cancer phenotype, screens for new drugs that alter the phenotype are devised. With this approach, the drug target need not be known and need not be represented in the original expression screening platform, nor does the level of transcript for the target protein need to change.

In a preferred embodiment, as outlined above, screens are done on individual genes and gene products (proteins). That is, having identified a particular differentially expressed gene as important in a particular state, screening of modulators of either the expression of the gene or the gene product itself are done. The gene products of differentially expressed genes are sometimes referred to herein as “ovarian cancer-associated proteins” or a “ovarian cancer modulatory protein”. The ovarian cancer modulatory protein are a fragment, or alternatively, be the full length protein to the fragment encoded by the nucleic acids referred to in Tables 1-3. Preferably, the ovarian cancer modulatory protein is a fragment. In a preferred embodiment, the ovarian cancer amino acid sequence which is used to determine sequence identity or similarity is encoded by a nucleic acid referred to in Tables 1-3. In another embodiment, the sequences are naturally occurring allelic variants of a protein encoded by a nucleic acid referred to in Tables 1-3. In another embodiment, the sequences are sequence variants as further described herein.

Preferably, the ovarian cancer modulatory protein is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, i.e., to cysteine.

In one embodiment the ovarian cancer-associated proteins are conjugated to an immunogenic agent as discussed herein. In one embodiment the ovarian cancer-associated protein is conjugated to BSA.

Measurements of ovarian cancer polypeptide activity, or of ovarian cancer or the ovarian cancer phenotype are performed using a variety of assays. For example, the effects of the test compounds upon the function of the ovarian cancer polypeptides are measured by examining parameters described above. A suitable physiological change that affects activity are used to assess the influence of a test compound on the polypeptides of this invention. When the functional consequences are determined using intact cells or animals, one can also measure a variety of effects such as, in the case of ovarian cancer associated with tumours, tumour growth, tumour metastasis, neovascularization, hormone release, transcriptional changes to both known and uncharacterized genetic markers (e.g., northern blots), changes in cell metabolism such as cell growth or pH changes, and changes in intracellular second messengers such as cGMP. In tire assays of the invention, mammalian ovarian cancer polypeptide is typically used, e.g., mouse, preferably human.

Assays to identify compounds with modulating activity are performed in vitro. For example, a ovarian cancer polypeptide is first contacted with a potential modulator and incubated for a suitable amount of time, e.g., from 0.5 to 48 hours. In one embodiment, the ovarian cancer polypeptide levels are determined in vitro by measuring the level of protein or mRNA. The level of protein is measured using immunoassays such as western blotting, ELISA and the like with an antibody that selectively binds to the ovarian cancer polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system are devised using the ovarian cancer-associated protein promoter operably linked to a reporter gene such as luciferase, green fluorescent protein, CAT, or (beta-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens are done on individual genes and gene products (proteins). That is, having identified a particular differentially expressed gene as important in a particular state, screening of modulators of the expression of the gene or the gene product itself are done. The gene products of differentially expressed genes are sometimes referred to herein as “ovarian cancer-associated proteins.” The ovarian cancer-associated protein are a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants are further-screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the ovarian cancer-associated proteins are used in the assays.

Thus, in a preferred embodiment, the methods comprise combining a ovarian cancer-associated protein and a candidate compound, and determining the binding of the compound to the ovarian cancer-associated protein. Preferred embodiments utilize the human ovarian cancer-associated protein, although other mammalian proteins may also be used, e.g. for the development of animal models of human disease. In some embodiments, as outlined herein, variant or derivative ovarian cancer-associated proteins are used.

Generally, in a preferred embodiment of the methods herein, the ovarian cancer-associated protein or the candidate agent is non-diffusably bound to an insoluble support having isolated sample receiving areas (e.g. a microtiter plate, an array, etc.). The insoluble supports are made of any composition to which the compositions are bound, is readily separated from soluble material, and is otherwise compatible with the overall method of screening. The surface of such supports are solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. microtitre plates and arrays are especially convenient because a large number of assays are carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the ovarian cancer-associated protein is bound to the support, and a test compound is added to the assay. Alternatively, the candidate agent is bound to the support and the ovarian cancer-associated protein is added. Novel binding agents include specific antibodies, non-natural binding agents identified in screens of chemical libraries, peptide analogs, etc. Of particular interest are screening assays for agents that have a low toxicity for human cells. A wide variety of assays are used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the ovarian cancer-associated protein are done in a number of ways. In a preferred embodiment, the compound is labeled, and binding determined directly, e.g., by attaching all or a portion of the ovarian cancer-associated protein to a solid support, adding a labeled candidate agent (e.g., a fluorescent label), washing off excess reagent, and determining whether the label is present on the solid support. Various blocking and washing steps are utilized as appropriate.

In some embodiments, only one of the components is labeled, e.g., the proteins (or proteinaceous candidate compounds) are labeled. Alternatively, more than one component are labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e., a ovarian cancer-associated protein), such as an antibody, peptide, binding partner, ligand, etc. Under certain circumstances, there are competitive binding between the compound and the binding moiety, with the binding moiety displacing the compound. In one embodiment, the test compound is labeled. Either the compound, or the competitor, or both, is added first to the protein for a time sufficient to allow binding, if present. Incubations are performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the ovarian cancer-associated protein and thus is capable of binding to, and potentially modulating, the activity of the ovarian cancer-associated protein. In this embodiment, either component are labeled. Thus, e.g., if the competitor is labeled, the presence of label in the wash solution indicates displacement by the agent. Alternatively, if the test compound is labeled, the presence of the label on the support indicates displacement.

In an alternative preferred embodiment, the test compound is added first, with incubation and washing, followed by the competitor. The absence of binding by the competitor may indicate that the test compound is bound to the ovarian cancer-associated protein with a higher affinity. Thus, if the test compound is labeled, the presence of the label on the support, coupled with a lack of competitor binding, may indicate that the test compound is capable of binding to the ovarian cancer-associated protein.

In a preferred embodiment, the methods comprise differential screening to identity agents that are capable of modulating the activity of the ovarian cancer-associated proteins. In this embodiment, the methods comprise combining a ovarian cancer-associated protein and a competitor in a first sample. A second sample comprises a test compound, a ovarian cancer-associated protein, and a competitor. The binding of the competitor is determined for both samples, and a change, or difference in binding between the two samples indicates the presence of an agent capable of binding to the ovarian cancer-associated protein and potentially modulating its activity. That is, if the binding of the competitor is different in the second sample relative to the first sample, the agent is capable of binding to the ovarian cancer-associated protein.

Alternatively, differential screening is used to identify drug candidates that bind to the native ovarian cancer-associated protein, but cannot bind to modified ovarian cancer-associated proteins. The structure of the ovarian cancer-associated protein are modeled, and used in rational drug design to synthesize agents that interact with that site. Drug candidates that affect the activity of a ovarian cancer-associated protein are also identified by screening drugs for the ability to either enhance or reduce the activity of the protein.

Positive controls and negative controls are used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples are counted in a scintillation counter to determine the amount of bound compound.

A variety of other reagents are included in the screening assays. These include reagents like salts, neutral proteins, e.g. albumin, detergents, etc. which are used to facilitate optimal protein-protein binding and/or reduce non-specific or background interactions. Also reagents that otherwise improve the efficiency of the assay, such as protease inhibitors, nuclease inhibitors, anti-microbial agents, etc., are used. The mixture of components are added in an order that provides for the requisite binding.

In a preferred embodiment, the invention provides methods for screening for a compound capable of modulating the activity of a ovarian cancer-associated protein. The methods comprise adding a test compound, as defined above, to a cell comprising ovarian cancer-associated proteins. Preferred cell types include almost any cell. The cells contain a recombinant nucleic acid that encodes a ovarian cancer-associated protein. In a preferred embodiment, a library of candidate agents are tested on a plurality of cells.

In one aspect, the assays are evaluated in the presence or absence or previous or subsequent exposure of physiological signals, e.g. hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate ovarian cancer agents are identified. Compounds with pharmacological activity are able to enhance or interfere with the activity of the ovarian cancer-associated protein. Once identified, similar structures are evaluated to identify critical structural feature of the compound.

In one embodiment, a method of inhibiting ovarian cancer cell division is provided. The method comprises administration of a ovarian cancer inhibitor. In another embodiment, a method of inhibiting ovarian cancer is provided. The method comprises administration of a ovarian cancer inhibitor. In a further embodiment, methods of treating cells or individuals with ovarian cancer are provided. The method comprises administration of a ovarian cancer inhibitor.

In one embodiment, a ovarian cancer inhibitor is an antibody as discussed above. In another embodiment, the ovarian cancer inhibitor is an antisense molecule.

A variety of cell growth, proliferation, and metastasis assays are known to those of skill in the art, as described below.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumour suppressor genes, regenerate normal phenotype and require a solid substrate to attach and grow. Soft agar growth or colony formation in suspension assays are used to identify modulators of ovarian cancer sequences, which when expressed in host cells, inhibit abnormal cellular proliferation and transformation. A therapeutic compound would reduce or eliminate the host cells' ability to grow in stirred suspension culture or suspended in semisolid media, such as semi-solid or soft.

Techniques for soft agar growth or colony formation in suspension assays are described in Freshney, Culture of Animal Cells a Manual of Basic Technique (3rd ed., 1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This are detected morphologically by the formation of a disoriented monolayer of cells or rounded cells in foci within the regular pattern of normal surrounding cells. Alternatively, labeling index with (³H)-thymidine at saturation density are used to measure density limitation of growth. See Freshney (1994), supra. The transformed cells, when transfected with tumour suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a ovarian cancer-associated sequence and are grown for 24 hours at saturation density in non-limiting medium conditions. The percentage of cells labeling with (³H)-thymidine is determined autoradiographically. See, Freshney (1994), supra.

Growth Factor or Serum Dependence

Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. Growth factor or serum dependence of transformed host cells are compared with that of control. Tumor specific markers levels Tumor cells release an increased amount of certain factors (hereinafter “tumour specific markers”) than their normal counterparts. For example, plasminogen activator (PA) is released from human glioma at a higher level than from normal brain cells (see, e.g., Gullino, Angiogenesis, tumour vascularization, and potential interference with tumour growth. in Biological Responses in Cancer, pp. 178-184 (Mihich (ed.) 1985)). Similarly, Tumor angiogenesis factor (TAF) is released at a higher level in tumour cells than their normal counterparts. See, e.g., Folkman, Angiogenesis and Cancer, Sem Cancer Biol. (1992)). Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305 312 (1980); Gullino, Angiogenesis, tumour vascularization, and potential interference with tumour growth. in Biological Responses in Cancer, pp. 178-184 (Mihich (ed.) 1985); Freshney Anticancer Res.5:111-130 (1985).

Invasiveness into Matrigel

The degree of invasiveness into Matrigel-or some other extracellular matrix constituent are used as an assay to identify compounds that modulate ovarian cancer-associated sequences. Tumor cells exhibit a good correlation between malignancy and invasiveness of cells into Matrigel or some other extracellular matrix constituent. In this assay, tumourigenic cells are typically used as host cells. Expression of a tumour suppressor gene in these host cells would decrease invasiveness of the host cells.

Techniques described in Freshney (1994), supra, are used. Briefly, the level of invasion of host cells are measured by using filters coated with Matrigel or some other extracellular matrix constituent. Penetration into the gel, or through to the distal side of the filter, is rated as invasiveness, and rated histologically by number of cells and distance moved, or by prelabeling the cells with 125 1 and counting the radioactivity on the distal side of the filter or bottom of the dish. See, e.g., Freshney (1984), supra.

Tumor Growth In Vivo

Effects of ovarian cancer-associated sequences on cell growth are tested in transgenic or immune-suppressed mice. Knock-out transgenic mice are made, in which the ovarian cancer gene is disrupted or in which a ovarian cancer gene is inserted. Knock-out transgenic mice are made by insertion of a marker gene or other heterologous gene into the endogenous ovarian cancer gene site in the mouse genome via homologous recombination. Such mice can also be made by substituting the endogenous ovarian cancer gene with a mutated version of the ovarian cancer gene, or by mutating the endogenous ovarian cancer gene, e.g., by exposure to carcinogens.

A DNA construct is introduced Into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice are derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Alternatively, various immune-suppressed or immune-deficient host animals are used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) are used as a host. Transplantable tumour cells (typically about 10⁶ cells) injected into isogenic hosts will produce invasive tumours in a high proportions of cases, while normal cells of similar origin will not. In hosts which developed invasive tumours, cells expressing a ovarian cancer-associated sequences are injected subcutaneously. After a suitable length of time, preferably 4 to 8 weeks, tumour growth is measured (e.g. by volume or by its two largest dimensions) and compared to the control. Tumours that have a statistically significant reduction (using, e.g. Student's T test) are said to have inhibited growth.

Administration

therapeutic reagents of the invention are administered to patients, therapeutically. Typically, such proteins/polynucleotides and substances may preferably be combined with various components to produce compositions of the invention. Preferably the compositions are combined with a pharmaceutically acceptable carrier or diluent to produce a pharmaceutical composition (which are for human or animal use). Suitable carriers and diluents include isotonic saline solutions, for example phosphate-buffered saline. The composition of the invention are administered by direct injection. The composition are formulated for parenteral, intramuscular, intravenous, subcutaneous, intraocular, oral, vaginal or transdermal administration. Typically, each protein are administered at a dose of from 0.01 to 30 mg/kg body weight, preferably from 0.1 to 10 mg/kg, more preferably from 0.1 to 1 mg/kg body weight.

Polynucleotides/vectors encoding polypeptide components for use in modulating the activity of the ovarian cancer-associated proteins/polynucleotides are administered directly as a naked nucleic acid construct. When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques' for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition are formulated for parenteral, intramuscular, intravenous, subcutaneous, oral, intraocular or transdermal administration.

The pharmaceutical compositions are administered in a range of unit dosage forms depending on the method of administration. For example, unit dosage forms suitable for oral administration include, powder, tablets, pills, capsules and lozenges. Orally administered dosage forms will typically be formulated to protect the active ingredient from digestion and may therefore be complexed with appropriate carrier molecules and/or packaged in an appropriately resistant carrier. Suitable carrier molecules and packaging materials/barrier materials are known in the art.

The compositions of the invention are administered for therapeutic or prophylatic treatments. In therapeutic applications, compositions are administered to a patient suffering from a disease (e.g. ovarian cancer) in an amount sufficient to cure or at least partially ameliorate the disease and its complications. An amount adequate to accomplish this is defined as a “therapeutically effective dose”. An amount of the composition that is capable of preventing or slowing the development of cancer in a patient is referred to as a “prophylactically effective dose”.

The routes of administration and dosages described are intended only as a guide since a skilled practitioner will be able to determine readily the optimum route of administration and dosage for any particular patient and condition.

The present invention is further described with reference to the accompanying drawings and the following non-limiting examples.

### EXAMPLE 1

### Gene Expression Profiling to Identify Differentially-Expressed Genes in Ovarian Cancer

1. Tissue Bank and Database

Tissue was collected from patients undergoing treatment at the GCC, we have established an Ovarian Cancer Tissue Bank and Clinical Database that currently holds data on over 400 cases treated at the GCC between 1986 and 2002. Tissue (currently 149 fresh/frozen and 292 archival fixed paraffin-embedded samples) was acquired from patients undergoing cytoreductive surgery and does not interfere with the collection of tissue for the normal processing of diagnostic specimens. Patient consent, included in all our studies, was collected prior to surgery. Tissue specimens and their associated pathology reports were coded in order to maintain patient confidentiality. Uncoded data was electronically and/or physically locked with restricted access by appropriate senior investigators only. Clinical (diagnosis, treatment, residual disease) and pathological data (tumour grade, stage) were collected and updated (disease recurrence, patient survival) at regular intervals. This study has ethical approval from the South Eastern Sydney Area Health Service Research Ethics Committee, Australia. Clinical data and tissue collection are ongoing.

2. Genetic Profiling of Ovarian Cancers

In order to identify those genes differentially regulated in epithelial ovarian cancer 51 ovarian cancer tumor samples were manually dissected from biological samples derived from subjects undergoing cytoreductive surgery. These samples comprised 8 endometrioid tumors, 4 mucinous tumors and 31 serous epithelial ovarian tumors, 12 corresponding omental deposits and 8 borderline (low-malignant potential) tumors.

RNA was isolated from the tumor samples in addition to 4 normal ovary samples using Trizol reagent (Life Technologies, Rockville, Md., USA) essentially according to manufacturer's instructions. RNA was then reverse transcribed using an oligo(dT) anchored oligonucleotide that additionally comprised a T7 promoter sequence. Isolated cDNA was then transcribed in vitro using the T7 MEGAscript kit (Ambion, Austin, Tex., USA) according to manufacturers instructions. Transcription was performed with biotinylated nucleotides (Bio-11-CTP and Bio-16-UTP) to enable detection of the transcribed cRNA.

Levels of gene expression in the cancer samples was then determined by analysing the transcribed cDNA samples using customized Affymetrix GeneChip® microarrays that comprise 59,618 oligonucleotide probe sets. These probe sets facilitate analysis of 46,000 gene clusters, representing over 90% of the predicted expressed human genome.

Data were normalized, and changes in gene expression detected using a ranked penalized t-statistic with p-values adjusted for multiple testing using the Holm procedure. Analysis was performed using the LIMMA package (available from Bioconductor, Biostatistics Unit of the Dana Farber Cancer Institute at the Harvard Medical School/Harvard School of Public Health).

Gene expression in 186 samples representing 52 different tissues of the body was also determined using the previously described methods to facilitate the identification of changes in gene expression that are specific for ovarian cancer.

Using this method 284 up-regulated transcripts and 186 down-regulated transcripts were identified.

In order to determine the efficacy of such a method of analysis for determining gene expression changes associated with ovarian cancer, those genes identified were compared to results of published expression profile studies. Using this method, 71 genes were identified in the present study that had been previously identified, including, for example, genes known to be over-expressed in ovarian cancer, such as, for example MUC1 and E-cadherin.

The ovarian cancer-associated genes and proteins set forth in Table 1 include sequences that are up-regulated or down-regulated in ovarian cancer subjects, including subjects suffering specifically from serous, encodmetrioid, mucinous or clear cell ovarian cancer, or non-invasive (borderline) ovarian cancers of any phenotype, and subjects that suffered from recurrences of ovarian cancer in the medium term, or died within the medium term.

Data presented in Table 2 indicate those genes that are expressed at significantly higher levels or significantly reduced levels in patients suffering from serous cancer relative tot he level of expression of the same genes in a normal or healthy subject.

### EXAMPLE 2

### Validation of Gene Expression Profiling Results using Tissue Microarrays

Each of the transcripts identified as being differentially-expressed specifically in ovarian cancer was then further analysed using in situ hybridization or immunohistochemical staining of tissue microarrays constructed from a large cohort of primary ovarian tumor tissue. Such analysis confirms upregulation, down-regulation or total loss of expression of the transcripts identified in the microarray analysis of tumor samples.

Furthermore, as each of the samples in the tissue microarray have been clinicopathologically characterized (for example to identify cancer grade and/or disease stage) and the subjects from whom the tumors were isolated continuously monitored (to detect for example, death or relapse of cancer), changes with gene expression were also analysed for correlation with such parameters in order to determine predictive changes in gene expression.

The relative intensity and percentage of cells staining was determined and evaluated for associations with clinical stage and grade of disease and disease relapse using the Kaplan Meier method and log-rank test, and by univariate and bivariate analyses in a Cox proportional hazards model for gene expression and other clinical and pathologic predictors of outcome to determine the potential independent prognostic value of the markers being assessed.

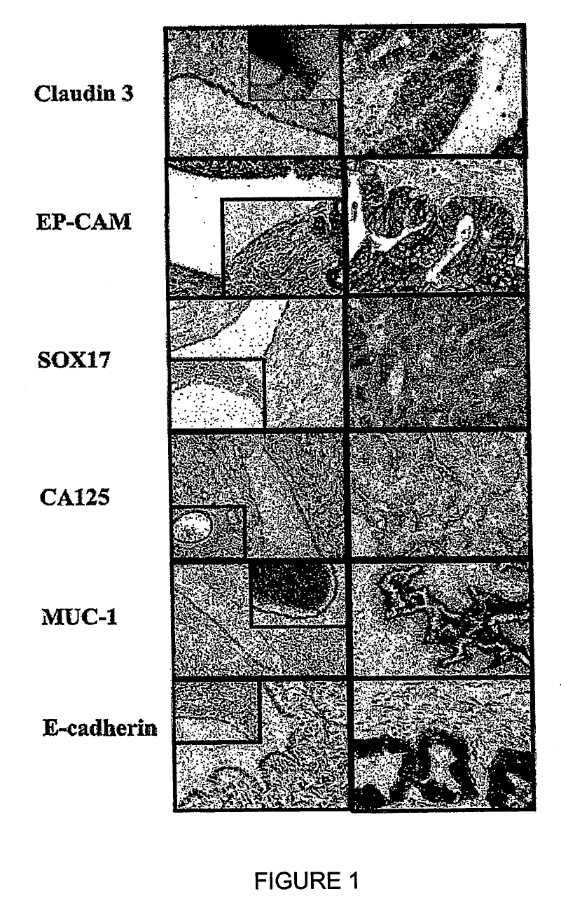

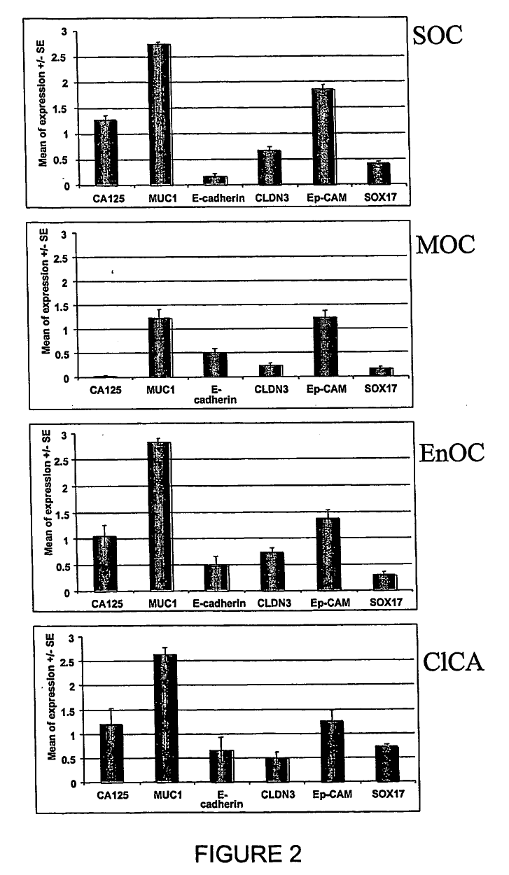

Immunohistochemical analysis has been performed on several genes identified in gene profiling analysis of ovarian cancer samples. For example, SOX17, Ep-CAM and claudin 3 were shown by gene profiling analysis to be specifically up-regulated in ovarian cancer compared to normal ovaries (FIG. 1 and FIG. 2). Using immunohistochemical analysis, it was determined that SOX17, EP-CAM and claudin 3 are upregulated in serous cancer, mucinous cancer, endometroid cancer and clear cell ovarian cancer.

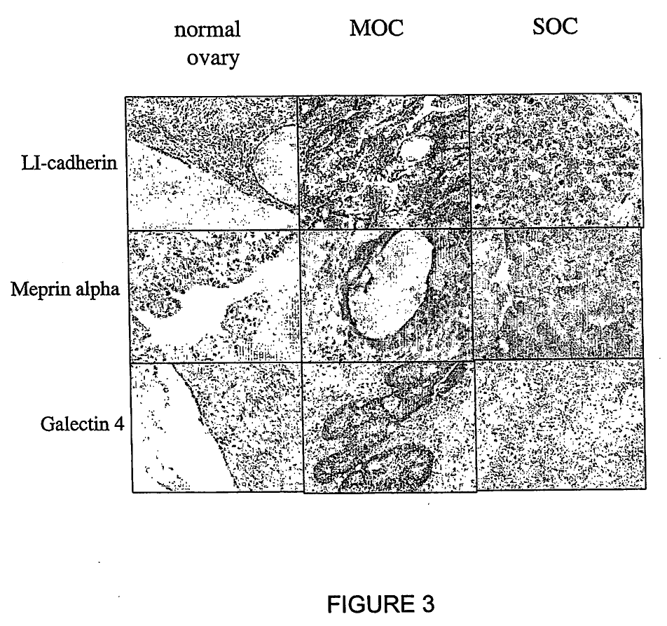

Furthermore, immunohistochemical analysis has been used to analyse the expression of several other genes that are specifically upregulated in mucinous ovarian cancer. In particular the expression of LI-cadherin (cadherin 17), meprin alpha and Galectin 4 as detected using immunohistochemistry is shown in FIG. 3. There was a significant increase in protein detected in the mucinous ovarian cancer samples compared to the normal ovary sample and serous ovarian cancer sample.

Immunohistochemical analysis was also performed to analyse the expression of three, genes that are known to be upregulated in ovarian cancer (CA125, MUC-1 and E-cadherin) (FIGS. 1 and 2).

### EXAMPLE 3′

### Identification of Prognostic Markers of Ovarian Cancer

Using a classical survival analysis to mine expression profiling data several genes that are associated with poor patient outcome (ie death or cancer relapse) have been identified (Tables 2 and 3). Such genes have clinical utility as prognostic indicators of disease.

Using detailed clinicopathological and postoperative data on all of the 51 patients included in our transcriptional profiling studies, including details of biochemical (eg. rising serum CA-125) and/or clinical recurrence of disease and overall survival, expression profiles were correlates with clinical parameters.

A preliminary survival analysis was performed on the 33 serous cancers within this cohort. The median follow-up time for these patients was 25.5 months from the date of primary laparotomy to the date of last follow-up or the date of death, and 21 of these patients (66%) were deceased from causes related to their malignancy.

Preliminary analysis of the expression profiles of these tumors identified several potential gene clusters that were associated with an increased risk of biochemical and clinical recurrence and overall survival, including the EDD gene (SEQ ID NO: 63). Exemplary prognostic markers for detecting ovarian cancer are shown in Tables 1 and 3. Preferred markers are indicated in Table 3.

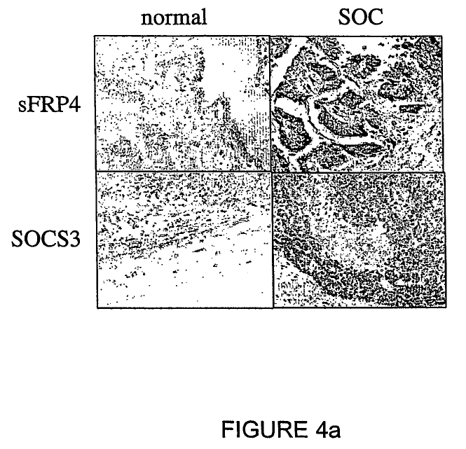

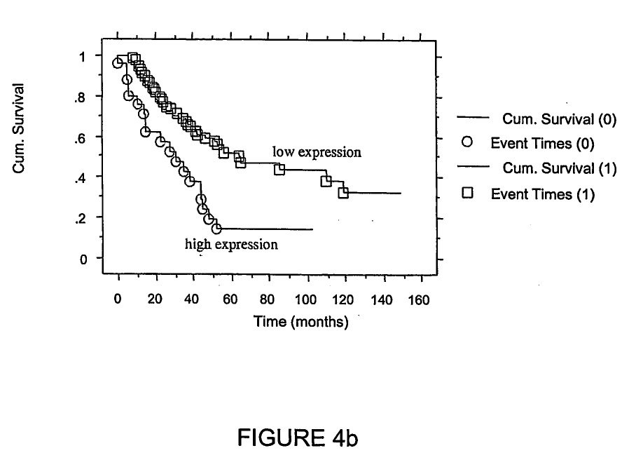

Using immunohistochemical analysis two genes have been confirmed to be upregulated in serous ovarian cancer. In particular, sFRP4, a negative signalling protein of the Wnt pathway, and SOCS3, a negative signaller of IL-6 induced signalling are specifically upregulated in serous ovarian cancer when compared to normal ovarian tissue (FIG. 4A).

Furthermore, using clinical patient data and correlating this information with gene expression levels using a Cox proportional hazards model, it has been shown that high expression of sFRP4 correlates with a poor outcome in patients (n=127) with serous ovarian cancer (p=0.0056) (FIG. 4B).

### EXAMPLE 4

### Validation of Gene Expression Profiling Results Using Quantitative RT-PCR

Candidate diagnostic genes are screened by quantitative RT-PCR against ovarian cancer cell lines to both validate the transcript profiling data (ie check their up- or down-regulation). Candidate diagnostic genes are screened using mRNA isolated from a panel of 9 ovarian tumour cell lines, (A2780, SKOV3, OVCAR-3, IGROV-1, CAOV3, OV-90, SW626, TOV-21 G and TOV-112D), in addition to several other tumour cell lines including lines derived from breast, prostate and colorectal tumours, and immortalised (non-transformed) human ovarian surface epithelial cells and a primary normal breast epithelial cell line (184).

Total RNA is isolated from the normal and tumour cell lines, reverse transcribed into cDNA and used as template in a quantitative PCR using a LightCycler system (Roche Diagnostics). The relative amount of each gene product is determined by comparison to a standard housekeeping gene (GAPDH).

### EXAMPLE 5

### Identification of Novel Genes for Diagnosis of Ovarian Cancer

We identified candidate genes with diagnostic potential from our list of aberrantly regulated genes by applying the following selection procedure: genes with a good transcript profile and low p-value (ie highly significantly up- or down-regulated in ovarian cancer, as determined in Example 1); and mapping to areas of the genome that have been shown to be amplified or lost in ovarian cancer. Accordingly, it is likely that these genes are involved in the development and progression of ovarian cancer (ie putative oncogenes and tumour suppressor genes). Additional parameters for analysis included known or putative function in oncogenesis (eg signal transduction, regulation of cellular proliferation, apoptosis etc); and association with other forms of other tumours. Genes identified in this analysis are shown in Table 3.

One method for the diagnosis of cancer comprises detecting modified DNA shed by the developing tumour into the blood stream. This can include the detection of mutations in both oncogenes and tumour suppressor genes involved in the development and progression of ovarian cancer. Furthermore, it has been recently shown that aberrant methylation of tumour suppressor genes, specifically hypermethylation of their gene promoters, frequently accompanies gene silencing in cancers, and indeed in some cases appears to be the predominant mechanism of gene silencing.

Combined with the knowledge of tumour nucleic acids circulating in the blood that reflect the biological characteristics of a tumour, the detection of methylation-specific tumour suppressor gene signatures for any given tumour type has promise as a specific and sensitive molecular test for detecting and monitoring cancer. Aberrant methylation is a frequent epigenetic event in epithelial ovarian cancer and many candidate tumour suppressor genes of epithelial ovarian cancer have been shown to be hypermethylated in epithelial ovarian cancer, such as, for example BRCA1.

In particular, expression of the candidate tumor suppressor gene MCC, has been shown to be down-regulated in epithelial ovarian cancer compared to normal ovarian tissue. MCC appears to be involved in critical cell growth regulatory processes and maps to a chromosomal region hypothesised as containing a tumor suppressor gene in ovarian cancer. Furthermore, we have identified a CpG island within the predicted promoter sequence of the MCC gene, a critical feature of genes that are subject to gene silencing by hypermethylation and a known characteristic of tumor suppressor genes. Taken together these data strongly implicate MCC as a candidate tumor suppressor gene involved in epithelial ovarian cancer.

TABLE 1
Genes having modified expression in subjects suffering from ovarian cancer

| | | | | |
|---|---|---|---|---|
| Accession number | UniGene Mapping | Gene symbol and title | Putative Function | P value |

a. upregulated genes

| | | | | |
|---|---|---|---|---|
| NM_002354 | Hs.692:235 | Ep-CAM; TACSTD1, tumor-associated calcium | Lymphocyte antigen, plasma membrane, tumor antigen. Member of | 0 |
| | | signal transducer 1; epithelial gylcoprotein | the GA733 family. C arcinoma-associated antigen expressed on most | |
| | | | normal epithelial cells and gastrointestinal carcinomas and functions | |
| | | | as a homotypic calcium-independent cell adhesion molecule. The | |
| | | | antigen is being used as a target for immunotherapy treatment of human carcinomas. | |
| BC006428 | Hs.15093:210; | HSPC195, hypothetical protein HSPC195 | Homo sapiens cDNA FLJ10920 fis, clone OVARC1000384-resourcerer. | 0 |
| | Hs.290304:1 | | | |
| NM_017697 | Hs.24743:94 | FLJ20171, hypothetical protein FLJ20171 | contains 3 RNA recognition motifs | 0 |
| AW419196 | Hs.257924:13 | FLJ13782, Hypothetical protein FLJ13782 | weakly similar to a drosophila transcription factor | 0 |
| AW630088 | Hs.76550:164 | MAL2 | Mal2 T-cell differentiation protein; found thru interaction with TPD52 | 0 |
| | | | which is overexpressed in breast cancer; 4 TM are involved in vesicle transport | |
| NM_004360 | Hs.194657:233 | CDH1, cadherin 1, type 1, E-cadherin (epithelial) | Tumor suppressor. Ca2+-dependent glycoprotein, mediates cell—cell | 0 |
| | | | interactions in epithelial cells. Mutations correlated with gastric, | |
| | | | breast, colorectal, thyroid and ovarian cancer. Loss of function | |
| | | | thought to contribute to progression in cancer by increasing | |
| | | | proliferation, invasion, and/or metastasis. The ectodomain of this | |
| | | | protein mediates bacterial adhesion to mammalian cells and the | |
| | | | cytoplasmic domain is required for internalization. | |
| NM_003761 | Hs.172684:89 | VAMP8, vesicle-associated membrane protein 8 | Early endosome, membrane fraction, non-selective vesicle docking, | 0 |
| | | (endobrevin) | non-selective vesicle transport, protein complex assembly, synaptic | |
| | | | vesicle. Member of a family involved in docking or fusion of synaptic | |
| | | | vesicles. Associated with the perinuclear vesicular structures of the | |
| | | | early endocytic compartment. | |
| NM_004415 | Hs.349499 | DSP, desmoplakin (DPI, DPII) | Cell shape and cell size control, cell—cell adherens junction, | 0 |
| | | | epidermal differentiation, intermediate filament, structural constituent | |
| | | | of cytoskeleton. Acts as a site of attachment for intermediate | |
| | | | filaments in desmosomes (intercellular junction in vertebrate | |
| | | | epithelial cells). Compound heterozygosity for non-sense and mis- | |
| | | | sense mutations underlies skin fragility/woolly hair syndrome. | |
| NM_013230 | Hs.286124:357; | CD24: CD24 antigen (small cell lung carcinoma | Plasma membrane, humoral defense mechanism. Cell surface | 0 |
| | Hs.375108 | cluster 4 antigen) | antigen; glycosyl phosphatidylinositol (GPI)-linked glycoprotein that | |
| | | | differentiates and activates granulocytes and B lymphocytes. | |
| NM_003710 | Hs.233950:84, Hs.182265:2, | SPINT1, serine protease inhibitor, Kunitz type 1. | Extracellular, membrane fraction, serine protease inhibitor. Member | 0 |
| | Hs.7771:1 | Hepatocyte growth factor activator inhibitor. | of the Kunitz family of serine protease inhibitors. Hepatocyte growth | |
| | | | factor activator inhibitor is a potent inhibitor specific for HGF activator | |
| | | | and is thought to be involved in regulation of proteolytic activation of | |
| | | | HGF in injured tissues. | |
| NM_153345 | Hs.17558:16 | FLJ90586, hypothetical protein | Function unknown | 0.0001 |
| NM_015238 | Hs.21543:36 | KIAA0869, KIAA0869 protein; KIBRA | Function unknown | 0.0002 |
| AI282759 | Hs.242463:1 | KRT8, keratin 8 | Cell structure, Cytoskeletal. May form intermediate filaments; type II | 0.0002 |
| | | | keratin, member of a family of structural proteins. Disruption of | |
| | | | mechanisms that normally regulate keratin expression in vivo could | |
| | | | be related to inflammatory and neoplastic pancreatic disorders | |
| | | | (Casanova 1999). | |
| AI393742 | Hs.199067:46 | ERBB3, v-erb-b2 erythroblastic leukemia viral | Transmembrane receptor protein tyrosine kinase, epidermal growth | 0.0002 |
| | | oncogene homolog 3 (avian) | factor receptor, integral plasma membrane protein, protein amino | |
| | | | acid phosphorylation. Member of the ERBB gene family of receptor | |
| | | | tyrosine kinases, elevated levels in certain human mammary tumor | |
| | | | cell lines. A receptor for heregulin, capable of mediating HGL- | |
| | | | stimulated tyrosine phosphorylation of itself. Epidermal growth factor | |
| | | | contains both positive and negative determinants for interaction with | |
| | | | ErbB-2/ErbB-3 heterodimers (Stortelers 2002) | |
| AW957300 | Hs.294142:167 | ESTs, Weakly similar to CYL1_HUMAN CYLICIN I | Function unknown | 0.0002 |
| | | [H. sapiens] | | |
| NM_012474; W70171 | Hs.75939:33, Hs.170864:1 | UMPK, uridine monophosphate kinase | Catalyzes the phosphorylation of uridine monophosphate to uridine | 0.0003 |
| | | | diphosphate. First step in production of pyrimidine nucleoside | |
| | | | triphosphates required for RNA and DNA synthesis. An allele of this | |
| | | | gene may play a role in mediating nonhumoral immunity to | |
| | | | Hemophilus influenzae type B. | |
| AA165082 | Hs.146388:47, Hs.113919:3 | MAP7, microtubule-associated protein 7 | Establishment and/or maintenance of cell polarity, microtubule | 0.0004 |
| | | | associated protein, microtubule cytoskeleton organization and | |
| | | | biogenesis, structural molecule. Predominantly expressed in cells of | |
| | | | epithelial origin. Involved in microtubule dynamics and cell | |
| | | | polarization and differentiation. Stabilizes microtubules, and may | |
| | | | modulate microtubule functions. Studies of the related mouse protein | |
| | | | suggest an essential role in microtubule function required for | |
| | | | spermatogenesis. | |
| AA284679 | Hs.25640:264, Hs.5372:2 | CLDN3, claudin 3 | Integral plasma membrane protein, pathogenesis, tight junction, | 0.0004 |
| | | | transmembrane receptor. Member of the claudin family of integral | |
| | | | membrane proteins; receptor for Clostridium perfringens enterotoxin; | |
| NM_004433 | Hs.166096:170 | ELF3, E74-like factor 3 (ets domain transcription | Embryogenesis and morphogenesis, transcription co-activator, | 0.0004 |
| | | factor, epithelial-specific) | transcription factor, transcription from Pol II promoter. ETS domain | |
| | | | transcriptional activator; activates expression of epithelial cell | |
| | | | specific genes. | |
| AW247252 | Hs.75514:181 | NP, nucleoside phosphorylase | DNA modification, nucleobase nucleoside nucleotide and nucleic | 0.0004 |
| | | | acid metabolism, purine-nucleoside phosphorylase. Enzyme purine | |
| | | | nucleoside phosphorylase together with adenosine deaminase | |
| | | | (ADA) serves a key role in purine catabolism, referred to as the | |
| | | | salvage pathway. Mutations in either enzyme result in a severe | |
| | | | combined immunodeficiency (SCID). | |
| NM_015925 | Hs.361379, | LISCH7, Liver-specific bHLH-Zip transcription | LISCH protein | 0.0004 |
| | Hs.95697:59, Hs.93649:1 | factor | | |
| NM_022454 | Hs.97984:22 | SOX17, SRY (sex determining region Y)-box 17 | Likely ortholog of mouse SRY-box containing gene 17; alias SOX17 | 0.0005 |
| AI124756 | Hs.5337:191 | IDH2, Isocitrate dehydrogenase 2 (NADP+), | Carbohydrate metabolism, mitochondrion | 0.0006 |
| | | mitochondrial | | |
| NM_003064 | Hs.313:273, Hs.297895:1 | SPP1, secreted phosphoprotein 1 (osteopontin, | Osteopontin (bone sialoprotein); bone and blood vessel extracellular | 0.0006 |
| | | bone sialoprotein I, early T-lymphocyte activation | matrix protein involved in calcification and atherosclerosis. Increased | |
| | | 1) | expression is associated with breast tumor metastasis (Urquidl | |
| | | | 2002). Role in HCC, especially in cancer-stromal interactions (Gotoh | |
| | | | 2002). Association between levels of a biomarker, osteopontin, and | |
| | | | ovarian cancer suggest its clinical usefulness (Kim 2002). | |
| BE382756 | Hs.169902:319, Hs.275406:1 | SLC2A1, Solute carrier family 2 (facilitated glucose | Glucose transporter, membrane fraction. SLC2A1/GLUT1 - | 0.0006 |
| | | transporter), member 1 | facilitated glucose transporter. Glucose transporter is an integral | |
| | | | membrane glycoprotein that is involved in transporting glucose into | |
| | | | most cells. 12 TMs. Role in transport of glucose across the blood- | |
| | | | brain barrier. Consistent marker of ovarian epithelial malignancy | |
| | | | (Kalir 2002). Marker for discriminating hepatocellular carcinoma from | |
| | | | other carcinomas (Zimmerman 2002). | |
| BE512730 | Hs.65114:718, Hs.279437:1 | KRT18, keratin 18 | Cell shape and cell size control, embryogenesis and morphogenesis, | 0.0006 |
| | | | intermediate filament, structural constituent of cytoskeleton. | |
| | | | Component of intermediate filaments; type I epidermal keratin, | |
| | | | strongly similar to murine Endo B. Expressed in single layer | |
| | | | epithelial tissues of the body. Mutations linked to cryptogenic | |
| | | | cirrhosis. | |
| NM_001769 | Hs.1244:227, Hs.230559:1, | CD9: CD9 antigen (p24) | Plasma membrane, integral plasma membrane protein. Member of | 0.0006 |
| | Hs.242020:1 | | the transmembrane 4 superfamily (TM4SF); may mediate platelet | |
| | | | activation and aggregation. Cell surface glycoprotein that is known to | |
| | | | complex with integrins and other transmembrane 4 superfamily | |
| | | | proteins. | |
| AI791905; | Hs.95549:147, Hs.229556:1 | FLJ20273, RNA-binding protein | Contains four RNA recognition motifs (RRM, RBD, or RNP) | 0.0007 |
| NM_019027 | | | | |
| NM_006103 | Hs.2719:108, Hs.54451:1 | WFDC2, WAP four-disulfide core domain 2 | Endopeptidase inhibitor, extracellular space, proteolysis and | 0.0009 |
| | | | peptidolysis, spermatogenesis. Epididymis-specific secreted protein; | |
| | | | may have a role in sperm maturation; arelong to a family of | |
| | | | extracellular proteinase inhibitors. Expressed in pulmonary epithelial | |
| | | | cells, and also expressed in some ovarian cancers. | |
| U81961 | Hs.438580 | SCNN1A, sodium channel, nonvoltage-gated 1 | Amiloride-sensitive sodium channel, excretion, integral plasma | 0.0009 |
| | | alpha | membrane protein, membrane fraction, sodium transport. Alpha | |
| | | | subunit of the amiloride-sensitive epithelial sodium channel; | |
| | | | functions in nonvoltage-gated channel | |
| X69699; NM_013952 | Hs.73149:72, Hs.213008:1 | PAX8, paired box gene 8 | Histogenesis and organogenesis, embryogenesis and | 0.0009 |
| | | | morphogenesis, thyroid-stimulating hormone receptor, transcription | |
| | | | factor. Member of the paired domain family of nuclear transcription | |
| | | | factors; are involved in the ribosome assembly, required for normal | |
| | | | thyroid development. PAX genes play critical roles during fetal | |
| | | | development and cancer growth. | |
| AI027643 | Hs.120912:12 | ESTs | Function unknown | 0.001 |
| AA173992 | Hs.7956:28 | ESTs | Function unknown | 0.0011 |
| AB018249 | Hs.10458:10 | SCYA16, small inducible cytokine subfamily A | Antimicrobial humoral response (sensu invertebrata), cell—cell | 0.0011 |
| | | (Cys—Cys), member 16. | signaling, chemokine chemotaxis. Cytokine A16; lymphocyte and | |
| | | | monocyte chemoattractant. | |
| NM_014791 | Hs.184339:27 | MELK, likely ortholog of maternal embryonic | KIAA0175 gene product; serine/threonine protein kinase domain | 0.0011 |
| | | leucine zipper kinase. | | |
| NM_030674 | Hs.18272:81 | SLC38A1, solute carrier family 38, member 1 | amino acid transporter A1 (ATA1), likely ortholog of mouse N-system | 0.0012 |
| | | | amino acid transporter protein NAT2. | |
| NM_005682 | Hs.6527:201 | GPR56, G protein-coupled receptor 56 | cell adhesion, cell—cell signalling, G-protein linked receptor, integral | 0.0012 |
| | | | plasma membrane protein, G-protein linked receptor protein | |
| | | | signalling pathway. Member of the G protein-coupled receptor family; | |
| | | | similar to secretin and calcitonin receptors. 7 transmembrane | |
| | | | domains, a mucin-like domain and cysteine box in the N-terminal | |
| | | | region. Expressed in range of tissues, highest levels in thyroid, | |
| | | | selectively within the monolayer of cuboidal epithelial cells of the | |
| | | | smaller, more actively secreting follicies of human thyroid. | |
| | | | Differentially expressed in melanoma cell lines with different | |
| | | | metastatic potential (Zendman et al 1999). | |
| AI669760 | Hs.188881:6, Hs.199354:1 | ESTs | dbEST Library Tissue Type restricted to prostate | 0.0013 |
| NM_001730 | Hs.84728:127 | KLF5, Kruppel-like factor 5 (Intestinal) | RNA polymerase II transcription factor, transcription from Pol II | 0.0014 |
| | | | promoter. Zinc finger transcriptional activator; localizes to the | |
| | | | nucleus and binds the epidermal growth factor response element, | |
| | | | binds GC boxes. | |
| AI355761 | Hs.242463:2 | KRT8, keratin 8 | Cell structure, Cytoskeletal. May form Intermediate filaments; type II | 0.0014 |
| | | | keratin, member of a family of structural proteins. Disruption of | |
| | | | mechanisms that normally regulate keratin expression in vivo could | |
| | | | be related to inflammatory and neoplastic pancreatic disorders | |
| | | | (Casanova 1999). | |
| BE019020 | Hs.85838:171 | SLC16A3, solute carrier family 16 (monocarboxylic | Integral plasma membrane protein, membrane fraction, | 0.0015 |
| | | acid transporters), member 3 (MCT3) | monocarboxylic acid transport, monocarboxylic acid transporter. | |
| | | | Member of monocarboxylate transporter family; may function as a | |
| | | | transporter (MCT3). | |
| NM_001307 | Hs.278562:101 | CLDN7, claudin 7 | Integral membrane protein, tight junction. Similar to murine Cldn7; | 0.0016 |
| NM_002266 | Hs.159557:394 | KPNA2, karyopherin alpha 2 (RAG cohort 1, | DNA metabolism, G2 phase of mitotic cell cycle. NLS-bearing | 0.0016 |
| | | importin alpha 1) | substrate-nucleus import, cytoplasm, importin alpha-subunit, nuclear | |
| | | | localization sequence binding, nucleoplasm, regulation of DNA | |
| | | | recombination, spindle pole body and microtubule cycle (sensu | |
| | | | Saccharomyces). Karyopherin alpha 2 (importin alpha 1); subunit of | |
| | | | the NLS (nuclear localization signal) receptor. KPNA2 protein | |
| | | | interacts with the NLSs of DNA heilcase Q1 and SV40 T antigen and | |
| | | | are involved in the nuclear transport of proteins. KPNA2 also may | |
| | | | play a role in V(D)J recombination. | |
| AW176120 | Hs.9061:77 | MGC2477, hypothetical protein MGC2477 | function unknown | 0.0016 |
| BE265489 | Hs.3123:49 | LLGL2, lethal giant larvae (Drosophila) homolog 2 | Cytoskeleton, structural molecule. May associate with nonmuscle | 0.0016 |
| | | | myosin II heavy chain. cDNA source cancer cell lines. 57% ID to | |
| | | | m. musculus 1920362A tumor suppressor gene mgl1 | |
| BE279383 | Hs.26557:77 | PKP3, plakophilin 3 | Cell adhesion, intercellular junction. Desmosomal plaque proteins | 0.0016 |
| | | | are members of the ‘armadillo-repeat’ multigene family and have | |
| | | | important functions in cytoskeleton/cell membrane interactions. | |
| J05581; NM_002456 | Hs.89603:128, Hs.296789:1 | MUC1, mucin 1, transmembrane | Integral plasma membrane protein. Cell surface mucin glycoprotein | 0.0016 |
| | | | expressed by most glandular and ductal epithelial cells and some | |
| | | | hematapoietic cell lineages. Alterations in glycosylation in epithelial | |
| | | | cancer cells. Marker for hepatocellular carcinoma. MUC1 metabolic | |
| | | | complex conserved in tumor-derived and normal epithelial cells. | |
| | | | Expression predictor of surgical outcome in mass-forming | |
| | | | intrahepatic cholangiocarcinoma. Tyrosine kinase c-Src constitutes a | |
| | | | bridge between cystic fibrosis transmembrane regulator channel | |
| | | | failure and MUC1 overexpression in cystic fibrosis. | |
| AA531276 | Hs.59509:9 | ESTs (unnamed protein product) | Function unknown | 0.0017 |
| AW167128 | Hs.231934:3 | ESTs; weakly similar to A57717 transcription factor | Function unknown | 0.0018 |
| | | EC2 | | |
| AW368226 | Hs.67928:25, Hs.229840:1 | Ets-related transcription factor, ESX, epithelium- | Embryogenesis and morphogenesis, transcription co-activator, | 0.0021 |
| | | restricted Ets protein ESX-not in Unigene, but | transcription factor, transcription from Pol II promoter. | |
| | | found using resourcerer. | | |
| AK000733 | Hs.23900:82 | RACGAP1, Rac GTPase activating protein 1 | Strongly similar to murine Racgap1 GTPase-activating protein for | 0.0024 |
| | | | rac. The plexin-B1/Rac interaction inhibits PAK activation and | |
| | | | enhances Sema4D ligand binding | |
| NM_014736 | Hs.81892:95 | KIAA0101 gene product | function unknown; no signficant hits with Superfamily | 0.0025 |
| NM_014586 | Hs.109437:17 | HUNK, hormonally upregulated neu tumor- | Developmental processes, protein serine/threonine kinase, signal | 0.0025 |
| | | associated kinase | transduction, protein kinase containing SNF1 (fam of | |
| | | | serine/threonine kinases) domain; progesterone and estradiol | |
| | | | regulated. Similar to murine Hunk. | |
| AI885516 | Hs.95612:31, Hs.251688:1 | desmocollin type 2a, desmocollin 2, isoform Dsc2b | Cell adhesion, intercellular junction | 0.0027 |
| | | preproprotein; desmosomal glycoprotein II/III; | | |
| | | desmocollin-3-not in Unigene, but found using | | |
| | | resourcerer. | | |
| AW194426 | Hs.20726:17 | ESTs | Function unknown | 0.0027 |
| NM_001982 | Hs.199067:83, Hs.167386:1 | ERBB3, HER3 (c-erb-B3), v-erb-b2 erythroblastic | Epidermal growth factor receptor, integral plasma membrane protein, | 0.0028 |
| | | leukemia viral oncogene homalog 3 (avian) | protein amino acid phosphorylation. Member of the ERBB gene | |
| | | | family of receptor tyrosine kinases, elevated levels in certain human | |
| | | | mammary tumor cell lines. A receptor for heregulin, capable of | |
| | | | mediating HGL-stimulated tyrosine phosphorylation of itself. | |
| NM_007019 | Hs.93002:85 | UBE2C, ubiquitin carrier protein E2-C | Ubiquitin-dependent protein degradation, degradation of cyclin, | 0.0031 |
| | | | protein modification, positive control of cell proliferation. Subunit of a | |
| | | | complex with ubiquitin ligase activity; complex that exhibits cyclin- | |
| | | | selective ubiquitin ligase activity. | |
| BE184455 | Hs.251754:128, Hs.245742:1 | SLPI, secretory leukocyte protease inhibitor | Plasma protein, proteinase inhibitor. Secreted inhibitor which | 0.0034 |
| | | (antileukoproteinase) | protects epithelial tissues from serine proteases. Found in various | |
| | | | secretions including seminal plasma, cervical mucus, and bronchial | |
| | | | secretions, has affinity for trypsin, leukocyte elastase, and cathepsin | |
| | | | G. Its inhibitory effect contributes to the immune response by | |
| | | | protecting epithelial surfaces from attack by endogenous proteolytic | |
| | | | enzymes; the protein is also thought to have broad-spectrum anti- | |
| | | | biotic activity. | |
| Y00815; NM_002840 | Hs.75216:262, Hs.228792:1, | PTPRF, protein tyrosine phosphatase, receptor | Cell adhesion, integral plasma membrane protein, transmembrane | 0.0035 |
| | Hs.245063:1 | type, F | receptor protein, tyrosine phosphatase signaling pathway. Receptor- | |
| | | | type protein tyrosine phosphatase F; interacts with the insulin | |
| | | | receptor; has Ig-like and FN-III repeats in the extracellular domain | |
| AA706017 | Hs.119944:14 | ESTs | Function unknown | 0.0038 |
| AA256641 | Hs.238894:24 | ESTs, Highly similar to S02392 alpha-2- | Function unknown | 0.0041 |
| | | macroglobulin receptor precursor | | |
| AW055308 | Hs.31803:15 | ESTs, Weakly similar to TRHY_HUMAN | Function unknown | 0.0043 |
| | | TRICHOHYALI [H. sapiens] | | |
| AI301558 | Hs.290801:35, | EST | Function unknown | 0.0044 |
| | Hs.356228 | | | |
| T18997 | Hs.180372:119; | BCL2-like 1, Homo sapiens cDNA FLJ20750 fis, | Function unknown | 0.0044 |
| | Hs.394609 | clone HEP05174 (hypothetical protein | | |
| AI798863 | Hs.87191:8 | ESTs | Function unknown | 0.0049 |
| J03258 | Hs.2062:146 | VDR, vitamin D (1,25-dihydroxyvitamin D3) | DNA binding, signal transduction, vitamin D3 receptor. Zinc-finger | 0.0049 |
| | | receptor | DNA-binding transcription factor. Genetic polymorphism determines | |
| | | | bone mineral density. Stat1-vitamin D receptor interactions | |
| | | | antagonize 1,25-dihydroxyvitamin D transcriptional activity and | |
| | | | enhance stat1-mediated transcription. | |
| AA151647 | Hs.68877:141, Hs.228686:1 | CYBA, cytochrome b-245, alpha polypeptide | Cytochrome b, membrane, mitochondrion, superoxide metabolism. | 0.005 |
| | | | Alpha-subunit of cytochrome b245, primary component of the | |
| | | | microbicidal oxidase system of phagocytes. CYBA deficiency is | |
| | | | associated with chronic granulomatous disease (CGD). | |
| AI538613 | Hs.135657:8 | TMPRSS3 Transmembrane protease, serine 3 | Integral membrane protein, proteolysis and peptidolysis. Contains a | 0.0051 |
| | | | serine protease domain, a transmembrane domain, a LDL receptor- | |
| | | | like domain, and a scavenger receptor cysteine-rich domain. Serine | |
| | | | proteases are known to be involved in a variety of biological | |
| | | | processes, whose malfunction often leads to human diseases and | |
| | | | disorders. Expressed in fetal cochlea and many other tissues, and is | |
| | | | thought to be involved in the development and maintenance of the | |
| | | | inner ear or the contents of the perilymph and endolymph. Missense | |
| | | | mutations in autosomal recessive sensorineural deafness. Identified | |
| | | | as a tumor associated gene that is overexpressed in ovarian tumors. | |
| NM_018000 | Hs.79741:18 | FLJ10116, hypothetical protein FLJ10116 | Function unknown | 0.0051 |
| NM_144724 | Hs.124740:18 | hypothetical protein FLJ30532 | 59% identity to human Zinc finger protein 91 | 0.0051 |
| AJ278016 | Hs.55565:35 | ANKRD3, ankyrin repeat domain 3 | ATP binding, protein amino acid phospharylation, protein binding, | 0.0055 |
| | | | protein serine/threonine kinase. | |
| NM_013994 | Hs.75562:147 | DDR1, discoldin domain receptor family, member 1 | Cell adhesion, integral plasma membrane protein, transmembrane | 0.0055 |
| | | | receptor, protein tyrosine kinase. Epithelial-specific receptor protein | |
| | | | tyrosine kinase; are involved in cell adhesion; has putative discoldin | |
| | | | motifs in extracellular domain. DDR1 (CD167a) is a RTK that is | |
| | | | widely expressed in normal and transformed epithelial cells and is | |
| | | | activated by various types of collagen. | |
| T09997:NM_001312 | Hs.70327:196, Hs.211478:1 | CRIP-2, cysteine-rich protein 2 | Zn-finger LIM domain protein; 208-amino acid protein containing 2 | 0.0055 |
| | | | LIM domains | |
| BE302796 | Hs.105097:115 | TK1, thymidine kinase 1, soluble | Cytoplasm, thymidine kinase. Generates thymidylate for DNA | 0.006 |
| | | | synthesis. TK1 gene expression together with TS, TP and DPD gene | |
| | | | expression may play important roles in influencing the malignant | |
| | | | behavior of epithelial ovarian cancer (Fujiwaki R 2002). | |
| NM_001067 | Hs.156346:184, Hs.270810:2 | TOP2A, topoisomerase (DNA) II alpha (170 kD) | DNA binding, DNA topoisomerase (ATP-hydrolyzing), nucleus. DNA | 0.006 |
| | | | topoisomerase II alpha; may relax DNA torsion upon replication or | |
| | | | transcription. Involved in processes such as chromosome | |
| | | | condensation, chromatid separation, and the relief of torsional stress | |
| | | | that occurs during DNA transcription and replication. Catalyzes the | |
| | | | transient breaking and rejoining of two strands of duplex DNA. The | |
| | | | gene encoding this enzyme functions as the target for several | |
| | | | anticancer agents and a variety of mutations in this gene have been | |
| | | | associated with the development of drug resistance. Reduced | |
| | | | activity of this enzyme may also play a role in ataxia-telangiectasia. | |
| U46455 | Hs.252189:148, Hs.248217:1 | SDC4, syndecan 4 (amphiglycan, ryudocan) | Integral plasma membrane. proteoglycan syndecan. Syndecans are | 0.0061 |
| | | | transmembrane heparan sulfate proteoglycans that appear to act as | |
| | | | receptors or coreceptors involved in intracellular communication. | |
| | | | Members of the MYC gene family and 4 members of the syndecan | |
| | | | gene family are closely situated on 4 different chromosomes. | |
| M79141 | Hs.13234:39 | ESTs | Function unknown | 0.0062 |
| AI955040 | Hs.301584:5, Hs.265398:3 | ESTs, Moderately similar to hypothetical protein | Function unknown | 0.0065 |
| | | FLJ20378 [Homo sapiens] [H. sapiens] | | |
| NM_005560 | Hs.11669:81, Hs.231010:1 | LAMA5, laminin, alpha 5 | Basement lamina, structural molecule. Widely expressed in adult | 0.0066 |
| | | | tissues, with highest levels in lung, heart, and kidney. Fifth member | |
| | | | of the alpha subfamily of vertebrate laminin chains. Possible | |
| | | | basement membrane protein; contains laminin EGF-like domain, two | |
| | | | extracellular laminin G domains. | |
| BE563085 | Hs.833:97 | ISG15, interferon-stimulated protein, 15 kDa | Cell-cell signaling, cytoplasm, extracellular space, protein binding. | 0.0068 |
| | | | Protein that is induced by interferon. | |
| BE278288 | Hs.155048:119 | LU, Lutheran blood group (Auberger b antigen | Blood group antigen, cell adhesion, integral plasma membrane | 0.0069 |
| | | included) | protein, signal transduction, transmembrane receptor. Lutheran | |
| | | | blood group glycoprotein; may play role in cell—cell, cell-matrix | |
| | | | adhesion, signal transduction; member of the Ig superfamily, has | |
| | | | integrin-binding motifs, SH3 domains. | |
| NM_020859 | Hs.278628:52 | ShrmL, Shroom-related protein (KIAA1481 | Amiloride-sensitive sodium channel (weakly similar to Mus musculus | 0.0074 |
| | | protein) | PDZ domain actin binding protein) | |
| AI262789 | Hs.93659:52 | ERP70, protein disulfide isomerase related protein | Endoplasmic reticulum lumen, protein secretion. Strongly similar to | 0.008 |
| | | (calcium-binding protein, intestinal-related) | rat Rn.4070 (CABP2); may bind calcium. | |
| NM_006147 | Hs.11801:77 | IRF6, interferon regulatory factor 6 | Member 6 of the interferon regulatory factor transcription factor | 0.0082 |
| | | | family; has low similarity to IRF4, which is a lymphocytic transcription | |
| | | | factor that stimulates B cell proliferation. | |
| R61463 | Hs.16165:50 | LAK-4P, expressed in activated T/LAK | expressed in activated T/LAK lymphocytes | 0.0082 |
| | | lymphocytes | | |
| AI878857; | Hs.109706:285 | HN1, hematological and neurological expressed 1 | Strongly similar to murine Hn1 | 0.0087 |
| NM_016185 | | protein | | |
| AK001783 | Hs.73239:37 | FLJ10901, hypothetical protein FLJ10901 | B link shows some homology to KIAA1294 but no known function | 0.009 |
| AC004770 | Hs.4756:99 | FEN1, flap structure-specific endonuclease 1 | DNA repair enzyme, DNA replication, UV protection, double-strand | 0.0093 |
| | | | break repair, double-stranded DNA binding, double-stranded DNA | |
| | | | specific exodeoxyribonuclease, endonuclease, fatty acid | |
| | | | desaturation, membrane fraction. Removes 5′ overhanging flaps in | |
| | | | DNA repair and processes the 5′ ends of Okazaki fragments in | |
| | | | lagging strand DNA synthesis. | |
| AI567421 | Hs.273330:137 | AGRN: agrin | Agrin is a neuronal aggregating factor that induces the aggregation | 0.0093 |
| | | | of acetylcholine receptors and other postsynaptic proteins on muscle | |
| | | | fibers and is crucial for the formation of the neuromuscular junction. | |
| | | | Acts at the nerve-muscle synapse in the glomerular basal membrane | |
| | | | and on T-lymphocytes. | |
| AW161386 | Hs.13561:49 | MGC4692: hypothetical protein MGC4692 | Function unknown | 0.0103 |
| M85430 | Hs.155191:546 | VIL2, villin 2 (ezrin) | Cytoskeletal anchoring, microvillus. Regulates cell adhesion and | 0.0106 |
| | | | cortical morphogenesis. The cytoplasmic peripheral membrane | |
| | | | protein encoded by this gene functions as a protein-tyrosine kinase | |
| | | | substrate in microvilli. As a member of the ERM protein family, this | |
| | | | protein serves as an intermediate between the plasma membrane | |
| | | | and the actin cytoskeleton. It plays a key role in cell surface structure | |
| | | | adhesion, migration, and organization. | |
| AW250380 | Hs.109059:124, Hs.24756:11 | MRPL12, mitochondrial ribosomal protein L12 | Protein synthesis, General cellular role, Ribosomal subunit, | 0.0114 |
| | | | Mitochondrial, RNA-binding protein, Ribosome-associated. | |
| AI733848; | Hs.71935:13 | ZNF339, zinc finger protein 339 | Zinc finger protein | 0.0115 |
| NM_021220 | | | | |
| AF111856; | Hs.105039:48 | SLC34A2, solute carrier family 34 (sodium | SLC34A2: solute carrier family 34 (sodium phosphate), member 2; | 0.0121 |
| NM_006424 | | phosphate), member 2 | contains 8 predicted TMs and a cysteine-rich N-terminal region. | |
| | | | Type 2 sodium-dependent phosphate transporter. member of the | |
| | | | renal type II co-transporter family. | |
| BE386983; | Hs.343214 | CKLFSF7; chemokine-like factor super family 7 | chemokine-like factor gene superfamily; transmb 4 superfamily | 0.0131 |
| NM_138410 | | | | |
| AA433988 | Hs.98502:8 | MUC16, mucin 16, CA125 | Mucin 16. Allas CA125 ovarian cancer antigen | 0.0137 |
| AW248314 | Hs.9622:83 | MRPS18A, mitochondrial ribosomal protein S18A | Mitochondrial small ribosomal subunit, protein biosynthesis, | 0.0149 |
| | | | structural constituent of ribosomeribosomal mitochondrial protein | |
| | | | S18A | |
| AA454501 | Hs.43666:65 | PTP4A3, protein tyrosine phosphatase type IVA, | Prenylated protein tyrosine phosphatase. PTPs are cell signaling | 0.016 |
| | | member 3 | molecules that play regulatory roles in a variety of cellular processes. | |
| | | | Strong similarity to murine Ptp4a3 (Mm.4124). Overexpression of | |
| | | | this gene in mammalian cells was reported to inhibit angiotensin-II | |
| | | | induced cell calcium mobilization and promote cell growth. PRL3 | |
| | | | (PTP4A3) expressed at high levels cancer metastases (Saha et al. | |
| | | | 2001), PRL3 gene is important for colorectal cancer metastasis. | |
| U33446 | Hs.75799:116 | PRSS8, protease, serine, 8 (prostasin) | Extracellular space, plasma membrane, serine type peptidase. A | 0.0166 |
| | | | trypsinogen, member of the trypsin family of serine proteases. Highly | |
| | | | expressed in prostate epithelia, one of several proteolytic enzymes | |
| | | | found in seminal fluid. Protease-mediated regulation of sodium | |
| | | | absorption is a function of human airway epithelia, and prostasin is a | |
| | | | likely candidate for this activity. | |
| X98654 | Hs.93837:43 | PITPNM, phosphatidylinositol transfer protein, | Brain development, lipid metabolism, membrane fraction, | 0.0167 |
| | | membrane-associated | phosphatidylinositol transporter, phototransduction. Catalyzes the | |
| | | | transfer of phosphatidylinositol between membranes; similar to | |
| | | | Drosophila rdgB. | |
| AI660149 | Hs.44865:39, Hs.300819:19, | LEF1, Lymphoid enhancer-binding factor-1 | Very strongly similar to murine Lef1; may act as a transcription | 0.0172 |
| | Hs.293904:14 | | factor. Expressed in pre-B and T cells. Binds to T-cell receptor-alpha | |
| | | | enhancer and confers maximal enhancer activity. A target gene | |
| | | | ectopically activated in colon cancer, from selective activation of a | |
| | | | promoter for a full-length LEF1 isoform that binds beta-cateniri | |
| | | | (HOVANES 2001). | |
| AF098158; | Hs.9329:152 | C20orf1, chromosome 20 open reading frame 1 | ATP binding, GTP binding, cell proliferation, mitosis, nucleus spindle. | 0.0183 |
| NM_012112 | | | Proliferation-associated nuclear protein; associates with the spindle | |
| | | | pole and mitotic spindle during mitosis | |
| AB014551 | Hs.155120:101, | ARHGEF2, rho/rac guanine nucleotide exchange | Cell shape and cell size control, cell surface receptor linked signal | 0.0206 |
| | Hs.337774 | factor (GEF) 2 | transduction, guanyl-nucleotide exchange factor, microtubule | |
| | | | cytoskeleton. Rho GTPases play a fundamental role in numerous | |
| | | | cellular processes that are initiated by extracellular stimuli that work | |
| | | | through G protein coupled receptors. The encoded protein may form | |
| | | | complex with G proteins and stimulate Rho-dependent signals. | |
| | | | Rho/Rac guanine nucleotide exchange factor (GEF) 2; associates | |
| | | | with microtubules, stimulates GTP binding on Rac and Rho | |
| AI278023 | Hs.89986:24, Hs.290780:1 | ESTs | Function unknown | 0.0208 |
| Z95152 | Hs.178695:25, Hs.79107:1 | MAPK13, mitogen-activated protein kinase 13 | MAP kinase, antimicrobial humoral response (sensu invertebrata), | 0.0217 |
| | | | cell surface receptor, signal transduction, chemotaxis, stress | |
| | | | response. MAP kinases act as an integration point for multiple | |
| | | | biochemical signals, and are involved in a wide variety of cellular | |
| | | | processes such as proliferation, differentiation, transcription | |
| | | | regulation and development. Are activated by proinflammatory | |
| | | | cytokines and cellular stress. Transcription factor ATF2, and | |
| | | | microtubule dynamics regulator stathmin are substrates of this | |
| | | | kinase. | |
| AW840171 | Hs.265398:7 | ESTs, Moderately similar to hypothetical protein | Function unknown | 0.0222 |
| | | FLJ20378 [Homo sapiens] [H. sapiens] | | |
| D49441 | Hs.155981:53 | MSLN, mesothelin | Cell adhesion, cell surface antigen, membrane. Pre-pro- | 0.0225 |
| | | | megakaryocyte potentiating factor. An antibody that reacts with | |
| | | | ovarian cancers and mesotheliomas was used to isolate a cell | |
| | | | surface antigen named mesothelin. Although the function of | |
| | | | mesothelin is unknown, it may play a role in cellular adhesion and is | |
| | | | present on mesothelium, mesotheliomas, and ovarian cancers. | |
| AW797437 | Hs.69771:262, Hs.444:1, | EST, CM1-UM0039-030400-173-a09 | Function unknown | 0.0229 |
| | Hs.294163:1 | | | |
| BE396290 | Hs.5097:261 | SYNGR2, synaptogyrin 2 | Integral plasma membrane protein, member of a family of | 0.0229 |
| | | | transmembrane synaptic vesicle proteins, specialized secretory | |
| | | | organelles that store neurotransmitters in nerve terminals, and | |
| | | | release them by fusing with the presynaptic plasma membrane | |
| | | | during exocytosis. | |
| AI656166; | Hs.7331 | ASRGL1; asparaginase like 1 | glycoprotein catabolism | 0.02 |
| NM_025080 | | | | |
| NM_002145 | Hs.2733:25 | HOXB2, homeo box B2, Hox2H protein | Circulation, developmental processes, transcription factor. Member | 0.024 |
| | | | of homeodomain family of DNA binding proteins; may regulate gene | |
| | | | expression, morphogenesis, and differentiation. Genes of the HOXB | |
| | | | (or HOX2) complex are expressed specifically in | |
| | | | erythromegakaryocytic cell lines, some are expressed only in | |
| | | | hematopoietic progenitors. | |
| AW959311 | Hs.87019:8; | Hypothetical protein DKFZp434J037 | probable serine/threonine protein kinase; KIAA0537 | 0.0251 |
| | Hs.172012 | | | |
| NM_000269 | Hs.118638:166, Hs.276104:1, | NME1, non-metastatic cells 1, protein (NM23A) | Transcription factor and nucleoside diphosphate kinase; has a role in | 0.0257 |
| | Hs.276127:1, Hs.276246:1 | | the transcriptional regulation of c-myc expression. Mutations in | |
| | | | NME1 have been identified in aggressive neuroblastomas. | |
| AA379597 | Hs.5199:87, Hs.277192:1 | HSPC150, HSPC150 protein similar to ubiquitin- | Similar to ubiquitin conjugating enzyme | 0.0259 |
| | | conjugating enzyme | | |
| BE148235 | Hs.193063:100 | Homo sapiens cDNA FLJ14201 fis, clone | high homology to ARP-3 actin-like protein | 0.0259 |
| | | NT2RP3002955 | | |
| AI683243; AI587638 | Hs.97258 | ESTs | Mod similarity to S29539 ribosomal protein L13a | 0.03 |
| AF111713 | Hs.286218:64 | JAM1, junctional adhesion molecule | Cell motility, inflammatory response, intercellular junction. Role in the | 0.0261 |
| | | | regulation of tight junction assembly in epithelia. Ligation of JAM is | |
| | | | required for reovirus-induced activation of NF-kappa-B and | |
| | | | apoptosis. Role in lymphocyte homing. | |
| BE391635 | Hs.75725:450, Hs.274751:1, | TAGLN2, transgelin 2 | Complex assembly protein. Homolog of the protein transgelin, which | 0.0275 |
| | Hs.277482:1, Hs.277468:1 | | is one of the earliest markers of differentiated smooth muscle. | |
| | | | Function not yet determined. Are an actin-binding protein. | |
| D14697 | Hs.77393:201, Hs.247769:1 | FDPS, famesyl diphosphate synthase (famesyl | Famesyl pyrophosphate synthetase (famesyl diphosphate | 0.0276 |
| | | pyrophosphate synthetase, | synthase); part of the cholesterol synthesis pathway. | |
| | | dimethylallyltranstransferase, | | |
| | | geranyltranstransferase) | | |
| AW194364 | Hs.94814 | MGC2865, Hypothetical protein MGC2865 | Function unknown. | 0.0295 |
| T47364 | Hs.278613:145 | IFI27, interferon, alpha-inducible protein 27 | Integral membrane protein. Isolated from estradiol-treated human | 0.03 |
| | | | breast carcinoma cells. Induced by interferon-alpha in human cell | |
| | | | lines of different origin, expression is independent of the presence of | |
| | | | estradiol receptor in the cells. | |
| U17760 | Hs.301103:71, Hs.75517:24, | LAMB3, Laminin, beta 3 (nicein (125 kD), kalinin | Epidermal differentiation, laminin-5, structural molecule. Member of | 0.0304 |
| | Hs.199068:1 | (140 kD), BM600 (125 kD)) (Accn NM_000228) | a family of basement membrane proteins. LAMB3 serves as the beta | |
| | | | chain in laminin-5. Mutations in LAMB3 have been identified as the | |
| | | | cause of various types of epidermolysis bullosa. | |
| AU076517 | Hs.184276:142 | SLC9A3R1, solute carrier family 9 | Actin cytoskeleton, protein complex assembly. Regulatory cofactor of | 0.0312 |
| | | (sodium/hydrogen exchanger), isoform 3 | the NHE3 (SLC9A3) sodium/hydrogen antiporter, interacts with | |
| | | regulatory factor 1 | merlin (NF2) and ERM family members; has two PDZ domains. | |
| | | | Structural determinants in interaction of beta 2 adrenergic and | |
| | | | platelet-derived growth factor receptors | |
| AW880841 | Hs.96908, | PIG11, p53-induced protein | Negative control of cell proliferation, stress response. May generate | 0.0314 |
| | Hs.74427:112 | | or respond to oxidative stress, may have a role in p53-dependent | |
| | | | apoptosis Polyak K, Xia Y, Zweier JL, Kinzier KW, Vogelstein B. A | |
| | | | model for p53-induced apoptosis. Nature. 1997 Sep | |
| | | | 18; 389(6648): 300-5. | |
| H24185 | Hs.92918:91 | BM-009, hypothetical protein BM-009 | Function unknown | 0.0314 |
| BE614410 | Hs.23044:51 | MGC16386, hypothetical protein, similar to RIKEN | Function unknown. | 0.0326 |
| | | cDNA | | |
| H16423 | Hs.82685:37 | CD47: CD47 antigen (Rh-related antigen, integrin- | Oncogenesis, plasma membrane, plasma glycoprotein, cell—cell | 0.0336 |
| | | associated signal transducer) | matrix adhesion, integral plasma membrane proteoglycan, integrin | |
| | | | receptor signal signalling pathway. Similar to Rh-antigen; may | |
| | | | interact with integrins and have a role in intracellular calcium | |
| | | | increase during cell adhesion. | |
| AU076611; | Hs.154672:123 | MTHFD2, methylene tetrahydrofolate | Electron transporter, methenyltetrahydrofolate cyclohydrolase, | 0.0342 |
| NM_006636 | | dehydrogenase (NAD+ dependent); | mitochondrion. encodes a nuclear-encoded mitochondrial | |
| | | methenyltetrahydrofolate cyclohydrolase | bifunctional enzyme with methylenetetrahydrofolate dehydrogenase | |
| | | | and methenyltetrahydrofolate cyclohydrolase activities. may provide | |
| | | | formyltetrahydrofolate for formylmethionyl tRNA synthesis; involved | |
| | | | in initiation of mitochondrial protein synthesis. | |
| AI859390 | Hs.288940:49 | TMEM8, five-span transmembrane protein M83; | Integral plasma membrane protein. Type I transmembrane protein; | 0.0345 |
| | | type I transmembrane protein | contains five membrane-spanning domains | |
| AA159216 | Hs.55505:57 | FLJ20442, hypothetical protein FLJ20442 | Contains a dual specificity protein phosphatase catalytic domain; | 0.0354 |
| | | | 34% similar to protein-tyrosine phosphatase | |
| AF119665; | Hs.184011:156 | PP, pyrophosphatase (inorganic) | Inorganic diphosphatase, phosphate metabolism. Catalyzes the | 0.0358 |
| NM_021129 | | | hydrolysis of pyrophosphate to inorganic phosphate | |
| BE513613; | Hs.11538:275 | ARPC1B, actin related protein 2/3 complex, | Cell motility, structural constituent of cytoskeleton. Arp2/3 complex, | 0.0387 |
| NM_005720 | | subunit 1A (41 kD) | subunit 1A; involved in assembly of the actin cytoskeleton, may have | |
| | | | a role in protrusion of lamellipodia | |
| NM_012153 | Hs.182339 | EHF: ets homologous factor | DNA binding, tumor suppressor, cell proliferation, developmental | 0.0404 |
| | | | processes, transcription activating factor. Member of the ESE | |
| | | | subfamily of Ets transcription factors | |
| AW772298 | Hs.21103:40, Hs.266784:2, | Homo sapiens mRNA; cDNA DKFZp564B076 | Alias coat protein gamma-cop | 0.0423 |
| | Hs.102950:1 | (from clone DKFZp564B076) | | |
| H16646 | Hs.118666:66 | PP591, hypothetical protein PP591 | Hypothetical protein PP591 (Novel Human cDNA clones with | 0.043 |
| | | | function of inhibiting cancer cell growth; unpublished) | |
| AA279661 | Hs.83753:244, Hs.301236:3 | SNRPB, small nuclear ribonucleoprotein | Spliceosome, mRNA splicing, small nuclear ribonucleoprotein. U1 | 0.0446 |
| | | polypeptides B and B1 | and U2 snRNP protein; component of snRNP complexes, required | |
| | | | units of the spliceosome | |
| BE001596 | Hs.85266:102 | ITGB4, integrin, beta 4 | Cell adhesion receptor, integrin, invasive growth, oncogenesis. Beta | 0.0453 |
| | | | 4 subunit of integrin; involved in cell—cell and cell-matrix interactions; | |
| | | | member of a family of cell-surface proteins. Binding of beta 4 to | |
| | | | plectin is essential for the proper formation and function of | |
| | | | hemidesmosomes. | |
| BE246444 | Hs.283685:148, Hs.232028:2 | FLJ20396, hypothetical protein FLJ20396 | 100%/175aa unnamed protein g7020468 | 0.0453 |
| X54942 | Hs.83758:34 | CKS2, CDC28 protein kinase 2 | Cell proliferation, regulation of CDK activity. Similar to S. pombe | 0.0478 |
| | | | p13suc1; binds and regulates CDK-cyclin complexes. expressed in | |
| | | | different patterns through the cell cycle in HeLa cells, which reflects | |
| | | | specialized role for the encoded protein. | |
| AA305599 | Hs.238205:36 | PRO2013, hypothetical protein PRO2013 | Function unknown | 0.0483 |
| AF019226 | Hs.8036:84 | RAB3D, member RAS oncogene family | RAB small monomeric GTPase, hemocyte development. GTP- | 0.0485 |
| | | | binding protein; are involved in vesicle transport; member of the RAB | |
| | | | family of small GTPases. Alias GOV, that is overexpressed in | |
| | | | glioblastoma multiforme tissue as compared to normal brain tissue. | |
| | | | GOV is also highly expressed in recurrent glioma, colon tumor | |
| | | | metastatic to brain, breast tumors, prostate tumors, and several | |
| | | | tumor cell lines | |
| NM_001949 | Hs.1189:65, Hs.296939:2 | E2F3, E2F transcription factor 3 | Protein binding, transcription factor, transcription initiation from Pol II | 0.049 |
| | | | promoter. Involved in cell cycle regulation, binds retinoblastoma | |
| | | | protein (Rb). E2F family plays a crucial role in the control of cell | |
| | | | cycle and action of tumor suppressor proteins and is also a target of | |
| | | | the transforming proteins of small DNA tumor viruses. | |
| AF217513 | Hs.279905:73, Hs.283649:4 | ANKT, nucleolar protein ANKT | clone HQ0310 PRO0310p1 nucleolar protein ANKT - no functional | 0.0504 |
| | | | data | |
| AW513143 | Hs.98367:8 | ESTs | Expressed in uterus | 0.0535 |
| AJ245671 | Hs.12844:73 | EGFL6, EGF-like-domain; multiple 6 | Cell cycle, oncogenesis, integrin ligand, extracellular space. Member | 0.0568 |
| | | | of the epidermal growth factor (EGF) repeat superfamily; contains an | |
| | | | EGF-like-domain. Expressed early during development, and its | |
| | | | expression has been detected in lung and meningioma tumors. | |
| AA084248 | Hs.85339:64 | GPR39, G protein-coupled receptor 39 | G-protein linked receptor, G-protein coupled receptor protein | 0.19 |
| | | | signaling pathway, integral plasma membrane protein. | |
| U62801 | Hs.79361:65 | KLK6, kallikrein 6 (neurosin, zyme) | Serine type peptidase, pathogenesis. Neurosin (protease M, zyme); | 0.0159 |
| | | | a serine protease that cleaves amyloid precursor protein (APP). | |
| | | | Growing evidence suggests that many kallikreins are implicated in | |
| | | | carcinogenesis and some have potential as novel cancer and other | |
| | | | disease biomarkers. | |
| D49441 | Hs.155981:53 | MSLN, mesothelin | Cell adhesion, cell surface antigen, membrane. Pre-pro- | 0.147 |
| | | | megakaryocyte potentiating factor. An antibody that reacts with | |
| | | | ovarian cancers and mesotheliomas was used to isolate a cell | |
| | | | surface antigen named mesothelin. Although the function of | |
| | | | mesothelin is unknown, it may play a role in cellular adhesion and is | |
| | | | present on mesothelium, mesotheliomas, and ovarian cancers. | |
| X51630 | Hs.1145:22, Hs.296851:1 | WT1, Wilms tumor 1 | Nucleus, transcription factor, transcription regulation. 4 Zn finger | 0.2938 |
| | | | domains. Functions in kidney and gonad proliferation and | |
| | | | differentiation. Mutations in this gene are associated with the | |
| | | | development of Wilms tumors in the kidney or with abnormalities of | |
| | | | the genitourinary tract. | |
| AB018305 | Hs.5378:149 | SPON1, spondin 1, (f-spondin) extracellular matrix | Extracellular matrix protein. Very strongly similar to rat F-spondin | 0.3394 |
| | | protein | (Rn.7546); may have a role in the growth and guidance of axons. | |
| AA433988 | Hs.98502:8 | MUC16, mucin 16, CA125 | Mucin 16. Alias CA125 ovarian cancer antigen | 0.6568 |
| NM_006149 | Hs.5302:132 | LGALS4, lectin, galactoside-binding, soluble, 4 | Lectin, cytosol, cell adhesion, plasma membrane. Binds to beta | 0.0001 |
| | | (galectin 4) | galactoside, involved in cell adhesion, cell growth regulation, | |
| | | | inflammation, immunomodulation, apoptosis and metastasis; | |
| | | | member of a family of lectins. LGALS4 is an S-type lectin that is | |
| | | | strongly underexpressed in colorectal cancer. | |
| AA315933 | Hs.120879:17 | Homo sapiens, clone MGC: 32871 | Function unknown | 0.0001 |
| | | IMAGE: 4733535, mRNA, complete cds | | |
| U47732 | Hs.84072:110 | TM4SF3, transmembrane 4 superfamily member 3 | Integral plasma membrane protein, lysosome, pathogenesis, protein | 0.0028 |
| | | | amino acid glycosylation, signal transducer, tumor antigen. Cell | |
| | | | surface glycoprotein defined by the monoclonal antibody CO-029 is | |
| | | | a 27- to 34-kD membrane protein expressed in gastric, colon, rectal, | |
| | | | and pancreatic carcinomas but not in most normal tissues | |
| NM_005588 | Hs.179704 | MEP1A, meprin A alpha, PABA peptide hydrolase | metalloprotease located apically and secreted by epithelial cells in | 0.01 |
| | | | normal colon; degrades broad range of ECM components in vitro; | |
| | | | proposed role in tumour progression by facilitating migration, | |
| | | | intravasation and metastasis | |
| AW503395 | Hs.5541:112 | ATP2A3, ATPase, Ca++ transporting, ubiquitous | Endoplasmic reticulum, adenosinetriphosphatase, small molecule | 0.0154 |
| | | | transport, calcium-transporting ATPase, integral plasma membrane | |
| | | | protein. Sarco/endoplasmic reticulum Ca2+-ATPase; pumps calcium. | |
| NM_004063 | Hs.89436:50 | CDH17, cadherin 17, LI cadherin (liver-intestine) | Cell adhesion, integral plasma membrane protein, membrane | 0.0172 |
| | | | fraction, small molecule transport, transporter. Member of the | |
| | | | cadherin family of calcium-dependent glycoproteins; facilitates | |
| | | | uptake of peptide-based drugs, may mediate cell—cell interactions. | |
| | | | Component of the gastrointestinal tract and pancreatic ducts, | |
| | | | intestinal proton-dependent peptide transporter in the first step in | |
| | | | oral absorption of many medically important peptide-based drugs. | |
| AI073913 | Hs.100686:20 | LOC155465, anterior gradient protein 3 | Oncogenesis | 0.0266 |
| AI928445 | Hs.92254:80 | SYTL2: synaptotagmin-like 2 | Synaptotagmin-like protein of the C2 domain-containing family of | 0.08 |
| | | | proteins. Although the specific function of the synaptotagmin-like | |
| | | | proteins is unknown, a role in regulation of synaptic vesicle | |
| | | | trafficking via their C2 domains has been suggested. Region of weak | |
| | | | similarity to murine Gph | |
| W40460 | Hs.144442:5 | PLA2G10: phospholipase A2, group X | Extracellular, secreted phospholipase A2. Group X secretory | 0.1888 |
| | | | phospholipase_a2; hydrolyzes the phospholipid sn-2 ester bond; | |
| | | | member of the phospholipase family | |
| AA132961 | Hs.212533:4 | Homo sapiens cDNA: FLJ22572 fis, clone | Function unknown | 0.1965 |
| | | HSI02313 | | |
| AF111856 | Hs.105039:48 | SLC34A2, solute carrier family 34 (sodium | SLC34A2: solute carrier family 34 (sodium phosphate), member 2; | 0.5078 |
| | | phosphate), member 2 | contains 8 predicted TMs and a cysteine-rich N-terminal region. | |
| | | | Type 2 sodium-dependent phosphate transporter. member of the | |
| | | | renal type II co-transporter family. | |
| AA143654 | | zo65a02.r1 Stratagene pancreas (#937208) Homo | Function unknown | 0.036 |
| | | sapiens cDNA clone IMAGE: 591722 5′, mRNA | | |
| | | sequence | | |

b. prognostic Indicators

| | | | | |
|---|---|---|---|---|
| AA046217 | Hs.105370:2 | ESTs | Function unknown | 0.00 |
| NM_015902 | | EDD: Homo sapiens progestin induced protein | Soluble fraction, cell proliferation, ubiquitin- protein ligase, ubiquitin | 0.00 |
| | | (DD5), mRNA. VERSION NM_020967.1 GI | conjugating enzyme, ubiquitin-dependent protein degradation. | |
| | | | Member of the HECT family of proteins; may function as an E3 | |
| | | | ubiquitin-protein ligase. This gene is localized to chromosome 8q22, | |
| | | | a locus disrupted in a variety of cancers. This gene potentially has a | |
| | | | role in regulation of cell proliferation or differentiation. | |
| T83882 | Hs.97927:20 | ESTs | Function unknown | 0.01 |
| #(NOCAT) | | NM_001615*: Homo sapiens actin, gamma 2, | Structural protein of muscle. Gamma 2 actin; enteric-type, smooth | 0.01 |
| | | smooth muscle, enteric (ACTG2), mRNA. variant | muscle cell actin. | |
| | | 1, mRNA. | | |
| AB040888 | | Homo sapiens mRNA for KIAA1455 protein, partial | Function unknown | 0.01 |
| | | cds | | |
| AA628980 | Hs.192371:3 | DSCR8 | Function unknown | 0.01 |
| | | down syndrome critical region protein DSCR8 | | |
| AI623351 | Hs.172148:51 | ESTs | Function unknown | 0.01 |
| AW614420 | Hs.204354:383 | ARHB | RHO small monomeric GTPase, RHO protein signal transduction, | 0.01 |
| | | ras homolog gene family, member B | peripheral plasma membrane protein. Ras-related GTP binding | |
| | | | protein of the rho subfamily, member B; may regulate assembly of | |
| | | | actin stress fibers and focal adhesions; very strongly similar to | |
| | | | murine Arhb. | |
| AA243499 | Hs.104800:23 | hypothetical protein FLJ10134 | Highly similar to murine p19.5; are a membrane protein | 0.01 |
| AF251237 | Hs.112208:16 | GAGED2 | GAGE genes are expressed in a variety of tumors and in some fetal | 0.01 |
| | | XAGE-1 protein | and reproductive tissues. This gene is strongly expressed in Ewing's | |
| | | | sarcoma, alveolar rhabdomyosarcoma and normal testis. The protein | |
| | | | encoded by this gene contains a nuclear localization signal and | |
| | | | shares a sequence similarity with other GAGE/PAGE proteins. | |
| | | | Because of the expression pattern and the sequence similarity, this | |
| | | | protein also belongs to a family of CT (cancer-testis) antigens. | |
| AI970797 | Hs.64859:16 | ESTs | Function unknown | 0.01 |
| AF145713 | Hs.61490:51 | SCHIP1 | Cytoplasm. Associates with the neurofibromatosis type 2 protein | 0.01 |
| | | schwannomin-interacting protein 1 | schwannomin (NF2); contains a colled-coil domain\|Proteome | |
| X78565 | Hs.289114:173, Hs.74637:1 | TNC | Cell adhesion, extracellular matrix, cell adhesion receptor, ligand | 0.01 |
| | | hexabrachion (tenascin C, cytotactin) | binding or carrier. Hexabrachion (tenascin c), an extracellular matrix | |
| | | | glycoprotein; has epidermal growth factor-like repeats | |
| T97307 | | gb:ye53h05.s1 Soares fetal liver spleen 1NFLS | Function unknown | 0.01 |
| | | Homo sapiens cDNA clone IMAGE: 121497 3′, | | |
| | | mRNA sequence. | | |
| BE243845 | Hs.75511:418 | CTGF | Cell motility, plasma membrane, soluble fraction, response to | 0.01 |
| | | connective tissue growth factor | wounding, extracellular matrix, extracellular space, epidermal | |
| | | | differentiation, cell growth and maintenance, insulin-like growth | |
| | | | factor binding, insulin-like growth factor receptor binding protein. | |
| | | | Connective tissue growth factor; binds IGF, may have a role in | |
| | | | regulating normal and neoplastic cell growth | |
| AW068302 | Hs.182183:214, Hs.325474:172, | CALD1 | Cytoskeleton, actin binding, calmodulin binding, tropomyosin | 0.01 |
| | Hs.283080:7 | caldesmon 1 | binding. Protein of unknown function. Actomyosin regulatory protein, | |
| | | | non-muscle form | |
| AL133561 | Hs.241426:5 | DKFZP434B061 protein | Function unknown | 0.01 |
| BE313555 | Hs.7252:158 | RAI17 | Function unknown | 0.02 |
| | | retinoic acid induced 17 | | |
| X07820 | Hs.2258:1 | MMP10 | Zinc binding, extracellular space, extracellular matrix, | 0.02 |
| | | matrix metalloproteinase 10 (MMP10; stromelysin | metalloendopeptidase, proteolysis and peptidolysis. Stromelysin 2; | |
| | | 2) | matrix metalloprotease that degrades connective tissue | |
| AI973016 | Hs.15725:77 | IER5 | Function unknown. A related mouse gene may play an important role | 0.02 |
| | | immediate early response 5 | in mediating the cellular response to mitogenic signals. | |
| AF084545 | | Homo sapiens versican Vint isoform, mRNA, | Function unknown | 0.02 |
| | | partial cds | | |
| U41518 | Hs.74602:146, Hs.767:1 | AQP1 | Excretion, water transport, water transporter, Integral plasma | 0.02 |
| | | aquaporin 1 (channel-forming integral protein, | membrane protein. Aquaporin 1 (channel-forming integral protein); | |
| | | 28 kD) | member of a family of water-transporters | |
| Z11894 | | H. sapiens rearranged mRNA for immunoglobulin | | 0.02 |
| | | kappa chain (VNJ) | | |
| AW138190 | Hs.180248:8 | ZNF124 | DNA binding. C2H2 zinc-finger protein 124 | 0.02 |
| | | zinc finger protein 124 (HZF-16) | | |
| BE086548 | Hs.42346:83, Hs.6975:42 | MYOZ2 | calcineurin-binding protein calsarcin-1 | 0.02 |
| | | myozenin 2 | | |
| W47196 | Hs.166172:50 | ARNT | Nucleus, transcription factor, transcription co-activator, transcription, | 0.02 |
| | | aryl hydrocarbon receptor nuclear translocator | DNA-dependent, protein-nucleus import, translocation, aryl | |
| | | | hydrocarbon receptor nuclear translocator. Aryl hydrocarbon | |
| | | | receptor nuclear translocator; used in receptor translocation from | |
| | | | cytosol to nucleus | |
| AI796870 | Hs.54277:76 | DXS9928E | Nucleus. Has many charged residues and a possible nuclear | 0.02 |
| | | DNA segment on chromosome X (unique) 9928 | localization signal | |
| | | expressed sequence | | |
| X02761 | Hs.287820:73, Hs.321592:1 | FN1 | Cell adhesion, cell motility, cell adhesion, soluble fraction, signal | 0.02 |
| | | fibronectin 1 | transduction, extracellular matrix, extracellular space. Fibronectin 1; | |
| | | | member of family of proteins found in plasma and extracellular | |
| | | | matrix | |
| AW968613 | Hs.79428:166 | BNIP3 | Anti-apoptosis, apoptosis inhibitor. Bcl2-related protein 3; binds | 0.02 |
| | | BCL2/adenovirus E1B 19 kD-interacting protein 3 | antiapoptotic viral E1B 19 kDa protein and cellular Bcl2 protein | |
| AW972565 | Hs.32399:24 | ESTs, Weakly similar to S51797 vasodilator- | Function unknown | 0.02 |
| | | stimulated phosphoprotein [H. sapiens] | | |
| AF045229 | Hs.82280:81 | RGS10 | Regulator of G protein signaling (RGS) family members are | 0.02 |
| | | regulator of G-protein signalling 10 | regulatory molecules that act as GTPase activating proteins (GAPs) | |
| | | | for G alpha subunits of heterotrimeric G proteins. RGS proteins are | |
| | | | able to deactivate G protein subunits of the Gi alpha, Go alpha and | |
| | | | Gq alpha subtypes. They drive G proteins into their inactive GDP- | |
| | | | bound forms. | |
| AW953853 | Hs.292833:19 | PAEP | Developmental processes. Placental protein 14 (Glycodelin); | 0.02 |
| | | progestagen-associated endometrial protein | member of lipocalin superfamily, highly similar to beta-lactoglobulins | |
| | | (placental protein 14, pregnancy-associated | | |
| | | endometrial alpha-2-globulin, alpha uterine | | |
| | | protein) | | |
| U52426 | Hs.74597:75, Hs.157615:3 | STIM1 | Integral plasma membrane protein, positive control of cell | 0.02 |
| | | stromal interaction molecule 1 | proliferation. Very strongly similar to murine Stim1; are a | |
| | | | transmembrane stromal cell protein | |
| F06700 | Hs.7879:115 | IFRD1 | Myoblast determination. Strongly similar to rat interferon-related | 0.02 |
| | | interferon-related developmental regulator 1 | developmental regulator 1; may play a role in muscle differentiation | |
| AI798863 | Hs.87191:8 | ESTs | Function unknown | 0.03 |
| NA | | C4001170: gi\|6863176\|gb\|AAF30402.1\|AF109924_1 | | 0.03 |
| | | (AF109924) sulfatase 1 precursor [Helix poma | | |
| H52761 | Hs.141475:24 | Homo sapiens cDNA clone IMAGE: 178663 | Function unknown | 0.03 |
| BE546947 | Hs.44276:43 | HOXC10 | Embryogenesis and morphogenesis, positive control of cell | 0.03 |
| | | homeo box C10 | proliferation, RNA polymerase II transcription factor. Homeobox C10, | |
| | | | member of the homeobox developmental regulator family; binds with | |
| | | | HOXA13 and HOXC13 to the Lamin B2 origin; ortholog of Drosophila | |
| | | | Abdominal-B | |
| AU076643 | Hs.313:257, Hs.329910:1 | SPP1 | Ossification, extracellular matrix, skeletal development. Osteopontin | 0.03 |
| | | secreted phosphoprotein 1 (osteopontin, bone | (bone sialoprotein); bone and blood vessel extracellular matrix | |
| | | sialoprotein I, early T-lymphocyte activation 1) | protein involved in calcification and atherosclerosis | |
| #(NOCAT) | | NM_015902*: Homo sapiens progestin induced | | 0.03 |
| | | protein (DD5), mRNA. VERSION NM_020967.1 GI | | |
| U20536 | Hs.3280:20 | CASP6 | Induction of apoptosis, cysteina-type peptidase, proteolysis and | 0.03 |
| | | caspase 6, apoptosis-related cysteine protease | peptidolysis. Caspase 6; a cysteine (thiol) protease; related to the | |
| | | | ICE-subfamily of caspases | |
| AA581602 | Hs.41840:7 | ESTs | Function unknown | 0.03 |
| AJ245210 | | gb: Homo sapiens mRNA for Immunoglobulin | Function unknown | 0.03 |
| | | gamma heavy chain variable region, partial, clone | | |
| | | 1A-4G21. | | |
| X65965 | | H. sapiens SOD-2 gene for manganese superoxide | | 0.03 |
| | | dismutase | | |
| AI806770 | Hs.30258:9 | ESTs | Function unknown | 0.03 |
| BE386490 | Hs.279663:51 | PIR | Nucleus, transcription co-factor, transcription from Pol II promoter. | 0.03 |
| | | Pirin | Putative cofactor of the NFI/CTF1 transcriptional activator | |
| AW581992 | Hs.301434:104, Hs.329017:1 | KIAA1387 | Function unknown | 0.03 |
| | | KIAA1387 protein | | |
| U77534 | | Human clone 1A11 immunoglobulin variable | Function unknown | 0.03 |
| | | region (VH5-D-JH4) gene, partial cds | | |
| AL034417 | Hs.11169:194, Hs.10958:1, | Gene 33/Mig-6 | Function unknown | 0.03 |
| | Hs.74137:1 | | | |
| L10343 | Hs.112341:96, Hs.1968:1 | Homo sapiens elafin precursor, gene, complete | Function unknown | 0.03 |
| | | cds | | |
| AW518944 | Hs.76325:80, Hs.231299:1 | IGJ | Linker protein for immunoglobulin alpha and mu polypeptides | 0.03 |
| | | immunoglobulin J polypeptide, linker protein for | | |
| | | immunoglobulin alpha and mu polypeptides | | |
| W28729 | Hs.236510:6 | Human retina cDNA randomly primed sublibrary | Function unknown | 0.03 |
| | | Homo sapiens cDNA, mRNA sequence | | |
| AI640160 | Hs.74131:4 | ARSE | Arylsulfatase, skeletal development. Arylsulfatase E; likely involved | 0.03 |
| | | arylsulfatase E (chondrodysplasia punctata 1) | in warfarin embryopathy. | |
| U11862 | Hs.75741:62 | ABP1 | Metabolism, peroxisome, amine oxidase, drug binding. Diamine | 0.03 |
| | | amiloride binding protein 1 (amine oxidase | oxidase (D-amino-acid oxidase histaminase, amiloride-binding | |
| | | (copper-containing)) | protein); deaminates putrescine and histamine | |
| AW295980 | Hs.252741:3 | ESTs | Function unknown | 0.03 |
| X59135 | Hs.156110:4 | H. sapiens mRNA for immunoglobulin 0-81VL | | 0.03 |
| BE466173 | Hs.379794 | Homo sapiens mRNA; cDNA DKFZp686N0118 | Function unknown | 0.03 |
| | | (from clone DKFZp686N0118) | | |
| #(NOCAT) | | Target Exon | | 0.03 |
| AI354722 | Hs.127216:24 | hypothetical protein FLJ13465 | Function unknown | 0.04 |
| M90464 | Hs.169825:45, Hs.408:1 | Human collagen type IV alpha 5 chain (COL4A5) | Function unknown | 0.04 |
| | | gene, 5′ end | | |
| AA829286 | Hs.332053:48, Hs.336462:10 | SAA1 | Inflammatory response, high-density lipoprotein. Member of the | 0.04 |
| | | serum amyloid A1 | serum amyloid A protein family; member of high density | |
| | | | apolipoproteins. | |
| AI333771 | Hs.82204:8, Hs.228363:1 | ESTs | Function unknown | 0.04 |
| BE465867; | Hs.197751:66 | DAAM1 | The protein encoded by this gene contains FH domains and belongs | 0.04 |
| NM_014992 | | dishevelled associated activator of morphogenesis 1 | to a novel FH protein subfamily implicated in cell polarity, thought to | |
| | | | function as a scaffolding protein. | |
| BE616902 | Hs.285313:145, Hs.4055:43 | COPEB | A trancriptional activator, capable of activating transcription | 0.04 |
| | | core promoter element binding protein | approximately 4-fold either on homologous or heterologous | |
| | | | promoters. The DNA binding and transcriptional activity of this | |
| | | | protein, in conjunction with its expression pattern, suggests that this | |
| | | | protein may participate in the regulation and/or maintenance of the | |
| | | | basal expression of pregnancy-specific glycoprotein gene and | |
| | | | possibly other TATA box-less genes. | |
| AA430373 | | gb: zw20f11.s1 Soares ovary tumor NbHOT Homo | Function unknown | 0.04 |
| | | sapiens cDNA clone IMAGE: 769869 3′ similar to | | |
| | | gb: M63438 IG KAPPA CHAIN PRECURSOR V-III | | |
| | | REGION (HUMAN);, mRNA sequence. | | |
| R27430 | Hs.271565:3 | ESTs | Function unknown | 0.04 |
| BE387335 | Hs.283713:68 | CTHRC1 | Function unknown | 0.04 |
| | | collagen triple helix repeat containing 1 | | |
| AW264102 | Hs.39168:16 | ESTs | Function unknown | 0.04 |
| NA | | Target Exon | Function unknown | 0.04 |
| AW952323 | Hs.129908:39 | KIAA0591 protein | Function unknown | 0.04 |
| AA088177 | Hs.172870:13 | ESTs | Function unknown | 0.04 |
| BE614567 | Hs.19574:123 | MGC5469 | Function unknown | 0.04 |
| | | hypothetical protein MGC5469 | | |
| AL079658 | Hs.338207:139, Hs.146559:1 | FRAP1 | DNA repair, DNA recombination, cell cycle control, 1- | 0.04 |
| | | FK506 binding protein 12-rapamycin associated | phosphatidylinositol 3-kinase, inositol/phosphatidylinositol kinase, | |
| | | protein 1 | FKBP-rapamycin associated protein; phosphatidylinositol kinase that | |
| | | | may mediate rapamycin inhibition of the cell cycle progression | |
| | | | through G1 | |
| NM_002776 | Hs.69423:46, Hs.275464:1 | KLK10 | Extracellular, serine-type peptidase. Putative serine protease | 0.04 |
| | | kallikrein 10 (KLK10) (PRSSL1) (nes1) | | |
| BE261944 | Hs.118625:62 | CYB561 | Energy pathways, secretory vesicle, cytochrome b5 reductase, | 0.04 |
| | | cytochrome b-561 | secretory vesicle membrane, integral plasma membrane protein. | |
| | | | Cytochrome b561; serves as a biological marker for adrenergic | |
| | | | secretory vesicles | |
| NM_006379 | Hs.171921:50 | SEMA3C | Drug resistance, immune response, cell growth and maintenance. | 0.04 |
| | | sema domain, immunoglobulin domain (Ig), short | Semaphorin E; member of a protein family involved in neuronal | |
| | | basic domain, secreted, (semaphorin) 3C | growth cone guidance | |
| AI002238 | Hs.11482:19 | SFRS11 | Nucleus, mRNA splicing, mRNA processing, pre-mRNA splicing | 0.04 |
| | | splicing factor, arginine/serine-rich 11 | factor, May have a role in pre-mRNA splicing; contains | |
| | | | arginine/serine-rich domain and an RRM domain | |
| #(NOCAT) | | ENSP00000231844*: Ecotropic virus integration 1 | | 0.04 |
| | | site protein. | | |
| X81789 | Hs.77897:149 | SF3A3 | Nucleus, spliceosome, mRNA splicing, mRNA processing, pre- | 0.04 |
| | | splicing factor 3a, subunit 3, 60 kD | mRNA splicing factor. Spliceosome-associated protein 3a, subunit 3; | |
| | | | component of the essential heterotrimeric splicing factor SF3a; | |
| | | | contains a zinc finger | |
| NM_002122 | Hs.198253:21 | HLA-DQA1 | Pathogenesis, class II major histocompatibility complex antigen. | 0.00 |
| | | major histocompatibility complex, class II, DQ | Alpha 1 chain of HLA-DQ1 class II molecule (Ia antigen); complex | |
| | | alpha 1 | binds peptides and presents them to CD4+ T lymphocytes\|Proteome | |
| AB001914 | | Homo sapiens PACE4 gene, exon 23-25, | Function unknown | 0.04 |
| | | complete cds | | |
| AA311919 | Hs.69851:24 | NOLA1 | Involved in various aspects of rRNA processing and modification. | 0.04 |
| | | nucleolar protein family A, member 1 (H/ACA small | Localize to the dense fibrillar components of nucleoli and to coiled | |
| | | nucleolar RNPs) | (Cajal) bodies in the nucleus. | |
| AI381750 | Hs.283437:122, Hs.10065:58 | HTGN29 protein | Function unknown | 0.04 |
| #(NOCAT) | | NM_000636*: Homo sapiens superoxide dismutase | Mitochondrion, oxidative stress response, manganese superoxide | 0.04 |
| | | 2, mitochondrial (SOD2), mRNA. expression) | dismutase. Manganese superoxide dismutase; intramitochondrial | |
| | | (RFX2), mRNA. | free radical scavenging enzyme; has strong similarity to murine | |
| | | | Sod2. | |
| AA292998 | Hs.163900:25 | ESTs | Function unknown | 0.04 |
| BE439580 | Hs.75498:40 | SCYA20 | Chemokine, chemotaxis, immune response, signal transduction, | 0.04 |
| | | small inducible cytokine subfamily A (Cys—Cys), | extracellular space, cell—cell signalling, inflammatory response, | |
| | | member 20 | antimicrobial humoral response. Cytokine A20 (exodus); chemotactic | |
| | | | factor for lymphocytes, but not a chemotactic factor for monocytes | |
| AI677897 | Hs.76640:124 | RGC32 | Cytoplasm, cell cycle regulator, regulation of CDK activity. Strongly | 0.04 |
| | | RGC32 protein | similar to RGC-32. | |
| #(NOCAT) | | Target Exon | Function unknown | 0.04 |
| N72403 | | Homo sapiens cDNA clone IMAGE: 245132 | Function unknown | 0.05 |
| BE003054 | Hs.1695:46 | MMP12 | Zinc binding, cell motility, macrophage elastase, extracellular matrix, | 0.05 |
| | | matrix metalloproteinase 12 (macrophage | proteolysis and peptidolysis. Matrix metalloprotease; degrades | |
| | | elastase) | elastin | |
| AL035588 | Hs.153203:26, Hs.23391:1 | Human DNA sequence from clone 696P19 on | Function unknown | 0.05 |
| | | chromosome 6p12.3-21.2. Contains the gene for | | |
| | | TFEB, an NPM1 (Nucleophosmin, Numatrin) | | |
| | | pseudogene and the MDFI gene for MyoD family | | |
| | | inhibitor (myogenic repressor I-MF). Contains | | |
| | | ESTs, STSs, GSSs and two putative CpG islands, | | |
| | | complete sequence | | |
| AI080491 | Hs.93270:3 | ESTs, Moderately similar to S65657 alpha-1C- | Function unknown | 0.05 |
| | | adrenergic receptor splice form 2 [H. sapiens] | | |
| AW770994 | Hs.30340:125 | hypothetical protein KIAA1165 | Function unknown | 0.05 |
| H24177 | Hs.75262:69, Hs.238912:1 | CTSO | Cysteine-type endopeptidase, proteolysis and peptidolysis. | 0.05 |
| | | cathepsin O | Cathepsin O; cysteine (thiol) protease | |
| AF146781 | Hs.20450:29 | BCM-like membrane protein precursor | Function unknown | 0.05 |
| NM_001955 | Hs.2271:45, Hs.306:1 | EDN1 | Circulation, peptide hormone, soluble fraction, signal transduction, | 0.05 |
| | | endothelin 1 | extracellular space, cell—cell signalling, blood pressure regulation, | |
| | | | positive control of cell proliferation. Preproendothelin 1; precursor of | |
| | | | the hormone endothelin 1 | |
| AI680737 | Hs.289068:204, Hs.326198:1 | TCF4 | Nucleus, RNA polymerase II transcription factor, transcription | 0.05 |
| | | transcription factor 4 | regulation from Pol II promoter. Transcriptional activator; interacts | |
| | | | with ITF1 (TCF3); contains basic helix-loop-helix domain\|Proteome | |
| AI752666 | Hs.76669:183 | NNMT | Nicotinamide N-methyltransferase; catalyzes the N-methylation of | 0.05 |
| | | nicotinamide N-methyltransferase | nicotinamide and other pyridines, structurally-related drugs and | |
| | | | xenobiotics\|Proteome | |
| AA505445 | Hs.300697:21 | IGHG3 | Constant region of heavy chain of IgG3 | 0.05 |
| | | immunoglobulin heavy constant gamma 3 (G3m | | |
| | | marker) | | |
| BE246649; | Hs.345728 | SOCS3 | suppression of IL-6 mediated signalling | 0.02 |
| NM_003955 | | STAT induced STAT-inhibitor 3; suppressor of | | |
| | | cytokine signalling 3 | | |
| M86849 | Hs.323733:62, Hs.300816:5 | GJB2 gap junction protein, beta 2, 26 kD (connexin | Hearing, connexon, plasma membrane, connexon channel, cell—cell | 0.00 |
| | | 26) | signalling, small molecule transport. Connexin 26; gap junction | |
| | | | protein expressed in various tissues including cochlea. | |
| AW963419 | Hs.155223:20 | STC2 stanniocalcin 2 | Peptide hormone, cell—cell signalling, glycopeptide hormone, | 0.00 |
| | | | nutritional response pathway, cell surface receptor linked signal | |
| | | | transduction. Stanniocalcin 2; may regulate metal ion homeostasis | |
| | | | and inhibits phosphate uptake. | |
| BE298665 | Hs.14846:132 | Homo sapiens mRNA; cDNA DKFZp564D016 | Function unknown | 0.00 |
| | | (from clone | | |
| AK000637 | Hs.46624:11 | HSPC043 HSPC043 protein | Function unknown | 0.00 |
| BE077546 | Hs.31447:27 | ESTs, Moderately similar to A46010 X-linked | Function unknown | 0.00 |
| | | retinopathy protein [H. sapiens] | | |
| T97307 | | gb: ye53h05.s1 Soares fetal liver spleen 1NFLS | Function unknown | 0.00 |
| | | Homo sapiens cDNA clone IMAGE: 121497 3′, | | |
| | | mRNA sequence. | | |
| R24601 | Hs.108300:46 | Homo sapiens adenylosuccinate synthetase | Function unknown | 0.00 |
| | | isozyme (ADSS) mRNA, complete cds | | |
| BE090176 | Hs.179902:95 | Interim-CDw92 antigen | choline transporter-like protein | 0.00 |
| AA393907 | Hs.97179:22 | ESTs | Function unknown | 0.00 |
| W28729 | Hs.236510:6 | Homo sapiens mRNA; cDNA DKFZp666D074 | Function unknown | 0.00 |
| | | (from clone DKFZp666D074) | | |
| BE313754 | Hs.13350:52 | Homo sapiens mRNA; cDNA DKFZp586D0918 | Function unknown | 0.01 |
| AW673081 | Hs.54828:9 | ESTs | Function unknown | 0.01 |
| AA356694 | Hs.94011:42, Hs.7744:2, | HCA4 Hepatocellular carcinoma-associated | Function unknown | 0.01 |
| | Hs.231043:1 | protein HCA4 | | |
| L08239 | Hs.5326:11 | MG61 Porcupine | amino acid system N transporter 2; | 0.01 |
| BE397649 | Hs.94109:40 | Homo sapiens cDNA FLJ34399 fis, clone | Function unknown | 0.01 |
| | | HCHON2001359 | | |
| NM_012317 | Hs.45231:36 | LDOC1 Leucine zipper, down-regulated in cancer 1 | Nucleus, negative control of cell proliferation. Nuclear protein; | 0.01 |
| | | | contains a leucine zipper-like motif | |
| NM_000947 | Hs.74519:20 | PRIM2A primase, polypeptide 2A (58 kD) | DNA primase, DNA replication, priming, alpha DNA | 0.01 |
| | | | polymerase: primase complex. Subunit of DNA primase polypeptide | |
| | | | 2A; part of the DNA polymerase alpha-primase complex | |
| AJ250562 | Hs.82749:133 | Homo sapiens partial TM4SF2 gene for | Function unknown | 0.01 |
| | | tetraspanin protein, exon 1 and joined CDS | | |
| AL040183 | Hs.123484:24, Hs.326906:1 | Homo sapiens mRNA; cDNA DKFZp686E1934 | Function unknown | 0.01 |
| | | (from clone DKFZp686E1934) | | |
| BE207573 | Hs.83321:32 | NMB neuromedin B | Peptide hormone, soluble fraction, signal transduction, cell—cell | 0.01 |
| | | | signalling. Precursor of neuromedin B, a C-terminally amidated | |
| | | | peptide hormone; similar to bombesin | |
| BE564162 | Hs.250820:45 | FLJ14827 hypothetical protein FLJ14827 | Function unknown | 0.01 |
| BE439580 | Hs.75498:40 | SCYA20 Small inducible cytokine subfamily A | Chemokine, chemotaxis, immune response, signal transduction, | 0.01 |
| | | (Cys—Cys), member 20 | extracellular space, cell—cell signalling, inflammatory response, | |
| | | | antimicrobial humoral response. Cytokine A20 (exodus); chemotactic | |
| | | | factor for lymphocytes, but not a chemotactic factor for monocytes | |
| AW067800 | Hs.155223:52 | STC2 stanniocalcin 2 | Peptide hormone, cell—cell signalling, glycopeptide hormone, | 0.01 |
| | | | nutritional response pathway, cell surface receptor linked signal | |
| | | | transduction. Stanniocalcin 2; may regulate metal ion homeostasis | |
| | | | and inhibits phosphate uptake. | |
| AA569756 | Hs.87803:10 | Homo sapiens cDNA FLJ30156 fis, clone | Function unknown | 0.01 |
| | | BRACE2000487 | | |
| AW138190 | Hs.180248:8 | ZNF124 zinc finger protein 124 (HZF-16) | DNA binding. C2H2 zinc-finger protein 124 | 0.01 |
| AF126245 | Hs.14791:48 | ACAD8 acyl-Coenzyme A dehydrogenase family, | Lipid metabolism, acyl-CoA dehydrogenase. Member of the acyl- | 0.01 |
| | | member 8 | Coenzyme A dehydrogenase family; alpha, beta-dehydrogenates | |
| | | | acyl-CoA esters | |
| L10343 | Hs.112341:96, Hs.1968:1 | Homo sapiens elafin precursor, gene, complete | elastase-specific inhibitor in bronchial secretions | 0.01 |
| | | cds | | |
| NM_002514 | Hs.235935:38 | NOV nephroblastoma overexpressed gene | Insulin-like growth factor receptor binding protein. Insulin-like growth | 0.01 |
| | | | factor binding protein; may play a role in nephrogenesis | |
| AI863735 | Hs.186755:3 | ESTs | Function unknown | 0.01 |
| NM_005397 | Hs.16426:160, Hs.248780:1 | PODXL podocalyxin-like | Integral plasma membrane protein. Transmembrane protein similar | 0.01 |
| | | | to rodent podocalyxins | |
| W26391 | Hs.301206:100 | KIF3B kinesin family member 3B | Plus-end kinesin, microtubule motor, anterograde axon cargo | 0.01 |
| | | | transport, plus-end-directed kinesin ATPase, determination of left- | |
| | | | right asymmetry. Similar to murine Kif3b; may have a role in | |
| | | | intracellular organelle transport, may act in left-right determination in | |
| | | | embryogenesis; are a microtubule-associated motor protein | |
| H15474 | Hs.132898:156 | FADS1 fatty acid desaturase 1 | C-5 sterol desaturase, fatty acid desaturation, integral membrane | 0.01 |
| | | | protein. Delta-5 desaturase; catalyzes production of polyenoic fatty | |
| | | | acids such as arachldonic acid | |
| U51166 | Hs.173824:106 | TDG Thymine-DNA glycosylase | DNA repair, nucleoplasm, damaged DNA binding, base-excision | 0.01 |
| | | | repair, G/T-mismatch-specific thymine-DNA glycosylase. Thymine- | |
| | | | DNA glycosylase; excises uracil and thymine from mispairs with | |
| | | | guanidine | |
| AA243499 | Hs.104800:23 | FLJ10134 hypothetical protein FLJ10134 | Highly similar to murine p19.5; are a membrane protein | 0.01 |
| AW408807 | Hs.34497:46 | FLJ22116 hypothetical protein FLJ22116 | Function unknown | 0.01 |
| AI738719 | Hs.198427:98 | HK2 Hexokinase 2 | Hexokinase, cell cycle control, glucose catabolism, glucose | 0.01 |
| | | | metabolism, mitochondrial outer membrane. Hexokinase II; converts | |
| | | | aldo- and keto-hexose sugars to the hexose-6-phosphate | |
| AB040888 | Hs.41793:110 | Homo sapiens mRNA for KIAA1455 protein, partial | Function unknown | 0.01 |
| | | cds | | |
| BE313077 | Hs.93135:40, Hs.228357:1 | Homo sapiens cDNA FLJ39971 fis, clone | Function unknown | 0.01 |
| | | SPLEN2028066 | | |
| AI677897 | Hs.76640:124 | RGC32 RGC32 protein | Cytoplasm, cell cycle regulator, regulation of CDK activity. Strongly | 0.01 |
| | | | similar to RGC-32 | |
| C14898 | Hs.192986:5 | ESTs | Function unknown | 0.01 |
| AI821730 | Hs.116524:7 | Homo sapiens cDNA FLJ35800 fis, clone | Function unknown | 0.01 |
| | | TESTI2005933 | | |
| AF007393 | Hs.177574:111 | PRKRIR protein-kinase, interferon-inducible | Stress response, protein binding, signal transduction, translational | 0.01 |
| | | double stranded RNA dependent inhibitor, | regulation, negative control of cell proliferation. Regulates interferon- | |
| | | repressor of (P58 repressor) | induced protein kinase PKR (PRKR) activity by binding and inhibiting | |
| | | | the PKR-regulator P58IPK (PRKRI) | |
| H65423 | Hs.17631:42 | DKFZP434E2135 hypothetical protein | Function unknown | 0.01 |
| | | DKFZp434E2135 | | |
| N46243 | Hs.110373:26 | ESTs, Highly similar to T42626 secreted leucine- | Function unknown | 0.01 |
| | | rich repeat-containing protein SLIT2 - mouse | | |
| | | (fragment) [M. musculus] | | |
| AA095971 | Hs.198793:56, Hs.309674:7 | Homo sapiens cDNA: FLJ22463 fis, clone | Function unknown | 0.01 |
| | | HRC10126 | | |
| U20350 | Hs.78913:33 | CX3CR1 chemokine (C—X3—C) receptor 1 | Virulence, chemotaxis, coreceptor, cell adhesion, plasma | 0.01 |
| | | | membrane, chemokine receptor, response to wounding, cellular | |
| | | | defense response, integral plasma membrane protein, G-protein | |
| | | | linked receptor protein signalling pathway. CX3C chemokine | |
| | | | receptor; G protein-coupled receptor, mediates leukocyte migration | |
| | | | and adhesion, binds the CX3C chemokine fractalkine and signals | |
| | | | through a pertussis toxin sensitive G-protein | |
| NM_005756 | Hs.184942:18 | GPR64 G protein-coupled receptor 64 | Spermatogenesis, G-protein linked receptor, integral plasma | 0.01 |
| | | | membrane protein, G-protein linked receptor protein signalling | |
| | | | pathway. Member of the G protein-coupled receptor family | |
| D19589 | Hs.13453:87 | FLJ14753 hypothetical protein FLJ14753 | Function unknown | 0.02 |
| AW957446 | Hs.301711:74 | ESTs | Function unknown | 0.02 |
| AW294647 | Hs.233634:40 | C20orf39 chromosome 20 open reading frame 39 | Function unknown | 0.02 |
| BE159718 | Hs.85335:46 | Homo sapiens, clone IMAGE: 4513159, mRNA | Function unknown | 0.02 |
| AI888490 | Hs.55902:22 | EDG3 endothelial differentiation, sphingolipid G- | Lipid binding, plasma membrane, inflammatory response, G-protein | 0.02 |
| | | protein-coupled receptor, 3 | linked receptor, embryogenesis and morphogenesis, integral plasma | |
| | | | membrane protein, positive control of cell proliferation, cytostolic | |
| | | | calcium ion concentration elevation, G-protein linked receptor protein | |
| | | | signalling pathway. Lysosphingolipid receptor, a G protein-coupled | |
| | | | receptor; activates calcium flux and serum response element driven | |
| | | | transcription | |
| AA022569 | Hs.29802:35, Hs.271785:1 | ESTs | Function unknown | 0.02 |
| BE147740 | Hs.104558:21 | ESTs, Moderately similar to hypothetical protein | Function unknown | 0.02 |
| | | FLJ20378 [Homo sapiens] | | |
| AI798863 | Hs.87191:8 | ESTs | Function unknown | 0.02 |
| BE464341 | Hs.21201:18 | Interim-DKFZP566B0846: nectin 3 | Low similarity to PVRL1; are a membrane glycoprotein; contains an | 0.02 |
| | | | immunoglobulin (Ig) domain | |
| AL080235 | Hs.35861:34, Hs.289068:1 | RIS1 Ras-induced senescence 1 | Rat brain specific binding protein | 0.02 |
| AI557212 | Hs.17132:102, Hs.330782:1 | ESTs | Function unknown | 0.02 |
| X75208 | Hs.2913:41 | EPHB3 EphB3 | Signal transduction, integral plasma membrane protein, | 0.02 |
| | | | transmembrane receptor protein tyrosine kinase. Eph-related | |
| | | | receptor tyrosine kinase B3 | |
| AA628980 | Hs.192371:3 | DSCR8 Down syndrome critical region protein | Melanoma-testis-associated protein 2 | 0.02 |
| | | DSCR8 | | |
| BE242587 | Hs.118651:39 | HHEX hematopoietically expressed homeobox | Nucleus, DNA binding, transcription factor, developmental | 0.02 |
| | | | processes, antimicrobial humoral response. Member of the | |
| | | | homeodomain family of DNA binding proteins; may regulate gene | |
| | | | expression, morphogenesis, and differentiation | |
| NM_005512 | Hs.151641:65 | GARP glycoprotein A repetitions predominant | Integral plasma membrane protein. Putative transmembrane cell | 0.02 |
| | | | surface protein; has an extracellular domain comprised largely of | |
| | | | leucine-rich repeats | |
| AW953853 | Hs.292833:19 | PAEP progestagen-associated endometrial protein | Developmental processes. Placental protein 14 (Glycodelin); | 0.02 |
| | | (placental protein 14, pregnancy-associated | member of lipocalin superfamily, highly similar to beta-lactoglobulins | |
| | | endometrial alpha-2-globulin, alpha uterine | | |
| | | protein) | | |
| AU076611 | Hs.154672:122 | MTHFD2 methylene tetrahydrofolate | Mitochondrion, electron transporter, methenyltetrahydrofolate | 0.02 |
| | | dehydrogenase (NAD dependent), | cyclohydrolase, methylenetetrahydrofolate dehydrogenase. NAD- | |
| | | methenyltetrahydrofolate cyclohydrolase | dependent methylene tetrahydrofolate dehydrogenase- | |
| | | | cyclohydrolase; may provide formyltetrahydrofolate for | |
| | | | formylmethionyl tRNA synthesis; involved in initiation of | |
| | | | mitochondrial protein synthesis | |
| AW968613 | Hs.79428:166 | BNIP3 BCL2/adenovirus E1B 19 kD-interacting | Anti-apoptosis, apoptosis inhibitor. Bcl2-related protein 3; binds | 0.02 |
| | | protein 3 | antiapoptotic viral E1B 19 kDa protein and cellular Bcl2 protein | |
| AL353944 | Hs.50115:14 | Homo sapiens mRNA; cDNA DKFZp761J1112 | Function unknown | 0.02 |
| | | (from clone DKFZp761J1112) | | |
| BE614149 | Hs.20814:29, Hs.306626:27 | LOC51072: C21orf19-like protein | Function unknown | 0.02 |
| AA292998 | Hs.163900:25 | ESTs | Highly similar to winged helix/forkhead transcription factor | 0.02 |
| H12912 | Hs.274691:138 | AK3 adenylate kinase 3 | Nucleobase, nucleoside, nucleotide and nucleic acid metabolism. | 0.02 |
| | | | Adenylate kinase 3; strongly similar to murine Ak4 | |
| AA188763 | Hs.36793:4 | SLC12A8 solute carrier family 12 | Solute carrier family 12 (potassium/chloride transporters), member 8 | 0.02 |
| | | (potassium/chloride transporters), member 8 | | |
| AK000596 | Hs.3618:56 | HPCAL1 hippocalcin-like 1 | Calcium-binding protein with similarity to hippocalin (human HPCA); | 0.02 |
| | | | expressed only in the brain. | |
| AI970797 | Hs.64859:16 | ESTs | Function unknown | 0.02 |
| AW519204 | Hs.40808:22 | ESTs | Function unknown | 0.02 |
| Z42387 | Hs.83883:114 | TMEPAI transmembrane, prostate androgen | Function unknown | 0.02 |
| | | induced RNA | | |
| AF145713 | Hs.61490:51 | SCHIP1 schwannomin-interacting protein 1 | Cytoplasm. Associates with the neurofibromatosis type 2 protein | 0.02 |
| | | | schwannomin (NF2); contains a coiled-coil domain | |
| AA972412 | Hs.13755:41 | FBXW2 f-box and WD-40 domain protein 2 | Protein modification, ubiquitin-protein ligase, proteolysis and | 0.02 |
| | | | peptidolysis, ubiquitin conjugating enzyme. F-box and WD-40 | |
| | | | domain protein 2; putative SCF ubiquitin ligase subunit involved in | |
| | | | protein degradation; contains a WD-40 domain and an F-box | |
| AK001564 | Hs.104222:139, Hs.296267:4 | Homo sapiens cDNA FLJ10702 fis, clone | Member of the ADP-ribosylation factor (ARF) family; putative GTP- | 0.02 |
| | | NT2RP3000759, weakly similar to ADP- | binding protein involved in protein trafficking | |
| | | RIBOSYLATION FACTOR | | |
| AW959861 | Hs.290943:28 | ESTs | Function unknown | 0.02 |
| BE313555 | Hs.7252:158 | RAI17 retinoic acid induced 17 | Function unknown | 0.02 |
| W25005 | Hs.24395:199 | zb67e02.r1 Soares_fetal_lung_NbHL19W Homo | Function unknown | 0.02 |
| | | sapiens cDNA clone IMAGE: 308666 5′, mRNA | | |
| | | sequence | | |
| AI193356 | Hs.160316:3 | ESTs | Function unknown | 0.02 |
| AF111106 | Hs.3382:223 | PPP4R1 Protein phosphatase 4, regulatory | Protein phosphatase | 0.02 |
| | | subunit 1 | | |
| AI130740 | Hs.6241:116 | PIK3R1 phosphoinositide-3-kinase, regulatory | A family of enzymes that phosphorylate the 3′-hydroxyl of | 0.02 |
| | | subunit, polypeptide 1 (p85 alpha) | phosphatidylinositol (Ptdins). | |
| AA985190 | Hs.246875:42 | FLJ20059 hypothetical protein FLJ20059 | Contains four Kelch motif domains | 0.02 |
| BE221880 | Hs.268555:144 | XRN2 5′-3′ exoribonuclease 2 | Nucleus, nuclease, recombination, RNA catabolism, RNA | 0.03 |
| | | | processing. 5′-3′ Exoribonuclease; similar to Schizosaccharomyces | |
| | | | pombe Dhp1p | |
| AF084545 | | Homo sapiens versican Vint isoform, mRNA, | Function unknown | 0.03 |
| | | partial cds | | |
| R26584 | Hs.267993:43 | TAPBP-R: TAP binding protein related | Has low similarity to TAPBP (Tapasin); contains two immunoglobulin | 0.03 |
| | | | (Ig) domains\|Proteome | |
| AW247380 | Hs.12124:116 | ELAC2 elaC homolog 2 (E. coli) | putative prostate cancer susceptibility protein | 0.03 |
| AA364261 | Hs.131365:7 | ESTs | Weakly similar to T31613 hypothetical protein Y50E8A.I - | 0.03 |
| | | | Caenorhabditis elegans [C. elegans] | |
| U25849 | Hs.75393:141 | ACP1 Human red cell-type low molecular weight | Acid phosphatase | 0.03 |
| | | acid phosphatase (ACP1) gene, exon 6 and 7, | | |
| | | complete cds | | |
| AF262992 | Hs.123159:14 | SPAG4 Sperm associated antigen 4 | Spermatogenesis, structural protein. Sperm associated antigen 4; | 0.03 |
| | | | predicted ortholog of rat SPAG4, which interacts with rat ODF27, the | |
| | | | 27 kDa outer dense fiber protein of elongating spermatids | |
| AW342140 | Hs.182545:1 | ESTs, Weakly similar to POL2_MOUSE | Function unknown | 0.03 |
| | | Retrovirus-related POL polyprotein | | |
| AL133572 | Hs.199009:58 | PCCX2 protein containing CXXC domain 2 | DNA-binding protein with PHD finger and CXXC domain, is regulated | 0.03 |
| | | | by proteolysis. | |
| AI497778 | Hs.20509:4 | HBXAP Hepatitis B virus x associated protein | Weakly similar to Drosophila CG8677 | 0.03 |
| AI745379 | Hs.42911:31 | TAF13 TAF13 RNA polymerase II, TATA box | TFIID complex, protein binding, transcription factor, general RNA | 0.03 |
| | | binding protein (TBP)-associated factor, 18 kD | polymerase II transcription factor. TBP-associated factor K; | |
| | | | component of TFIID complexes containing TAFII30 (TAF2H) | |
| U51712 | Hs.13775:135 | LAGY: lung cancer-associated Y protein | The protein encoded by this gene is a lung cancer associated | 0.03 |
| | | | protein. The function of the protein is not known. Multiple | |
| | | | alternatively spliced transcript variants have been described for this | |
| | | | gene but some of their full length sequence has not been | |
| | | | determined. | |
| AW375974 | Hs.156704:4 | ESTs | Function unknown | 0.03 |
| AF251237 | Hs.112208:16 | GAGED2 G antigen, family D, 2 | GAGE genes are expressed in a variety of tumors and in some fetal | 0.03 |
| | | | and reproductive tissues. This gene is strongly expressed in Ewing's | |
| | | | sarcoma, alveolar rhabdomyosarcoma and normal testis. The protein | |
| | | | encoded by this gene contains a nuclear localization signal and | |
| | | | shares a sequence similarity with other GAGE/PAGE proteins. | |
| | | | Because of the expression pattern and the sequence similarity, this | |
| | | | protein also belongs to a family of CT (cancer-testis) antigens. | |
| NM_000636 | | Homo sapiens superoxide dismutase 2, | Mitochondrion, oxidative stress response, manganese superoxide | 0.02 |
| | | mitochondrial (SOD2), mRNA. expression) | dismutase. Manganese superoxide dismutase; intramitochondrial | |
| | | (RFX2), mRNA. | free radical scavenging enzyme; has strong similarity to murine | |
| | | | Sod2. | |
| AA130986 | Hs.271627:1 | ESTs | Function unknown | 0.01 |
| AA216363 | Hs.262958:48, Hs.327737:2 | DKFZP434B044 hypothetical protein | Function unknown | 0.01 |
| | | DKFZp434B044 | | |
| AA628980 | Hs.192371:3 | DSCR8 down syndrome critical region protein | Function unknown | 0.00 |
| | | DSCR8 | | |
| AA811657 | Hs.220913:9 | Homo sapiens cDNA FLJ40827 fis, clone | Function unknown | 0.02 |
| | | TRACH2011500 | | |
| AA897108 | | gb: am08a06.s1 Soares_NFL_T_GBC_S1 Home | Function unknown | 0.01 |
| | | sapiens cDNA clone 3′, mRNA sequence | | |
| AB040888 | Hs.41793:110 | Homo sapiens mRNA for KIAA1455 protein, partial | Function unknown | 0.02 |
| | | cds | | |
| AF212225 | Hs.283693:104 | Homo sapiens BM022 mRNA, complete cds | Function unknown | 0.02 |
| AI089575 | Hs.9071:52 | ESTs | Function unknown | 0.02 |
| AI282028 | Hs.25205:10 | ESTs | Function unknown | 0.02 |
| AI368826 | Hs.30654:15 | FLJ10849: hypothetical protein FLJ10849 | Moderately similar to members of the septin family | 0.02 |
| AI718702 | Hs.308026:11, Hs.194490:6 | HLA-DRB3 major histocompatibility complex, class | Signal transduction, integral plasma membrane protein, class II | 0.02 |
| | | II, DR beta 5 | major histocompatibility complex antigen. Beta 3 chain of HLA-DR; | |
| | | | subunit of MHC class II molecule, complex binds peptides and | |
| | | | presents them to CD4+ T lymphocytes | |
| AI827248 | Hs.224398:3 | Homo sapiens cDNA FLJ11469 fis, clone | Function unknown | 0.01 |
| | | HEMBA1001658 | | |
| AK002039 | Hs.26243:38 | MRVI1 murine retrovirus integration site 1 homolog | Oncogenesis, tumor suppressor, endoplasmic reticulum membrane. | 0.02 |
| | | | Similar to human MLRP; may act as a tumor suppressor | |
| AL109791 | Hs.241559:3 | Homo sapiens mRNA full length insert cDNA clone | Function unknown | 0.00 |
| | | EUROIMAGE 151432 | | |
| AW090198 | Hs.4779:29 | LOC127829: hypothetical protein BC015408 | Function unknown | 0.01 |
| AW296454 | Hs.24743:92 | FLJ20171: hypothetical protein FLJ2017 | Contains three RNA recognition motifs (RRM, RBD, or RNP) | 0.02 |
| AW445034 | Hs.256578:4 | ESTs | Function unknown | 0.00 |
| AW452948 | Hs.257631:3 | ESTs | Function unknown | 0.01 |
| AW470411 | Hs.288433:27 | HNT: neurotrimin | Cell adhesion, neuronal cell recognition, integral plasma membrane | 0.02 |
| | | | protein. Neurotrimin; may function as a GPI-anchored neural cell | |
| | | | adhesion molecule; member of the immunoglobulin superfamily | |
| AW885727 | Hs.301570:22 | FST follistatin | Developmental processes. Follistatin; inhibits the release of follicle- | 0.01 |
| | | | stimulating hormone (FSH) | |
| AW970859 | Hs.313503:4 | ESTs | Function unknown | 0.02 |
| AW979189 | Hs.283367:3 | ESTs | Function unknown | 0.01 |
| BE165866 | Hs.83623:66 | Human XIST, coding sequence “a” mRNA (locus | XIST mRNA | 0.01 |
| | | DXS399E) | | |
| BE175582 | | gb: RC5-HT0580-100500-022-C01 HT0580 Homo | Function unknown | 0.01 |
| | | sapiens cDNA, mRNA sequence | | |
| BE242587 | Hs.118651:39 | HHEX hematopoletically expressed homeobox | Nucleus, DNA binding, transcription factor, developmental | 0.01 |
| | | | processes, antimicrobial humoral response. Member of the | |
| | | | homeodomain family of DNA binding proteins; may regulate gene | |
| | | | expression, morphogenesis, and differentiation\| | |
| BE271927 | Hs.87385:31, Hs.307940:4 | LOC115416: hypothetical protein BC012331 | Function unknown | 0.01 |
| BE439580 | Hs.75498:40 | SCYA20 small inducible cytokine subfamily A | Chemokine, chemotaxis, immune response, signal transduction, | 0.02 |
| | | (Cys—Cys), member 20 | extracellular space, cell—cell signalling, inflammatory response, | |
| | | | antimicrobial humoral response. Cytokine A20 (exodus); chemotactic | |
| | | | factor for lymphocytes, but not a chemotactic factor for monocytes | |
| BE464016 | Hs.238956:35 | Homo sapiens cDNA FLJ37793 fis, clone | Function unknown | 0.02 |
| | | BRHIP3000473 | | |
| D63216 | Hs.153684:137 | FRZB frizzled-related protein | Membrane, extracellular, skeletal development. Frizzled-related | 0.02 |
| | | | protein; similar to frizzled family of receptors | |
| F34856 | Hs.292457:120 | Homo sapiens, clone MGC: 16362 | Function unknown | 0.02 |
| | | IMAGE: 3927795, mRNA, complete cds | | |
| M83822 | Hs.62354:112 | LRBA LPS-responsive vesicle trafficking, beach | May mediate protein-protein interactions; contains two WD domains | 0.02 |
| | | and anchor containing | (WD-40 repeats) and a beige/BEACH domain\|Proteome | |
| N33937 | Hs.10336:6 | ESTs | Function unknown | 0.01 |
| N49068 | Hs.93966:4 | ESTs | Function unknown | 0.01 |
| N51357 | Hs.260855:62 | NSE1: NSE1 | Function unknown | 0.02 |
| N80486 | Hs.39911:17 | Homo sapiens mRNA for FLJ00089 protein, partial | Function unknown | 0.02 |
| | | cds | | |
| NM_000954 | Hs.8272:265, Hs.332355:1 | PTGDS prostaglandin D2 synthase (21 kD, brain) | Membrane, prostaglandin-D synthase. Glutathione-independent | 0.02 |
| | | | prostaglandin D2 synthase; membrane associated, catalyzes | |
| | | | synthesis of prostaglandin D; member of the lipocalin family of | |
| | | | transporters | |
| NM_005756 | Hs.184942:18 | GPR64 G protein-coupled receptor 64 | Spermatogenesis, G-protein linked receptor, integral plasma | 0.02 |
| | | | membrane protein, G-protein linked receptor protein signalling | |
| | | | pathway. Member of the G protein-coupled receptor family | |
| NM_016652 | Hs.268281:61 | CRNKL1 Cm, crooked neck-like 1 (Drosophila) | Function unknown | 0.02 |
| R26584 | Hs.267993:43 | TAPBP-R: TAP binding protein related | Has low similarity to TAPBP (Tapasin); contains two immunoglobulin | 0.01 |
| | | | (lg) domains | |
| R31178 | Hs.287820:6 | FN1 fibronectin 1 | Cell adhesion, cell motility, cell adhesion, soluble fraction, signal | 0.02 |
| | | | transduction, extracellular matrix, extracellular space. Fibronectin 1; | |
| | | | member of family of proteins found in plasma and extracellular | |
| | | | matrix | |
| W05391 | Hs.83623:8 | Homo sapiens cDNA FLJ30298 fis, clone | Function unknown | 0.02 |
| | | BRACE2003172 | | |
| W25005 | Hs.24395:199 | zb67e02.r1 Soares_fetal_lung_NbHL19W Homo | Function unknown | 0.01 |
| | | sapiens cDNA clone IMAGE: 308666 5′, mRNA | | |
| | | sequence | | |
| W45393 | Hs.55888:15 | ATF7 activating transcription factor 7 | Transcription factor. Leucine zipper DNA-binding protein; recognizes | 0.02 |
| | | | a cAMP response element (CRE), involved in the regulation of | |
| | | | adenovirus Ela-responsive and cellular cAMP-inducible promoters | |
| W68815 | Hs.301885:20 | Homo sapiens cDNA FLJ33794 fis, clone | Function unknown | 0.01 |
| | | CTONG1000009 | | |
| X65965 | | H. sapiens SOD-2 gene for manganese superoxide | Mitochondrion, oxidative stress response, manganese superoxide | 0.01 |
| | | dismutase | dismutase. Manganese superoxide dismutase; intramitochondrial | |
| | | | free radical scavenging enzyme; has strong similarity to murine | |
| | | | Sod2. | |
| X76732 | Hs.3164:58 | NUCB2 nucleobindin 2 | Cytosol, DNA binding, plasma membrane, calcium binding, | 0.02 |
| | | | extracellular space. Nucleobindin 2; may bind DNA and calcium; has | |
| | | | DNA-binding and EF-hand domains, and a leucine-zipper | |
| Z45051 | Hs.22920:25 | C20orf103 chromosome 20 open reading frame | Low similarity to a region of murine Lamp1\|Proteome | 0.02 |
| | | 103 | | |

c. downregulated genes

| | | | | |
|---|---|---|---|---|
| NM_022117 | Hs.136164:23 | SE20-4, cutaneous T-cell lymphoma-associated | Cutaneous T-cell lymphoma-associated tumor antigen se20-4se20- | 0 |
| | | tumor antigen se20-4se20-4 | 4; differentially expressed nucleolar TGF-beta1 target protein | |
| | | | (DENTT); also known as CDA1 | |
| NM_005460 | Hs.24948:32, | SNCAIP, synuclein, alpha interacting protein | Cytoplasm, pathogenesis, protein binding. Synphilin-1; promotes | 0 |
| | Hs.300445:4 | (synphilin) | formation of cytosolic inclusions in neurons (SNCAIP). Synuclein | |
| | | | alpha interacting protein contains several protein-protein interaction | |
| | | | domains and interacts with alpha synuclein in neurons. Mutations of | |
| | | | SNCAIP have been linked to Parkinson disease. | |
| NM_002387 | Hs.1345:5 | MCC, mutated in colorectal cancers | Receptor, signal transduction, tumor suppressor. Similar to the G | 0 |
| | | | protein-coupled m3 muscarinic acetylcholine receptor. MCC is a | |
| | | | candidate for the putative colorectal tumor suppressor gene. The | |
| | | | MCC gene product are involved in early stages of colorectal | |
| | | | neoplasia in both sporadic and familial tumors. | |
| AI745249 | Hs.23650:30 | Homo sapiens, clone MGC: 9889 IMAGE: 3868330 | Function unknown | 0.0009 |
| AI694200 | Hs.356620, | ESTs | Function unknown | 0.0442 |
| | Hs.227913:11 | | | |

TABLE 2
Genes having modified expression in serous ovarian cancer relative to normal ovarian tissue

| Accession number | UniGene Mapping | Gene symbol and title | Putative Function | Ratio |
|---|---|---|---|---|
| M25809 | Hs.64173 | ATP6V1B1, ATPase, H+ transporting, lysosomal | Subunit B1 (beta subunit) of a vacuolar-type H+-ATPase 1; apical proton | 1062.30 |
| | | 56/58 kD, V1 subunit B, isoform 1 (Renal tubular | pump that mediates distal nephron acid secretion | |
| | | acidosis with deafness) | | |
| AW959311 | Hs.172012 | DKFZP434J037: hypothetical protein DKFZp434J037 | Function unknown | 227.83 |
| H16423 | Hs.82685 | Homo sapiens mRNA; cDNA DKFZp313F0317 (from | Function unknown | 74.54 |
| | | clone DKFZp313F0317) | | |
| AI733848 | Hs.71935 | ZNF339, zinc finger protein 339 | Zinc finger protein | 55.13 |
| AW055308 | Hs.31803 | NAC1, transcriptional repressor NAC1 | Function unknown | 52.63 |
| AF034102 | Hs.32951 | SLC29A2, solute carrier family 29 (nucleoside | Nitrobenzylthioinosine-insensitive equilibrative nucleoside transporter 2; | 44.34 |
| | | transporters), member 2 | may act in the uptake of purine and pyrimidine nucleosides | |
| AI791905 | Hs.95549 | FLJ20273: RNA-binding protein | Contains four RNA recognition motifs (RRM, RBD, or RNP) | 43.21 |
| AW296454 | Hs.24743 | FLJ20171: hypothetical protein FLJ20171 | Contains three RNA recognition motifs (RRM, RBD, or RNP) | 38.91 |
| Z43989 | Hs.82141 | Human clone 23612 mRNA sequence | Function unknown | 37.89 |
| AL043980 | Hs.7886 | PELI1, pellino homolog 1 (Drosophila) | Pellino protein | 35.20 |
| BE514982 | Hs.38991 | S100A2, S100 calcium binding protein A2 | S100 calcium-binding protein A2; Interacts with target proteins to link | 34.53 |
| | | | extracellular stimuli and cellular responses; member of the S100 | |
| | | | tissue/cell specific Ca2+-binding protein family | |
| | | Target Exon | Function unknown | 34.02 |
| AI811807 | Hs.108646 | Homo sapiens cDNA FLJ12534 fis, clone | Function unknown | 32.34 |
| | | NT2RM4000244 | | |
| U90441 | Hs.3622 | P4HA2, procollagen-proline, 2-oxoglutarate 4- | Alpha 2 subunit of prolyl 4-hydroxylase; catalyzes the formation of 4- | 32.24 |
| | | dioxygenase (proline 4-hydroxylase), alpha | hydroxyproline in collagens | |
| T98226 | Hs.171952 | OCLN, occludin | This gene encodes an integral membrane protein which is located at | 31.56 |
| | | | tight junctions. This protein are involved in the formation and | |
| | | | maintenance of the tight junction. | |
| R35343 | Hs.24968 | Human DNA sequence from clone RP1-233G16 on | | 31.22 |
| | | chromosome Xq22.1-23. Contains the 5′ part of a | | |
| | | novel gene, ESTs, STSs, GSSs and a putative CpG | | |
| | | island | | |
| BE247295 | Hs.78452 | SLC20A1, solute carrier family 20 (phosphate | Sodium-dependent phosphate symporter; acts as a cell-surface receptor | 30.16 |
| | | transporter), member 1 | for gibbon ape leukemia virus | |
| AB037734 | Hs.4993 | PCDH19, protocadherin | Protocadherin | 29.90 |
| | | C5000394*: gi\|12737280\|ref\|Xp_006682.2\| keratin 18 | Function unknown | 29.30 |
| | | [Homo sapiens]\|\|6633 | | |
| AF212223 | Hs.25010 | Homo sapiens BM025 mRNA, complete cds | Function unknown | 28.85 |
| AA902656 | Hs.21943 | NIF3L1, NIF3 (Ngg1 interacting factor 3, S. pombe | Amyotrophic lateral sclerosis 2 (juvenile) chromosome region, candidate 1 | 27.73 |
| | | homolog)-like 1 | | |
| X14008 | Hs.234734 | Human lysozyme gene (EC 3.2.1.17) | Lysozyme | 27.66 |
| AA570256 | | LOC116238: hypothetical protein BC014072 | Function unknown | 27.52 |
| AA137152 | Hs.286049 | PSA, phosphoserine aminotransferase | The protein encoded by this gene is likely a phosphoserine | 25.57 |
| | | | aminotransferase, based on similarity to proteins in mouse, rabbit, and | |
| | | | Drosophila. Alternative splicing of this gene results in two transcript | |
| | | | variants encoding different isoforms. | |
| BE621807 | | TM4SF1, transmembrane 4 superfamily member 1 | L6 antigen; member of the transmembrane 4 superfamily (TM4SF) | 25.40 |
| AB041036 | Hs.57771 | KLK11, kallikrein 11 | Trypsin-like serine protease; has serine protease activity | 25.05 |
| F13386 | Hs.7888 | Homo sapiens clone 23736 mRNA sequence | Function unknown | 22.50 |
| AA158177 | Hs.118722 | FUT8, fucosyltransferase 8 (alpha (1,6) | N-linked glycosylation, oligosaccharide biosynthesis, glycoprotein 6- | 21.90 |
| | | fucosyltransferase) | alpha-L-fucosyltransferase. Alpha(1,6)fucosyltransferase (GDP-L-Fuc:N- | |
| | | | acetyl-beta-D-glucosaminide:alpha1-6 fucosyltransferase); transfers | |
| | | | fucose to N-linked type complex glycopeptides from GDP-Fuc; functions | |
| | | | in asparagine-linked glycoprotein oligosaccharide synthesis | |
| BE267045 | Hs.75064 | TBCC, tubulin-specific chaperone c | Tubulin-specific chaperone c; cofactor in the folding pathway of beta- | 21.49 |
| | | | tubulin, mediates the release of beta-tubulin polypeptides committed to | |
| | | | the native state | |
| | | NM_005936: Homo sapiens myeloid/lymphoid or | Function unknown | 20.46 |
| | | mixed-lineage leukemia (trithorax (Drosophila) | | |
| | | homolog); translocated to, 4 (MLLT4), mRNA. | | |
| AA150864 | Hs.790 | MGST1, microsomal glutathione S-transferase 1 | Microsome, glutathione transferase. Microsomal glutathione S- | 20.35 |
| | | | transferase; catalyzes the conjugation of glutathione to electrophilic | |
| | | | compounds; member of a family of detoxication enzymes. | |
| AW955632 | Hs.66666 | EST367702 MAGE resequences, MAGD Homo | Function unknown | 20.26 |
| | | sapiens cDNA, mRNA sequence | | |
| AW837046 | Hs.6527 | QV1-LT0037-150200-069-e09 LT0037 Homo sapiens | Function unknown | 19.60 |
| | | cDNA, mRNA sequence | | |
| AA286887 | Hs.24724 | MFHAS1, malignant fibrous histiocytoma amplified | The primary structure of its product includes an ATP/GTP-binding site, | 19.16 |
| | | sequence 1 | three leucine zipper domains, and a leucine-rich tandem repeat, which | |
| | | | are structural or functional elements for interactions among proteins | |
| | | | related to the cell cycle, and which suggest that overexpression might be | |
| | | | oncogenic with respect to MFH. | |
| AW401864 | Hs.18720 | PDCD8: programmed cell death 8 (apoptosis-inducing | Mitochondrial apoptosis-inducing factor; flavoprotein inducing chromatin | 19.01 |
| | | factor) | condensation and DNA fragmentation | |
| AA196241 | Hs.73980 | zp98f03.r1 Stratagene muscle 937209 Homo sapiens | Function unknown | 18.82 |
| | | cDNA clone IMAGE: 628253 5′ similar to gb: M19309 | | |
| | | TROPONIN T, SLOW SKELETAL MUSCLE | | |
| | | ISOFORMS (HUMAN);, mRNA sequence | | |
| NM_004998 | Hs.82251 | MYO1E, myosin IE | Highly similar to class I myosin; may bind proline-rich peptides; contains | 18.62 |
| | | | an Src homology 3 (SH3) and myosin head domain (motor domain) | |
| AW873704 | Hs.320831 | C20orf72: chromosome 20 open reading frame 72 | Function unknown | 18.19 |
| AW361666 | Hs.49500 | KIAA0746: KIAA0746 protein | Function unknown | 18.05 |
| BE174595 | Hs.366 | PTS, 6-pyruvoyltetrahydropterin synthase | 6-Pyruvoyltetrahydropterin synthase; synthesizes tetrahydrobiopterin, | 17.28 |
| | | | activity requires sepiapterin reductase, Mg2+, and NADPH | |
| M31669 | Hs.1735 | Human inhibin beta-B-subunit gene, exon 2, and | Function unknown | 16.24 |
| | | complete cds | | |
| AK001714 | Hs.95744 | FLJ10852, hypothetical protein similar to ankyrin | Are involved in protein-protein interactions; has five ankyrin repeats and | 16.09 |
| | | repeat-containing priotein AKR1 | a DHHC-type zinc finger or NEW1 domain | |
| AU076517 | Hs.184276 | AU076517 Sugano cDNA library Homo sapiens cDNA | Function unknown | 16.05 |
| | | clone ColF3365 similar to 5′-end region of Homo | | |
| | | sapiens ezrin-radixin-moesin binding phosphoprotein- | | |
| | | 50 mRNA, mRNA sequence | | |
| NM_006456 | Hs.288215 | STHM, sialyltransferase | Low similarity to beta-galactosidase a-2,3-sialytransferase SIAT4B; | 15.93 |
| | | | member of the sialyltransferase family | |
| BE148235 | Hs.193063 | Homo sapiens cDNA FLJ14201 fis, clone | Function unknown | 15.91 |
| | | NT2RP3002955 | | |
| AV653729 | Hs.8185 | SQRDL: sulfide dehydrogenase like (yeast) | Sulfide dehydrogenase like | 15.35 |
| AL119671 | Hs.1420 | FGFR3, fibroblast growth factor receptor 3 | Fibroblast growth factor receptor 3; receptor tyrosine kinase that binds | 14.62 |
| | | (achondroplasia, thanatophoric dwarfism) | acidic and basic FGF | |
| AA393071 | Hs.182579 | LAP3, leucine aminopeptidase | Leucine aminopeptidase | 14.60 |
| AL048753 | Hs.303649 | CCL2, chemokine (C—C motif) ligand 2 | Cytokine A 2; chemotactic factor for monocytes | 14.37 |
| AI868872 | Hs.282804 | CP, ceruloplasmin (ferroxidase) | Ceruloplasmin; ferrous oxidase, binds copper in plasma and maintains | 14.07 |
| | | | iron homeostasis | |
| NM_004419 | Hs.2128 | DUSP5, dual specificity phosphatase 5 | Mitogen inducible dual specificity protein phosphatase 5; | 14.05 |
| | | | dephosphorylates extracellular signal-regulated kinase | |
| AW969587 | Hs.86366 | EST381664 MAGE resequences, MAGK Homo | Function unknown | 13.75 |
| | | sapiens cDNA, mRNA sequence | | |
| AW161449 | Hs.72290 | WNT7A, wingless-type MMTV integration site family, | Very strongly similar to murine Wnt7a; may have a role in limb | 13.48 |
| | | member 7A | development and sexual dimorphism; member of the Wnt family of cell | |
| | | | signalling proteins | |
| BE409838 | Hs.194657 | CDH1, cadherin 1, type 1, E-cadherin (epithelial) | E-cadherin (uvomorulin); Ca2+-dependent glycoprotein, mediates cell- | 12.92 |
| | | | cell Interactions in epithelial cells | |
| BE540274 | Hs.239 | FOXM1, forkhead box M1 | Cell-cycle regulated HNF-3/fork head; a transcriptional regulator | 12.86 |
| AF022375 | Hs.73793 | VEGF, vascular endothelial growth factor | Vascular endothelial growth factor; induces endothelial cell proliferation | 12.79 |
| | | | and vascular permeability | |
| AW369278 | Hs.23412 | FLJ20160: hypothetical protein FLJ20160 | Function unknown | 12.73 |
| AF147204 | Hs.89414 | CXCR4, chemokine (C—X—C motif), receptor 4 (fusin) | CXC chemokine receptor (fusin); G protein-coupled receptor binds CXC | 12.56 |
| | | | cytokines, mediates intracellular calcium flux | |
| BE242818 | Hs.311609 | DDX39, DEAD/H (Asp-Glu-Ala-Asp/His) box | Strongly similar to human D6S81E; member of the DEAD/H box ATP- | 12.43 |
| | | polypeptide 39 | dependent RNA helicase family | |
| NM_014791 | Hs.184339 | MELK, maternal embryonic leucine zipper kinase | Leucine zipper kinase | 12.25 |
| U38847 | Hs.151518 | TARBP1, TAR (HIV) RNA binding protein 1 | Binds to the HIV-1 TAR RNA regulatory element, may function alone or | 12.22 |
| | | | with HIV-1 Tat to disengage RNA polymerase II during transcriptional | |
| | | | elongation; has a leucine zipper | |
| AW953575 | Hs.303125 | EST365645 MAGE resequences, MAGC Homo | Function unknown | 12.21 |
| | | sapiens cDNA, mRNA sequence | | |
| AI949095 | Hs.67776 | ESTs, Weakly similar to T22341 hypothetical protein | Homo sapiens, clone IMAGE: 5455669, mRNA, partial cds | 12.08 |
| | | F47B8.5 - Caenorhabditis elegans [C. elegans] | | |
| BE274530 | Hs.273333 | FLJ10986, hypothetical protein FLJ10986 | Member of the FGGY carbohydrate kinase family | 11.75 |
| AB020676 | Hs.21543 | KIAA0869 protein | Function unknown | 11.73 |
| | | Target Exon | Function unknown | 11.69 |
| H48299 | Hs.26126:33 | CLDN10, claudin 10 | Cell adhesion, integral plasma membrane protein, tight junction. | 11.67 |
| T34530 | Hs.4210 | Homo sapiens cDNA FLJ13069 fis, clone | Function unknown | 11.50 |
| | | NT2RP3001752 | | |
| NM_022454 | Hs.97984 | SOX17, SRY (sex determining region Y)-box 17 | SRY-related HMG-box transcription factor SOX17 | 11.42 |
| AA737033 | Hs.7155 | Homo sapiens, clone IMAGE: 4428577, mRNA, partial | Function unknown | 10.79 |
| | | cds | | |
| AA433988 | Hs.98502:8 | MUC16, mucin 16, CA125 | Mucin 16, Alias CA125 ovarian cancer antigen | 10.52 |
| H91282 | Hs.286232 | Homo sapiens cDNA: FLJ23190 fis, clone LNG12190 | Function unknown | 10.50 |
| AW005054 | Hs.47883 | LOC57118: CamKI-like protein kinase | CamKI-like protein kinase; granulocyte-specific protein kinase that | 10.49 |
| | | | activates ERK/MAP kinase activity; similar to Ca(2+)-calmodulin- | |
| | | | dependent kinase I (CamKI) | |
| X69699 | Hs.73149 | PAX8, paired box gene 8 | Member of the paired domain family of nuclear transcription factors; are | 10.39 |
| | | | involved in the ribosome assembly, required for normal thyroid | |
| | | | development | |
| AW382987 | Hs.88474:42 | Homo sapiens cDNA, mRNA sequence | Function unknown | 10.21 |
| AW957446 | Hs.301711 | Homo sapiens, clone MGC: 23936 IMAGE: 3838595, | Function unknown | 10.12 |
| | | mRNA, complete cds | | |
| AA361562 | Hs.178761 | POH1: 26S proteasome-associated pad1 homolog | Ubiquitin-dependent protein degradation | 10.01 |
| AA834626 | | RAD54L, RAD54 (S. cerevisiae)-like | Has likely roles in mitotic and meiotic DNA recombination and repair; | 9.85 |
| | | | member of SNF2/SWI2 family of DNA-dependent ATPases | |
| AI878927 | Hs.79284 | MEST, mesoderm specific transcript (mouse) homolog | Mesoderm specific protein; member of the alpha/beta hydrolase fold | 9.83 |
| | | | family | |
| AW074266 | Hs.23071 | LOC85439: stonin 2 | Stonin 2 | 9.74 |
| NM_000947 | Hs.74519 | PRIM2A, primase, polypeptide 2A (58 kD) | Subunit of DNA primase polypeptide 2A; part of the DNA polymerase | 9.72 |
| | | | alpha-primase complex | |
| NM_006187 | Hs.56009 | OAS3, 2′-5′-oligoadenylate synthetase 3 (100 kD) | Member of the 2′5′-oligoadenylate synthetase family | 9.68 |
| AW276858 | Hs.81256 | S100A4, S100 calcium binding protein A4 (calcium | Calcyclin (metastasis-associated) (S100 calcium-binding protein A4); | 9.66 |
| | | protein, calvasculin, metastasin, murine placental | interacts with targets to link extracellular stimull and cellular responses; | |
| | | homolog) | member of the S100 family of tissue-specific calcium-binding proteins | |
| T18997 | Hs.180372 | LOC139231: hypothetical protein BC016683 | Function unknown | 9.49 |
| AA262294 | Hs.180383 | DUSP6, dual specificity phosphatase 6 | Dual specificity protein phosphatase 6; selectively dephosphorylates and | 9.48 |
| | | | inactivates MAP kinase | |
| AA220238 | Hs.94986 | RPP38: ribonuclease P (38 kD) | Nucleus, ribonuclease P. Subunit p38 of ribonuclease P | 9.41 |
| | | | ribonucleoprotein; processes 5′ ends of precursor tRNAs | |
| AW505308 | Hs.75812 | PCK2, phosphoenolpyruvate carboxykinase 2 | Phosphoenolpyruvate carboxykinase 2; forms phosphoenolpyruvate by | 9.38 |
| | | (mitochondrial) | decarboxylation of oxaloacetate at the rate-limiting step of | |
| | | | gluconeogenesis | |
| AI186431 | Hs.296638 | PLAB: prostate differentiation factor | Macrophage inhibitory cytokine; member of a subgroup of the TGF-beta | 9.12 |
| | | | superfamily | |
| AI095718 | Hs.135015 | Homo sapiens cDNA FLJ40906 fis, clone | Function unknown | 9.04 |
| | | UTERU2004698, highly similar to Mus musculus | | |
| | | mRNA for thrombospondin type 1 domain | | |
| W70171 | Hs.75939 | UMPK, uridine monophosphate kinase | The protein encoded by this gene catalyzes the phosphorylation of | 8.97 |
| | | | uridine monophosphate to uridine diphosphate. This is the first step in | |
| | | | the production of the pyrimidine nucleoside triphosphates required for | |
| | | | RNA and DNA synthesis. In addition, an allele of this gene may play a | |
| | | | role in mediating nonhumoral immunity to Hemophilus influenzae type B. | |
| AI580935 | Hs.105698 | Homo sapiens cDNA FLJ31553 fis, clone | Function unknown | 8.90 |
| | | NT2RI2001178 | | |
| AB040914 | Hs.278628 | ShrmL: Shroom-ralated protein | Shroom-related protein | 8.87 |
| AU076611 | Hs.154672 | MTHFD2, methylene tetrahydrofolate dehydrogenase | NAD-dependent methylene tetrahydrofolate dehydrogenase- | 8.71 |
| | | (NAD+ dependent), methenyltetrahydrofolate | cyclohydrolase; may provide formyltetrahydrofolate for formylmethionyl | |
| | | cyclohydrolase | tRNA synthesis; Involved in initiation of mitochondrial protein synthesis | |
| AI089660 | Hs.323401 | LOC84661: dpy-30-like protein | dpy-30-like protein | 8.71 |
| D13666 | Hs.136348:228, Hs.80988:2 | OSF-2: osteoblast specific factor 2 (fasciclin I-like) | Cell adhesion, skeletal development. Putative bone adhesion protein; | 8.64 |
| | | | similar to the insect protein fasciclin I | |
| AI798863 | Hs.87191 | ESTs | Function unknown | 8.52 |
| U78093 | Hs.15154 | SRPX, sushi-repeat-containing protein, X chromosome | Putative membrane protein with short consensus repeat (sushi) domains | 8.51 |
| AI669760 | Hs.188881 | ESTs | Function unknown | 8.37 |
| AI375726 | Hs.279918 | MGC2198: hypothetical protein MGC2198 | Function unknown | 8.37 |
| AW271106 | Hs.133294 | ESTs | Function unknown | 8.30 |
| AK001782 | Hs.15093 | HSPC195: hypothetical protein HSPC195 | Function unknown | 8.18 |
| AF019226 | Hs.8036 | RAB3D, RAB3D, member RAS oncogene family | GTP-binding protein; are involved in vesicle transport; member of the | 7.94 |
| | | | RAB family of small GTPases | |
| AW968343 | Hs.24255 | LOC150696: prominin-related protein | Prominin-related protein | 7.90 |
| AF111856 | Hs.105039 | SLC34A2, solute carrier family 34 (sodium phosphate), | Sodium-dependent phosphate transporter; member of the renal type II | 7.87 |
| | | member 2 | co-transporter family | |
| AA863360 | Hs.26040 | Homo sapiens, clone MGC: 40051 IMAGE: 5243005, | Function unknown | 7.75 |
| | | mRNA, complete cds | | |
| NM_005764 | Hs.271473 | DD96: epithelial protein up-regulated in carcinoma, | Up-regulated in malignant epithelial cells of renal cell carcinomas, and in | 7.75 |
| | | membrane associated protein 17 | carcinomas of colon, breast and lung | |
| AW360901 | Hs.183047 | MGC4399: mitochondrial carrier protein | Mitochondrial carrier protein MGC4399 | 7.71 |
| AL353944 | Hs.50115 | Homo sapiens mRNA; cDNA DKFZp761J1112 (from | Function unknown | 7.69 |
| | | clone DKFZp761J1112) | | |
| H59799 | Hs.42644 | TXNL2, thioredoxin-like 2 | Member of the thioredoxin family; has region of moderate similarity to | 7.65 |
| | | | glutaredoxin-like proteins | |
| NM_002984 | Hs.75703 | CCL4, chemokine (C—C motif) ligand 4 | Cytokine A4 | 7.64 |
| AA642452 | Hs.130881 | BCL11A, B-cell CLL/lymphoma 11A (zinc finger | May bind nucleic acids; contains three C2H2 type zinc finger domains | 7.61 |
| | | protein) | | |
| AA789081 | Hs.4029 | GAS41: giloma-amplified sequence-41 | Similar to the transcription factors AF-9 and ENL | 7.46 |
| H13032 | Hs.103378 | MGC11034, hypothetical protein MGC11034 | Function unknown | 7.42 |
| BE384836 | Hs.3454 | KIAA1821: KIAA1821 protein | KIAA1821 protein | 7.40 |
| AW067800 | Hs.155223 | STC2, stanniocalcin 2 | Stanniocalcin 2; may regulate metal ion homeostasis and inhibits | 7.36 |
| | | | phosphate uptake | |
| T55979 | Hs.115474 | RFC3, replication factor C (activator 1) 3 (38 kD) | Subunit of replication factor C (activator 1) 3; activator of DNA | 7.35 |
| | | | polymerases | |
| AJ278016 | Hs.55565 | ANKRD3, ankyrin repeat domain 3 | Ortholog of mouse protien kinase C-associated kinase, putative gene, | 7.25 |
| | | | ankirin like, possible dual-specificity Ser/Thr/Tyr kinase domain | |
| | | NM_025080: Homo sapiens hypothetical protein | Function unknown | 7.22 |
| | | FLJ22316 (FLJ22316), mRNA. VERSION | | |
| | | NM_025079.1 GI: 13376631 | | |
| AA084248 | Hs.85339:64 | GPR39, G protein-coupled receptor 39 | GPR39, G protein-coupled receptor 39 | 7.15 |
| BE620738 | Hs.173125 | PPIF, peptidylprolyl isomerase F (cyclophilin F) | Cyclophilin F (peptidylprolyl isomerase F); binds the immunosuppressant | 7.06 |
| | | | drug cyclosporin A | |
| AF072873 | Hs.114218 | FZD6, frizzled (Drosophila) homolog 6 | frizzled-6; may function in tissue polarity, development and | 7.04 |
| | | | carcinogenesis; similar to frizzled receptor family, has seven | |
| | | | transmembrane domains | |
| AA852773 | Hs.334838 | KIAA1866 protein | KIAA1866 protein | 6.99 |
| R07566 | Hs.73817 | CCL3, chemokine (C—C motif) ligand 3 | Macrophage inflammatory protein 1 alpha; chemokine | 6.98 |
| NM_005211 | Hs.174142 | CSF1R, colony stimulating factor 1 receptor, formerly | Macrophage colony stimulating factor tyrosine kinase receptor; involved | 6.79 |
| | | McDonough feline sarcoma viral (v-fms) oncogene | in regulation of growth and differentiation of myeloid cells | |
| | | homolog | | |
| AI752666 | Hs.76669 | NNMT, nicotinamide N-methyltransferase | Nicotinamide N-methyltransferase; catalyzes the N-methylation of | 6.52 |
| | | | nicotinamide and other pyridines, structurally-related drugs and | |
| | | | xenobiotics | |
| AF182294 | Hs.241578 | LOC51691: U6 snRNA-associated Sm-like protein | Member of the Sm family; core constituent of snRNP complexes | 6.50 |
| | | LSm8 | | |
| AA457211 | Hs.8858 | BAZ1A, bromodomain adjacent to zinc finger domain, | May bind DNA and act as a chromatin-mediated transcriptional | 6.48 |
| | | 1A | regulator; contains a bromodomain and a PHD-finger | |
| W40262 | Hs.146310 | zc79f02.s1 Pancreatic Islet Homo sapiens cDNA clone | Function unknown | 6.47 |
| | | IMAGE: 328539 3′, mRNA sequence | | |
| AB033091 | Hs.74313 | KIAA1265 protein | Function unknown | 6.45 |
| AA292998 | Hs.163900 | ESTs, Highly similar to winged helix/forkhead | Function unknown | 6.36 |
| | | transcription factor [Homo sapiens] [H. sapiens] | | |
| BE613269 | Hs.21893 | DKFZp761N0624: hypothetical protein | Function unknown | 6.35 |
| | | DKFZp761N0624 | | |
| H25836 | Hs.301527 | ESTs, Moderately similar to unknown [Homo sapiens] | Function unknown | 6.27 |
| | | [H. sapiens] | | |
| AL037228 | Hs.82043 | NUDT5, nudix (nucleoside diphosphate linked molety | NDP-sugar hydrolase; converts ADP-ribose to AMP or ribose 5- | 6.25 |
| | | X)-type motif 5 | phosphate; contains a MulT motif | |
| AV662037 | Hs.124740 | FLJ30532: hypothetical protein FLJ30532 | Function unknown | 6.21 |
| AI674383 | Hs.22891 | wc38h08.x1 NCI_CGAP_Pr28 Homo sapiens cDNA | Function unknown | 6.20 |
| | | clone IMAGE: 2320959 3′, mRNA sequence | | |
| AW342140 | Hs.182545 | ESTs, Weakly similar to POL2_MOUSE Retrovirus- | Function unknown | 6.18 |
| | | related POL polyprotein [Contains: Reverse | | |
| | | transcriptase; Endonuclease] [M. musculus] | | |
| BE560135 | Hs.5232 | HSPC125, HSPC125 protein | Function unknown | 6.17 |
| BE409857 | Hs.69499 | HSPC132: hypothetical protein HSPC132 | Moderately similar to a region of S. cerevisiae Ykl053c-ap | 6.16 |
| AW972542 | Hs.289008 | LOC116150: hypothetical protein, MGC: 7199 | Function unknown | 6.16 |
| AI523755 | Hs.59236 | DKFZP434L0718: hypothetical protein | Function unknown | 6.16 |
| | | DKFZp434L0718 | | |
| NM_014056 | Hs.7917 | DKFZP564K247: DKFZP564K247 protein | Function unknown | 6.08 |
| AI857607 | Hs.181301 | CTSS, cathepsin S | Cathepsin S; lysosomal cysteine (thiol) protease that cleaves elastin | 6.04 |
| AW247529 | Hs.6793 | PAFAH1B3, platelet-activating factor acetylhydrolase, | Platelet-activating factor acetylhydrolase gamma; may play a role in | 5.98 |
| | | isoform lb, gamma subunit (29 kD) | brain development | |
| AK000868 | Hs.5570 | Homo sapiens cDNA FLJ10006 fis, clone | Function unknown | 5.92 |
| | | HEMBA1000168, weakly similar to CYLICIN I | | |
| AF053551 | Hs.31584 | MTX2, metaxin 2 | Very strongly similar to murine metaxin 2 (Mm.12941); are involved in | 5.91 |
| | | | mitochondrial protein import | |
| AI538613 | Hs.298241 | TMPRSS3, Transmembrane protease, serine 3 | The encoded protein contains a serine protease domain, a | 5.86 |
| | | | transmembrane domain, a LDL receptor-like domain, and a scavenger | |
| | | | receptor cysteine-rich domain. This gene was identified as a tumor | |
| | | | associated gene that is overexpressed in ovarian tumors. | |
| U48508 | Hs.89631 | Human skeletal muscle ryanodine receptor gene | Function unknown | 5.86 |
| | | (RYR1), exons 103, 104, 105, 106, and complete cds | | |
| T69387 | Hs.76364 | AIF1, allograft inflammatory factor 1 | Allograft Inflammatory factor 1; cytokine inducible protein associated with | 5.86 |
| | | | vascular injury | |
| AC005954 | Hs.25527 | Homo sapiens chromosome 19, cosmid R28784, | Function unknown | 5.86 |
| | | complete sequence | | |
| AB037805 | Hs.88442 | KIAA1384 protein | Function unknown | 5.84 |
| AL031427 | Hs.40094 | Human DNA sequence from clone 167A19 on | Function unknown | 5.83 |
| | | chromosome 1p32.1-33. Contains three genes for | | |
| | | novel proteins, the DIO1 gene for type I iodothyronine | | |
| | | deiodinase (EC 3.8.1.4, TXDI1, ITDI1) and an HNRNP | | |
| | | A3 (Heterogenous Nuclear Ribonucleoprotein A3, | | |
| | | FBRNP) pseudogene. | | |
| AA340864 | Hs.278562 | CLDN7, claudin 7 | Similar to murine Cldn7; are an integral membrane protein | 5.76 |
| X89984 | Hs.211563 | BCL7A, B-cell CLL/lymphoma 7A | Similar to the actin-binding protein caldesmon; serine-rich | 5.74 |
| AI355761 | Hs.242463 | qt94a11.x1 NCI_CGAP_Co14 Homo sapiens cDNA | Function unknown | 5.73 |
| | | clone IMAGE: 1962908 3′ similar to gb: X74929 | | |
| | | KERATIN, TYPE II CYTOSKELETAL 8 (HUMAN);, | | |
| | | mRNA sequence | | |
| AA376409 | Hs.10862 | Homo sapiens cDNA: FLJ23313 fis, clone HEP11919 | Function unknown | 5.71 |
| AA310162 | Hs.169248 | HCS: cytochrome c | Somatic cytochrome c (heart cytochrome c) | 5.67 |
| AW015534 | Hs.217493 | ANXA2, annexin A2 | Annexin II (lipocortin-2); enhances osteoclast formation and bone | 5.64 |
| | | | resorption; member of the annexin protein family | |
| AA326108 | Hs.53631:82 | BHLHB3: basic helix-loop-helix domain containing, | Basic helix-loop-helix (bHLH) transcription factors (e.g., DEC1, also | 5.64 |
| | | class B, 3 | called BHLHB2; 604256) are related to Drosophila hairy/enhancer of | |
| | | | split proteins. They are involved in the control of proliferation and | |
| | | | development during differentiation, particularly in neurons. | |
| AA120865 | Hs.23136 | ESTs, Highly similar to THYA_HUMAN Prothymosin | Function unknown | 5.62 |
| | | alpha [H. sapiens] | | |
| AK000517 | Hs.6844 | NALP2: NALP2 protein | Protein with low similarity to murine Op1 | 5.54 |
| Z36842 | Hs.57548 | H. sapiens (xs85) mRNA, 209 bp | Function unknown | 5.53 |
| AA831552 | Hs.268016 | Homo sapiens cDNA: FLJ21243 fis, clone COL01164 | | 5.50 |
| AL137578 | Hs.27607 | Homo sapiens mRNA; cDNA DKFZp564N2464 (from | Function unknown | 5.50 |
| | | clone DKFZp564N2464) | | |
| AA316181 | Hs.61635 | STEAP, six transmembrane epithelial antigen of the | Six transmembrane epithelial antigen of the prostate; prostate-specific | 5.46 |
| | | prostate | cell-surface antigen | |
| X03635 | Hs.1657 | ESR1, estrogen receptor 1 | Estrogen receptor, nuclear receptor transcription factor activated by | 5.42 |
| | | | ligand-binding, involved in hormone-mediated inhibition of gene | |
| | | | expression | |
| AI557280 | Hs.184270 | PT2.1_15_G11.r tumor2 Homo sapiens cDNA 3′, | Function unknown | 5.41 |
| | | mRNA sequence | | |
| AW248508 | Hs.279727 | Homo sapiens cDNA FLJ14035 fis, clone | Function unknown | 5.40 |
| | | HEMBA1004638 | | |
| N90866 | Hs.276770 | CDW52, CDW52 antigen (CAMPATH-1 antigen) | CAMPATH-1 antigen; GPI-anchored protein | 5.39 |
| U83115 | Hs.161002 | AIM1, absent in melanoma 1 | Member of the beta gamma-crystallin superfamily of proteins; | 5.35 |
| | | | Interactions with the cytoskeleton | |
| AB007860 | Hs.12802 | DDEF2, development and differentiation enhancing | GTPase-activating protein; Interacts with members of the Arf and Src | 5.35 |
| | | factor 2 | family | |
| Z46223 | Hs.176663 | H. sapiens DNA for immunoglobulin G Fc receptor IIIB | Immunoglobulin G Fc receptor | 5.31 |
| BE264974 | Hs.6566 | TRIP13; thyroid hormone receptor interactor 13 | Interacts with ligand binding domain of thyroid hormone receptor and | 5.30 |
| | | | with human papillomavirus type 16 (HPV16) E1 | |
| AA194422 | Hs.22564 | MYO6, myosin VI | Motor, hearing, myosin ATPase, structural protein. Class 6 myosin; | 5.27 |
| | | | motor protein; very strongly similar to murine Myo6 | |
| AF134157 | Hs.169487 | MAFB, v-maf musculoaponeurotic fibrosarcoma | Very strongly similar to murine Krml; may function as a basic domain- | 5.25 |
| | | oncogene homolog B (avian) | leucine zipper transcription factor | |
| AA232119 | Hs.16085 | SH120: putative G-protein coupled receptor | putative G-protein coupled receptor | 5.25 |
| W58353 | Hs.285123 | OSBPL10, oxysterol binding protein-like 10 | Member of the oxysterol-binding protein (OSBP) family; may bind | 5.21 |
| | | | oxygenated derivatives of cholesterol | |
| AW167128 | Hs.231934 | ESTs, Weakly similar to A57717 transcription factor | Function unknown | 5.19 |
| | | EC2 - human [H. sapiens] | | |
| U70370 | Hs.84136 | PITX1, paired-like homeodomain transcription factor 1 | Member of the homeodomain family of DNA binding proteins; may | 5.18 |
| | | | regulate gene expression and control cell differentiation | |
| N55669 | Hs.333823 | MRPL13, mitochondrial ribosomal protein L13 | Protein of the large 60S ribosomal subunit | 5.17 |
| BE298446 | Hs.305890 | BCL2L1, BCL2-like 1 | BCL2-related protein; alternative form bcl-xiong inhibits apoptosis and | 5.17 |
| | | | bcl-xshort induces apoptosis | |
| AW136551 | Hs.181245 | Homo sapiens cDNA FLJ12532 fis, clone | Function unknown | 5.15 |
| | | NT2RM4000200 | | |
| AW250380 | Hs.109059 | HGS, hepatocyte growth factor-regulated tyrosine | Zinc-finger protein; interacts with STAM, undergoes tyrosine | 5.13 |
| | | kinase substrate | phosphorylation in response to IL2, CSF2, or HGF | |
| AW002565 | Hs.124660 | Homo sapiens cDNA: FLJ21763 fis, clone COLF6967 | Function unknown | 5.13 |
| AI697274 | Hs.105435 | GMDS, GDP-mannose 4,6-dehydratase | GDP-mannose-4,6-dehydratase; epimerase converts GDP-mannose to | 5.11 |
| | | | GDP-mannose-4-keto-6-D-deoxymannose, plays a role in the synthesis | |
| | | | of fucosylated oligosaccharides | |
| NM_003878 | Hs.78619 | GGH, gamma-glutamyl hydrolase (conjugase, | Gamma-glutamyl hydrolase; has greater exopeptidase activity on | 5.11 |
| | | folylpolygammaglutamyl hydrolase) | methotrexate pentaglutamate than on diglutamate | |
| AF052112 | Hs.12540 | LYPLA1, lysophospholipase I | Lysophospholipid-specific lysophospholipase 1; hydrolyzes | 5.09 |
| | | | lysophosphatidyl choline | |
| AV654694 | Hs.82316 | IFI44, interferon-induced protein 44 | Member of the family of interferon-alpha/beta inducible proteins; may | 5.09 |
| | | | mediate the antiviral action of interferon | |
| R24601 | | Home sapiens adenylosuccinate synthetase isozyme | Adenylosuccinate synthetase | 5.07 |
| | | (ADSS) mRNA, complete cds | | |
| BE019020 | Hs.85838 | Homo sapiens cDNA clone IMAGE: 2963945 5′ similar | Function unknown | 5.04 |
| | | to TR: O15427 O15427 MONOCARBOXYLATE | | |
| | | TRANSPORTER.;, mRNA sequence | | |
| AW163799 | Hs.198365 | BPGM, 2,3-bisphosphoglycerate mutase | 2,3-bisphosphoglycerate mutase; has synthase, mutase, and | 5.04 |
| | | | phosphatase activities, controls 2,3-diphosphoglycerate metabolism, | |
| | | | which is an effector for haemoglobin | |
| AA278921 | Hs.1908 | PRG1, proteoglycan 1, secretory granule | Secretory granule proteoglycan 1 | 5.02 |
| NM_003726 | Hs.19126 | SCAP1, src family associated phosphoprotein 1 | Src kinase-associated phosphoprotein; acts as an adaptor protein; | 5.02 |
| | | | contains a pleckstrin homology domain and an SH3 domain | |
| AA281167 | Hs.111911 | ESTs, Weakly similar to T06291 extensin homolog | Function unknown | 5.02 |
| | | T9E8.80 - Arabidopsis thaliana [A. thaliana] | | |
| | | C9000306*: gi\|12737280\|ref\|XP_006682.2\| keratin 18 | Function unknown | 5.01 |
| | | [Homo sapiens]\|\|6633 | | |
| AF098158 | Hs.9329 | C20orf1, chromosome 20 open reading frame 1 | Proliferation-associated nuclear protein; associates with the spindle pole | 5.00 |
| | | | and mitotic spindle during mitosis | |
| AA101043 | Hs.151254:19 | KLK7, kallikrein 7 (chymotryptic; stratum comeum) | Epidermal differentiation. Stratum comeum chymotryptic enzyme; serine | 4.87 |
| | | | protease. Growing evidence suggests that many kallikreins are | |
| | | | Implicated in carcinogenesis and some have potential as novel cancer | |
| | | | and other disease biomarkers. Thought to be involved in the proteolysis | |
| | | | of intercellular cohesive structures preceding desquamation, which is the | |
| | | | shedding of the outermost layer of the epidermis. | |
| AF017986 | Hs.31386:185 | Homo sapiens secreted apoptosis related protein 1 | Function unknown | 4.12 |
| | | (SARP1) mRNA, partial cds. | | |
| AW960564 | Hs.3337:137 | TM4SF1: transmembrane 4 superfamily member 1 | Pathogenesis, plasma membrane, call proliferation, N-linked | 3.62 |
| | | | glycosylation, integral membrane protein, integral plasma membrane | |
| | | | protein. L6 antigen; member of the transmembrane 4 superfamily | |
| | | | (TM4SF). The proteins mediate signal transduction events that play a | |
| | | | role in the regulation of cell development, activation, growth and motility. | |
| | | | This encoded protein is a cell surface antigen and is highly expressed in | |
| | | | different carcinomas. | |
| W29092 | Hs.7678:40 | CRABP1 Cellular retinoic acid binding protein 1 | Cytoplasm, retinoid binding, signal transduction, developmental | 3.34 |
| | | | processes. Cellular retinoic acid-binding protein 1; are involved in | |
| | | | delivering retinoic acid to the nucleus, assumed to play an important role | |
| | | | in retinoic acid-mediated differentiation and proliferation processes. | |
| H93366 | Hs.7567:84 | Homo sapiens cDNA: FLJ21962 fis, clone HEP05564 | Function unknown | 3.29 |
| D49441 | Hs.155981:53 | MSLN, mesothelin | Cell adhesion, cell surface antigen, membrane. Pre-pro-megakaryocyte | 3.14 |
| | | | potentiating factor. An antibody that reacts with ovarian cancers and | |
| | | | mesotheliomas was used to isolate a cell surface antigen named | |
| | | | mesothelin. Although the function of mesothelin is unknown, it may play | |
| | | | a role in cellular adhesion and is present on mesothelium, | |
| | | | mesothellomas, and ovarian cancers. | |
| AA214228 | Hs.127751:21, Hs.78006:5 | C20orf180: chromosome 20 open reading frame 180 | Region of high similarity to tyrosine-phosphorylated protein DOK1 | 2.99 |
| M31126 | Hs.272620:1 | PSG9: pregnancy specific beta-1-glycoprotein 9 | Pregnancy, extracellular, plasma glycoprotein. Member of the | 2.82 |
| | | | pregnancy-specific glycoprotein (PSG) and CEA families. | |
| U62801 | Hs.79361:65 | KLK6, kallikrein 6 (neurosin, zyme) | Serine type peptidase, pathogenesis. Neurosin (protease M, zyme); a | 2.77 |
| | | | sarine protease that cleaves amyloid precursor protein (APP). Growing | |
| | | | evidence suggests that many kallikreins are implicated in carcinogenesis | |
| | | | and some have potential as novel cancer and other disease biomarkers. | |
| AK001536 | Hs.285803:6 | Hamo sapiens cDNA FLJ12852 fis, clone | Function unknown | 2.73 |
| | | NT2RP2003445 | | |
| NM_014767 | Hs.74583:140 | KIAA0275: KIAA0275 gene product | Function unknown | 2.72 |
| NM_000699 | Hs.75733:129, Hs.278399:100, Hs.274376:1 | AMY2A: amylase, alpha 2A; pancreatic | Alpha-amylase, extracellular space, carbohydrate metabolism. | 2.71 |
| | | | Pancreatic alpha-amylase 2A (1,4-alpha-D-glucan glucanohydrolase); | |
| | | | cleaves internal a-1,4 bonds between glucose monomers to digest | |
| | | | starch. | |
| AA430348 | Hs.288837:40 | Homo sapiens cDNA FLJ2927 fis, clone | Function unknown | 2.69 |
| | | NT2RP2004743 | | |
| X51630 | Hs.1145:22, Hs.296851:1 | WT1, Wilms tumor 1 | Nucleus, transcription factor, transcription regulation. 4 Zn finger | 2.58 |
| | | | domains. Functions in kidney and gonad proliferation and differentiation. | |
| | | | Mutations in this gene are associated with the development of Wilms | |
| | | | tumors in the kidney or with abnormalities of the genitourinary tract. | |
| BE393948 | Hs.50915:17 | KLK5, kallikrein 5 | Serine type peptidase, epidermal differentiation, extracellular space. | 2.34 |
| | | | Stratum corneum tryptic enzyme (kallikrein-like protein); may function in | |
| | | | epidermal stratum corneum desquamation and turnover. Expression in | |
| | | | prostate cancer negatively correlated with cancer aggressiveness | |
| | | | (Yousef 2002) | |
| NM_002776 | Hs.69423:46 | KLK10, kallikrein 10 | Putative serine protease. Expressed in normal breast tissue and benign | 2.24 |
| | | | lesions, with loss of expression during tumor progression (Dhar 2001). | |
| | | | SNPs associated with prostate, breast, testicular, and ovarian cancers | |
| | | | (Bharaj 2002). | |
| NM_000954 | Hs.8272:294 | PTGDS: prostaglandin D2 synthase (21 kD, brain) | Membrane, prostaglandin-D synthase. Glutathione-independent | 2.15 |
| | | | prostaglandin D2 synthase; membrane associated, catalyzes synthesis | |
| | | | of prostaglandin D; member of the lipocalin family of transporters. | |
| AB029000 | Hs.70823:109, Hs.297970:48 | KIAA1077: sulfatase FP | Function unknown | 2.04 |
| AL044315 | Hs.173094:70 | KIAA1750; KIAA1750 protein | Function unknown | 0.95 |
| AA334592 | Hs.79914:337 | LUM: lumican | Vision, proteoglycan, extracellular matrix, cartilage condensation, | 0.93 |
| | | | extracellular matrix glycoprotein. Member of the specialized collagens | |
| | | | and SLRP_family | |
| S79895 | Hs.83942:248 | CTSK: cathepsin K (pycnodysostosis) | Lysosome, cathepsin K, cysteine-type peptidase, proteolysis and | 0.91 |
| | | | peptidolysis. Cathepsin K (cathepsin O), a cysteine (thiol) protease; | |
| | | | involved in bone remodeling and reabsorption | |
| AI091195 | Hs.65029:120 | Homo sapiens cDNA clone IMAGE: 1566910 3′, mRNA | Function unknown | 0.91 |
| | | sequence | | |
| AF026692; | Hs.105700:83, Hs.278611:3 | SFRP4: secreted frizzled-related protein 4 | Member of the SFRP family that contains a cysteine-rich domain | 0.73 |
| NM_003014 | | | homologous to the putative Wnt-binding site of Frizzled proteins. SFRPs | |
| | | | act as soluble modulators of Wnt signaling. The expression of SFRP4 in | |
| | | | ventricular myocardium correlates with apoptosis related gene | |
| | | | expression. | |
| AI683243 | Hs.97258:15 | ESTs, Moderately similar to S29539 ribosomal protein | Function unknown | −2.96 |
| | | L13a, cytosolic | | |
| AI267700 | Hs.111128:7 | Homo sapiens, clone IMAGE: 4106329, mRNA | Function unknown | −5.71 |
| AA291377 | Hs.50831:23 | Homo sapiens Ly-6 antigen/uPA receptor-like domain- | Function unknown | −6.78 |
| | | containing protein mRNA, complete cds | | |
| AI420213 | Hs.149722.3 | cDNA clone IMAGE: 2094208 3′, mRNA sequence | Function unknown | −8.52 |
| AJ245671 | Hs.12844:73 | EGFL6, EGF-like-domain; multiple 6 | Cell cycle, oncogenesis, integrin ligand, extracellular space. Member of | −9.44 |
| | | | the epidermal growth factor (EGF) repeat superfamily; contains an EGF- | |
| | | | like-domain. Expressed early during development, and its expression | |
| | | | has been detected in lung and meningioma tumors. | |
| AB018305 | Hs.5378:149 | SPON1, spondin 1, (f-spondin) extracellular matrix | Extracellular matrix protein. Very strongly similar to rat F-spondin | −12.55 |
| | | protein | (Rn.7546); may have a role in the growth and guidance of axons. | |
| AW872527 | Hs.59761:19 | ESTs; Weakly similar to DAP1_HUMAN DEATH- | Function unknown | −14.17 |
| | | ASSOCIATED PROTEIN 1 | | |
| AF129755 | Hs.117772:9, Hs.88474:1 | Homo sapiens prostaglandin endoperoxide H | Function unknown | −21.34 |
| | | synthase-1 mRNA, partial 3′ untranslated region. | | |
| AI023799 | Hs.163242:5 | Homo sapiens cDNA clone IMAGE: 1655725 3′ similar | Function unknown | −41.34 |
| | | to contains MER20.t2 MER20 repetitive element;, | | |
| | | mRNA sequence | | |

TABLE 3
Preferred diagnostic and prognostic markers for detecting ovarian cancer or a recurrence thereof
or survival of a subject suffering from ovarian cancer

| | | | | | | |
|---|---|---|---|---|---|---|
| | Unigene | | | | Chromosome | P |
| Accession Number | Mapping | Gene Name | Function | SEQ ID NO: | Location | value |

A. DOWN-REGULATED GENES

| | | | | | | |
|---|---|---|---|---|---|---|
| AI631024; | Hs.24948:32; | SNCAIP, synuclein, alpha | Cytoplasm, pathogenesis, protein binding. Synphilin-1; | SEQ ID NO: 1 (DNA) | 5q23.2 | 0 |
| NM_005460 | Hs.300445:4 | interacting protein (synphilin) | promotes formation of cytosolic inclusions in neurons | SEQ ID NO: 2 (PRT) | | |
| | | | (SNCAIP). Synuclein alpha interacting protein contains | | | |
| | | | several protein-protein interaction domains and | | | |
| | | | interacts with alpha synuclein in neurons. Mutations of | | | |
| | | | SNCAIP have been linked to Parkinson disease. | | | |
| NM_002387 | Hs.1345:5 | MCC, mutated in | Receptor, signal transduction, tumor suppressor. | SEQ ID NO: 3 (DNA) | 5q22.2 | 0 |
| | | colorectal cancers | Similar to the G protein-coupled m3 muscarinic | SEQ ID NO: 4 (PRT) | | |
| | | | acetylcholine receptor. MCC is a candidate for the | | | |
| | | | putative colorectal tumor suppressor gene. The MCC | | | |
| | | | gene product are involved in early stages of colorectal | | | |
| | | | neoplasia in both sporadic and familial tumors. | | | |
| AI420582; | Hs.136164:23 | SE20-4, | Cutaneous T-cell lymphoma-associated tumor antigen | SEQ ID NO: 5 (DNA) | unmapped | 0 |
| NM_022117 | | cutaneous T-cell | se20-4se20-4; differentially expressed nucleolar TGF-beta1 target protein | SEQ ID NO: 6 (PRT) | | |
| | | lymphoma- | (DENTT); also known as CDA1 | | | |
| | | associated tumor | | | | |
| | | antigen se20- | | | | |
| | | 4se20-4 | | | | |

B. UP-REGULATED GENES

| | | | | | | |
|---|---|---|---|---|---|---|
| BC006428; | Hs.15093:210, Hs.290304:1 | HSPC195, | Homo sapiens cDNA FLJ10920 fis, clone | SEQ ID NO: 7 (DNA) | 5q31.2 | 0 |
| NM_016463 | | hypothetical | OVARC1000384-resourcerer. | SEQ ID NO: 8 (PRT) | | |
| | | protein HSPC195 | | | | |
| NM_017697 | Hs.24743:94 | FLJ20171, | contains 3 RNA recognition motifs | SEQ ID NO: 9 (DNA) | 8q22.1 | 0 |
| | | hypothetical | | SEQ ID NO: 10 (PRT) | | |
| | | protein FLJ20171 | | | | |
| AW630088; | Hs.76550:164 | MAL2 | Mal2 T-cell differentiation protein: found thru interaction | SEQ ID NO: 11 (DNA) | 8q24.12 | 0 |
| NM_001306 | | | with TPD52 which is overexpressed in breast cancer; 4 | SEQ ID NO: 12 (PRT) | | |
| | | | TM are involved in vesicle transport | | | |
| NM_015238 | Hs.21543:36 | KIAA0869, | Function unknown | SEQ ID NO: 13 (DNA) | 5q34 | 0.0002 |
| | | KIAA0869 protein; | | SEQ ID NO: 14 (PRT) | | |
| | | KIBRA | | | | |
| AA284679 | Hs.25640:264, Hs.5372:2 | CLDN3, claudin 3 | Integral plasma membrane protein, pathogenesis, tight | SEQ ID NO: 15 (DNA) | 7q11.23 | 0.0004 |
| | | | junction, transmembrane receptor. Member of the | SEQ ID NO: 16 (PRT) | | |
| | | | claudin family of integral membrane proteins; receptor | | | |
| | | | for Clostridium perfringens enterotoxin; | | | |
| NM_022454 | Hs.97984:22 | SOX17, SRY (sex | Likely ortholog of mouse SRY-box containing gene 17; | SEQ ID NO: 17 (DNA) | 8q11.23 | 0.0005 |
| | | determining | alias SOX17 | SEQ ID NO: 18 (PRT) | | |
| | | region Y)-box 17 | | | | |
| NM_005682 | Hs.6527:201 | GPR56, G | cell adhesion, cell-cell signalling, G-protein linked | SEQ ID NO: 19 (DNA) | 16q13 | 0.0012 |
| | | protein-coupled | receptor, integral plasma membrane protein, G-protein | SEQ ID NO: 20 (PRT) | | |
| | | receptor 56 | linked receptor protein signalling pathway. Member of | | | |
| | | | the G protein-coupled receptor family; similar to secretin | | | |
| | | | and calcitonin receptors. 7 transmembrane domains, a | | | |
| | | | mucin-like domain and cysteine box in the N-terminal | | | |
| | | | region. Expressed in range of tissues, highest levels in | | | |
| | | | thyroid, selectively within the monolayer of cuboidal | | | |
| | | | epithelial cells of the smaller, more actively secreting | | | |
| | | | follicles of human thyroid. Differentially expressed in | | | |
| | | | melanoma cell lines with different metastatic potential | | | |
| | | | (Zendman et al 1999). | | | |
| NM_001307 | Hs.278562:101 | CLDN7, claudin 7 | Integral membrane protein, tight junction. Similar to | SEQ ID NO: 21 (DNA) | 17p13.1 | 0.0016 |
| | | | murine Cldn7; | SEQ ID NO: 22 (PRT) | | |
| NM_014736 | Hs.81892:95 | KIAA0101 gene | function unknown; no signficant hits with Superfamily | SEQ ID NO: 23 (DNA) | 15q31 | 0.0025 |
| | | product | | SEQ ID NO: 24 (PRT) | | |
| BE184455; | Hs.251754:128, Hs.245742:1 | SLPI, secretory | Plasma protein, proteinase inhibitor. Secreted inhibitor | SEQ ID NO: 25 (DNA) | 20q13.12 | 0.0034 |
| NM_003064 | | leukocyte | which protects epithelial tissues from serine proteases. | SEQ ID NO: 26 (PRT) | | |
| | | protease inhibitor | Found in various secretions including seminal plasma, | | | |
| | | (antileukoproteinase) | cervical mucus, and bronchial secretions, has affinity for | | | |
| | | | trypsin, leukocyte elastase, and cathepsin G. Its | | | |
| | | | inhibitory effect contributes to the immune response by | | | |
| | | | protecting epithelial surfaces from attack by | | | |
| | | | endogenous proteolytic enzymes; the protein is also | | | |
| | | | thought to have broad-spectrum anti-biotic activity. | | | |
| NM_013994 | Hs.75562:147 | DDR1, discoidin | Cell adhesion, integral plasma membrane protein, | SEQ ID NO: 27 (DNA) | 6p21.33 | 0.0055 |
| | | domain receptor | transmembrane receptor, protein tyrosine kinase. | SEQ ID NO: 28 (PRT) | | |
| | | family, member 1 | Epithelial-specific receptor protein tyrosine kinase; are | | | |
| | | | involved in cell adhesion; has putative discoidin motifs | | | |
| | | | in extracellular domain. DDR1 (CD167a) is a RTK that | | | |
| | | | is widely expressed in normal and transformed | | | |
| | | | epithelial cells and is activated by various types of | | | |
| | | | collagen. | | | |
| NM_001067 | Hs.156348:184, Hs.270810:2 | TOP2A, | DNA binding, DNA topoisomerase (ATP-hydrolyzing), | SEQ ID NO: 29 (DNA) | 17q21.2 | 0.006 |
| | | topoisomerase | nucleus. DNA topoisomerase II alpha; may relax DNA | SEQ ID NO: 30 (PRT) | | |
| | | (DNA) II alpha | torsion upon replication or transcription. Involved in | | | |
| | | (170 kD) | processes such as chromosome condensation, | | | |
| | | | chromatid separation, and the relief of torsional stress | | | |
| | | | that occurs during DNA transcription and replication. | | | |
| | | | Catalyzes the transient breaking and rejoining of two | | | |
| | | | strands of duplex DNA. The gene encoding this enzyme | | | |
| | | | functions as the target for several anticancer agents | | | |
| | | | and a variety of mutations in this gene have been | | | |
| | | | associated with the development of drug resistance. | | | |
| | | | Reduced activity of this enzyme may also play a role in | | | |
| | | | ataxia-telanglectasia. | | | |
| BE386983; | Hs.343214 | CKLFSF7; | chemokine-like factor gene superfamily; transmb 4 | SEQ ID NO: 31 (DNA) | 3p23 | 0.0131 |
| NM_138410 | | chemokine-like | superfamily | SEQ ID NO: 32 (PRT) | | |
| | | factor super family 7 | | | | |
| AF098158; | Hs.9329:152 | C20orf1, | ATP binding, GTP binding, cell proliferation, mitosis, | SEQ ID NO: 33 (DNA) | | 0.0183 |
| NM_012112 | | chromosome 20 | nucleus spindle. Proliferation-associated nuclear | SEQ ID NO: 34 (PRT) | | |
| | | open reading | protein; associates with the spindle pole and mitotic | | | |
| | | frame 1 | spindle during mitosis | | | |
| NM_001769 | Hs.1244:227, Hs.230559:1, | CD9: CD9 antigen | Plasma membrane, integral plasma membrane protein. | SEQ ID NO: 35 (DNA) | 12p13.31 | 0.0006 |
| | Hs.242020:1 | (p24) | Member of the transmembrane 4 superfamily (TM4SF); | SEQ ID NO: 36 (PRT) | | |
| | | | may mediate platelet activation and aggregation. Cell | | | |
| | | | surface glycoprotein that is known to complex with | | | |
| | | | integrins and other transmembrane 4 superfamily | | | |
| | | | proteins. | | | |
| NM_020859 | Hs.278628:52 | ShrmL, Shroom- | Amiloride-sensitive sodium channel (weakly similar to | SEQ ID NO: 37 (DNA) | | 0.0074 |
| | | related protein | Mus musculus PDZ domain actin binding protein) | SEQ ID NO: 38 (PRT) | | |
| | | (KIAA1481 | | | | |
| | | protein) | | | | |
| NM_004433 | Hs.166096:170 | ELF3, E74-like | Embryogenesis and morphogenesis, transcription co- | SEQ ID NO: 39 (DNA) | 1q32.1 | 0.0004 |
| | | factor 3 (ets | activator, transcription factor, transcription from Pol II | SEQ ID NO: 40 (PRT) | | |
| | | domain | promoter. ETS domain transcriptional activator; | | | |
| | | transcription | activates expression of epithelial cell specific genes. | | | |
| | | factor, epithelial- | | | | |
| | | specific) | | | | |
| AI791905; | Hs.95549:147, Hs.229556:1 | FLJ20273, RNA- | Contains four RNA recognition motifs (RRM, RBD, or | SEQ ID NO: 41 (DNA) | | 0.0007 |
| NM_019027 | | binding protein | RNP) | SEQ ID NO: 42 (PRT) | | |
| X69699; | Hs.73149:72, Hs.213008:1 | PAX8, paired box | Histogenesis and organogenesis, embryogenesis and | SEQ ID NO: 43 (DNA) | 2q13 | 0.0009 |
| NM_013952 | | gene 8 | morphogenesis, thyroid-stimulating hormone receptor, | SEQ ID NO: 44 (PRT) | | |
| | | | transcription factor. Member of the paired domain family | | | |
| | | | of nuclear transcription factors; are involved in the | | | |
| | | | ribosome assembly, required for normal thyroid | | | |
| | | | development, PAX genes play critical roles during fetal | | | |
| | | | development and cancer growth. | | | |
| AI301558 | Hs.290801:35, | EST | Function unknown | SEQ ID NO: 45 (DNA) | | 0.0044 |
| | Hs.356228 | | | | | |
| NM_018000 | Hs.79741:18 | FLJ10116, | Function unknown | SEQ ID NO: 46 (DNA) | 2q35 | 0.0051 |
| | | hypothetical | | SEQ ID NO: 47 (PRT) | | |
| | | protein FLJ10116 | | | | |
| NM_144724 | Hs.124740:18 | hypothetical | 59% identity to human Zinc finger protein 91 | SEQ ID NO: 48 (DNA) | 5q13.12 | 0.0051 |
| | | protein FLJ30532 | | SEQ ID NO: 49 (PRT) | | |
| AF111856; | Hs.105039:48 | SLC34A2, solute | SLC34A2: solute carrier family 34 (sodium phosphate), | SEQ ID NO: 50 (DNA) | 4p15.2 | 0.0121 |
| NM_006424 | | carrier family 34 | member 2; contains 8 predicted TMs and a cysteine- | SEQ ID NO: 51 (PRT) | | |
| | | (sodium | rich N-terminal region. Type 2 sodium-dependent | | | |
| | | phosphate), | phosphate transporter. member of the renal type II co- | | | |
| | | member 2 | transporter family. | | | |
| AW959311 | Hs.87019:8; | EST | probable serine/threonine protein kinase; KIAA0537 | SEQ ID NO: 52 (DNA) | 1q32.1 | 0.0251 |
| | Hs.172012 | DKFZp434J037 | | | | |
| AF111713 | Hs.286218:64 | JAM1, junctional | Cell motility, inflammatory response, intercellular | SEQ ID NO: 53 (DNA) | | 0.0261 |
| | | adhesion | junction. Role in the regulation of tight junction | SEQ ID NO: 54 (PRT) | | |
| | | molecule | assembly in epithelia. Ligation of JAM is required for | | | |
| | | | reovirus-induced activation of NF-kappa-B and | | | |
| | | | apoptosis. Role in lymphocyte homing. | | | |
| AU076611; | Hs.154672:123 | MTHFD2, | Electron transporter, methenyltetrahydrofolate | SEQ ID NO: 55 (DNA) | 2p13.1 | 0.0342 |
| NM_006636 | | methylene | cyclohydrolase, mitochondrion, encodes a nuclear- | SEQ ID NO: 56 (PRT) | | |
| | | tetrahydrofolate | encoded mitochondrial bifunctional enzyme with | | | |
| | | dehydrogenase | methylenetetrahydrofolate dehydrogenase and | | | |
| | | (NAD+ | methenyltetrahydrofolate cyclohydrolase activities. may | | | |
| | | dependent); | provide formyltetrahydrofolate for formylmethionyl tRNA | | | |
| | | methenyltetrahydrofolate | synthesis; involved in initiation of mitochondrial protein | | | |
| | | cyclohydrolase | synthesis. | | | |

C. UP-REGULATED GENES IN MUCINOUS OVARIAN CANCER ONLY

| | | | | | | |
|---|---|---|---|---|---|---|
| AA584890; | Hs.5302:132 | LGALS4, lectin, | Lectin, cytosol, cell adhesion, plasma membrane. Binds | SEQ ID NO: 57 (DNA) | 19q13.2 | 0.0001 |
| NM_006149 | | galactoside- | to beta galactoside, involved in cell adhesion, cell | SEQ ID NO: 58 (PRT) | | |
| | | binding, soluble, 4 | growth regulation, inflammation, immunomodulation, | | | |
| | | (galectin 4) | apoptosis and metastasis; member of a family of | | | |
| | | | lectins. LGALS4 is an S-type lectin that is strongly | | | |
| | | | underexpressed in colorectal cancer. | | | |
| | Hs.89436:50 | CDH17, cadherin | Cell adhesion, integral plasma membrane protein, | SEQ ID NO: 59 (DNA) | 8q22.1 | 0.0172 |
| | | 17, LI cadherin | membrane fraction, small molecule transport, | SEQ ID NO: 60 (PRT) | | |
| | | (liver-intestine) | transporter. Member of the cadherin family of calcium- | | | |
| | | | dependent glycoproteins; facilitates uptake of peptide- | | | |
| | | | based drugs, may mediate cell-cell interactions. | | | |
| | | | Component of the gastrointestinal tract and pancreatic | | | |
| | | | ducts, intestinal proton-dependent peptide transporter in | | | |
| | | | the first step in oral absorption of many medically | | | |
| | | | important peptide-based drugs. | | | |
| NM_005588 | Hs.179704 | MEP1A, meprin A | metalloprotease located apically and secreted by | SEQ ID NO: 61 (DNA) | 6p12 | 0.01 |
| | | alpha, PABA | epithelial cells in normal colon; degrades broad range of | SEQ ID NO: 62 (PRT) | | |
| | | peptide hydrolase | ECM components in vitro; proposed role in tumour | | | |
| | | | progression by facilitating migration, intravasation and | | | |
| | | | metastasis | | | |

D. PROGNOSTIC MARKERS FOR SURVIVAL OR RECURRENCE

| | | | | | | |
|---|---|---|---|---|---|---|
| NM_015092 | Hs.278428 | DD5; EDD | Homo sapiens progestin induced protein (DD5), mRNA. | SEQ ID NO: 63 (DNA) | | 0.00 |
| | | | EDD; Soluble fraction, cell proliferation, ubiquitin— | SEQ ID NO: 64 (PRT) | | |
| | | | protein ligase, ubiquitin conjugating enzyme, ubiquitin- | | | |
| | | | dependent protein degradation. Member of the HECT | | | |
| | | | family of proteins; may function as an E3 ubiquitin- | | | |
| | | | protein ligase. This gene is localized to chromosome | | | |
| | | | 8q22, a locus disrupted in a variety of cancers. This | | | |
| | | | gene potentially has a role in regulation of cell | | | |
| | | | proliferation or differentiation. | | | |
| BE465867; | Hs.197751:66 | DAAM1 | dishevelled associated activator of morphogenesis 1 | SEQ ID NO: 65 (DNA) | 8q22.3 | 0.04 |
| NM_014992 | | | The protein encoded by this gene contains FH domains | SEQ ID NO: 66 (PRT) | | |
| | | | and belongs to a novel FH protein subfamily implicated | | | |
| | | | in cell polarity, thought to function as a scaffolding | | | |
| | | | protein. | | | |
| AA381553; | Hs.198253:21 | HLA1QA | major histocompatibility complex, class II, DQ alpha 1 | SEQ ID NO: 67 (DNA) | 14q23.1 | 0.00 |
| NM_002122 | | | Pathogenesis, class II major histocompatibility complex | SEQ ID NO: 68 (PRT) | | |
| | | | antigen. Alpha 1 chain of HLA-DQ1 class II molecule (la | | | |
| | | | antigen); complex binds peptides and presents them to | | | |
| | | | CD4+ T lymphocytes\|Proteome | | | |
| AF026692; | Hs.105700:83, Hs.278611:3 | SFRP4: secreted | Member of the SFRP family that contains a cysteine- | SEQ ID NO: 69 (DNA) | 6p21.3 | 0.73 |
| NM_003014 | | frizzled-related | rich domain homologous to the putative Wnt-binding | SEQ ID NO: 70 (PRT) | 7p14 | |
| | | protein 4 | site of Frizzled proteins. SFRPs act as soluble | | | |
| | | | modulators of Wnt signaling. The expression of SFRP4 | | | |
| | | | in ventricular myocardium correlates with apoptosis | | | |
| | | | related gene expression. | | | |
| AW015534; | Hs.217493 | ANXA2, annexin | Annexin II (lipocortin-2); enhances osteoclast formation | SEQ ID NO: 71 (DNA) | 15q21-22 | 0.00 |
| NM_004039 | | A2 | and bone resorption; member of the annexin protein | SEQ ID NO: 72 (PRT) | | |
| | | | family | | | |
| BE24669; | Hs.345728 | SOCS3 | STAT induced STAT-inhibitor 3; suppressor of cytokine | SEQ ID NO: 73 (DNA) | 17q25.3 | 0.02 |
| NM_003955 | | | signalling 3; suppression of IL-6 mediated signalling | SEQ ID NO: 74 (PRT) | | |
| AI677897; | Hs.76640 | RGC32 | RGC32, hypothetical protein, unknown function | SEQ ID NO: 75 (DNA) | 13q13.3 | 0.04 |
| NM_014059 | | | | SEQ ID NO: 76 (PRT) | | |
| AA829286; | Hs.332053 | SAA1, serum | Serum amyloid A1; high density lipoapoprotein; role in | SEQ ID NO: 77 (DNA) | 11p15.1 | 0.04 |
| NM_000331 | | amyloid A1 | cholesterol metabolism; inflammatory response | SEQ ID NO: 78 (PRT) | | |
| AA243499; | Hs.104800 | FLJ10134, | Unknown | SEQ ID NO: 79 (DNA) | 3q12.3 | 0.01 |
| NM_018004 | | hypothetical | | SEQ ID NO: 80 (PRT) | | |
| | | protein | | | | |
| M88849; | Hs.323733 | GJB2, gap | Cellular gap junctions; mutations cause some forms of | SEQ ID NO: 81 (DNA) | 13q11-12 | 0.00 |
| NM_004004 | | junction protein | deafness | SEQ ID NO: 82 (PRT) | | |
| | | beta2; connexin | | | | |
| | | 26 | | | | |
| NM_002514 | Hs.235935 | NOV1; | Role in cell adhesion and migration in endothelial cells; | SEQ ID NO: 83 (DNA) | 8q24.1 | 0.01 |
| | | Nephroblastoma | promotes cell survival | SEQ ID NO: 84 (PRT) | | |
| | | overexpressed | | | | |
| | | gene | | | | |

TABLE 4
Correlation of expression between normal ovarian surface epithelium
(OSE), non-invasive tumors (borderline, BL) and ovarian cancer
(CA) as determined by ANOVA

| | | | E- | | Ep- | |
|---|---|---|---|---|---|---|
| | CA125 | MUC-1 | cadherin | CLDN3 | CAM | SOX17 |
| OSE vs IC | <0.0001 | <0.0001 | 0.7251 | 0.6132 | 0.1573 | 0.0854 |
| OSE vs. BL | 0.1765 | <0.0001 | 0.0307 | 0.3633 | 0.0005 | 0.2287 |
| OSE vs. CA | 0.5443 | <0.0001 | 0.1687 | 0.0008 | <0.0001 | 0.6900 |
| IC vs. BL | <0.0001 | <0.0001 | 0.1116 | 0.7849 | 0.0913 | 0.2530 |
| IC vs. CA | <0.0001 | 0.2707 | 0.4147 | 0.0071 | 0.0002 | 0.0544 |
| BL vs. CA | 0.0001 | <0.0001 | 0.0615 | <0.0001 | 0.0011 | 0.0152 |

TABLE 5
Correlation of gene expression with patient outcome (univariate analysis ie., expression
alone without the influence of covariates)
Univariate analysis for clinicopathological variables and CLDN3, Ep-CAM, SOX17,
CA125, MUC1 and E-cadherin immunoreactivity with survival and relapse in 156
patients with epithelial ovarian cancer

| | Disease Specific Survival | | Relapse Free Survival | |
|---|---|---|---|---|
| | Univariate | | Univariate | |
| Variable | Hazards ratio (95% CI) | p-value | Hazards ratio (95% CI) | p-value |
| Pathological tumor stage | | | | |
| Stage 1-3bvs. 3c-4b | 5.89 (3.214-10.79) | <0.0001 | 7.37 (3.26-16.63) | <0.0001 |
| Tumor grade | | | | |
| BL and G1vs. G2 and G3 | 5.508 (2.745-11.052) | <0.0001 | 7.02 (2.76-17.82) | <0.0001 |
| Age | | | | |
| <50 vs. >=50 | 0.533 (0.288-0.988) | 0.0458 | 0.62 (0.29-1.33) | 0.2221 |
| Residual Disease | | | | |
| RD<1 cm vs. >=1 cm | 4.192 (2.671-6.580) | <0.0001 | 4.17 (2.30-7.55) | <0.0001 |
| CA125 level at diagnosis | | | | |
| CA125 <500vs. >500 U/ml | 1.843 (1.102-3.080) | 0.0197 | 2.292 (1.19-4.40) | 0.0128 |
| Performance Status | | | | |
| PS<1 vs. >1 | 0.270 (0.133-0.549) | 0.0003 | 0.53 (0.16-1.74) | 0.2965 |
| CLDN3 expression | | | | |
| Membranous Score 0vs. >0 | 2.794 (1.012-7.718) | 0.0474 | 2.521 (0.908-6.998) | 0.0758 |
| Membranous Score <1vs. >1 | 1.309 (0.763-2.246) | 0.3285 | 1.952 (1.103-3.457) | 0.0217 |
| Ep-CAM expression | | | | |
| Membranous Score <1vs. >1 | 1.460 (0.809-2.634) | 0.2093 | 2.041 (0.997-4.177) | 0.0509 |
| Membranous Score <2vs. >2 | 1.041 (0.634-1.711) | 0.873 | 1.449 (0.845-2.487) | 0.1779 |
| SOX17 expression | | | | |
| Nuclear membranous Score 0vs. >0 | 0.839 (0.514-1.368) | 0.481 | 1.311 (0.728-2.358) | 0.3667 |
| Nuclear membranous Score <1vs. >1 | 1.407 (0.615-3.218) | 0.4183 | 1.037 (0.380-2.829) | 0.9437 |
| CA125 expression | | | | |
| Membranous apical Score 0vs. >0 | 2.581 (1.393-4.781) | 0.0026 | 2.725 (1.218-6.093) | 0.0146 |
| Membranous apical Score <1vs. >1 | 1.637 (1.045-2.564) | 0.0313 | 1.298 (0.731-2.307) | 0.3737 |
| MUC1 expression | | | | |
| Membranous apical Score 0vs. >0 | 2.479 (0.343-17.898) | 0.368 | NA | |
| Membranous apical Score <1vs. >1 | 3.745 (1.176-11.926) | 0.0254 | 6.432 (1.562-26.483) | 0.0099 |
| Membranous apical Score <2vs. >2 | 1.814 (0.898-3.664) | 0.0969 | 3.893 (1.552-9.766) | 0.0038 |
| E-cadherin expression | | | | |
| Membranous Score 0vs. >0 | 0.806 (0.493-1.318) | 0.3892 | 0.837 (0.477-1.467) | 0.5341 |
| Membranous Score <1vs. >1 | 1.331 (0.532-3.333) | 0.5411 | 0.847 (0.263-2.731) | 0.7814 |
| Membranous Score <2vs. >2 | 0.593 (0.082-4.284) | 0.6041 | 0.913 (0.125-6.646) | 0.9284 |

TABLE 6
Correlation of gene expression with patient outcome (multivariate analysis ie looking
at expression incorporating the influence of covariates)
Multivariate analysis for univariate significant clinicopathological variables and CLDN3,
Ep-CAM, SOX17, CA125, MUC1 and E-cadherin immunoreactivity with survival
and relapse in 156 patients with epithelial ovarian cancer

| | Disease Specific Survival | | Relapse Free Survival | |
|---|---|---|---|---|
| | Multivariate | | Univariate | |
| Variable | Hazards ratio (95% CI) | p-value | Hazards ratio (95% CI) | p-value |
| Pathological tumor stage | | | | |
| Stage 1-3b vs. 3c-4b | 5.66 (2.467-13.012) | <0.0001 | 5.192 (1.860-14.496) | 0.0017 |
| Tumor grade | | | | |
| BL and G1 vs. G2 and G3 | 4.919 (2.080-11.633) | 0.0003 | 7.989 (2.385-26.760) | 0.0008 |
| Age | | | | |
| <50 vs. >=50 | 0.951 (0.482-1.877) | 0.8853 | | |
| Residual Disease | | | | |
| RD<1 cm vs. >=1 cm | 2.974 (1.783-4.959) | <0.0001 | 2.779 (1.433-5.393) | 0.0025 |
| CA125 level at diagnosis | | | | |
| CA125 <500 vs. >500 U/ml | 1.148 (0.625-2.109) | 0.6563 | 1.289 (0.659-2.520) | 0.4587 |
| Performance Status | | | | |
| PS<1 vs. >1 | 0.286 (0.136-0.601) | 0.0009 | | |
| CLDN3 expression | | | | |
| Membranous Score 0 vs. >0 | 1.165 (0.325-4.183) | 0.8145 | | |
| Membranous Score <1 vs. >1 | | | 0.953 (0.473-1.919) | 0.8918 |
| CA125 expression | | | | |
| Membranous apical Score 0 vs. >0 | 0.917 (0.415-2.025) | 0.8302 | 0.693 (0.271-1.768) | 0.4427 |
| Membranous apical Score <1 vs. >1 | 1.664 (0.976-2.837) | 0.0612 | | |
| MUC1 expression | | | | |
| Membranous apical Score 0 vs. >0 | | | | |
| Membranous apical Score <1 vs. >1 | 0.678 (0.255-1.804) | 0.4361 | | |
| Membranous apical Score <2 vs. >2 | | | | |

1. (canceled)

2. A method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having ovarian cancer indicates that the subject being tested has an ovarian cancer, and wherein said nucleic acid probe comprises a sequence selected from the group consisting of:
(i) a sequence comprising at least about 20 contiguous nucleotides from a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(ii) a sequence that hybridizes under at least low stringency hybridization conditions to at least about 20 contiguous nucleotides from a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(iii) a sequence that is at least about 80% identical to a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 46, 48, 50, 52, 53, 55, 57, 59, 61, 63, 65, 67, 69, 71, 73, 75, 77, 79, 81 and 83;
(iv) a sequence that encodes an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 47, 49, 51, 54, 56, 58, 60, 62, 64, 66, 68, 70, 72, 74, 76, 78, 80, 82 and 84; and
(v) a sequence that is complementary to any one of the sequences set forth in (i) or (ii) or (iii) or (iv).

3. (canceled)

4. The method of claim 2 wherein the hybridization is enhanced in the sample from the subject being tested compared to the hybridization obtained for a sample from a control subject not having ovarian cancer.

5. The method of claim 2 wherein the hybridization is reduced in the sample from the subject being tested compared to the hybridization obtained for a sample from a control subject not having ovarian cancer.

6. A method of diagnosing an ovarian cancer in a human or animal subject being tested said method comprising contacting a biological sample from said subject being tested with a nucleic acid probe for a time and under conditions sufficient for hybridization to occur and then detecting the hybridization wherein an enhanced level of hybridization of the probe for the subject being tested compared to the hybridization obtained for a control subject not having ovarian cancer indicates that the subject being tested has an ovarian ovarian cancer, and wherein said nucleic acid probe comprises a sequence selected from the group consisting of:
(i) a sequence comprising at least about 20 contiguous nucleotides from a nucleic acid set forth in Table 1 or 2 other than a nucleic acid having an Accession Number selected from the group consisting of NM_{—}022117, NM_{—}005460, NM_{—}002387, AI745249 and AI694200;
(ii) a sequence that hybridizes under at least low stringency hybridization conditions to at least about 20 contiguous nucleotides from a nucleic acid set forth in Table 1 or 2 other than a nucleic acid having an Accession Number selected from the group consisting of NM_{—}022117, NM_{—}005460, NM_{—}002387, AI745249 and AI694200;
(iii) a sequence that is at least about 80% identical to (i) or (ii);
(iv) a sequence that encodes a polypeptide encoded by a nucleic acid set forth in Table 1 or 2 other than a nucleic acid having an Accession Number selected from the group consisting of NM_{—}022117, NM_{—}005460, NM_{—}002387, AI745249 and AI694200; and
(v) a sequence that is complementary to any one of the sequences set forth in (i) or (ii) or (iii) or (iv).

7. (canceled)

8. A method of diagnosing an ovarian cancer in a human or animal subject being tested said method comprising contacting a biological sample from said subject being tested with a nucleic acid probe for a time and under conditions sufficient for hybridization to occur and then detecting the hybridization wherein a reduced level of hybridization of the probe for the subject being tested compared to the hybridization obtained for a control subject not having ovarian cancer indicates that the subject being tested has an ovarian ovarian cancer, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of NM_{—}022117, NM_{—}005460, NM_{—}002387, AI745249 and AI694200;
(ii) a sequence that hybridizes under at least low stringency hybridization conditions to at least about 20 contiguous nucleotides from a nucleic acid set forth in Table 1 and having an Accession Number selected from the group consisting of NM_{—}022117, NM_{—}005460, NM_{—}002387, AI745249 and AI694200;
(iii) a sequence that is at least about 80% identical to (i) or (ii);
(iv) a sequence that encodes a polypeptide encoded by a nucleic acid set forth in Table 1 and having an Accession Number selected from the group consisting of NM_{—}022117, NM_{—}005460, NM_{—}002387, AI745249 and AI694200; and
(v) a sequence that is complementary to any one of the sequences set forth in (i) or (ii) or (iii) or (iv).

9. (canceled)

10. The method of claim 2 wherein the ovarian cancer that is diagnosed is an epithelial ovarian cancer.

11. The method of claim 2 wherein the ovarian cancer that is diagnosed is selected from the group consisting of serous ovarian cancer, non-invasive ovarian cancer, mixed phenotype ovarian cancer, mucinous ovarian cancer, endometrioid ovarian cancer, clear cell ovarian cancer, papillary serous ovarian cancer, Brenner cell and undifferentiated adenocarcinoma.

12. The method according to claim 11 wherein the ovarian cancer that is diagnosed is selected from the group consisting of serous ovarian cancer, mucinous ovarian cancer, endometrioid ovarian cancer and clear cell ovarian cancer.

13. A method of diagnosing a serous ovarian cancer in a human or animal subject being tested said method comprising contacting a biological sample from said subject being tested with a nucleic acid probe for a time and under conditions sufficient for hybridization to occur and then detecting the hybridization wherein a modified level of hybridization of the probe for the subject being tested compared to the hybridization obtained for a control subject not having ovarian cancer indicates that the subject being tested has a serous ovarian cancer, and wherein said nucleic acid probe comprises a sequence selected from the group consisting of:
(i) a sequence comprising at least about 20 contiguous nucleotides from a nucleic acid set forth in Table 2 or as set forth in Table 1 and having an Accession Number selected from the group consisting of: U62801, D49441, X51630, And AB018305;
(ii) a sequence that hybridizes under at least low stringency hybridization conditions to at least about 20 contiguous nucleotides from a nucleic acid set forth in Table 2 or as set forth in Table 1 and having an Accession Number selected from the group consisting of: U62801, D49441, X51630, And AB018305;
(iii) a sequence that is at least about 80% identical to (i) or (ii);
(iv) a sequence that encodes a polypeptide encoded by a nucleic acid set forth in Table 2 or as set forth in Table 1 and having an Accession Number selected from the group consisting of: U62801, D49441, X51630, And AB018305; and
(v) a sequence that is complementary to any one of the sequences set forth in (i) or (ii) or (iii) or (iv).

14. A method of diagnosing a mucinous ovarian cancer in a human or animal subject being tested said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a mucinous ovarian cancer, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA132961 and AF111856;
(ii) a sequence that hybridizes under at least low stringency hybridization conditions to at least about 20 contiguous nucleotides from a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA 132961 and AF111856;
(iii) a sequence that is at least about 80% identical to (i) or (ii);
(iv) a sequence that encodes a polypeptide encoded by a nucleic acid set forth in Table 1 and having an Accession Number selected from the group consisting of: NM_{—}006149, AA315933, U47732, NM 005588, AW503395, NM_{—}004063, AI073913, AI928445,
NM_{—}022454, W40460, AA132961 and AF111856; and
(v) a sequence that is complementary to any one of the sequences set forth in (i) or (ii) or (iii) or (iv).

15. The method of claim 14 wherein the nucleic acid probe comprises a sequence selected from the group consisting of:
(i) a sequence comprising at least about 20 contiguous nucleotides from SEQ ID NO: 57 or 59 or 61;
(ii) a sequence that hybridizes under at least low stringency hybridization conditions to at least about 20 contiguous nucleotides from SEQ ID NO: 57 or 59 or 61;
(iii) a sequence that is at least about 80% identical to SEQ ID NO: 57 or 59 or 61;
(iv) a sequence that encodes the amino acid sequence set forth in SEQ ID NO: 58 or 60 or 62; and
(v) a sequence that is complementary to any one of the sequences set forth in (i) or (ii) or (iii) or (iv).

16. The method of claim 2 comprising performing a PCR reaction.

17. The method of claim 21 comprising performing a nucleic acid hybridization.

18. (canceled)

19. A method of diagnosing an ovarian cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a sequence having at least about 80% identity to a sequence selected from the group consisting of SEQ ID NOs: 2, 4, 6, 8, 10, 12, 14, 16, 18, 20, 22, 24, 26, 28, 30, 32, 34, 36, 38, 40, 42, 44, 47, 49, 51, 54, 56, 58, 60, 62, 64, 66, 68, 70, 72, 74, 76, 78, 80, 82 and 84.

20. (canceled)

21. A method of diagnosing an ovarian cancer in a human or animal subject being tested said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a polypeptide encoded by a nucleic acid set forth in Table 1 or 2 other than a nucleic acid having an Accession Number selected from the group consisting of NM_{—}022117, NM_{—}005460, NM_{—}002387, AI745249 and AI694200.

22. (canceled)

23. A method of diagnosing an ovarian cancer in a human or animal subject being tested said method comprising contacting a biological sample from said subject being tested with an antibody for a time and under conditions sufficient for an antigen-antibody complex to form and then detecting the complex wherein a reduced level of the antigen-antibody complex for the subject being tested compared to the amount of the antigen-antibody complex formed for a control subject not having ovarian cancer indicates that the subject being tested has an ovarian cancer, and wherein said antibody binds to a polypeptide comprising an amino acid sequence comprising at least about 10 contiguous amino acid residues of a polypeptide encoded by a nucleic acid set forth in Table 1 and having an Accession Number selected from the group consisting of NM_{—}022117, NM_{—}005460, NM_{—}002387, AI745249 and AI694200.

24. (canceled)

25. The method of claim 19 wherein the ovarian cancer that is diagnosed is an epithelial ovarian cancer.

26. The method of claim 19 wherein the ovarian cancer that is diagnosed is selected from the group consisting of serous ovarian cancer, non-invasive ovarian cancer, mixed phenotype ovarian cancer, mucinous ovarian cancer, endometrioid ovarian cancer, clear cell ovarian cancer, papillary serous ovarian cancer, Brenner cell and undifferentiated adenocarcinoma.

27. (canceled)

28. A method of diagnosing a serous ovarian cancer in a human or animal subject being tested said method comprising contacting a biological sample from said subject being tested with an antibody for a time and under conditions sufficient for an antigen-antibody complex to form and then detecting the complex wherein a modified level of the antigen-antibody complex for the subject being tested compared to the amount of the antigen-antibody complex formed for a control subject not having ovarian cancer indicates that the subject being tested has a serous ovarian cancer, and wherein said antibody binds to a polypeptide comprising an amino acid sequence comprising at least about 10 contiguous amino acid residues of a polypeptide encoded by a nucleic acid set forth in Table 2 or as set forth in Table 1 and having an Accession Number selected from the group consisting of: U62801, D49441, X51630, And AB018305.

29. A method of diagnosing a mucinous ovarian cancer in a human or animal subject being tested said method comprising contacting a biological sample from said subject being tested with an antibody for a time and under conditions sufficient for an antigen-antibody complex to form and then detecting the complex wherein a reduced level of the antigen-antibody complex for the subject being tested compared to the amount of the antigen-antibody complex formed for a control subject not having ovarian cancer indicates that the subject being tested has a mucinous ovarian cancer, and wherein said antibody binds to a polypeptide comprising an amino acid sequence comprising at least about 10 contiguous amino acid residues of a polypeptide encoded by a nucleic acid set forth in Table 1 and having an Accession Number selected from the group consisting of: NM_{—}006149, AA315933, U47732, NM_{—}005588, AW503395, NM_{—}004063, AI073913, AI928445, NM_{—}022454, W40460, AA132961 and AF111856.

30. (canceled)

31. A method of detecting an ovarian cancer-associated antibody in a biological sample the method comprising contacting the biological sample with a polypeptide encoded by a polynucleotide that selectively hybridizes to a sequence at least 80% identical to a sequence as shown in Tables 1-3, wherein the polypeptide specifically binds to the ovarian cancer-associated antibody.

32. The method according to claim 2 wherein the biological sample is contacted with a plurality of nucleic acid probes.

33. The method of claim 2 wherein the subject being tested is a patient undergoing a therapeutic regimen to treat ovarian cancer.

34. The method of claim 2 wherein the subject being tested is a subject suspected of having ovarian cancer.

35. A method of monitoring the efficacy of a therapeutic treatment of ovarian cancer, the method comprising:
(i) providing a biological sample from a patient undergoing the therapeutic treatment; and
(ii) determining the level of a ovarian cancer-associated transcript in the biological sample by contacting the biological sample with a polynucleotide that selectively hybridizes to a sequence having at least about 80% identity to a sequence as shown in any one of Tables 1-3, thereby monitoring the efficacy of the therapy.

36. (canceled)

37. A method of monitoring the efficacy of a therapeutic treatment of ovarian cancer, the method comprising:
(i) providing a biological sample from a patient undergoing the therapeutic treatment; and
(ii) determining the level of a ovarian cancer-associated antibody in the biological sample by contacting the biological sample with a polypeptide encoded by a polynucleotide that selectively hybridizes to a sequence at least 80% identical to a sequence as shown in Tables 1-3, wherein the polypeptide specifically binds to the ovarian cancer-associated antibody, thereby monitoring the efficacy of the therapy.

38. (canceled)

39. A method of monitoring the efficacy of a therapeutic treatment of ovarian cancer, the method comprising:
(i) providing a biological sample from a patient undergoing the therapeutic treatment; and
(ii) determining the level of a ovarian cancer-associated polypeptide in the biological sample by contacting the biological sample with an antibody, wherein the antibody specifically binds to a polypeptide encoded by a polynucleotide that selectively hybridizes to a sequence at least 80% identical to a sequence as shown in Tables 1-3, thereby monitoring the efficacy of the therapy.

40-43. (canceled)

44. A method of determining the likelihood of survival of a subject suffering from an ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a poor probability of survival, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955;
(ii) a sequence that hybridizes under at least low stringency hybridization conditions to at least about 20 contiguous nucleotides from a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955;
(iii) a sequence that is at least about 80% identical to (i) or (ii);
(iv) a sequence that encodes a polypeptide encoded by a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955; and
(v) a sequence that is complementary to any one of the sequences set forth in (i) or (ii) or (iii) or (iv).

45. (canceled)

46. A method of determining the likelihood of survival of a subject suffering from an ovarian cancer, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has has a poor probability of survival, and wherein said antibody binds to a polypeptide comprising an amino acid sequence comprising at least about 10 contiguous amino acid residues of a sequence encoded by a nucleic acid set forth in Table 1 and having an Accession Number selected from the group consisting of: NM_{—}003014, AA046217, NM_{—}015902, T83882, AB040888, AA628980, AI623351, AW614420, AA243499, AF251237, AI970797, AF145713, X78565, T97307, BE243845, AW068302, AL133561, BE313555, X07820, AI973016, AF084545, U41518, Z11894, AW138190, BE086548, W47196, AI796870, X02761, AW968613, AW972565, AF045229, AW953853, U52426, F06700, AI798863, H52761, BE546947, AU076643, U20536, AA581602, AJ245210, X65965, AI806770, BE386490, AW581992, U77534, AL034417, L10343, AW518944, W28729, AI640160, U11862, AW295980, X59135, BE466173, AI354722, M90464, AA829286, AI333771, BE465867, NM_{—}014992, BE616902, AA430373, R27430, BE387335, AW264102, AW952323, AA088177, BE614567, AL079658, NM_{—}002776, BE261944, NM_{—}006379, AI002238, X81789, NM_{—}002122, AB001914, AA311919, AI381750, AA292998, BE439580, AI677897, N72403, BE003054, AL035588, AI080491, AW770994, H24177, AF146761, NM_{—}001955, AI680737, AI752666, AA505445, BE246649, and NM_{—}003955.

47. (canceled)

48. A method of determining the likelihood of survival of a subject suffering from a serous ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a poor probability of survival, and wherein said nucleic acid probe comprises a sequence selected from the group consisting of:
(i) a sequence comprising at least about 20 contiguous nucleotides from a nucleic acid comprising the nucleotide sequence set forth in SEQ ID NO: 71 or 73;
(ii) a sequence that hybridizes under at least low stringency hybridization conditions to at least about 20 contiguous nucleotides from a nucleic acid comprising the nucleotide sequence set forth in SEQ ID NO: 71 or 73;
(iii) a sequence that is at least about 80% identical to (i) or (ii) and encoding an sFRP protein or a SOCS3 protein;
(iv) a sequence that encodes a polypeptide comprising the amino acid sequence set forth in SEQ ID NO: 72 or 74; and
(v) a sequence that is complementary to any one of the sequences set forth in (i) or (ii) or (iii) or (iv).

49. A method of determining the likelihood of survival of a subject suffering from a serous ovarian cancer, said method comprising contacting a biological sample from said subject being tested with an antibody for a time and under conditions sufficient for an antigen-antibody complex to form and then detecting the complex wherein an enhanced level of the antigen-antibody complex for the subject being tested compared to the amount of the antigen-antibody complex formed for a control subject not having ovarian cancer indicates that the subject being tested has a poor probability of survival, and wherein said antibody binds to an sFRP polypeptide comprising the amino acid sequence set forth in SEQ ID NO: 72 or a SOCS3 polypeptide comprising the amino acid sequence set forth in SEQ ID NO: 74.

50. A method of determining the likelihood of survival of a subject suffering from a serous ovarian cancer, said method comprising contacting a biological sample from said subject being tested with at least two antibodies for a time and under conditions sufficient for antigen-antibody complexes to form and then detecting the complexes wherein an enhanced level of the antigen-antibody complexes for the subject being tested compared to the amount of the antigen-antibody complexes formed for a control subject not having ovarian cancer indicates that the subject being tested has a poor probability of survival, and wherein one antibody binds to an sFRP polypeptide comprising the amino acid sequence set forth in SEQ ID NO: 72 and wherein one antibody binds to a SOCS3 polypeptide comprising the amino acid sequence set forth in SEQ ID NO: 74.

51-53. (canceled)

54. A method of determining the likelihood that a subject will suffer from a recurrence of an ovarian cancer, said method comprising contacting a biological sample from said subject being tested with a nucleic acid probe for a time and under conditions sufficient for hybridization to occur and then detecting the hybridization wherein an elevated level of hybridization of the probe for the subject being tested compared to the hybridization obtained for a control subject not having ovarian cancer indicates that the subject being tested has a high probability of recurrence, and wherein said nucleic acid probe comprises a sequence selected from the group consisting of:
(i) a sequence comprising at least about 20 contiguous nucleotides from a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM_{—}000947, AJ250562, AL040183, BE207573, BE564162, BE439580, AW067800, AA569756, AW138190, AF126245, L10343, NM_{—}002514, AI863735, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N51357, N80486, NM_{—}000954, NM_{—}005756, NM_{—}016652, R26584, R31178, W05391, W25005, W45393, W68815, X65965, X76732 and Z45051,
(ii) a sequence that hybridizes under at least low stringency hybridization conditions to at least about 20 contiguous nucleotides from a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM_{—}000947, AJ250562, AL040183, BE207573, BE564162, BE439580, AW067800, AA569756, AW138190, AF126245, L10343, NM_{—}002514, AI863735, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N51357, N80486, NM_{—}000954, NM_{—}005756, NM_{—}016652, R26584, R31178, W05391, W25005, W45393, W68815, X65965, X76732 and Z45051;
(iii) a sequence that is at least about 80% identical to (i) or (ii);
(iv) a sequence that encodes a polypeptide encoded by a nucleic acid set forth in Table 1 and having an Accession Number selected from the group consisting of: M86849, AW963419, BE298665, AK000637, BE077546, T97307, R24601, BE090176, AA393907, W28729, BE313754, AW673081, AA356694, L08239, BE397649, NM_{—}012317, NM_{—}000947, AJ250562, AL040183, BE207573, BE564162, BE439580, AW067800, AA569756, AW138190, AF126245, L10343, NM_{—}002514, AI863735, NM_{—}005397, W26391, H15474, U51166, AA243499, AW408807, AI738719, AB040888, BE313077, AI677897, C14898, AI821730, AF007393, H65423, N46243, AA095971, U20350, NM_{—}005756, D19589, AW957446, AW294647, BE159718, AI888490, AA022569, BE147740, AI798863, BE464341, AL080235, AI557212, X75208, AA628980, BE242587, NM_{—}005512, AW953853, AU076611, AW968613, AL353944, BE614149, AA292998, H12912, AA188763, AK000596, AI970797, AW519204, Z42387, AF145713, AA972412, AK001564, AW959861, BE313555, W25005, AI193356, AF111106, AI130740, AA985190, BE221880, AF084545, R26584, AW247380, AA364261, U25849, AF262992, AW342140, AL133572, AI497778, AI745379, U51712, AW375974, AF251237, NM_{—}000636, AA130986, AA216363, AA628980, AA811657, AA897108, AB040888, AF212225, AI089575, AI282028, AI368826, AI718702, AI827248, AK002039, AL109791, AW090198, AW296454, AW445034, AW452948, AW470411, AW885727, AW970859, AW979189, BE165866, BE175582, BE242587, BE271927, BE439580, BE464016, D63216, F34856, M83822, N33937, N49068, N51357, N80486, NM_{—}000954, NM_{—}005756, NM_{—}016652, R26584, R31178, WO5391, W25005, W45393, W68815, X65965, X76732 and Z45051; and
(v) a sequence that is complementary to any one of the sequences set forth in (i) or (ii) or (iii) or (iv).

55-59. (canceled)

60. A method for identifying a compound that modulates an ovarian cancer-associated polypeptide, the method comprising:
(i) contacting the compound with a ovarian cancer-associated polypeptide, the polypeptide encoded by a polynucleotide that selectively hybridizes to a sequence at least 80% identical to a sequence as shown in Tables 1-3; and
(ii) determining the functional effect of the compound upon the polypeptide.

61. A method for determining a candidate compound for the treatment of ovarian cancer comprising:
(i) administering a test compound to a mammal having ovarian cancer or a cell isolated therefrom;
(ii) comparing the level of gene expression of a polynucleotide that selectively hybridizes to a sequence at least 80% identical to a sequence as shown in Tables 1-3 in a treated cell or mammal with the level of gene expression of the polynucleotide in a control cell or mammal, wherein a test compound that modulates the level of expression of the polynucleotide is a candidate for the treatment of ovarian cancer.

62. An assay device for use in the diagnosis or prognosis of ovarian cancer, said device comprising a plurality of polynucleotides immobilized to a solid phase, wherein each of said polynucleotides consists of a gene as listed in any one of Tables 1-3.

63. (canceled)

64. An assay device for use in the diagnosis or prognosis of ovarian cancer, said device comprising a plurality of different antibodies immobilized to a solid phase, wherein each of said antibodies binds to a polypeptide listed in Tables 1-3.

65-69. (canceled)

70. A method of diagnosing an ovarian cancer in a human or animal subject being tested said method comprising determining aberrant methylation in a promoter sequence that regulates expression of a tumor suppressor gene in a biological sample from said subject compared to the methylation of the promoter in nucleic acid obtained for a control subject not having ovarian cancer wherein said aberrant methylation indicates that the subject being tested has an ovarian ovarian cancer.

71-73. (canceled)

74. The method according to claim 19 wherein the biological sample is contacted with a plurality of antibodies.

75. The method of claim 19 wherein the subject being tested is a patient undergoing a therapeutic regimen to treat ovarian cancer.

76. The method of claim 19 wherein the subject being tested is a subject suspected of having ovarian cancer.


SEQUENCE LISTING (84 sequences)

SEQ ID NO: 1 - DNA, 3432 bp, homo sapiens
  CDS (94)..(2850)
gtcagtcccttcgcgctcctgagccgccggcgcgccgggcgccccgggaatttataagtatttgaccgtactcaaaatgtgcaaggaagaataatggaagcccctgaataccttgatttggatgaaattgactttagtgatgacatatcttattcagtcacatcactcaagacgatcccagaactgtgccgaagatgtgatacgcaaaacgaagacagatcagcttctagctctagctggaattgtggcatctcaactcttattacaaacacgcaaaagcccacaggaatcgctgatgtgtacagtaagttccgcccagtgaagcgggtttcgccactgaaacatcagccagagactctggagaacaatgaaagtgatgaccaaaagaaccagaaagtggttgagtaccagaaagggggtgagtctgacctgggcccccagcctcaggagcttggccctggagatggagtgggcggcccaccaggtaagagctctgagcccagcacatcgctgggtgaactggagcactacgacctcgacatggatgagattctggatgtgccttatattaaatccagtcagcagcttgcctcttttaccaaggtgacttcagaaaaaagaattttgggcttatgcacaaccatcaatggcctttctggcaaagcctgctctacaggaagttctgagagctcatcatccaacatggcaccattttgtgttctttctcccgtgaaaagccctcacttgagaaaagcatcagctgtcatccacgaccagcacaagctgtccactgaagaaaccgagatctcacctcctctggttaaatgtggctctgcatatgagcctgaaaaccagagtaaagacttcctaaacaagacatttagtgatcctcatggtcgaaaagttgagaagacaacaccagactgccagctcagggccttccacctacaatcctcagcagcagaatccaaaccagaagagcaggtcagtggcctaaaccggaccagctcccaaggcccagaagaaaggagtgagtatctgaaaaaagtgaaaagcatcttgaacattgttaaagaaggacagatctctctcctgccacacctagctgcagacaatctagacaaaattcacgacgaaaatggaaacaatctattacatattgcggcgtcacagggacacgcagagtgtctacagcacctcacttctttgatgggagaagactgcctcaatgagcgcaacactgagaagttgactccagcaggcctggccattaagaatggtcagttggagtgcgtacgctggatggtgagcgaaacagaagccattgcagaactgagttgttctaaggattttccaagccttattcattacgcaggttgctatggccaggaaaagattcttctgtggcttcttcagtttatgcaagaacagggcatctcgttggatgaagtagaccaggatggcaacagtgccgttcacgtagcctcacagcatggctaccttggatgcatacagaccttggttgaatatggagcaaatgtcaccatgcagaaccacgctggggaaaagccctcccagagcgccgagcggcaggggcacaccctgtgctccaggtacctggtggtggtggagacctgcatgtcgctggcctctcaagtggtgaagttaaccaagcagctaaaggaacaaacagtagaacgtgtcacgctgcagaaccaactccaacaatttctagaagcccagaaatcagagggcaagtcactcccttcttcacccagttcaccatcctcacctgcctccagaaagtcccagtggaaatctccagatgcagatgatgattctgtagccaaaagcaagccaggagtccaagaggggattcaggttcttggaagcctgtcagcctccagccgggctagacccaaagcaaaagatgaagattctgataaaatcttacgccagttattgggaaaggaaatctcagaaaatgtctgcacccaggaaaaactgtccttggaattccaggatgctcaggcttcctctagaaattctaaaaagatcccactggagaagagggaactgaagttagccaggctgagacagctgatgcagaggtcactgagtgagtctgacacagactccaacaactctgaggaccccaagactaccccagtgaggaaggctgaccgaccaaggccgcagcccattgtagaaagcgtagagagtatggacagcgcagaaagcctgcacctgatgattaagaaacacaccttggcatcagggggacgcaggtttcctttcagcatcaaggcctccaaatccctggatggccacagcccatctcccacctcagagagcagcgaaccagacttagaatcccagtatccaggctcagggagtattcctccaaaccagccctctggtgaccctcagcagcccagccctgacagtactgctgcccagaaagttgccacaagtcccaagagtgccctcaagtctccatcttccaagcgtaggacatctcagaacttaaaactgagagttacctttgaggagcctgtggtgcagatggagcagcctagccttgaactgaatggagaaaaagacaaagataagggcaggactctccagcggacctccacaagtaacgaatcgggggatcaactgaaaaggccttttggagcctttcgatctatcatggagacactaagtggcaaccaaaacaataataataactaccaggcagccaaccagctgaaaacctctacattgcccttgacctcacttgggaggaagacagatgccaagggaaaccctgccagctccgctagcaaaggaaagaataaggcagcataatgacatcaatagaaaaatgaagaaatcctacagcataaagcacattgctgagccagagtcaaaagaactcttcttgtaaatcactttttaaattttctctcactgatgccctttggaaattattggaaatttctggactatcctctttggaaagagaaccatgaaaacaatgcctcaccagcagaagaacagaatatcaggatgccttaaatttatagtagtagactgtaaaagattcattttggggtgatatctgtatatataacttgtttttttaaaagatgccgtttaaaagcatgattgggaaaatgtacgttttttaagagtagattgattcaccctacccacaggacattcaccaagccactgataccattttatatttcatcaattgcatgagtatttgctaatgttgattgaacctccctttccccataatgtgggcagatttggctcagctccttcatgagatcaggtcagtggtattgtttctgtcaagagtgttttttctgtcatttctactttttgtataaaggaaataaaacaatgttaacagccaaaaaaaaaaaaaaaaaaaaa

SEQ ID NO: 2 - protein, 919 aa, homo sapiens
MEAPEYLDLDEIDFSDDISYSVTSLKTIPELCRRCDTQNEDRSASSSSWNCGISTLITNTQKPTGIADVYSKFRPVKRVSPLKHQPETLENNESDDQKNQKVVEYQKGGESDLGPQPQELGPGDGVGGPPGKSSEPSTSLGELEHYDLDMDEILDVPYIKSSQQLASFTKVTSEKRILGLCTTINGLSGKACSTGSSESSSSNMAPFCVLSPVKSPHLRKASAVIHDQHKLSTEETEISPPLVKCGSAYEPENQSKDFLNKTFSDPHGRKVEKTTPDCQLRAFHLQSSAAESKPEEQVSGLNRTSSQGPEERSEYLKKVKSILNIVKEGQISLLPHLAADNLDKIHDENGNNLLHIAASQGHAECLQHLTSLMGEDCLNERNTEKLTPAGLAIKNGQLECVRWMVSETEAIAELSCSKDFPSLIHYAGCYGQEKILLWLLQFMQEQGISLDEVDQDGNSAVHVASQHGYLGCIQTLVEYGANVTMQNHAGEKPSQSAERQGHTLCSRYLVVVETCMSLASQVVKLTKQLKEQTVERVTLQNQLQQFLEAQKSEGKSLPSSPSSPSSPASRKSQWKSPDADDDSVAKSKPGVQEGIQVLGSLSASSRARPKAKDEDSDKILRQLLGKEISENVCTQEKLSLEFQDAQASSRNSKKIPLEKRELKLARLRQLMQRSLSESDTDSNNSEDPKTTPVRKADRPRPQPIVESVESMDSAESLHLMIKKHTLASGGRRFPFSIKASKSLDGHSPSPTSESSEPDLESQYPGSGSIPPNQPSGDPQQPSPDSTAAQKVATSPKSALKSPSSKRRTSQNLKLRVTFEEPVVQMEQPSLELNGEKDKDKGRTLQRTSTSNESGDQLKRPFGAFRSIMETLSGNQNNNNNYQAANQLKTSTLPLTSLGRKTDAKGNPASSASKGKNKAA

SEQ ID NO: 3 - DNA, 4181 bp, homo sapiens
  CDS (221)..(2707)
cctcctgcagcaatggctcgtccgtgaaacgcgagccacggctgctctttttaagagtgcctgcatcctccgtttgcgcttcgcaactgtcctgggtgaaaatggctgtctagactaaaatgtggcagaagggaccaagcagtggatattgagcctgtgaagtccaactcttaagctccgagacctgggggactgagagcccagctctgaaaagtgcatcatgaattccggagttgccatgaaatatggaaacgactcctcggccgagctgagtgagctccattcagcagccctggcatcactaaagggagatatagtggaacttaataaacgtctccagcaaacagagagggaacgggaccttctggaaaagaaattggccaaggcacagtgcgagcagtcccacctcatgagagagcatgaggatgtccaggagcgaacgacgcttcgctatgaggaacgcatcacagagctccacagcgtcattgcggagctcaacaagaagatagaccgtctgcaaggcaccaccatcagggaggaagatgagtactcagaactgcgatcagaactcagccagagccaacacgaggtcaacgaggactctcgaagcatggaccaagaccagacctctgtctctatccccgaaaaccagtctaccatggttactgctgacatggacaactgcagtgacctgaactcagaactgcagagggtgctgacagggctggagaatgttgtctgcggcaggaagaagagcagctgcagcctctccgtggccgaggtggacaggcacattgagcagctcaccacagccagcgagcactgtgacctggctattaagacagtcgaggagattgagggggtgcttggccgggacctgtatcccaacctggctgaagagaggtctcggtgggagaaggagctggctgggctgagggaagagaatgagagcctgactgccatgctgtgcagcaaagaggaagaactgaaccggactaaggccaccatgaatgccatccgggaagagcgggaccggctccggaggcgggtcagagagcttcaaactcgactacagagcgtgcaggccacaggtccctccagccctggccgcctcacttccaccaaccgcccgattaaccccagcactggggagctgagcacaagcagcagcagcaatgacattcccatcgccaagattgctgagagggtgaagctatcaaagacaaggtccgaatcgtcatcatctgatcggccagtcctgggctcagaaatcagtagcataggggtatccagcagtgtggctgaacacctggcccactcacttcaggactgctccaatatccaagagattttccaaacactctactcacacggatctgccatctcagaaagcaagattagagagtttgaggtggaaacagaacggctgaatagccggattgagcacctcaaatcccaaaatgacctcctgaccataaccttggaggaatgtaaaagcaatgctgagaggatgagcatgctggtgggaaaatacgaatccaatgccacagcgctgaggctggccttgcagtacagcgagcagtgcatcgaagcctacgaactcctcctggcgctggcagagagtgagcagagcctcatcctggggcagttccgagcggcgggcgtggggtcctcccctggagaccagtcgggggatgaaaacatcactcagatgctcaagcgagctcatgactgccggaagacagctgagaacgctgccaaggccctgctcatgaagctggacggcagctgtgggggagcctttgccgtggccggctgcagcgtgcagccctgggagagcctttcctccaacagccacaccagcacaaccagctccacagccagtagttgcgacaccgagttcactaaagaagacgagcagaggctgaaggattatatccagcagctcaagaatgacagggctgcggtcaagctgaccatgctggagctggaaagcatccacatcgatcctctcagctatgacgtcaagcctcggggagacagccagaggctggatctggaaaacgcagtgcttatgcaggagctcatggccatgaaggaggagatggccgagttgaaggcccagctctacctactggagaaagagaagaaggccctggagctgaagctgagcacgcgggaggcccaggagcaggcctacctggtgcacattgagcacctgaagtccgaggtggaggagcagaaggagcagcggatgcgatccctcagctccaccagcagcggcagcaaagataaacctggcaaggagtgtgctgatgctgcctccccagctctgtccctagctgaactcaggacaacgtgcagcgagaatgagctggctgcggagttcaccaacgccattcgtcgagaaaagaagttgaaggccagagttcaagagctggtgagtgccttggagagactcaccaagagcagtgaaatccgacatcagcaatctgcagagttcgtgaatgatctaaagcgggccaacagcaacctggtggctgcctatgagaaagcaaagaaaaagcatcaaaacaaactgaagaagttagagtcgcagatgatggccatggtggagagacatgagacccaagtgaggatgctcaagcaaagaatagctctgctagaggaggagaactccaggccacacaccaatgaaacttcgctttaatcagcactcacgcaccggagttctgcccatgggaagtaaactgcagcaggccactggggacagaagggcccatgtacttgttgggaggaggaggaaagggaaggctggcaggtaggtcggcacttggacaatggagtgccccaactcaacccttggggtgactggccatggtgacattgtggactgtatccagaggtgcccgctcttccctcctgggcccacaacagcgtgtaaacacatgttctgtgcctgctcagcagagcctcgtttctgctttcagcactcactctccccctcctcttctggtctggcggctgtgcatcagtgggatcccagacatttgtttctgtaagattttccattgtatcctctttttggtagatgctgggctcatcttctagaatctcgtttctcctctttcctcctgcttcatgggaaaacagacctgtgtgtgcctccagcatttaaaaggactgctgatttgtttactacagcaaggctttggtttccaagtcccgggtctcaactttaagatagaggcggccataagaggtgatctctgggagttataggtcatgggaagagcgtagacaggtgttacttacagtcccagatacactaaagttacaaacagaccaccaccaggactgtgcctgaacaattttgtattgagagaataaaaacttccttcaatcttcattttggaggcagggctgggaagggagcgctctcttgattctgggatttctccctctcagtggagccttattaatatccaagacttagagctgggaatctttttgatacctgtagtggaactaaaattctgtcaggggtttcttcaagagctgagaaacattattagcacttcccgccccagggcactacataattgctgttctgctgaatcaaatctcttccacatgggtgcatttgtagctctggacctgtctctacctaaggacaagacactgaggagatactgaacattttgcaaaacttatcacgcctacttaagagtgctgtgtaacccccagttcaagacttagctcctgttgtcatgacggggacagagtgagggaatggtagttaaggcttcttttttgcccccagatacatggtgatggttagcatatggtgcttaaaaggttaaatttcaagcaaaatgcttacagggctaggcagtaccaaagtaactgaattatttcaggaaggtcttcaatcttaaaacaaattcattattctttttcagttttacctcttctctctcagttctacactgatacacttgaaggaccatttactgtttttttctgtagcaccagagaatccatccaaagttccctatgaaaaatgtgttccattgccatagctgactacaaattaaagttgaggaggtttctgcatagagtctttatgtccataagctacgggtaggtctattttcagagcatgatacaaattccacagg

SEQ ID NO: 4 - protein, 829 aa, homo sapiens
MNSGVAMKYGNDSSAELSELHSAALASLKGDIVELNKRLQQTERERDLLEKKLAKAQCEQSHLMREHEDVQERTTLRYEERITELHSVIAELNKKIDRLQGTTIREEDEYSELRSELSQSQHEVNEDSRSMDQDQTSVSIPENQSTMVTADMDNCSDLNSELQRVLTGLENVVCGRKKSSCSLSVAEVDRHIEQLTTASEHCDLAIKTVEEIEGVLGRDLYPNLAEERSRWEKELAGLREENESLTAMLCSKEEELNRTKATMNAIREERDRLRRRVRELQTRLQSVQATGPSSPGRLTSTNRPINPSTGELSTSSSSNDIPIAKIAERVKLSKTRSESSSSDRPVLGSEISSIGVSSSVAEHLAHSLQDCSNIQEIFQTLYSHGSAISESKIREFEVETERLNSRIEHLKSQNDLLTITLEECKSNAERMSMLVGKYESNATALRLALQYSEQCIEAYELLLALAESEQSLILGQFRAAGVGSSPGDQSGDENITQMLKRAHDCRKTAENAAKALLMKLDGSCGGAFAVAGCSVQPWESLSSNSHTSTTSSTASSCDTEFTKEDEQRLKDYIQQLKNDRAAVKLTMLELESIHIDPLSYDVKPRGDSQRLDLENAVLMQELMAMKEEMAELKAQLYLLEKEKKALELKLSTREAQEQAYLVHIEHLKSEVEEQKEQRMRSLSSTSSGSKDKPGKECADAASPALSLAELRTTCSENELAAEFTNAIRREKKLKARVQELVSALERLTKSSEIRHQQSAEFVNDLKRANSNLVAAYEKAKKKHQNKLKKLESQMMAMVERHETQVRMLKQRIALLEEENSRPHTNETSL

SEQ ID NO: 5 - DNA, 2830 bp, homo sapiens
  CDS (130)..(2208)
aattcggcacgaggagagctggttgcgtgagtctcctcagctctgcttaccggtgcgactagcggcagcgacgcggctaaaagcgaaggggcgagtgcgagtcccctgagctgtacgaacgcggtcgccatggaccgcccagatgaggggcctccggccaagacccgccgcctgagcagctccgagtctccacagcgcgacccgcccccgccgccgccgccgccgccgctcctccgactgccgctgcctccaccccagcagcgcccgaggctccaggaggaaacggaggcggcacaggtgctggccgatatgaggggggtgggactgggccccgcgctgcccccgccgcctccctatgtcattctcgaggagggggggatccgcgcatacttcacgctcggtgctgagtgtcccggctgggattctaccatcgagtcggggtatggggaggcgcccccgcccacggagagcctggaagcactccccactcctgaggcctcgggggggagcctggaaatcgattttcaggttgtacagtcgagcagttttggtggagagggggccctagaaacctgtagcgcagtggggtgggcgccccagaggttagttgacccgaagagcaaggaagaggcgatcatcatagtggaggatgaggatgaggatgagcgggagagtatgaggagcagcaggaggcggcggcggcggcggaggaggaagcagaggaaggtgaagagggaaagcagagagagaaatgccgagaggatggagagcatcctgcaggcactggaggatattcagctggatctggaggcagtgaacatcaaggcaggcaaagccttcctgcgtctcaagcgcaagttcatccagatgcgaagacccttcctggagcgcagagacctcatcatccagcatatcccaggcttctgggtcaaagcattcctcaaccaccccagaatttcaattttgatcaaccgacgtgatgaagacattttccgctacttgaccaatctgcaggtacaggatctcagacatatctccatgggctacaaaatgaagctgtacttccagactaacccctacttcacaaacatggtgattgtcaaggagttccagcgcaaccgctcaggccggctggtgtctcactcaaccccaatccgctggcaccggggccaggaaccccaggcccgtcgtcacgggaaccaggatgcgagccacagctttttcagctggttctcaaaccatagcctcccagaggctgacaggattgctgagattatcaagaatgatctgtgggttaaccctctacgctactacctgagagaaaggggctccaggataaagagaaagaagcaagaaatgaagaaacgtaaaaccaggggcagatgtgaggtggtgatcatggaagacgcccctgactattatgcagtggaagacattttcagcgagatctcagacattgatgagacaattcatgacatcaagatctctgacttcatggagaccaccgactacttcgagaccactgacaatgagataactgacatcaatgagaacatctgcgacagcgagaatcctgaccacaatgaggtccccaacaacgagaccactgataacaacgagagtgctgatgaccacgaaaccactgacaacaatgagagtgcagatgacaacaacgagaatcctgaagacaataacaagaacactgatgacaacgaagagaaccctaacaacaacgagaacacttacggcaacaacttcttcaaaggtggcttctggggcagccatggcaacaaccaggacagcagcgacagtgacaatgaagcagatgaggccagtgatgatgaagataatgatggcaacgaaggtgacaatgagggcagtgatgatgatggcaatgaaggtgacaatgaaggcagcgatgatgacgacagagacattgagtactatgagaaagttattgaagactttgacaaggatcaggctgactacgaggacgtgatagagatcatctcagacgaatcagtggaagaagagggcattgaggaaggcatccagcaagatgaggacatctatgaggaaggaaactatgaggaggaaggaagtgaagatgtctgggaagaaggggaagattcggacgactctgacctagaggatgtgcttcaggtcccaaacggttgggccaatccggggaagagggggaaaaccggataagggttttccccttttggggatcacctctctgtatcccccacccactatcccatttgccctcctcctcagctagggccacgcggccccacattgcacttctggggggtgaccgacttcgtacacgggtttaaagtttatttttatggtttagtcattgcagagttcttattttggggggagggaaagggggctagtccccttcttttggccctccgcccccgcaggcttctgtgtgctgctaactgtatttattgtgatgccttggtcagggcccctctacccacttctcccagtcagttgtggccccagcccctctccctgtgctgtgtggagtggacaccctgacccccgaagcggggagggccgctgtggccttcgtcacagccgcgcagtgcccatggaggcgctgctgccaccttcctctcccaagttctttctccatccctctcctcttcccgccgcgccgctagcccgcctcggtgtctatgcaaggccgcttcgccattgcggtattctttgcggtattcttgtccccgtcccccagaaggctcgcctctccccgtggaccctgttaatcccaataaaattctgagcaagttcaaaaaaaaaaaaaaaaaaa

SEQ ID NO: 6 - protein, 693 aa, homo sapiens
MDRPDEGPPAKTRRLSSSESPQRDPPPPPPPPPLLRLPLPPPQQRPRLQEETEAAQVLADMRGVGLGPALPPPPPYVILEEGGIRAYFTLGAECPGWDSTIESGYGEAPPPTESLEALPTPEASGGSLEIDFQVVQSSSFGGEGALETCSAVGWAPQRLVDPKSKEEAIIIVEDEDEDERESMRSSRRRRRRRRRKQRKVKRESRERNAERMESILQALEDIQLDLEAVNIKAGKAFLRLKRKFIQMRRPFLERRDLIIQHIPGFWVKAFLNHPRISILINRRDEDIFRYLTNLQVQDLRHISMGYKMKLYFQTNPYFTNMVIVKEFQRNRSGRLVSHSTPIRWHRGQEPQARRHGNQDASHSFFSWFSNHSLPEADRIAEIIKNDLWVNPLRYYLRERGSRIKRKKQEMKKRKTRGRCEVVIMEDAPDYYAVEDIFSEISDIDETIHDIKISDFMETTDYFETTDNEITDINENICDSENPDHNEVPNNETTDNNESADDHETTDNNESADDNNENPEDNNKNTDDNEENPNNNENTYGNNFFKGGFWGSHGNNQDSSDSDNEADEASDDEDNDGNEGDNEGSDDDGNEGDNEGSDDDDRDIEYYEKVIEDFDKDQADYEDVIEIISDESVEEEGIEEGIQQDEDIYEEGNYEEEGSEDVWEEGEDSDDSDLEDVLQVPNGWANPGKRGKTG

SEQ ID NO: 7 - DNA, 2632 bp, homo sapiens
  CDS (931)..(1611)
ggcggctgagcctgagcggggatgtagaggcggcggcagcagaggcggcactggcggcaagagcagacgcccgagccgagcgagaagagcggcagagccttatcccctgaagccgggccccgcgtcccagccctgcccagcccgcgcccagccatgcgcgccgcctgctgagtccgggcgccgcacgctgagccctccgcccgcgagccgcgctcagctcgggggtgattagttgctttttgttgttttttaatttgggccgcggggagggggaggaggggcaggtgctgcaggctcccccccctccccgcctcgggccagccgcggcggcgcgactcgggctccggacccgggcactgctggcggctggagcggagcgcaccgcggcggtggtgcccagagcggagcgcagctccctgccccgcccctccccctcggcctcgcggcgacggcggcggtggcggcttggacgactcggagagccgagtgaagacatttccacctggacacctgaccatgtgcctgccctgagcagcgaggcccaccaggcatctctgttgtgggcagcagggccaggtcctggtctgtggaccctcggcagttggcaggctccctctgcagtggggtctgggcctcggccccaccatgtcgagcctcggcggtggctcccaggatgccggcggcagtagcagcagcagcaccaatggcagcggtggcagtggcagcagtggcccaaaggcaggagcagcagacaagagtgcagtggtggctgccgccgcaccagcctcagtggcagatgacacaccaccccccgagcgtcggaacaagagcggtatcatcagtgagcccctcaacaagagcctgcgccgctcccgcccgctctcccactactcttcttttggcagcagtggtggtagtggcggtggcagcatgatgggcggagagtctgctgacaaggccactgcggctgcagccgctgcctccctgttggccaatgggcatgacctggcggcggccatggcggtggacaaaagcaaccctacctcaaagcacaaaagtggtgctgtggccagcctgctgagcaaggcagagcgggccacggagctggcagccgagggacagctgacgctgcagcagtttgcgcagtccacagagatgctgaagcgcgtggtgcaggagcatctcccgctgatgagcgaggcgggtgctggcctgcctgacatggaggctgtggcaggtgccgaagccctcaatggccagtccgacttcccctacctgggcgctttccccatcaacccaggcctcttcattatgaccccggcaggtgtgttcctggccgagagcgcgctgcacatggcgggcctggctgagtaccccatgcagggagagctggcctctgccatcagctccggcaagaagaagcggaaacgctgcggcatgtgcgcgccctgccggcggcgcatcaactgcgagcagtgcagcagttgtaggaatcgaaagactggccatcagatttgcaaattcagaaaatgtgaggaactcaaaaagaagccttccgctgctctggagaaggtgatgcttccgacgggagccgccttccggtggtttcagtgacggcggcggaacccaaagctgccctctccgtgcaatgtcactgctcgtgtggtctccagcaagggattcgggcgaagacaaacggatgcacccgtctttagaaccaaaaatattctctcacagatttcattcctgtttttatatatatattttttgttgtcgttttaacatctccacgtccctagcataaaaagaaaaagaaaaaaatttaaactgctttttcggaagaacaacaacaaaaaagaggtaaagacgaatctataaagtaccgagacttcctgggcaaagaatggacaatcagtttccttcctgtgtcgatgtcgatgttgtctgtgcaggagatgcagtttttgtgtagagaatgtaaattttctgtaaccttttgaaatctagttactaataagcactactgtaatttagcacagtttaactccaccctcatttaaacttcctttgattctttccgaccatgaaatagtgcatagtttgcctggagaatccactcacgttcataaagagaatgttgatggcgccgtgtagaagccgctctgtatccatccacgcgtgcagagctgccagcagggagctcacagaaggggagggagcaccaggccagctgagctgcacccacagtcccgagactgggatcccccaccccaacagtgattttggaaaaaaaaatgaaagttctgttcgtttatccattgcgatctggggagccccatctcgatatttccaatcctggctacttttcttagagaaaataagtcctttttttctggccttgctaatggcaacagaagaaagggcttctttgcgtggtcccctgctggtgggggtgggtccccagggggccccctgcggcctgggcccccctgcccacggccagcttcctgctgatgaacatgctgtttgtattgttttaggaaaccaggctgttttgtgaataaaacgaatgcatgtttgtgtcacgaaaaaaaaaaaaaaaaaaaaaaaaaaaaaaaaaaaaaaaaaaaaa

SEQ ID NO: 8 - protein, 227 aa, homo sapiens
MMGGESADKATAAAAAASLLANGHDLAAAMAVDKSNPTSKHKSGAVASLLSKAERATELAAEGQLTLQQFAQSTEMLKRVVQEHLPLMSEAGAGLPDMEAVAGAEALNGQSDFPYLGAFPINPGLFIMTPAGVFLAESALHMAGLAEYPMQGELASAISSGKKKRKRCGMCAPCRRRINCEQCSSCRNRKTGHQICKFRKCEELKKKPSAALEKVMLPTGAAFRWFQ

SEQ ID NO: 9 - DNA, 2140 bp, homo sapiens
  CDS (58)..(1131)
gaattcaagaaatgttgccctggttcacctgatattgacaaactggacgttgccacaatgacagagtatttaaattttgagaagagtagttcagtctctcgatatggagcctctcaagttgaagatatggggaatataattttagcaatgatttcagagccttataatcacaggttttcagatccagagagagtgaattacaagtttgaaagtggaacttgcagcaagatggaacttattgatgataacaccgtagtcagggcacgaggtttaccatggcagtcttcagatcaagatattgcaagattcttcaaaggactcaatattgccaagggaggtgcagcactttgtctgaatgctcagggtcgaaggaacggagaagctctggttaggtttgtaagtgaggagcaccgagacctagcactacagaggcacaaacatcacatggggacccggtatattgaggtttacaaagcaacaggtgaagatttccttaaaattgctggtggtacttccaatgaggtagcccagtttctctccaaggaaaatcaagtcattgttcgcatgcgggggctccctttcacggccacagctgaagaagtggtggccttctttggacagcattgccctattactgggggaaaggaaggcatcctctttgtcacctacccagatggtaggccaacaggggacgcttttgtcctctttgcctgtgaggaatatgcacagaatgcgttgaggaagcataaagacttgttgggtaaaagatacattgaactcttcaggagcacagcagctgaagttcagcaggtgctgaatcgattctcctcggcccctctcattccacttccaacccctcccattattccagtactacctcagcaatttgtgccccctacaaatgttagagactgtatacgccttcgaggtcttccctatgcagccacaattgaggacatcctggatttcctgggggagttcgccacagatattcgtactcatggggttcacatggttttgaatcaccagggccgcccatcaggagatgcctttatccagatgaagtctgcggacagagcatttatggctgcacagaagtgtcataaaaaaaaacatgaaggacagatatgttgaagtctttcagtgttcagctgaggagatgaactttgtgttaatggggggcactttaaatcgaaatggcttatccccaccgccatgtaagttaccatgcctgtctcctccctcctacacatttccagctcctgctgcagttattcctacagaagctgccatttaccagccctctgtgattttgaatccacgagcactgcagccctccacagcgtactacccagcaggcactcagctcttcatgaactacacagcgtactatcccagccccccaggttcgcctaatagtcttggctacttccctacagctgctaatcttagcggtgtccctccacagcctggcacggtggtcagaatgcagggcctggcctacaatactggagttaaggaaattcttaacttcttccaaggttaccagtgtttgaaagatgtatggtgatcttgaaacctccagacacaagaaaacttctagcaaattcaggggaagtttgtctacactcaggctgcagtattttcagcaaacttgattggacaaacgggcctgtgccttatcttttggtggagtgaaaaagtttgagctagtgaagccaaatcgtaacttacagcaagcagcatgcagcatacctggctctttgctgattgcaaataggcatttaaaatgtgaatttggaatcagatgtctccattacttccagttaaagtggcatcataggtgtttcctaagttttaagtcttggataaaaactccaccagtgtctaccatctccaccatgaactctgttaaggaagcttcatttttgtatattcccgctcttttctcttcatttccctgtcttctgcataatcatgccttcttgctaagtaattcaagcataagatcttggaataataaaatcacaatcttaggagaaagaataaaattgttattttcccagtctcttggccatgatgatatcttatgattaaaaacaaattaaattttaaaacacctgaaaaaaaaaaaaaaaaa

SEQ ID NO: 10 - protein, 358 aa, homo sapiens
MTEYLNFEKSSSVSRYGASQVEDMGNIILAMISEPYNHRFSDPERVNYKFESGTCSKMELIDDNTVVRARGLPWQSSDQDIARFFKGLNIAKGGAALCLNAQGRRNGEALVRFVSEEHRDLALQRHKHHMGTRYIEVYKATGEDFLKIAGGTSNEVAQFLSKENQVIVRMRGLPFTATAEEVVAFFGQHCPITGGKEGILFVTYPDGRPTGDAFVLFACEEYAQNALRKHKDLLGKRYIELFRSTAAEVQQVLNRFSSAPLIPLPTPPIIPVLPQQFVPPTNVRDCIRLRGLPYAATIEDILDFLGEFATDIRTHGVHMVLNHQGRPSGDAFIQMKSADRAFMAAQKCHKKKHEGQIC

SEQ ID NO: 11 - DNA, 2808 bp, homo sapiens
  CDS (80)..(607)
ggcggcggcggcaggagcccgggaggcggaggcgggaggcggcggcggcgcgcggagacgcagcagcggcagcggcagcatgtcggccggcggagcgtcagtcccgccgcccccgaaccccgccgtgtccttcccgccgccccgggtcaccctgcccgccggccccgacatcctgcggacctactcgggcgccttcgtctgcctggagattctgttcgggggtcttgtctggattttggttgcctcctccaatgttcctctacctctactacaaggatgggtcatgtttgtgtccgtgacagcgtttttcttttcgctcctctttctgggcatgttcctctctggcatggtggctcaaattgatgctaactggaacttcctggattttgcctaccattttacagtatttgtcttctattttggagcctttttattggaagcagcagccacatccctgcatgatttgcattgcaatacaaccataaccgggcagccactcctgagtgataaccagtataacataaacgtagcagcctcaatttttgcctttatgacgacagcttgttatggttgcagtttgggtctggctttacgaagatggcgaccgtaacactccttagaaactggcagtcgtatgttagtttcacttgtctactttatatgtctgatcaatttggataccattttgtccagatgcaaaaacattccaaaagtaatgtgtttagtagagagagactctaagctcaagttctggtttatttcatggatggaatgttaattttattatgatattaaagaaatggccttttattttacatctctcccctttttccctttccccctttattttcctccttttctttctgaaagtttccttttatgtccataaaatacaaatatattgttcataaaaaattagtatcccttttgtttggttgctgagtcacctgaaccttaattttaattggtaattacagcccctaaaaaaaacacatttcaaataggcttcccactaaactctatattttagtgtaaaccaggaattggcacactttttttagaatgggccagatggtaaatatttatgcttcacggtccatacagtctctgtcacaactattcagttctgctagtatagcgtgaaagcagctatacacaatacagaaatgaatgagtgtggttatgttctaataaaacttatttataaaaacaaggggaggctgggtttagcctgtgggccatagtttgtcaaccactggtgtaaaaccttagttatatatgatctgcattttcttgaactgatcattgaaaacttataaacctaacagaaaagccacataatatttagtgtcattatgcaataatcacattgcctttgtgttaatagtcaaatacttacctttggagaatacttacctttggaggaatgtataaaatttctcaggcagagtcctggatataggaaaaagtaatttatgaagtaaacttcagttgcttaatcaaactaatgatagtctaacaactgagcaagatcctcatctgagagtgcttaaaatgggatccccagagaccattaaccaatactggaactggtatctagctactgatgtcttactttgagtttatttatgcttcagaatacagttgtttgccctgtgcatgaatatacccatatttgtgtgtggatatgtgaagcttttccaaatagagctctcagaagaattaagtttttacttctaattattttgcattactttgagttaaatttgaatagagtattaaatataaagttgtagattcttatgtgtttttgtattagcccagacatctgtaatgtttttgcactggtgacagacaaaatctgttttaaaatcatatccagcacaaaaactatttctggctgaatagcacagaaaagtattttaacctacctgtagagatcctcgtcatggaaaggtgccaaactgttttgaatggaaggacaagtaagagtgaggccacagttcccaccacacgagggcttttgtattgttctactttttcagccctttactttctggctgaagcatccccttggagtgccatgtataagttgggctattagagttcatggaacatagaacaaccatgaatgagtggcatgatccgtgcttaatgatcaagtgttacttatctaataatcctctagaaagaaccctgttagatcttggtttgtgataaaaatataaagacagaagacatgaggaaaaacaaaaggtttgaggaaatcaggcatatgactttatacttaacatcagatcttttctataatatcctactactttggttttcctagctccataccacacacctaaacctgtattatgaattacatattacaaagtcataaatgtgccatatggatatacagtacattctagttggaatcgtttactctgctagaatttaggtgtgagattttttgtttcccaggtatagcaggcttatgtttggtggcattaaattggtttctttaaaatgctttggtggcacttttgtaaacagattgcttctagattgttacaaaccaagcctaagacacatctgtgaatacttagatttgtagcttaatcacattctagacttgtgagttgaatgacaaagcagttgaacaaaaattatggcatttaagaatttaacatgtcttagctgtaaaaatgagaaagtgttggttggttttaaaatctggtaactccatgatgaaaagaaatttattttatacgtgttatgtctctaataaagtattcatttgataaaaaaaaaaaaaaa

SEQ ID NO: 12 - protein, 176 aa, homo sapiens
MSAGGASVPPPPNPAVSFPPPRVTLPAGPDILRTYSGAFVCLEILFGGLVWILVASSNVPLPLLQGWVMFVSVTAFFFSLLFLGMFLSGMVAQIDANWNFLDFAYHFTVFVFYFGAFLLEAAATSLHDLHCNTTITGQPLLSDNQYNINVAASIFAFMTTACYGCSLGLALRRWRP

SEQ ID NO: 13 - DNA, 4171 bp, homo sapiens
  CDS (67)..(3405)
ctggagccgctgagcccccgctgcggccgggagctgcatgggggagcgccggcagcgcttgggaagatgccccggccggagctgcccctgccggagggctgggaggaggcgcgcgacttcgacggcaaggtctactacatagaccacacgaaccgcaccaccagctggatcgacccgcgggacaggtacaccaaaccgctcacctttgctgactgcattagtgatgagttgccgctaggatgggaagaggcatatgacccacaggttggagattacttcatagaccacaacaccaaaaccactcagattgaggatcctcgagtacaatggcggcgggagcaggaacatatgctgaaggattacctggtggtggcccaggaggctctgagtgcacaaaaggagatctaccaggtgaagcagcagcgcctggagcttgcacagcaggagtaccagcaactgcatgccgtctgggagcataagctgggctcccaggtcagcttggtctctggttcatcatccagctccaagtatgaccctgagatcctgaaagctgaaattgccactgcaaaatcccgggtcaacaagctgaagagagagatggttcacctccagcacgagctgcagttcaaagagcgtggctttcagaccctgaagaaaatcgataagaaaatgtctgatgctcagggcagctacaaactggatgaagctcaggctgtcttgagagaaacaaaagccatcaaaaaggctattacctgtggggaaaaggaaaagcaagatctcattaagagccttgccatgttgaaggacggcttccgcactgacagggggtctcactcagacctgtggtccagcagcagctctctggagagttcgagtttcccgctaccgaaacagtacctggatgtgagctcccagacagacatctcgggaagcttcggcatcaacagcaacaatcagttggcagagaaggtcagattgcgccttcgatatgaagaggctaagagaaggatcgccaacctgaagatccagctggccaagcttgacagtgaggcctggcctggggtgctggactcagagagggaccggctgatccttatcaacgagaaggaggagctgctgaaggagatgcgcttcatcagcccccgcaagtggacccagggggaggtggagcagctggagatggcccggaagcggctggaaaaggacctgcaggcagcccgggacacccagagcaaggcgctgacggagaggttaaagttaaacagtaagaggaaccagcttgtgagagaactggaggaagccacccggcaggtggcaactctgcactcccagctgaaaagtctctcaagcagcatgcagtccctgtcctcaggcagcagccccggatccctcacgtccagccggggctccctggttgcatccagcctggactcctccacttcagccagcttcactgacctctactatgacccctttgagcagctggactcagagctgcagagcaaggtggagttcctgctcctggagggggccaccggcttccggccctcaggctgcatcaccaccatccacgaggatgaggtggccaagacccagaaggcagagggaggtggccgcctgcaggctctgcgttccctgtctggcaccccaaagtccatgacctccctatccccacgttcctctctctcctccccctccccaccctgttcccctctcatggctgaccccctcctggctggtgatgccttcctcaactccttggagtttgaagacccggagctgagtgccactctttgtgaactgagccttggtaacagcgcccaggaaagataccggctggaggaaccaggaacggagggcaagcagctgggccaagctgtgaatacggcccaggggtgtggcctgaaagtggcctgtgtctcagccgccgtatcggacgagtcagtggctggagacagtggtgtgtacgaggcttccgtgcagagactgggtgcttcagaagctgctgcatttgacagtgacgaatcggaagcagtgggtgcgacccgaattcagattgccctgaagtatgatgagaagaataagcaatttgcaatattaatcatccagctgagtaacctttctgctctgttgcagcaacaagaccagaaagtgaatatccgcgtggctgtccttccttgctctgaaagcacaacctgcctgttccggacccggcctctggacgcctcagacactctagtgttcaatgaggtgttctgggtatccatgtcctatccagcccttcaccagaagaccttaagagtcgatgtctgtaccaccgacaggagccatctggaagagtgcctgggaggcgcccagatcagcctggcggaggtctgccggtctggggagaggtcgactcgctggtacaaccttctcagctacaaatacttgaagaagcagagcagggagctcaagccagtgggagttatggcccctgcctcagggcctgccagcacggacgctgtgtctgctctgttggaacagacagcagtggagctggagaagaggcaggagggcaggagcagcacacagacactggaagacagctggaggtatgaggagaccagtgagaatgaggcagtagccgaggaagaggaggaggaggtggaggaggaggagggagaagaggatgttttcaccgagaaagcctcacctgatatggatgggtacccagcattaaaggtggacaaagagaccaacacggagaccccggccccatcccccacagtggtgcgacctaaggaccggagagtgggcaccccgtcccaggggccatttcttcgagggagcaccatcatccgctctaagaccttctccccaggaccccagagccagtacgtgtgccggctgaatcggagtgatagtgacagctccactctgtccaaaaagccaccttttgttcgaaactccctggagcgacgcagcgtccggatgaagcggccttcctcggtcaagtcgctgcgctccgagcgtctgatccgtacctcgctggacctggagttagacctgcaggcgacaagaacctggcacagccaactgacccaggagatctcggtgctgaaggagctcaaggagcagctggaacaagccaagagccacggggagaaggagctgccacagtggttgcgtgaggacgagcgtttccgcctgctgctgaggatgctggagaagcggcagatggaccgagcggagcacaagggtgagcttcagacagacaagatgatgagggcagctgccaaggatgtgcacaggctccgaggccagagctgtaaggaacccccagaagttcagtctttcagggagaagatggcatttttcacccggcctcggatgaatatcccagctctctctgcagatgacgtctaatcgccagaaaagtatttcctttgttccactgaccaggctgtgaacattgactgtggctaaagttatttatgtggtgttatatgaaggtactgagtcacaagtcctctagtgctcttgttggtttgaagatgaaccgactttttagtttgggtcctactgttgttattaaaaacagaacaaaaacaaaacacacacacacacaaaaacagaaacaaaaaaaaccagcattaaaataataagattgtatagtttgtatatttaggagtgtatttttgggaaagaaaatttaaatgaactaaagcagtattgagttgctgctcttcttaaaatcgtttagattttttttggtttgtacagctccaccttttagaggtcttactgcaataagaagtaatgcctgggggacggtaatcctaataggacgtcccgcacttgtcacagtacagctaatttttcctagttaacatattttgtacaatattaaaaaaatgcacagaaaccattgggggggattcagaggtgcatccacggatcttcttgagctgtgacgtgtttttatgtggctgcccaacgtggagcgggcagtgtgataggctgggtgggctaagcagcctagtctatgtgggtgacaggccacgctggtctcagatgcccagtgaagccactaacatgagtgaggggagggctgtggggaactccattcagttttatctccatcaataaagtggcctttcaaaaagaaaaaaaaaaaaaaaaaaaaaaa

SEQ ID NO: 14 - protein, 1113 aa, homo sapiens
MPRPELPLPEGWEEARDFDGKVYYIDHTNRTTSWIDPRDRYTKPLTFADCISDELPLGWEEAYDPQVGDYFIDHNTKTTQIEDPRVQWRREQEHMLKDYLVVAQEALSAQKEIYQVKQQRLELAQQEYQQLHAVWEHKLGSQVSLVSGSSSSSKYDPEILKAEIATAKSRVNKLKREMVHLQHELQFKERGFQTLKKIDKKMSDAQGSYKLDEAQAVLRETKAIKKAITCGEKEKQDLIKSLAMLKDGFRTDRGSHSDLWSSSSSLESSSFPLPKQYLDVSSQTDISGSFGINSNNQLAEKVRLRLRYEEAKRRIANLKIQLAKLDSEAWPGVLDSERDRLILINEKEELLKEMRFISPRKWTQGEVEQLEMARKRLEKDLQAARDTQSKALTERLKLNSKRNQLVRELEEATRQVATLHSQLKSLSSSMQSLSSGSSPGSLTSSRGSLVASSLDSSTSASFTDLYYDPFEQLDSELQSKVEFLLLEGATGFRPSGCITTIHEDEVAKTQKAEGGGRLQALRSLSGTPKSMTSLSPRSSLSSPSPPCSPLMADPLLAGDAFLNSLEFEDPELSATLCELSLGNSAQERYRLEEPGTEGKQLGQAVNTAQGCGLKVACVSAAVSDESVAGDSGVYEASVQRLGASEAAAFDSDESEAVGATRIQIALKYDEKNKQFAILIIQLSNLSALLQQQDQKVNIRVAVLPCSESTTCLFRTRPLDASDTLVFNEVFWVSMSYPALHQKTLRVDVCTTDRSHLEECLGGAQISLAEVCRSGERSTRWYNLLSYKYLKKQSRELKPVGVMAPASGPASTDAVSALLEQTAVELEKRQEGRSSTQTLEDSWRYEETSENEAVAEEEEEEVEEEEGEEDVFTEKASPDMDGYPALKVDKETNTETPAPSPTVVRPKDRRVGTPSQGPFLRGSTIIRSKTFSPGPQSQYVCRLNRSDSDSSTLSKKPPFVRNSLERRSVRMKRPSSVKSLRSERLIRTSLDLELDLQATRTWHSQLTQEISVLKELKEQLEQAKSHGEKELPQWLREDERFRLLLRMLEKRQMDRAEHKGELQTDKMMRAAAKDVHRLRGQSCKEPPEVQSFREKMAFFTRPRMNIPALSADDV

SEQ ID NO: 15 - DNA, 1294 bp, homo sapiens
  CDS (198)..(857)
caaagccacaggcaggtgcaggcgcagccgcggcgagagcgtatggagccgagccgttagcgcgcgccgtcggtgagtcagtccgtccgtccgtccgtccgtcggggcgccgcagctcccgccaggcccagcggccccggcccctcgtctccccgcacccggagccacccggtggagcgggccttgccgcggcagccatgtccatgggcctggagatcacgggcaccgcgctggccgtgctgggctggctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtgctgttccttctcgccgccctgctcaccctcgtgccggtgtcctggtcggccaacaccattatccgggacttctacaaccccgtggtgcccgaggcgcagaagcgcgagatgggcgcgggcctgtacgtgggctgggcggccgcggcgctgcagctgctggggggcgcgctgctctgctgctcgtgtcccccacgcgagaagaagtacacggccaccaaggtcgtctactccgcgccgcgctccaccggcccgggagccagcctgggcacaggctacgaccgcaaggactacgtctaagggacagacgcagggagaccccaccaccaccaccaccaccaacaccaccaccaccaccgcgagctggagcgcgcaccaggccatccagcgtgcagccttgcctcggaggccagcccacccccagaagccaggaagcccccgcgctggactggggcagcttccccagcagccacggctttgcgggccgggcagtcgacttcggggcccagggaccaacctgcatggactgtgaaacctcacccttctggagcacggggcctgggtgaccgccaatacttgaccaccccgtcgagccccatcgggccgctgcccccatgctcgcgctgggcagggaccggcagccctggaaggggcacttgatatttttcaataaaagcctttcgttttgcaaaaaaaaaaaaaaaaaaaaaaaaaaaaaaaaaaaaaaaaaaaaa

SEQ ID NO: 16 - protein, 220 aa, homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 17 - DNA, 1853 bp, homo sapiens
  CDS (205)..(1446)
gcagtgtcactaggccggctgggggccctgggtacgctgtagaccagaccgcgacaggccagaacacgggcggcggcttcgggccgggagacccgcgcagccctcggggcatctcagtgcctcactccccaccccctcccccgggtcgggggaggcggcgcgtccggcggagggttgaggggagcggggcaggcctggagcgccatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtgacaggtccctgatccgccccagcctgcaggccagaagcagtgttacacacttcctggaggagctaaggaaatcctcagactcctgggtttttgttgttgctgttgttgttttttaaaaggtgtgttggcatataatttatggtaatttattttgtctgccacttgaacagtttgggggggtgaggtttcatttaaaatttgttcagagatttgtttcccatagttggattgtcaaaaccctatttccaagttcaagttaactagctttgaatgtgtcccaaaacagcttcctccatttcctgaaagtttattgatcaaagaaatgttgtcctgggtgtgttttttcaatcttctaaaaaataaaatctggaatcctgaaaaaaaaaaaaaaaaaaaaaaaaaaaa

SEQ ID NO: 18 - protein, 414 aa, homo sapiens [sequence omitted]

SEQ ID NO: 19 - DNA, 3702 bp, homo sapiens
  CDS (159)..(2237)
tggagggtctcgctctgtcacacaggctggagtgcagtggtgtgatcttggctcatcgtaacctccacctcccgggttcaagtgattctcatgcctcagcctcccgagtagctgggattacaggtggtgacttccaagagtgactccgtcggaggaaaatgactccccagtcgctgctgcagacgacactgttcctgctgagtctgctcttcctggtccaaggtgcccacggcaggggccacagggaagactttcgcttctgcagccagcggaaccagacacacaggagcagcctccactacaaacccacaccagacctgcgcatctccatcgagaactccgaagaggccctcacagtccatgcccctttccctgcagcccaccctgcttcccgatccttccctgaccccaggggcctctaccacttctgcctctactggaaccgacatgctgggagattacatcttctctatggcaagcgtgacttcttgctgagtgacaaagcctctagcctcctctgcttccagcaccaggaggagagcctggctcagggccccccgctgttagccacttctgtcacctcctggtggagccctcagaacatcagcctgcccagtgccgccagcttcaccttctccttccacagtcctccccacacggccgctcacaatgcctcggtggacatgtgcgagctcaaaagggacctccagctgctcagccagttcctgaagcatccccagaaggcctcaaggaggccctcggctgcccccgccagccagcagttgcagagcctggagtcgaaactgacctctgtgagattcatgggggacatggtgtccttcgaggaggaccggatcaacgccacggtgtggaagctccagcccacagccggcctccaggacctgcacatccactcccggcaggaggaggagcagagcgagatcatggagtactcggtgctgctgcctcgaacactcttccagaggacgaaaggccggagcggggaggctgagaagagactcctcctggtggacttcagcagccaagccctgttccaggacaagaattccagccacgtcctgggtgagaaggtcttggggattgtggtacagaacaccaaagtagccaacctcacggagcccgtggtgctcaccttccagcaccagctacagccgaagaatgtgactctgcaatgtgtgttctgggttgaagaccccacattgagcagcccggggcattggagcagtgctgggtgtgagaccgtcaggagagaaacccaaacatcctgcttctgcaaccacttgacctactttgcagtgctgatggtctcctcggtggaggtggacgccgtgcacaagcactacctgagcctcctctcctacgtgggctgtgtcgtctctgccctggcctgccttgtcaccattgccgcctacctctgctccagggtgcccctgccgtgcaggaggaaacctcgggactacaccatcaaggtgcacatgaacctgctgctggccgtcttcctgctggacacgagcttcctgctcagcgagccggtggccctgacaggctctgaggctggctgccgagccagtgccatcttcctgcacttctccctgctcacctgcctttcctggatgggcctcgaggggtacaacctctaccgactcgtggtggaggtctttggcacctatgtccctggctacctactcaagctgagcgccatgggctggggcttccccatctttctggtgacgctggtggccctggtggatgtggacaactatggccccatcatcttggctgtgcataggactccagagggcgtcatctacccttccatgtgctggatccgggactccctggtcagctacatcaccaacctgggcctcttcagcctggtgtttctgttcaacatggccatgctagccaccatggtggtgcagatcctgcggctgcgcccccacacccaaaagtggtcacatgtgctgacactgctgggcctcagcctggtccttggcctgccctgggccttgatcttcttctcctttgcttctggcaccttccagcttgtcgtcctctaccttttcagcatcatcacctccttccaaggcttcctcatcttcatctggtactggtccatgcggctgcaggcccggggtggcccctcccctctgaagagcaactcagacagcgccaggctccccatcagctcgggcagcacctcgtccagccgcatctaggcctccagcccacctgcccatgtgatgaagcagagatgcggcctcgtcgcacactgcctgtggcccccgagccaggcccagccccaggccagtcagccgcagactttggaaagcccaacgaccatggagagatgggccgttgccatggtggacggactcccgggctgggcttttgaattggccttggggactactcggctctcactcagctcccacgggactcagaagtgcgccgccatgctgcctagggtactgtccccacatctgtcccaacccagctggaggcctggtctctccttacaaaccctgggcccagccctcattgctgggggccaggccttggatcttgagggtctggcacatccttaatcctgtgcccctgcctgggacagaaatgtggctccagttgctctgtctctcgtggtcaccctgagggcactctgcatcctctgtcattttaacctcaggtggcacccagggcgaatggggcccagggcagaccttcagggccagagccctggcggaggagaggccctttgccaggagcacagcagcagctcgcctacctctgagcccaggccccctccctccctcagccccccagtcctccctccatcttccctggggttctcctcctctcccagggcctccttgctccttcgttcacagctgggggtccccgattccaatgctgttttttggggagtggtttccaggagctgcctggtgtctgctgtaaatgtttgtctactgcacaagcctcggcctgcccctgagccaggctcggtaccgatgcgtgggctgggctaggtccctctgtccatctgggcctttgtatgagctgcattgcccttgctcaccctgaccaagcacacgcctcagaggggccctcagcctctcctgaagccctcttgtggcaagaactgtggaccatgccagtcccgtctggtttccatcccaccactccaaggactgagactgacctcctctggtgacactggcctagagcctgacactctcctaagaggttctctccaagcccccaaatagctccaggcgccctcggccgcccatcatggttaattctgtccaacaaacacacacgggtagattgctggcctgttgtaggtggtagggacacagatgaccgacctggtcactcctcctgccaacattcagtctggtatgtgaggcgtgcgtgaagcaagaactcctggagctacagggacagggagccatcattcctgcctgggaatcctggaagacttcctgcaggagtcagcgttcaatcttgaccttgaagatgggaaggatgttctttttacgtaccaattcttttgtcttttgatattaaaaagaagtacatgttcattgtagagaatttggaaactgtagaagagaatcaagaagaaaaataaaaatcagctgttgtaatcgcctagcaaaaaaaaaaaaaaaaaaa

SEQ ID NO: 20 - protein, 693 aa, homo sapiens
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSHVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRVPLPCRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 21 - DNA, 1332 bp, homo sapiens
  CDS (427)..(1059)
ctgctggctcacctccgagccacctctgctgcgcaccgcagcctcggacctacagcccaggatactttgggacttgccggcgctcagaaacgcgcccagacggcccctccaccttttgtttgcctagggtcgccgagagcgcccggagggaaccgcctggccttcggggaccaccaattttgtctggaaccaccctcccggcgtatcctactccctgtgccgcgaggccatcgcttcactggaggggtcgatttgtgtgtagtttggtgacaagatttgcattcacctggcccaaaccctttttgtctctttgggtgaccggaaaactccacctcaagttttcttttgtggggctgccccccaagtgtcgtttgttttactgtagggtctcccgcccggcgcccccagtgttttctgagggcggaaatggccaattcgggcctgcagttgctgggcttctccatggccctgctgggctgggtgggtctggtggcctgcaccgccatcccgcagtggcagatgagctcctatgcgggtgacaacatcatcacggcccaggccatgtacaaggggctgtggatggactgcgtcacgcagagcacggggatgatgagctgcaaaatgtacgactcggtgctcgccctgtccgcggccttgcaggccactcgagccctaatggtggtctccctggtgctgggcttcctggccatgtttgtggccacgatgggcatgaagtgcacgcgctgtgggggagacgacaaagtgaagaaggcccgtatagccatgggtggaggcataattttcatcgtggcaggtcttgccaccttggtagcttgctcctggtatggccatcagattgtcacagacttttataaccctttgatccctaccaacattaagtatgagtttggccctgccatctttattggctgggcagggtctgccctagtcatcctgggaggtgcactgctctcctgttcctgtcctgggaatgagagcaaggctgggtaccgtgcaccccgctcttaccctaagtccaactcttccaaggagtatgtgtgacctgggatctccttgccccagcctgacaggctatgggagtgtctagatgcctgaaagggcctggggctgagctcagcctgtgggcagggtgccggacaaaggcctcctggtcactctgtccctgcactccatgtatagtcctcttgggttgggggtgggggggtgccgttggtgggagagacaaaaagagggagagtgtgctttttgtacagtaataaaaaataagtattgggaaaaaaaaaaaaaaaaaaaaaaaaaaaaaaaaaaaaa

SEQ ID NO: 22 - protein, 211 aa, homo sapiens
MANSGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGLATLVACSWYGHQIVTDFYNPLIPTNIKYEFGPAIFIGWAGSALVILGGALLSCSCPGNESKAGYRAPRSYPKSNSSKEYV

SEQ ID NO: 23 - DNA, 888 bp, homo sapiens
  CDS (75)..(407)
ggggccttgtccagtgaaacaccctcggctgggaagtcagttcgttctctcctctcctctcttcttgtttgaacatggtgcggactaaagcagacagtgttccaggcacttacagaaaagtggtggctgctcgagcccccagaaaggtgcttggttcttccacctctgccactaattcgacatcagtttcatcgaggaaagctgaaaataaatatgcaggagggaaccccgtttgcgtgcgcccaactcccaagtggcaaaaaggaattggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatagaactttctcattcatctttgaataacgtctccttgtttaccctggtattctagaatgtaaatttacataaatgtgtttgttccaattagctttgttgaacaggcatttaattaaaaaatttaggtttaaatttagatgttcaaaagtagttgtgaaatttgagaatttgtaagactaattatggtaacttagcttagtattcaatataatgcattgtttggtttcttttaccaaattaagtgtctagttcttgctaaaatcaagtcattgcattgtgttctaattacaagtatgttgtatttgagatttgcttagattgttgtactgctgccatttttattggtgtttgattattggaatggtgccatattgtcactccttctacttgctttaaaaagcagagttagatttttgcacattaaaaaaattcagtattaattaaacactaaaaaaaaaaaaaaaaaaaaaaaaaaaaaaa

SEQ ID NO: 24 - protein, 111 aa, homo sapiens
MVRTKADSVPGTYRKVVAARAPRKVLGSSTSATNSTSVSSRKAENKYAGGNPVCVRPTPKWQKGIGEFFRLSPKDSEKENQIPEEAGSSGLGKAKRKACPLQPDHTNDEKE

SEQ ID NO: 25 - DNA, 598 bp, homo sapiens
  CDS (23)..(418)
cagagtcactcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttgatcaatgaataaataaacgagcctatttctctttgcac

SEQ ID NO: 26 - protein, 132 aa, homo sapiens
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 27 - DNA, 3970 bp, homo sapiens
  CDS (337)..(3093)
ggcttaggaagtattaactgatctctgccctagttctcatgtgttaaatatggatagtaatagtatctaccttatgaagtgactgtgaagataaaattatggattctgtttaagggtttaggccagtgtctggcacaggggaagcattctaaaaatatagctgatgctgttaaacaatgactgttgttgttgttttactgttattatccccaaagcggcccattctgtctgttgctgtcagctatgactcagtcccctgattaacttacgcaccacccattttatcccctgcagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcgttgctgctctccaatccagcctaccgcctccttctggccacttacgcccgtccccctcgaggcccgggcccccccacacccgcctgggccaaacccaccaacacccaggcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccagcttctccttgttctccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggtcagctcaccgacgagcaggtcatcgagaacgcgggggagttcttccgggaccagggccggcagtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaa

SEQ ID NO: 28 - protein, 919 aa, homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNASFSLFSRNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGSAHRRAGHRERGGVLPGPGPAVYLSRPPACPQGLYELMLRCWSRESEQRPPFSQLHRFLAEDALNTV

SEQ ID NO: 29 - DNA, 5698 bp, homo sapiens
  CDS (127)..(4719)
aggttcaagtggagctctcctaaccgacgcgcgtctgtggagaagcggcttggtcgggggtggtctcgtggggtcctgcctgtttagtcgctttcagggttcttgagccccttcacgaccgtcaccatggaagtgtcaccattgcagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccagcaaatgtgggtttacgatgaagatgttggcattaactatagggaagtcacttttgttcctggtttgtacaaaatctttgatgagattctagttaatgctgcggacaacaaacaaagggacccaaaaatgtcttgtattagagtcacaattgatccggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtggtcgaaatggctatggagccaaattgtgtaacatattcagtaccaaatttactgtggaaacagccagtagagaatacaagaaaatgttcaaacagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtaaaaggatttcgtagttatgtggacatgtatttgaaggacaagttggatgaaactggtaactccttgaaagtaatacatgaacaagtaaaccacaggtgggaagtgtgtttaactatgagtgaaaaaggctttcagcaaattagctttgtcaacagcattgctacatccaagggtggcagacatgttgattatgtagctgatcagattgtgactaaacttgttgatgttgtgaagaagaagaacaagggtggtgttgcagtaaaagcacatcaggtgaaaaatcacatgtggatttttgtaaatgccttaattgaaaacccaacctttgactctcagacaaaagaaaacatgactttacaacccaagagctttggatcaacatgccaattgagtgaaaaatttatcaaagctgccattggctgtggtattgtagaaagcatactaaactgggtgaagtttaaggcccaagtccagttaaacaagaagtgttcagctgtaaaacataatagaatcaagggaattcccaaactcgatgatgccaatgatgcagggggccgaaactccactgagtgtacgcttatcctgactgagggagattcagccaaaactttggctgtttcaggccttggtgtggttgggagagacaaatatggggttttccctcttagaggaaaaatactcaatgttcgagaagcttctcataagcagatcatggaaaatgctgagattaacaatatcatcaagattgtgggtcttcagtacaagaaaaactatgaagatgaagattcattgaagacgcttcgttatgggaagataatgattatgacagatcaggaccaagatggttcccacatcaaaggcttgctgattaattttatccatcacaactggccctctcttctgcgacatcgttttctggaggaatttatcactcccattgtaaaggtatctaaaaacaagcaagaaatggcattttacagccttcctgaatttgaagagtggaagagttctactccaaatcataaaaaatggaaagtcaaatattacaaaggtttgggcaccagcacatcaaaggaagctaaagaatactttgcagatatgaaaagacatcgtatccagttcaaatattctggtcctgaagatgatgctgctatcagcctggcctttagcaaaaaacagatagatgatcgaaaggaatggttaactaatttcatggaggatagaagacaacgaaagttacttgggcttcctgaggattacttgtatggacaaactaccacatatctgacatataatgacttcatcaacaaggaacttatcttgttctcaaattctgataacgagagatctatcccttctatggtggatggtttgaaaccaggtcagagaaaggttttgtttacttgcttcaaacggaatgacaagcgagaagtaaaggttgcccaattagctggatcagtggctgaaatgtcttcttatcatcatggtgagatgtcactaatgatgaccattatcaatttggctcagaattttgtgggtagcaataatctaaacctcttgcagcccattggtcagtttggtaccaggctacatggtggcaaggattctgctagtccacgatacatctttacaatgctcagctctttggctcgattgttatttccaccaaaagatgatcacacgttgaagtttttatatgatgacaaccagcgtgttgagcctgaatggtacattcctattattcccatggtgctgataaatggtgctgaaggaatcggtactgggtggtcctgcaaaatccccaactttgatgtgcgtgaaattgtaaataacatcaggcgtttgatggatggagaagaacctttgccaatgcttccaagttacaagaacttcaagggtactattgaagaactggctccaaatcaatatgtgattagtggtgaagtagctattcttaattctacaaccattgaaatctcagagcttcccgtcagaacatggacccagacatacaaagaacaagttctagaacccatgttgaatggcaccgagaagacacctcctctcataacagactatagggaataccatacagataccactgtgaaatttgttgtgaagatgactgaagaaaaactggcagaggcagagagagttggactacacaaagtcttcaaactccaaactagtctcacatgcaactctatggtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattgaaaataagcctaagaaagaattaattaaagttctgattcagaggggatatgattcggatcctgtgaaggcctggaaagaagcccagcaaaaggttccagatgaagaagaaaatgaagagagtgacaacgaaaaggaaactgaaaagagtgactccgtaacagattctggaccaaccttcaactatcttcttgatatgcccctttggtatttaaccaaggaaaagaaagatgaactctgcaggctaagaaatgaaaaagaacaagagctggacacattaaaaagaaagagtccatcagatttgtggaaagaagacttggctacatttattgaagaattggaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaagaatgaaaatactgaaggaagccctcaagaagatggtgtggaactagaaggcctaaaacaaagattagaaaagaaacagaaaagagaaccaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaacaaaaacaaaattcacaatggatttggattcagatgaagatttctcagattttgatgaaaaaactgatgatgaagattttgtcccatcagatgctagtccacctaagaccaaaacttccccaaaacttagtaacaaagaactgaaaccacagaaaagtgtcgtgtcagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaagaaatccaagggggagagtgatgacttccatatggactttgactcagctgtggctcctcgggcaaaatctgtacgggcaaagaaacctataaagtacctggaagagtcagatgaagatgatctgttttaaaatgtgaggcgattattttaagtaattatcttaccaagcccaagactggttttaaagttacctgaagctcttaacttcctcccctctgaatttagtttggggaaggtgtttttagtacaagacatcaaagtgaagtaaagcccaagtgttctttagctttttataatactgtctaaatagtgaccatctcatgggcattgttttcttctctgctttgtctgtgttttgagtctgctttcttttgtctttaaaacctgatttttaagttcttctgaactgtagaaatagctatctgatcacttcagcgtaaagcagtgtgtttattaaccatccactaagctaaaactagagcagtttgatttaaaagtgtcactcttcctccttttctactttcagtagatatgagatagagcataattatctgttttatcttagttttatacataatttaccatcagatagaactttatggttctagtacagatactctactacactcagcctcttatgtgccaagtttttctttaagcaatgagaaattgctcatgttcttcatcttctcaaatcatcagaggccaaagaaaaacactttggctgtgtctataacttgacacagtcaatagaatgaagaaaattagagtagttatgtgattatttcagctcttgacctgtcccctctggctgcctctgagtctgaatctcccaaagagagaaaccaatttctaagaggactggattgcagaagactcggggacaacatttgatccaagatcttaaatgttatattgataaccatgctcagcaatgagctattagattcattttgggaaatctccataatttcaatttgtaaactttgttaagacctgtctacattgttatatgtgtgtgacttgagtaatgttatcaacgtttttgtaaatatttactatgtttttctattagctaaattccaacaattttgtactttaataaa

SEQ ID NO: 30 - protein, 1531 aa, homo sapiens
MEVSPLQPVNENMQVNKIKKNEDAKKRLSVERIYQKKTQLEHILLRPDTYIGSVELVTQQMWVYDEDVGINYREVTFVPGLYKIFDEILVNAADNKQRDPKMSCIRVTIDPENNLISIWNNGKGIPVVEHKVEKMYVPALIFGQLLTSSNYDDDEKKVTGGRNGYGAKLCNIFSTKFTVETASREYKKMFKQTWMDNMGRAGEMELKPFNGEDYTCITFQPDLSKFKMQSLDKDIVALMVRRAYDIAGSTKDVKVFLNGNKLPVKGFRSYVDMYLKDKLDETGNSLKVIHEQVNHRWEVCLTMSEKGFQQISFVNSIATSKGGRHVDYVADQIVTKLVDVVKKKNKGGVAVKAHQVKNHMWIFVNALIENPTFDSQTKENMTLQPKSFGSTCQLSEKFIKAAIGCGIVESILNWVKFKAQVQLNKKCSAVKHNRIKGIPKLDDANDAGGRNSTECTLILTEGDSAKTLAVSGLGVVGRDKYGVFPLRGKILNVREASHKQIMENAEINNIIKIVGLQYKKNYEDEDSLKTLRYGKIMIMTDQDQDGSHIKGLLINFIHHNWPSLLRHRFLEEFITPIVKVSKNKQEMAFYSLPEFEEWKSSTPNHKKWKVKYYKGLGTSTSKEAKEYFADMKRHRIQFKYSGPEDDAAISLAFSKKQIDDRKEWLTNFMEDRRQRKLLGLPEDYLYGQTTTYLTYNDFINKELILFSNSDNERSIPSMVDGLKPGQRKVLFTCFKRNDKREVKVAQLAGSVAEMSSYHHGEMSLMMTIINLAQNFVGSNNLNLLQPIGQFGTRLHGGKDSASPRYIFTMLSSLARLLFPPKDDHTLKFLYDDNQRVEPEWYIPIIPMVLINGAEGIGTGWSCKIPNFDVREIVNNIRRLMDGEEPLPMLPSYKNFKGTIEELAPNQYVISGEVAILNSTTIEISELPVRTWTQTYKEQVLEPMLNGTEKTPPLITDYREYHTDTTVKFVVKMTEEKLAEAERVGLHKVFKLQTSLTCNSMVLFDHVGCLKKYDTVLDILRDFFELRLKYYGLRKEWLLGMLGAESAKLNNQARFILEKIDGKIIIENKPKKELIKVLIQRGYDSDPVKAWKEAQQKVPDEEENEESDNEKETEKSDSVTDSGPTFNYLLDMPLWYLTKEKKDELCRLRNEKEQELDTLKRKSPSDLWKEDLATFIEELEAVEAKEKQDEQVGLPGKGGKAKGKKTQMAEVLPSPRGQRVIPRITIEMKAEAEKKNKKKIKNENTEGSPQEDGVELEGLKQRLEKKQKREPGTKTKKQTTLAFKPIKKGKKRNPWSDSESDRSSDESNFDVPPRETEPRRAATKTKFTMDLDSDEDFSDFDEKTDDEDFVPSDASPPKTKTSPKLSNKELKPQKSVVSDLEADDVKGSVPLSSSPPATHFPDETEITNPVPKKNVTVKKTAAKSQSSTSTTGAKKRAAPKGTKRDPALNSGVSQKPDPAKTKNRRKRKPSTSDDSDSNFEKIVSKAVTSKKSKGESDDFHMDFDSAVAPRAKSVRAKKPIKYLEESDEDDLF

SEQ ID NO: 31 - DNA, 1369 bp, homo sapiens
  CDS (237)..(761)
agtctaacttccgcgcatctacgagggccgggactgccgctacctttctggaaggcgccggccggccagtcaccggagggatcccgccagaggcggctccgcgtctccagcagcggggcagggacccgggcgccccgccctcgccagcgcccgcccccgcccgcccccggcccgccctctgtatctggcccctgggcagctgcccggggaggcggccagcgagctggggccgcgcaatgtcgcacggagccgggctcgtccgcaccacgtgcagcagcggcagcgcgctcggacccggggccggcgcggcccagcccagcgcgagccccttggaggggctgctggacctcagctacccccgcacccacgcggccctgctgaaagtggcgcaaatggtcaccctgctgattgccttcatctgtgtgcggagctccctgtggaccaactacagcgcctacagctactttgaagtggtcaccatttgcgacttgataatgatcctcgccttttacctggtccacctcttccgcttctaccgcgtgctcacctgtatcagctggcccctgtcggaacttctgcactatttaatcggtaccctgctcctcctcatcgcctccattgtggcagcttccaagagttacaaccagagcggactggtagccggagcgatctttggtttcatggccaccttcctctgcatggcaagcatatggctgtcctataagatctcgtgtgtaacccagtccacagatgcagccgtctgatgaggccacaacccctaggcccctcaggagctttgcagagaggaggacgtgtactccaggcgaggcctctggacctgtgttcctgtgccaaagtcctgtcaggctggtgggcaccaggaaaggcctgcaccctcttcctgctctcccaggaagccagctccctgagctcctgagccagccggaaactcttcctccagccttccggggagaacatccctcccattctgggaaaggaaagcagcctccagggaaatgttttctgccttcctgcttctagaaccacctcaggtactgatgaaccccacttagcacagctgaaggggtttgtgaatactcccgcctaaatcccttctacttcactcctcaggggagtgaagtgccttaagaaacaaagccctgtcctaatttatctagcttgtcagtccggtcttagagataccctctttcctgaagtgaggcgtgcctgtagaaacactatgtggtcagcctgtccccaaggagatcttgtgtctcctctccatctctgcctttgttaccagtgtgcatgtgtttgtgtgttttttaataaaatattgactcggccagttaaaaaaaaaaaaaaaaaa

SEQ ID NO: 32 - protein, 175 aa, homo sapiens
MSHGAGLVRTTCSSGSALGPGAGAAQPSASPLEGLLDLSYPRTHAALLKVAQMVTLLIAFICVRSSLWTNYSAYSYFEVVTICDLIMILAFYLVHLFRFYRVLTCISWPLSELLHYLIGTLLLLIASIVAASKSYNQSGLVAGAIFGFMATFLCMASIWLSYKISCVTQSTDAAV

SEQ ID NO: 33 - DNA, 3470 bp, homo sapiens
  CDS (458)..(2698)
ggctaggtgagccgtgggaagaaaagagggagcagctagggcgcgggtctccctcctcccggagtttggaacggctgaagttcaccttccagcccctagcgccgttcgcgccgctaggcctggcttctgaggcggttgcggtgctcggtcgccgcctaggcggggcagggtgcgagcaggggcttcgggccacgcttctcttggcgacaggattttgctgtgaagtccgtccgggaaacggaggaaaaaaagagttgcgggaggctgtcggctaataacggttcttgatacatatttgccagacttcaagatttcagaaaaggggtgaaagagaagattgcaactttgagtcagacctgtaggcctgatagactgattaaaccacagaaggtgacctgctgagaaaagtggtacaaatactgggaaaaacctgctcttctgcgttaagtgggagacaatgtcacaagttaaaagctcttattcctatgatgccccctcggatttcatcaatttttcatccttggatgatgaaggagatactcaaaacatagattcatggtttgaggagaaggccaatttggagaataagttactggggaagaatggaactggagggctttttcagggcaaaactcctttgagaaaggctaatcttcagcaagctattgtcacacctttgaaaccagttgacaacacttactacaaagaggcagaaaaagaaaatcttgtggaacaatccattccgtcaaatgcttgttcttccctggaagttgaggcagccatatcaagaaaaactccagcccagcctcagagaagatctcttaggctttctgctcagaaggatttggaacagaaagaaaagcatcatgtaaaaatgaaagccaagagatgtgccactcctgtaatcatcgatgaaattctaccctctaagaaaatgaaagtttctaacaacaaaaagaagccagaggaagaaggcagtgctcatcaagatactgctgaaaagaatgcatcttccccagagaaagccaagggtagacatactgtgccttgtatgccacctgcaaagcagaagtttctaaaaagtactgaggagcaagagctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcaagttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcgtttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgccccttacgactctcaccctctccccactttttttaaaaattttaaccagaaaataaagatagttaaatcctaagatagagattaagtcatggtttaaatgaggaacaatcagtaaatcagattctgtcctcttctctgcataccgtgaatttatagttaaggatccctttgctgtgagggtagaaaacctcaccaactgcaccagtgaggaagaagactgcgtggattcatggggagcctcacagcagccacgcagcaggctctgggtggggctgccgttaaggcacgttctttccttactggtgctgataacaacagggaaccgtgcagtgtgcattttaagacctggcctggaataaatacgttttgtctttccctcaaaaaaaaaaaaaaaaaaaaaaaaaaaaaaaaaaaaaaaaaaaaaaaaa

SEQ ID NO: 34 - protein, 747 aa, homo sapiens
MSQVKSSYSYDAPSDFINFSSLDDEGDTQNIDSWFEEKANLENKLLGKNGTGGLFQGKTPLRKANLQQAIVTPLKPVDNTYYKEAEKENLVEQSIPSNACSSLEVEAAISRKTPAQPQRRSLRLSAQKDLEQKEKHHVKMKAKRCATPVIIDEILPSKKMKVSNNKKKPEEEGSAHQDTAEKNASSPEKAKGRHTVPCMPPAKQKFLKSTEEQELEKSMKMQQEVVEMRKKNEEFKKLALAGIGQPVKKSVSQVTKSVDFHFRTDERIKQHPKNQEEYKEVNFTSELRKHPSSPARVTKGCTIVKPFNLSQGKKRTFDETVSTYVPLAQQVEDFHKRTPNRYHLRSKKDDINLLPSKSSVTKICRDPQTPVLQTKHRARAVTCKSTAELEAEELEKLQQYKFKARELDPRILEGGPILPKKPPVKPPTEPIGFDLEIEKRIQERESKKKTEDEHFEFHSRPCPTKILEDVVGVPEKKVLPITVPKSPAFALKNRIRMPTKEDEEEDEPVVIKAQPVPHYGVPFKPQIPEARTVEICPFSFDSRDKERQLQKEKKIKELQKGEVPKFKALPLPHFDTINLPEKKVKNVTQIEPFCLETDRRGALKAQTWKHQLEEELRQQKEAACFKARPNTVISQEPFVPKKEKKSVAEGLSGSLVQEPFQLATEKRAKERQELEKRMAEVEAQKAQQLEEARLQEEEQKKEELARLRRELVHKANPIRKYQGLEIKSSDQPLTVPVSPKFSTRFHC

SEQ ID NO: 35 - DNA, 1246 bp, homo sapiens
  CDS (112)..(795)
gaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcaaaaaaaaaaaaaaa

SEQ ID NO: 36 - protein, 228 aa, homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 37 - DNA, 7557 bp, homo sapiens
  CDS (958)..(6942)
ggttattaactgtaagactgtttgaattgtcaggtctttgtggtgagctgaattctgcatggcagtgcttactgtgcagaaaggttgcaatgcctcactttgccttaatccatgttcctctaacttccttactctttctctcaagaggaggcaagtggctgtggcggccgcagcagtggctgatcatcactgaaaataccaaagaaaagaactgagctgcctccttcatattttttccattgaggattaatttaccgtgctttttcattttctctacatcctgcaaaagtttttttctctcctaagaaacaaactatgaactgattgttgaaaaaaagaagtaaaaagttttagcacagcttctctgtctcttcgggacaagttagaaaattctgaagtgagccgaagcatagtaagtgctttctttctttttaagctacttctggggagggaggaggctattgtaatggtaaatttcactccgagaggaagaaagggttgataatcaatcaaaaatgaggtattcctttgagtatttgtgattttcttactatattgtaagatgcttttaattttctctgtaaaataggcagaaatggttttagtgtgtgtatgtgtgaaataaaagctcagaaaagcaatcttcagagcgccactgaaggaagttttgacgaacggagtagagatgtataccacttgggggcttcagtgagaacccagaattcctggaggaggatttacattcagaaatgttgaagtgaaaattccttctggttcagcatcttggagttcagcttggaagaacattttacgtatggaagaatttgcttctccaaacctctcttttggccattgtgtgtcctgaaggatgggacaacttgtgctgtagaagcactgcttgcctgagtttgcttcaggcattttaaatttaacttgagggatcatgtgtttggcatgatgaggaccactgaagacttccacaagcctagtgccacattaaactctaacacggccaccaagggaaggtacatttatctggaggcattcctggagggaggagctccctggggttttactctaaagggtggcctggagcacggagaaccattaatcatctctaaggtcgaagaagggggcaaagcagacaccctgagctccaaactgcaggctggggatgaggttgtgcacatcaatgaggtgactctgagcagctccagaaaggaggcagtttccctggtgaaaggatcctacaagaccctcaggctggtagtgcgcagagatgtgtgcacagacccaggccatgcagatactggtgcctctaacttcgtcagcccagaacacctcacctctggcccccagcacaggaaagcagcgtggtcaggaggggttaaacttcggctgaagcacaggtctagtgagcctgcaggccgacctcactcgtggcacacaactaaatctggggagaagcaacccgatgccagcatgatgcagatatctcagggtatgatcggccctccttggcaccaaagctaccattccagctcctctactagtgacctctccaactatgaccatgcttatctaaggcggagccctgaccagtgcagctcccaggggagcatggagagcctggagcccagtggggcatacccaccctgtcatctttcccctgccaagtccaccggcagcattgaccagctcagccacttccataacaagagagactcggcttacagctctttctccaccagttctagcatcctagagtatccacaccctggcatctctgcccgggagcgttcaggctccatggacaatacttctgctcgaggtggcctcctcgaagggatgaggcaggcagatattcgctatgtcaagacagtctatgacacccggaggggagtctcagcagagtatgaggtgaactcttcagccctgctgcttcaaggtagggaggcccgagcctcagcaaatggtcagggctatgataaatggtctaatattcctcggggcaagggagtgccacccccatcctggagccagcagtgccccagttccttggagactgccacggacaaccttcctcctaaggtgggtgcacccctgcctccagctcggagtgacagttacgcagcatttcggcaccgtgagcggcccagctcctggtctagccttgatcagaaacggctctgccggcctcaggcaaactctttaggctccctgaagtctccattcatagaggagcagctgcatactgtgctggagaagagtccagagaacagccccccagtgaagcccaagcataactatacccagaaggcccaacctggccaacctctgctgccgaccagcatctacgcggtaccttccctggagccacactttgcccaggtgcctcagccttctgtgagtagcaacggtatgctctaccctgcactggccaaggagagtggatacatagcccctcagggagcatgcaacaagatggctaccattgatgagaatgggaaccagaatggatctggcaggcctgggtttgccttctgccagcccttagaacatgacttgctgtccccagtggagaagaaaccagaagctacagccaagtatgtcccctccaaagtccatttctgttcagtgcctgaaaatgaggaggatgcctccctgaagagacatctcacacctccccaaggcaacagcccacattccaatgagagaaagagcacccacagtaacaaaccatcttctcatccccacagcctcaaatgccctcaggctcaggcctggcaagcgggtgaagacaagagatcttccaggctctcagagccctgggagggcgatttccaggaagaccacaatgccaacctctggaggaggctggagagagaaggcctaggccagagcctgtcaggcaactttggcaagaccaagtcagccttctcatctctccagaacattcctgagagtctgagaagacacagcagcctggagctaggccggggaacccaggagggttaccccgggggcaggcccacctgtgcagtcaacaccaaggcagaagaccctgggaggaaagccgctcctgacctcgggagccatctggaccggcaggtttcctacccgcggcccgaggggaggaccggtgcctcggcttctttcaacagcacagacccaagtcccgaagagccgcctgccccctcgcacccgcacacatccagtctgggccggagggggcccggcccaggcagcgcctcggctcttcagggctttcagtacgggaagccccactgctcggtgctggagaaggtctccaaattcgagcagcgagagcaagggagccagagaccgagtgtgggcggctctggttttggccataactataggccccacaggaccgtctcaacttccagtacttctgggaatgacttcgaggagacaaaagcacacattcgtttctctgagtcagctgaacccctaggcaacggggagcagcacttcaaaaacggggagctgaagttggaagaggcttcccggcagccctgcggtcagcagctgagcggaggagcgtcggacagcggccgtggcccccagaggccggacgctcggctcctccgtagccagagcaccttccagctctccagcgagccagagagggagcccgagtggcgggacaggcccggctcgcccgaatcgcccctgctggatgcccccttcagccgcgcctaccggaacagcatcaaggacgcacagtcccgtgtcttgggggccacctcctttcgacgtcgagacctggagctgggggcgcccgtggcgtcgaggtcctggcggccacggccttcctcggcccacgtggggctgcggagccccgaggcgtcggcctccgcctccccgcacacgccccgggagcggcacagcgtgacccctgctgagggcgacctggccaggcccgtgccccctgccgcccggagaggtgctcgccggcgcctgactcccgagcagaagaagcgctcctactcggagcccgagaagatgaacgaggtggggatcgtggaggaggccgaaccggcacccctgggcccgcagagaaatgggatgcgtttcccggagagcagcgtggccgaccggcgccgtctcttcgagcgcgatggcaaggcctgctccacgctcagcctgtcggggcccgagctgaagcagttccagcagagcgccctggcggactacatccagcgcaagaccggcaagcggcctacctccgccgccggctgcagcctccaggagcccgggccactgcgtgagcgcgcccagagtgcctacctccagcccggccccgcggcgctcgaaggctccggcctcgcctcggcctccagcttgagctcactgcgggagcccagcctgcagccccgcagggaggccacgctcctgccggccacagttgcagaaacccagcaggctccccgagatcgcagcagctccttcgccggtggccgccgcctcggggaacggcgacgcggggacctgcttagcggagcaaacggtggaacaaggggcacccagagaggggatgagacccccagggagccatcctcctggggggccagggccgggaagtccatgtcggccgaggacctgctggaacgctcggacgtccttgcgggccctgtccatgtgaggtccaggtcatctcccgccaccgcagacaagcgccaggatgtgcttttggggcaagacagtggctttggtcttgtgaaggatccatgttatttggctggtcctggatctaggtcactcagttgttcagaaagaggccaagaagagatgctgctgctcttccaccatctcacccctcgttggggtggttcaggctgcaaagccattggtgattcctccgttcctagtgaatgtcctggaaccctggaccatcagaggcaagccagtaggacaccctgccccaggccaccactggcaggaacgcaagggctggtcacagacaccagggctgcacccctgaccccaattggcacccctctgccttcagccattccctctggctactgctcacaggacggtcagacagggcgacagcctctcccgccctacacccctgccatgatgcacagaagcaatggtcacaccctgacccagcctcccggtccaagaggctgtgagggcgatggcccagagcatggggtagaagagggaacgaggaagagggtctcgctgcctcagtggccacctccttctcgagcaaagtgggcccacgcagccagagaggacagccttcctgaggaatcctcagcccctgattttgcaaacctgaagcactatcaaaaacagcagagtcttccaagtttatgcagcacttctgacccagacacacctcttggggccccgagcactccagggaggatctccctccgaatatctgagtctgtcctgcgggactccccgccacctcatgaggattatgaagacgaagtgtttgtgagggatccgcaccccaaggccacgtccagccccacatttgaacctcttcccccacccccacctcctccaccgagtcaggaaaccccggtgtatagcatggatgacttccctccacctcctccccacactgtatgtgaggcgcagctggacagtgaggatcccgaggggccacgccccagcttcaacaaactttctaaagtgacaattgcaagggaaaggcacatgcctggtgcagcccatgtggtaggtagtcagacactggcttccagactccaaacttctatcaagggttcagaggctgagtccacaccaccctccttcatgagcgttcacgcccaacttgctgggtctcttggtgggcagccagcacccatccagactcaaagcctcagccatgatccagtcagtggaactcagggtttagaaaagaaagtcagtcctgatcctcagaagagttcagaagacatcagaacagaggctttggccaaggaaattgtccaccaagacaaatctctagcagacattttggatccagactccaggctgaagacaacaatggacctgatggaaggtttgtttccccgagatgtgaacttgctgaaggaaaacagtgtaaagaggaaggccatacagagaactgtcagctcttcaggatgtgaaggcaagaggaatgaagacaaggaagcagtgagcatgttggttaactgccctgcctactacagtgtgtctgctcccaaggctgagctactgaacaaaatcaaagagatgccagcagaagtgaatgaggaagaggaacaggcagatgtcaatgaaaagaaggctgagctcattggaagtctcacccacaagctggagaccctccaggaggcgaaggggagcctgctcacggacatcaagctcaacaacgccctgggagaagaggtggaggctctgatcagcgagctctgcaagcccaatgagtttgacaagtataggatgttcataggggatttggacaaggtggtcaacctgctgctctccctctcggggcgtctagcccgtgttgagaatgtccttagcggccttggtgaagatgccagtaatgaagaaaggagctctctttacgagaaaaggaagatcctggctggtcagcatgaggatgcccgggagctgaaggagaacctggatcgcagggagcgagtagtgctgggcatcttggccaattacctttcagaggagcagctccaggactaccagcacttcgtgaaaatgaagtccacgctcctcattgagcaacggaagctggatgacaagatcaagctgggccaggagcaggtcaagtgtctgctggagagcctgccctcagatttcattcccaaggctggggccctggctctgcccccaaacctcacgagtgagcccattcctgctgggggctgtactttcagtggtattttcccaacattaacctctccactttaacctcttctaaaatacccaaccaaaagatcactgtttctctcaacactatttaatctgaaaaatgtttcagtacaaaccactgtttgaactatctgggttattggtgtttgttcctgatgaaaggaaaaaaattctctccaggaggaagcctttttccttcttgcccttcctgattgatcttctgagagctcgaatgctgctggacacgtacccctttctattattactttgtagtagaaagaaagttaatgaaactgagaactgattggagggtgtttgatcatttagtttttaacaggctgaggcaacatggatcagtgtgtgtccccctcaggaatgtatccacagtggccttccttgctggtgggcagtgtatcctgatggcagggtacaagtaccattaatgaagggtctgcaacataaagccttaaaaagacacacactaagaaaactgtaaaaccttgaacattgttatttatattttttaaaatggaaaagatcactatgtttgttgtgctaaccacttatttgattctgttttgtggtggacatagatgattacgtttgagctttgtattttgtgaaaaccttaatgaaatgaattccaaagat

SEQ ID NO: 38 - protein, 1995 aa, homo sapiens
MRTTEDFHKPSATLNSNTATKGRYIYLEAFLEGGAPWGFTLKGGLEHGEPLIISKVEEGGKADTLSSKLQAGDEVVHINEVTLSSSRKEAVSLVKGSYKTLRLVVRRDVCTDPGHADTGASNFVSPEHLTSGPQHRKAAWSGGVKLRLKHRSSEPAGRPHSWHTTKSGEKQPDASMMQISQGMIGPPWHQSYHSSSSTSDLSNYDHAYLRRSPDQCSSQGSMESLEPSGAYPPCHLSPAKSTGSIDQLSHFHNKRDSAYSSFSTSSSILEYPHPGISARERSGSMDNTSARGGLLEGMRQADIRYVKTVYDTRRGVSAEYEVNSSALLLQGREARASANGQGYDKWSNIPRGKGVPPPSWSQQCPSSLETATDNLPPKVGAPLPPARSDSYAAFRHRERPSSWSSLDQKRLCRPQANSLGSLKSPFIEEQLHTVLEKSPENSPPVKPKHNYTQKAQPGQPLLPTSIYAVPSLEPHFAQVPQPSVSSNGMLYPALAKESGYIAPQGACNKMATIDENGNQNGSGRPGFAFCQPLEHDLLSPVEKKPEATAKYVPSKVHFCSVPENEEDASLKRHLTPPQGNSPHSNERKSTHSNKPSSHPHSLKCPQAQAWQAGEDKRSSRLSEPWEGDFQEDHNANLWRRLEREGLGQSLSGNFGKTKSAFSSLQNIPESLRRHSSLELGRGTQEGYPGGRPTCAVNTKAEDPGRKAAPDLGSHLDRQVSYPRPEGRTGASASFNSTDPSPEEPPAPSHPHTSSLGRRGPGPGSASALQGFQYGKPHCSVLEKVSKFEQREQGSQRPSVGGSGFGHNYRPHRTVSTSSTSGNDFEETKAHIRFSESAEPLGNGEQHFKNGELKLEEASRQPCGQQLSGGASDSGRGPQRPDARLLRSQSTFQLSSEPEREPEWRDRPGSPESPLLDAPFSRAYRNSIKDAQSRVLGATSFRRRDLELGAPVASRSWRPRPSSAHVGLRSPEASASASPHTPRERHSVTPAEGDLARPVPPAARRGARRRLTPEQKKRSYSEPEKMNEVGIVEEAEPAPLGPQRNGMRFPESSVADRRRLFERDGKACSTLSLSGPELKQFQQSALADYIQRKTGKRPTSAAGCSLQEPGPLRERAQSAYLQPGPAALEGSGLASASSLSSLREPSLQPRREATLLPATVAETQQAPRDRSSSFAGGRRLGERRRGDLLSGANGGTRGTQRGDETPREPSSWGARAGKSMSAEDLLERSDVLAGPVHVRSRSSPATADKRQDVLLGQDSGFGLVKDPCYLAGPGSRSLSCSERGQEEMLLLFHHLTPRWGGSGCKAIGDSSVPSECPGTLDHQRQASRTPCPRPPLAGTQGLVTDTRAAPLTPIGTPLPSAIPSGYCSQDGQTGRQPLPPYTPAMMHRSNGHTLTQPPGPRGCEGDGPEHGVEEGTRKRVSLPQWPPPSRAKWAHAAREDSLPEESSAPDFANLKHYQKQQSLPSLCSTSDPDTPLGAPSTPGRISLRISESVLRDSPPPHEDYEDEVFVRDPHPKATSSPTFEPLPPPPPPPPSQETPVYSMDDFPPPPPHTVCEAQLDSEDPEGPRPSFNKLSKVTIARERHMPGAAHVVGSQTLASRLQTSIKGSEAESTPPSFMSVHAQLAGSLGGQPAPIQTQSLSHDPVSGTQGLEKKVSPDPQKSSEDIRTEALAKEIVHQDKSLADILDPDSRLKTTMDLMEGLFPRDVNLLKENSVKRKAIQRTVSSSGCEGKRNEDKEAVSMLVNCPAYYSVSAPKAELLNKIKEMPAEVNEEEEQADVNEKKAELIGSLTHKLETLQEAKGSLLTDIKLNNALGEEVEALISELCKPNEFDKYRMFIGDLDKVVNLLLSLSGRLARVENVLSGLGEDASNEERSSLYEKRKILAGQHEDARELKENLDRRERVVLGILANYLSEEQLQDYQHFVKMKSTLLIEQRKLDDKIKLGQEQVKCLLESLPSDFIPKAGALALPPNLTSEPIPAGGCTFSGIFPTLTSPL

SEQ ID NO: 39 - DNA, 2529 bp, homo sapiens
  CDS (203)..(1315)
gagagagagagagagagagaactagtctcgaggtcgacgcggccgcgaattcgcggccgcgtcgaccgaagacgcaggctctatttagagccgggtaggggagcgcacggccagatacctcagcgctacctggcggaactggatttctctcccgcctgccggcctgcctgccacagccggactccgccactccggtagcctcatggctgcaacctgtgagattagcaacatttttagcaactacttcagtgcgatgtacagctcggaggactccaccctggcctctgttccccctgctgccacctttggggccgatgacttggtactgaccctgagcaacccccagatgtcattggagggtacagagaaggccagctggttgggggaacagccccagttctggtcgaagacgcaggttctggactggatcagctaccaagtggagaagaacaagtacgacgcaagcgccattgacttctcacgatgtgacatggatggcgccaccctctgcaattgtgcccttgaggagctgcgtctggtctttgggcctctgggggaccaactccatgcccagctgcgagacctcacttccagctcttctgatgagctcagttggatcattgagctgctggagaaggatggcatggccttccaggaggccctagacccagggccctttgaccagggcagcccctttgcccaggagctgctggacgacggtcagcaagccagcccctaccaccccggcagctgtggcgcaggagccccctcccccggcagctctgacgtctccaccgcagggactggtgcttctcggagctcccactcctcagactccggtggaagtgacgtggacctggatcccactgatggcaagctcttccccagcgatggttttcgtgactgcaagaagggggatcccaagcacgggaagcggaaacgaggccggccccgaaagctgagcaaagagtactgggactgtctcgagggcaagaagagcaagcacgcgcccagaggcacccacctgtgggagttcatccgggacatcctcatccacccggagctcaacgagggcctcatgaagtgggagaatcggcatgaaggcgtcttcaagttcctgcgctccgaggctgtggcccaactatggggccaaaagaaaaagaacagcaacatgacctacgagaagctgagccgggccatgaggtactactacaaacgggagatcctggaacgggtggatggccggcgactcgtctacaagtttggcaaaaactcaagcggctggaaggaggaagaggttctccagagtcggaactgagggttggaactatacccgggaccaaactcacggaccactcgaggcctgcaaaccttcctgggaggacaggcaggccagatggcccctccactggggaatgctcccagctgtgctgtggagagaagctgatgttttggtgtattgtcagccatcgtccttggactcggagactatggcctcgcctccccaccctcctcttggaattacaagccctggggtttgaagctgactttatagctgcaagtgtatctccttttatctggtgcctcctcaaacccagtctcagacactaaatgcagacaacaccttcctcctgcagacacttggactgagccaaggaggcttgggaggccctagggagcaccgtgatggagaggacagagcaggggctccagcacttctttctggactggcgttcacctccctgctcagtgcttgggctccacgggcaggggtcagagcactccctaatttatgtgctatataaatatgtcagatgtacatagagatctattttttctaaaacattcccctccccactcctctcccacagagtgctggactgttccaggccctccagtgggctgatgctgggacccttaggatggggctcccagctcctttctcctgtgaatggaggcagagacctccaataaagtgccttctgggctttttctaacctttgtcttagctacctgtgtactgaaatttgggcctttggatcgaatatggtcaagaggttggaggggaggaaaatgaaggtctaccaggatgagggtgagggcaaaggctgacgaagaggggagttacagatttcctgtagcaggtgtgggcttacagacacatggactgggctgggaggcgagcaaaggaagcagctgagactgttggagaacgttacaagacttcatgcaagaaggacatgaactcagaacactgaggtcagaagcatctgctgtcatgacaccgctcgagtgaccttgaccttgaccaagtcttgtccttgtttaggactgatttttcctattaggctagggtttggacctgatgttctcaagatgtctagaattgcatggctggccttgtggaatagatggttttgcattccagccaagtgtgctgtaaactgtatatctgtaatatgaatcccagcttttgagtctgacaaaatcagagttaggatcttgtaaaggaaaaaaaaaaaaaaaaaa

SEQ ID NO: 40 - protein, 371 aa, homo sapiens
MAATCEISNIFSNYFSAMYSSEDSTLASVPPAATFGADDLVLTLSNPQMSLEGTEKASWLGEQPQFWSKTQVLDWISYQVEKNKYDASAIDFSRCDMDGATLCNCALEELRLVFGPLGDQLHAQLRDLTSSSSDELSWIIELLEKDGMAFQEALDPGPFDQGSPFAQELLDDGQQASPYHPGSCGAGAPSPGSSDVSTAGTGASRSSHSSDSGGSDVDLDPTDGKLFPSDGFRDCKKGDPKHGKRKRGRPRKLSKEYWDCLEGKKSKHAPRGTHLWEFIRDILIHPELNEGLMKWENRHEGVFKFLRSEAVAQLWGQKKKNSNMTYEKLSRAMRYYYKREILERVDGRRLVYKFGKNSSGWKEEEVLQSRN

SEQ ID NO: 41 - DNA, 4020 bp, homo sapiens
  CDS (240)..(1811)
gtcaattgccctttagtcccaggactaaccggaagcttctgcaacaggaggacattgaaaataagatggaacccatccacataaggatttgcctcaaagggcactgcaaaaattgaacagaggaatcccaaggaagctgcctgaatttgcctgtatactctcgttctgcgacttataaaggaccagacaaatcaaattagtggttttggtttccgccagctgtggatgcctttgacattatgaccgcagaggattccaccgcagccatgagcagtgactcggccgccgggtcctcggccaaggtgcccgagggcgtggcgggcgcgcccaacgaggcagcactgctggcgctgatggagcgcacgggctacagcatggtgcaagagaacgggcagcgcaagtacggcggcccaccgcccggctgggagggcccgcacccgcagcgtggctgcgaggtcttcgtgggcaagatcccgcgcgacgtgtacgaggacgagctggtgcccgtgttcgaggccgtgggccgcacctacgagctgcgcctcatgatggactttgacggcaagaaccgcggctacgccttcgtcatgtactgccacaagcacgaggccaagcgcgcagtgcgtgagctcaacaactacgagatccgcccgggccgcctgctcggcgtgtgctgcagcgtggacaactgccgcctcttcatcggcgggatccccaagatgaagaagcgcgaggaaatcctggaggagattgccaaggtcaccgagggcgtgctggacgtgatcgtctacgccagcgcggccgacaagatgaagaaccgcggcttcgccttcgtggagtacgagagccaccgcgcggctgccatggctcgccgcaagctcatgcctggccgcatccagctgtggggccaccagatcgccgtggactgggccgagcctgagatcgacgtggacgaggacgtgatggagaccgtgaagatcctctacgtgcgcaacctcatgatcgagaccaccgaggacaccatcaagaagagcttcggccagttcaaccccggctgcgtggagcgcgtcaagaagatccgcgactacgccttcgtgcacttcaccagccgcgaggatgccgtgcatgccatgaacaacctcaacggcactgagctggagggctcgtgcctggaggtcacgctggccaagcccgtggacaaggagcagtactcgcgctaccagaaggcagccaggggcggcggcgcggctgaggcagcgcagcagcccagctacgtgtactcctgcgacccctacacactggcctactacggctacccctacaacgcgctcattgggcccaacagggactactttgtgaaagtagccatccctgccattggggctcagtattccatgtttccagcagctccagcccctaaaatgattgaagatggcaaaatccacacagtggagcacatgatcagccccattgctgtgcagccagacccagccagtgctgctgccgccgcagccgcggccgcagccgccgcagccgctgtcattcccactgtgtcgacgccaccacctttccagggccgcccaataactccagtatacacggtggctccaaacgttcagagaattcctactgccgggatctacggggccagttacgtgccatttgctgctccagctacagccacgatcgccacactacagaagaacgcggcagccgcggccgccgtgtatggaggatacgcaggctacatacctcaggccttccctgctgctgccattcaggtccccatccccgacgtctaccagacatactgaggctggtgaccagcacgaagacagaccacacaaacaccactgaaggaacgcttgactatttatgaagaaggaacatgttggattcacacatgcaacctgaaagtgaagaatgttagcagatttatttctgaattattttatatacatgaagttttcactagttttttaagactattttcaacttagcatgcctacgttcatacatttccaaaagacttgcaatggttcgtgccttcattccatcttttaaaaatttgtatgctgtactacatttgtatagaggtttttgttgttgtttttttaaggatatattttcagtatgaaggttattttcttaacttctgcactccagagatttctattttgtagtaccttcaataatatatcaactatatattaaaaaagcacacttgaggagctagggaactattttgaaaaatatatacaatatttaaagatacaaacagtagtgcttaaaaatactacataaagcattattttaaaggttatactggaaagtgcaattttaaaatgagtaaaacctctgtatttctgctggcattaagggttgatggtgttaccatgtatcatcatggcggtactattttttaaaagaaattaaacactggatctctccttaagccaacattgaaaagacttgccgcacttctgagtccaaacactggaaagctctccttgccaccgttagccggggctcattctccatgtgccttagccttaaacatgcccccactcccacatctctcaccctgtcccctcctccccagattcccaatcccaccgcaatgtttggcaagcctaggactgataagtagctctgatagaggagctggtggcttttatacttcttcctgggtttttgttggggtttgttgtttcgttgttttttgttttttttttgtttggttggggaagtattgtcttctacgtgtgctattttcagtagcagagtaagcacaaggttttaatcgagttgcataagacacctttgcatagctatttaattgcccaatgtaaaactttaatgccatttctaatgcttttattcatttttgaagtatgagtttgtagggacaaagaatgtatgttatcgtagacaagacccccagagactcttttcagcagaaagttatgcttctagttgccttaccatgtttcttgcaaaactgtccatggtcctcaagggtgttggaaacattatgtttattaaatgggcctctcttcctttgctgtgcacttgatgggtgaactggattggggtgtgcacatccaggaggaggaggagagacctgtagaagtttaaagatagtttgtaaatatcttctaatgcttgtttttagtccttttatgttggagaagttcatggtatgtagtttaatgcaaaatgaaaccattttatttcaatgttattaaaaaggtttgttttattaggaagttaatgtattgttgcagtgttttgtgcctgtttaaaggcttttgtttagcagagtgaatgtaaaatacagtaaaatgttaagattgtcatctactttttaaaaaaaaatatcaacttggaattgttttttaaaggctcaatcaaggaagtgaggtgtgcaataaggtagcaagtaaaacgcagttgcgtttttatgtcatgttagagatccatacaattttccactcacgggatttttgttgatggctgaattcttgtggattcataagaggatcatgcccttagcaagtacttttgttttgttttaaattaagagattcccaaatgcctttttccccctcatcttgaaatgagatgagtttttatgtgtaagcaatatttatttaactattctataaaattattgagtgcctactgaggcctttaagcaccgctaacattcctttccatcattcttttgaatgacataaaataattgtgcaatgttcctgatgatgtaccccacagctgcattcaaactcaaatctgtgggaatgagtgactcgaccaaatgtaattcggatcagatcctcatcccctgactgtgtgaaaaaagtactctccttctagtgaaggattgtcacagagtttcactggatgaaactatgacccagtattcttactgtattttacatgtgcctgtaaattattttgccgaaataagaagaagaagaggaagaaagaacagtagaaaaaaaaaataaaaaaaaa

SEQ ID NO: 42 - protein, 524 aa, homo sapiens
MTAEDSTAAMSSDSAAGSSAKVPEGVAGAPNEAALLALMERTGYSMVQENGQRKYGGPPPGWEGPHPQRGCEVFVGKIPRDVYEDELVPVFEAVGRTYELRLMMDFDGKNRGYAFVMYCHKHEAKRAVRELNNYEIRPGRLLGVCCSVDNCRLFIGGIPKMKKREEILEEIAKVTEGVLDVIVYASAADKMKNRGFAFVEYESHRAAAMARRKLMPGRIQLWGHQIAVDWAEPEIDVDEDVMETVKILYVRNLMIETTEDTIKKSFGQFNPGCVERVKKIRDYAFVHFTSREDAVHAMNNLNGTELEGSCLEVTLAKPVDKEQYSRYQKAARGGGAAEAAQQPSYVYSCDPYTLAYYGYPYNALIGPNRDYFVKVAIPAIGAQYSMFPAAPAPKMIEDGKIHTVEHMISPIAVQPDPASAAAAAAAAAAAAAAVIPTVSTPPPFQGRPITPVYTVAPNVQRIPTAGIYGASYVPFAAPATATIATLQKNAAAAAAVYGGYAGYIPQAFPAAAIQVPIPDVYQTY

SEQ ID NO: 43 - DNA, 1451 bp, homo sapiens
  CDS (161)..(1354)
ttcagaaggaggagagacaccgggcccagggcaccctcgcgggcgggcggacccaagcagtgagggcctgcagccggccggccagggcagcggcaggcgcggcccggacctacgggaggaagccccgagccctcggcgggctgcgagcgactccccggcgatgcctcacaactccatcagatctggccatggagggctgaaccagctgggaggggcctttgtgaatggcagacctctgccggaagtggtccgccagcgcatcgtagacctggcccaccagggtgtaaggccctgcgacatctctcgccagctccgcgtcagccatggctgcgtcagcaagatccttggcaggtactacgagactggcagcatccggcctggagtgatagggggctccaagcccaaggtggccacccccaaggtggtggagaagattggggactacaaacgccagaaccctaccatgtttgcctgggagatccgagaccggctcctggctgagggcgtctgtgacaatgacactgtgcccagtgtcagctccattaatagaatcatccggaccaaagtgcagcaaccattcaacctccctatggacagctgcgtggccaccaagtccctgagtcccggacacacgctgatccccagctcagctgtaactcccccggagtcaccccagtcggattccctgggctccacctactccatcaatgggctcctgggcatcgctcagcctggcagcgacaagaggaaaatggatgacagtgatcaggatagctgccgactaagcattgactcacagagcagcagcagcggaccccgaaagcaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactggggcgcaacctctcgactcaccagacctaccccgtggtggcagctccgcccttttggatctgcagcaagtcggctccggggtcccgcccttcaatgcctttccccatgctgcctccgtgtacgggcagttcacgggccaggccctcctctcagggcgagagatggtggggcccacgctgcccggatacccaccccacatccccaccagcggacagggcagctatgcctcctctgccatcgcaggcatggtggcaggaagtgaatactctggcaatgcctatggccacaccccctactcctcctacagcgaggcctggggcttccccaactccagcttgctgagttccccatattattacagttccacatcaaggccgagtgcaccgcccaccactgccacggcctttgaccatctgtagttgccatggggacagtg

SEQ ID NO: 44 - protein, 398 aa, homo sapiens
MPHNSIRSGHGGLNQLGGAFVNGRPLPEVVRQRIVDLAHQGVRPCDISRQLRVSHGCVSKILGRYYETGSIRPGVIGGSKPKVATPKVVEKIGDYKRQNPTMFAWEIRDRLLAEGVCDNDTVPSVSSINRIIRTKVQQPFNLPMDSCVATKSLSPGHTLIPSSAVTPPESPQSDSLGSTYSINGLLGIAQPGSDKRKMDDSDQDSCRLSIDSQSSSSGPRKHLRTDAFSQHHLEPLECPFERQHYPEAYASPSHTKGEQGLYPLPLLNSTLDDGKATLTPSNTPLGRNLSTHQTYPVVAAPPFWICSKSAPGSRPSMPFPMLPPCTGSSRARPSSQGERWWGPRCPDTHPTSPPADRAAMPPLPSQAWWQEVNTLAMPMATPPTPPTARPGASPTPAC

SEQ ID NO: 45 - DNA, 326 bp, homo sapiens
tttcttttttgttacattcatttgattcagtcccttataaaccccacacctcataaacaagagattagaaactaaacaaaaaggggggcggggaaggaaattctagagtcgttctggtttgcaggtggttgcggtcacaaagagaaatcatcaagaatgttcacttggcatgtgtgaaagattcagggggtctgcagctgtttagtgttgatgcagttgggtcaaaagagtatcatgttagtcttctgtgggttttagggagggattatggagcctccctcccaccccactggctttcttgtgtcacagcctttatttctactccg

SEQ ID NO: 46 - DNA, 1534 bp, homo sapiens
  CDS (267)..(938)
tttgcgctcggggaattaaaagaggggaaaaaaagcccgaagaaaactcacgccccaaacaaaacgcaaggagaggagggcgcgcggcctgcagccctcgcccgcgtccccggccgcggcgtgatgcgcgcggaccagcccgcgacgcccgggctgccgctgtccccgcacctggacgctggcgcggtggccgcgccccagcctcgatcgctcgccgcggcgactcggccccaggcttccggcgccggtgggggccctcgctctccatggggctgagggactggctgagaaccgtgtgctgctgctgccggtgcgagtgcttggaggagcgcgccctgcctgagaaggagcccctcgtcagtgataacaatccatattcctcatttggagcaactctggtgagggatgatgagaagaatttatggagtatgccccatgatgtgtcccacacagaggcagacgacgacagaaccctgtacaatttgatagtcattcgtaatcagcaggccaaagactcagaggagtggcagaagctcaactatgatatccataccctgcggcaggttcgaagggaagtaagaaacagatggaagtgcatcttagaagatttaggttttcaaaaggaagctgactctttgttgtcagtgactaaactcagcaccatcagtgattctaaaaacacaaggaaagctcgagagatgttgttaaaactggctgaagaaaccagtattttcccaacaagttgggagctctcagagagatatctctttgttgtggaccgtctcattgcacttgatgctgcagaagagttctttaagcttgctcgtcgaacttaccccaagaagcctggggttccatgcctggcagatggccagaaagaactgcacctgtggggggacctctcatgcagacttgcacatatgcagggagtattgcactgaagatctttgctggaccttcttctcttcagaagataattttcaaaagggagcaatgctgtgaatgcagcttgcttctctctacagattgagaagtccagcttcaaaagttacttgccacttaagcaaggaacttgtcaagagatcatggttcatgttactgaaaagactttaaggatttgtaaggttaatccatagattgctgagaacaatggaaatatttttatttttacagattttgcacttctgaattcaggttaaaaactaacttgtatttagtctgcttagaggactgtgacttgaaaatttttatataccaatgagctttttggtagcgtccacaatgtttaaaatatttcataggcgagatccgtgttctccatttattaatgcattgtagaccaatttaactgctgtgtttcaggaaaattcttcctagtttaataagcaagctaaaagttttattttttatatttagtgcttaatctttgcctcatgttatgtaaaattagcctgcagatattttctctcaattctgtagactctcgcaagataaacattcaaacagtgaaacaaacaataaaataaataaacct

SEQ ID NO: 47 - protein, 224 aa, homo sapiens
MGLRDWLRTVCCCCRCECLEERALPEKEPLVSDNNPYSSFGATLVRDDEKNLWSMPHDVSHTEADDDRTLYNLIVIRNQQAKDSEEWQKLNYDIHTLRQVRREVRNRWKCILEDLGFQKEADSLLSVTKLSTISDSKNTRKAREMLLKLAEETSIFPTSWELSERYLFVVDRLIALDAAEEFFKLARRTYPKKPGVPCLADGQKELHLWGDLSCRLAHMQGVLH

SEQ ID NO: 48 - DNA, 2385 bp, homo sapiens
  CDS (71)..(1441)
gataccggtttcagagtcctgggcagcgtgcgcgctcttcctggcggctgcgcaggtgtgaaaatcacaaatgtcaaatgatggaagatccaggaatcgggacaggcgctacgatgaggtcccaagcgacctgccctatcaagataccaccataagaacccacccaattcttcatgacagtgagcgggcagtgagcgctgatcccttgccaccaccccctctcccattacagccaccattcggcccagacttctactcaagtgacacagaagaaccagctatagcgccagatctcaaaccagtaaggcgctttgtccctgactcctggaagaactttttcagagggaagaaaaaggaccccgaatgggataagccggtgtctgatatcaggtacatctccgatggagtggagtgttcaccaccagcctctccagcaagaccaaaccaccgttcgcccctcaactcctgcaaagatccctacggagggtcagaaggaacctttagttcccggaaagaggctgacgcagtgtttccccgggatccctatggatctctagaccgacacacacaaacagttcgaacatacagtgagaaggtggaggagtataacctgagatactcctacatgaagtcgtgggcaggcctgctgagaatactgggtgtggtggagctgcttttgggggccggtgtctttgcttgtgtcacagcttacattcacaaggacagtgagtggtacaacttgtttggatattcacaaccgtatggcatgggaggcgttggtggattgggcagtatgtatgggggctattactacactggccctaagaccccttttgtactcgtggttgctggattagcttggatcaccaccattattattctggttcttggcatgtccatgtattaccggaccattcttctggactctaattggtggcccctaactgaatttggaattaacgttgccttgtttattttgtatatggccgcagccatagtctatgtgaatgataccaaccgaggtggcctctgctactatccgttatttaatacaccagtgaatgcagtgttctgccgggtagaaggaggacagatagctgcaatgatcttcctgtttgtcaccatgatagtttatctcattagtgctttggtttgcctaaagttatggaggcatgaggcagctcggagacatagagaatatatggaacaacaggagataaatgagccatcattgtcatcgaaaaggaaaatgtgtgaaatggccaccagtggtgacagacaaagagactcagaagttaatttcaaggaactgagaacagcaaaaatgaaacctgaactactgagtggacacatccccccacgcccagctaatttttttgtatttttagtagagatggggtttcaccgtgttagccaggatgatctcgatctcctgacctcatgatccacccgcctcagcctcccaaagtgttgggattacaggcgtgagtcaccgcgcccagctggtattgcttttctattccctttggacatacatgctacagtcccacaatgtagcatttccttggaaactccctttttttttttttttgagatggagtttcgctcttgttgcccaggctggagtacagtggtatgatcttggctcactgcagcctctgcctcctgggttcaagcgattctcctgcctctgcctcccaagtagctgggattacaggcacccaccaccatgcccagctaattttttgtatttttagtagagacaggatttcactatgttggccaggttggtctcaagctcctgacctcagatgatctaccagcctcggccttctgaagtgctgggattcaggtgtgagccactgtgcccagcagggatgcttcatctttctaagaattatcttggctttggactttattcataaatgttttatttctgttagtatgaacaatagactgccttaacaaagtttttttttaaacaaaatcgttcttgttggattttattcagcagcatctatcatgtagataaattcccaggtgtagcattacagcttctgactaatatagctgccattcagacaattaatgttcaaagagttttctaaagtgataaaaccaaagaaaagcatgtggaaaagcagaagcttagaaagttgtggtcactgaatgcactccctggtttttatttgtcagtgaaatctttatgcattcattgttaatattttaattccatggctttgtaggctgtgctgtgtctgaaggggtaacacctagggaaacatgaggccccttatgggaccccccaaatggaacaacttcactttctcttttatgtattgagccctgtgttaacatttcacttaagaagagcaccagtgcttt

SEQ ID NO: 49 - protein, 457 aa, homo sapiens
MSNDGRSRNRDRRYDEVPSDLPYQDTTIRTHPILHDSERAVSADPLPPPPLPLQPPFGPDFYSSDTEEPAIAPDLKPVRRFVPDSWKNFFRGKKKDPEWDKPVSDIRYISDGVECSPPASPARPNHRSPLNSCKDPYGGSEGTFSSRKEADAVFPRDPYGSLDRHTQTVRTYSEKVEEYNLRYSYMKSWAGLLRILGVVELLLGAGVFACVTAYIHKDSEWYNLFGYSQPYGMGGVGGLGSMYGGYYYTGPKTPFVLVVAGLAWITTIIILVLGMSMYYRTILLDSNWWPLTEFGINVALFILYMAAAIVYVNDTNRGGLCYYPLFNTPVNAVFCRVEGGQIAAMIFLFVTMIVYLISALVCLKLWRHEAARRHREYMEQQEINEPSLSSKRKMCEMATSGDRQRDSEVNFKELRTAKMKPELLSGHIPPRPANFFVFLVEMGFHRVSQDDLDLLTS

SEQ ID NO: 50 - DNA, 2280 bp, homo sapiens
  CDS (64)..(2133)
cgggccaggtttccaggctcggccgccgcctccatcccagcacctgcggagggagcgctgaccatggctccctggcctgaattgggagatgcccagcccaaccccgataagtacctcgaaggggccgcaggtcagcagcccactgcccctgataaaagcaaagagaccaacaaaacagataacactgaggcacctgtaaccaagattgaacttctgccgtcctactccacggctacactgatagatgagcccactgaggtggatgacccctggaacctacccactcttcaggactcggggatcaagtggtcagagagagacaccaaagggaagattctctgtttcttccaagggattgggagattgattttacttctcggatttctctactttttcgtgtgctccctggatattcttagtagcgccttccagctggttggaggaaaaatggcaggacagttcttcagcaacagctctattatgtccaaccctttgttggggctggtgatcggggtgctggtgaccgtcttggtgcagagctccagcacctcaacgtccatcgttgtcagcatggtgtcctcttcattgctcactgttcgggctgccatccccattatcatgggggccaacattggaacgtcaatcaccaacactattgttgcgctcatgcaggtgggagatcggagtgagttcagaagagcttttgcaggagccactgtccatgacttcttcaactggctgtccgtgttggtgctcttgcccgtggaggtggccacccattacctcgagatcataacccagcttatagtggagagcttccacttcaagaatggagaagatgccccagatcttctgaaagtcatcactaagcccttcacaaagctcattgtccagctggataaaaaagttatcagccaaattgcaatgaacgatgaaaaagcgaaaaacaagagtcttgtcaagatttggtgcaaaacttttaccaacaagacccagattaacgtcactgttccctcgactgctaactgcacctccccttccctctgttggacggatggcatccaaaactggaccatgaagaatgtgacctacaaggagaacatcgccaaatgccagcatatctttgtgaatttccacctcccggatcttgctgtgggcaccatcttgctcatactctccctgctggtcctctgtggttgcctgatcatgattgtcaagatcctgggctctgtgctcaaggggcaggtcgccactgtcatcaagaagaccatcaacactgatttcccctttccctttgcatggttgactggctacctggccatcctcgtcggggcaggcatgaccttcatcgtacagagcagctctgtgttcacgtcggccttgacccccctgattggaatcggcgtgataaccattgagagggcttatccactcacgctgggctccaacatcggcaccaccaccaccgccatcctggccgccttagccagccctggcaatgcattgaggagttcactccagatcgccctgtgccactttttcttcaacatctccggcatcttgctgtggtacccgatcccgttcactcgcctgcccatccgcatggccaaggggctgggcaacatctctgccaagtatcgctggttcgccgtcttctacctgatcatcttcttcttcctgatcccgctgacggtgtttggcctctcgctggccggctggcgggtgctggttggtgtcggggttcccgtcgtcttcatcatcatcctggtactgtgcctccgactcctgcagtctcgctgcccacgcgtcctgccgaagaaactccagaactggaacttcctgccgctgtggatgcgctcgctgaagccctgggatgccgtcgtctccaagttcaccggctgcttccagatgcgctgctgctactgctgccgcgtgtgctgccgcgcgtgctgcttgctgtgtggctgccccaagtgctgccgctgcagcaagtgctgcgaggacttggaggaggcgcaggaggggcaggatgtccctgtcaaggctcctgagacctttgataacataaccattagcagagaggctcagggtgaggtccctgcctcggactcaaagaccgaatgcacggccttgtaggggacgccccagattgtcagggatggggggatggtccttgagttttgcatgctctcctccctcccacttctgcaccctttcaccacctcgaggagatttgctccccattagcgaatgaaattgatgcagtcctaaaaaaaaaaa

SEQ ID NO: 51 - protein, 690 aa, homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCYCCRVCCRACCLLCGCPKCCRCSKCCEDLEEAQEGQDVPVKAPETFDNITISREAQGEVPASDSKTECTAL

SEQ ID NO: 52 - DNA, 529 bp, homo sapiens
  MISC (393)..(393): residue at position 393 is A, T, C or G
  misc_feature (393)..(393): n is a, c, g, or t
ctgtttcttcaatggttctcttcccttttccatcctccaaacctggcctgagcctcctgaagttgctgctgtgaatctgaaagacttgaaaagcctccacctgctgtgtggacttcatctcaaggggcccagcctcctctggactccaccttggacctcagtgactcagaacttctgcctctaagctgctctaaagtccagactatggatgtgttctctaggccttcaggactctagaatgtccatatttatttttatgttcttggctttgtgttttaggaaaagtgaatcttgctgttttcaataatgtgaatgctatgttctgggaaaatccactatgacatctaagttttgtgtacagagagatatttttgcaactatttccaccttctnccacaaccccccacactccactccacactcttgagtctctttacctaatggtctctacctaatggaccctcgtggccaaaaagtccattaaaccagaaaggtgattggaaaaaaaaaaaaaaaaactcgagggggg

SEQ ID NO: 53 - DNA, 2100 bp, homo sapiens
  CDS (287)..(1183)
cgaggccgagagggcgggccagggccgcactccggagactcgcggttgctacgcgcaccatggctggagacggagtttcgctcttgatgcccagcaggctggagtgcgatggcgcgatttcggctcactgcaacctcctcctcccagaggtacttctcagccctctagctccaactgagaacccagccagtcaggaagtcgctacttcgggaacaccaaccaatcagggggccgtcacctgctgaaggagtccttcggcggctgttgtgtcgggagcctgatcgcgatggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaatgtgggggtcatcgtggcagccgtccttgtaaccctgattctcctgggaatcttggtttttggcatctggtttgcctatagccgaggccactttgacagaacaaagaaagggacttcgagtaagaaggtgatttacagccagcctagtgcccgaagtgaaggagaattcaaacagacctcgtcattcctggtgtgagcctggtcggctcaccgcctatcatctgcatttgccttactcaggtgctactggactctggcccctgatgtctgtagtttcacaggatgccttatttgtcttctacaccccacagggccccctacttcttcggatgtgtttttaataatgtcagctatgtgccccatcctccttcatgccctccctccctttcctaccactgctgagtggcctggaacttgtttaaagtgtttattccccatttctttgagggatcaggaaggaatcctgggtatgccattgacttcccttctaagtagacagcaaaaatggcgggggtcgcaggaatctgcactcaactgcccacctggctggcagggatctttgaataggtatcttgagcttggttctgggctctttccttgtgtactgacgaccagggccagctgttctagagtgggaattagaggctagagcggctgaaatggttgtttggtgatgacactggggtccttccatctctggggcccactctcttctgtcttcccatgggaagtgccactgggatccctctgccctgtcctcctgaatacaagctgactgacattgactgtgtctgtggaaaatgggagctcttgttgtggagagcatagtaaattttcagagaacttgaagcgaaaaggatttaaaaccgctgctctaaagaaaagaaaactggaggctgggcgcagtggctcacgcctgtaatcccagaggctgaggcaggcggatcacctgaggtcgggagttcgggatcagcctgaccaacatggagaaaccctgctggaaatacagagttagccaggcatggtggtgcatgcctgtagtcccagctgctcaggagcctggcaacaagagcaaaactccagctcaaaaaaaaaaaaaaaaaaaaaaaa

SEQ ID NO: 54 - protein, 299 aa, homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 55 - DNA, 2154 bp, homo sapiens
  CDS (77)..(1108)
atataaccgcgtggcccgcgcgcgcgcttccctcccggcgcagtcaccggcgcggtctatggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccagaccttttgagttcaactgatcaaaccaaaggaaaagtgttgctagagaaaattggggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtatggta

SEQ ID NO: 56 - protein, 344 aa, homo sapiens
MSALAARLLQPAHSCSLRLRPFHLAAVRNEAVVISGRKLAQQIKQEVRQEVEEWVASGNKRPHLSVILVGENPASHSYVLNKTRAAAVVGINSETIMKPASISEEELLNLINKLNNDDNVDGLLVQLPLPEHIDERRICNAVSPDKDVDGFHVINVGRMCLDQYSMLPATPWGVWEIIKRTGIPTLGKNVVVAGRSKNVGMPIAMLLHTDGAHERPGGDATVTISHRYTPKEQLKKHTILADIVISAAGIPNLITADMIKEGAAVIDVGINRVHDPVTAKPKLVGDVDFEGVRQKAGYITPVPGGVGPMTVAMLMKNTIIAAKKVLRLEEREVLKSKELGVATN

SEQ ID NO: 57 - DNA, 1117 bp, homo sapiens
  CDS (57)..(1025)
atctcccactcctgcagctcttctcacaggaccagccactagcgcagcctcgagcgatggcctatgtccccgcaccgggctaccagcccacctacaacccgacgctgccttactaccagcccatcccgggcgggctcaacgtgggaatgtctgtttacatccaaggagtggccagcgagcacatgaagcggttcttcgtgaactttgtggttgggcaggatccgggctcagacgtcgccttccacttcaatccgcggtttgacggctgggacaaggtggtcttcaacacgttgcagggcgggaagtggggcagcgaggagaggaagaggagcatgcccttcaaaaagggtgccgcctttgagctggtcttcatagtcctggctgagcactacaaggtggtggtaaatggaaatcccttctatgagtacgggcaccggcttcccctacagatggtcacccacctgcaagtggatggggatctgcaacttcaatcaatcaacttcatcggaggccagcccctccggccccagggacccccgatgatgccaccttaccctggtcccggacattgccatcaacagctgaacagcctgcccaccatggaaggacccccaaccttcaacccgcctgtgccatatttcgggaggctgcaaggagggctcacagctcgaagaaccatcatcatcaagggctatgtgcctcccacaggcaagagctttgctatcaacttcaaggtgggctcctcaggggacatagctctgcacattaatccccgcatgggcaacggtaccgtggtccggaacagccttctgaatggctcgtggggatccgaggagaagaagatcacccacaacccatttggtcccggacagttctttgatctgtccattcgctgtggcttggatcgcttcaaggtttacgccaatggccagcacctctttgactttgcccatcgcctctcggccttccagagggtggacacattggaaatccagggtgatgtcaccttgtcctatgtccagatctaatctattcctggggccataactcatgggaaaacagaattatcccctaggactcctttctaagcccctaataaaatgtctgagggtgtctc

SEQ ID NO: 58 - protein, 323 aa, homo sapiens
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 59 - DNA, 3697 bp, homo sapiens
  CDS (121)..(2616)
agggagtgttcccgggggagatactccagtcgtagcaagagtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccatagaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttgttaa

SEQ ID NO: 60 - protein, 832 aa, homo sapiens
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIEVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 61 - DNA, 2920 bp, homo sapiens
  CDS (10)..(2247)
cttgcagcaatggcttggattagatccacttgcattctcttttttaccttgctttttgcccacatagcagctgtaccgattaagcatcttcctgaagaaaatgtacatgatgcagattttggtgaacagaaggatatttcagaaatcaatttagctgcaggcttggacctctttcaaggggacatcctcttgcagaaatccagaaatggcctgagagacccaaacaccaggtggacgttccccattccttacatcttggctgataatttggggctgaatgctaaaggagccattctgtatgcctttgagatgttccgtctcaagtcctgtgtggatttcaagccctatgaaggagagagctcatatatcatatttcaacagtttgatgggtgctggtctgaggttggtgaccaacatgtgggacagaacatttccattggccaaggatgtgcctataaggccatcatagaacacgagatcctgcatgctttgggattttaccacgagcagtcaaggacggaccgggatgattatgtgaacatctggtgggaccaaattctttcaggttaccagcacaactttgacacctatgatgatagcttaatcacagacctcaatacaccctatgattatgagtctttgatgcactaccagcctttctcatttaacaagaatgcaagtgttcccaccatcacagccaagatccctgagtttaactccattatcggacaacgcctggatttcagtgccattgatttagagaggctgaaccgaatgtacaattgcaccacaactcacactcttttggaccactgtacttttgagaaggcaaacatctgtggaatgattcagggcaccagagatgacactgactgggcccatcaggacagtgctcaggctggagaagtggatcacaccttgttgggacaatgcacaggtgccggctacttcatgcagttcagcaccagctcggggtccgcggaagaggcagccctactggagtctcggattctttacccaaagaggaagcagcagtgcctgcaatttttctataaaatgacgggaagtccttcagacagactcgttgtctgggtcaggagggatgacagcacaggcaatgttcgcaagttggtgaaggtgcagacttttcaaggagatgatgaccacaattggaaaattgcccatgtggtgctcaaagaggaacagaagtttcgctaccttttccagggcacaaaaggcgaccctcagaactcaactgggggaatttacctagatgacatcactctgacagaaaccccctgccccacaggggtctggacagtccggaatttctcccaagtccttgagaacaccagcaaaggggacaagcttcagagccctcgattctacaattcggagggatatggttttggggtaactttatacccaaatagcagagaaagctctggttacttgagacttgcttttcatgtgtgcagtggggagaacgatgctatcctggagtggccggtagaaaacagacaggtgataattaccatccttgaccaggagcctgatgtccggaacaggatgtcctcaagcatggtgttcactacctcgaagtcgcacacatctccagcgataaatgacactgtcatctgggacaggccgtccagggtgggaacctatcatacagactgtaattgttttagaagcatcgacttgggctggagtggtttcatttcccaccaaatgctgaaaaggaggagtttcctgaaaaatgatgacctcatcatatttgtggactttgaagatatcacccacctcagccagactgaagttccctctaaaggcaaaagactgagcccccaaggcctcattctccaaggccaggagcagcaggtctccgaagaaggttcgggaaaggccatgttagaggaagccctacctgtcagcctgagccaggggcagcccagccgacagaagcggtcggtggagaacacaggccccctggaggaccataactggccacagtacttcagagacccatgtgacccaaacccttgccaaaatgacggcatctgtgtgaacgtgaaggggatggcgagctgcaggtgcatctctggacatgctttcttctacacgggggagcgctgtcagtcggccgaggtgcacggcagtgtcctgggcatggtgatcggaggcacggctggcgtgatcttcttgaccttctccatcatcgccatcctttcccaaaggccaaggaagtgacctgcctgctggcattggccagaccacagcagcacctcctccatgcaggccttaactttcccatgttcaatgcagtttggggcagcttttttatcagccttgctttggataggacctccaaggactaagcctccagccccatgtgtgacccttgtcatctctctgccccacataattatgttactttgctatgtgctcctaatgtatctagtgtgtcctgtgacaacactcatcacacttcattgtaaatcacttgttttattgactgtctttcctatagactgtaagctccatgagggcaggcacatgttgttctcattgaccgtgctggccccagtgcctagatgcatggctggcacattgttggcactcaacaatggttgaatgaataaaacaataaatgaatgaataactaagatatagaaactctcatttatattgcagattgaatatatatgatgaaattcttatgttgaatatgttagaatcaaatactcatttttcattagatacagtagtgtcatcactcttttaagatcttgttaaagatttcaaataaaggtacttctggcgagccaggctgcacagcatttgctttcctctgagattctaagagaaggcctttaataaatttaataaatattgagttagcaaaaaaaaaaaaaaaaaa

SEQ ID NO: 62 - protein, 746 aa, homo sapiens
MAWIRSTCILFFTLLFAHIAAVPIKHLPEENVHDADFGEQKDISEINLAAGLDLFQGDILLQKSRNGLRDPNTRWTFPIPYILADNLGLNAKGAILYAFEMFRLKSCVDFKPYEGESSYIIFQQFDGCWSEVGDQHVGQNISIGQGCAYKAIIEHEILHALGFYHEQSRTDRDDYVNIWWDQILSGYQHNFDTYDDSLITDLNTPYDYESLMHYQPFSFNKNASVPTITAKIPEFNSIIGQRLDFSAIDLERLNRMYNCTTTHTLLDHCTFEKANICGMIQGTRDDTDWAHQDSAQAGEVDHTLLGQCTGAGYFMQFSTSSGSAEEAALLESRILYPKRKQQCLQFFYKMTGSPSDRLVVWVRRDDSTGNVRKLVKVQTFQGDDDHNWKIAHVVLKEEQKFRYLFQGTKGDPQNSTGGIYLDDITLTETPCPTGVWTVRNFSQVLENTSKGDKLQSPRFYNSEGYGFGVTLYPNSRESSGYLRLAFHVCSGENDAILEWPVENRQVIITILDQEPDVRNRMSSSMVFTTSKSHTSPAINDTVIWDRPSRVGTYHTDCNCFRSIDLGWSGFISHQMLKRRSFLKNDDLIIFVDFEDITHLSQTEVPSKGKRLSPQGLILQGQEQQVSEEGSGKAMLEEALPVSLSQGQPSRQKRSVENTGPLEDHNWPQYFRDPCDPNPCQNDGICVNVKGMASCRCISGHAFFYTGERCQSAEVHGSVLGMVIGGTAGVIFLTFSIIAILSQRPRK

SEQ ID NO: 63 - DNA, 8838 bp, homo sapiens
  CDS (34)..(8430)
cgccctcgagtggaggacgagaaggaaagcaccatgacgtccatccatttcgtggttcacccgctgccgggcaccgaggaccagctcaatgacaggttacgagaagtttctgagaagctgaacaaatataatttaaacagccacccccctttgaatgtattggaacaggctactattaaacagtgtgtggtgggaccaaatcatgctgcctttcttcttgaggatggtagagtttgcaggattggtttttcagtacagccagacagattggaattgggtaaacctgataataatgatgggtcaaagttgaacagcaactcgggggcagggaggacgtcaaggcctggtaggacaagcgactctccatggtttctctcaggttctgagactctaggcaggctggcaggcaacaccttaggaagccgctggagttctggagtgggtggaagtggtggaggatcctctggtaggtcatcagctggagctcgagattcccgccggcagactcgagttattcggacaggacgggatcgagggtctgggcttttgggcagtcagccccagccagttattccagcatctgtcattccagaggagctgatttcacaggcccaagttgttttacaaggcaaatccagaagtgtcattattcgagaacttcagagaacaaatcttgatgtgaaccttgctgtaaataatttacttagccgggatgatgaagatggagatgatggggatgatacagccagcgaatcttatttgcctggagaggatcttatgtctctccttgatgccgacattcattctgcccacccaagtgtcattattgatgcagatgccatgttttctgaagacattagctattttggttacccttcttttcgtcgttcatcactttccaggctaggctcatctcgagttctccttcttcccttagagagagactctgagctgttgcgtgaacgtgaatccgttttacgtttacgtgaacgaaggtggcttgatggagcctcatttgataatgaaaggggttctaccagcaaggaaggagagccaaacttggataagaagaatacacctgttcaaagtccagtatctctaggagaagatttgcagtggtggcctgataaggatggaacaaaattcatctgtattggggctctgtattctgaacttctggctgtcagcagtaaaggagaactttatcagtggaaatggagtgaatctgagccttacagaaatgcccagaatccttcattacatcatccacgagcaacatttttggggttaaccaatgaaaagatagtcctcctgtctgcaaatagcataagagcaactgtagctacagaaaataacaaggttgctacatgggtggatgaaactttaagttctgtggcttctaaattagagcacactgctcagacttactctgaacttcaaggagagcggatagtttctttacattgctgtgccctttacacctgcgctcagctggaaaacagtttatattggtggggtgtagttccttttagtcaaaggaagaaaatgttagagaaagctagagcaaaaaataaaaagcctaaatccagtgctggtatttcttcaatgccgaacatcactgttggtacccaggtatgcttgagaaataatcctctttatcatgctggagcagttgcattttcaattagtgctgggattcctaaagttggtgtcttaatggagtcagtttggaatatgaatgacagctgtagatttcaacttagatctcctgaaagcttgaaaaacatggaaaaagctagcaaaactactgaagctaagcctgaaagtaagcaggagccagtgaaaacagaaatgggtcctccaccatctccagcatccacgtgtagtgatgcatcctcaattgccagcagtgcatcaatgccatacaaacgacgacggtcaacccctgcaccaaaagaagaggaaaaggtgaatgaagagcagtggtctcttcgggaagtggtttttgtggaagatgtcaagaatgttcctgttggcaaggtgctaaaagtagatggtgcctatgttgctgtaaaatttccaggaacctccagtaatactaactgtcagaacagctctggtccagatgctgacccttcttctctcctgcaggattgtaggttacttagaattgatgaattgcaggttgtcaaaactggtggaacaccgaaggttcccgactgtttccaaaggactcctaaaaagctttgtatacctgaaaaaacagaaatattagcagtgaatgtagattccaaaggtgttcatgctgttctgaagactggaaattgggtgcgatactgtatctttgatcttgctacaggaaaagcagaacaggaaaataattttcctacaagcagcattgctttccttggtcagaatgagaggaatgtagccattttcactgctggacaggaatctcccattattcttcgagatggaaatggtaccatctacccaatggccaaagattgcatgggaggaataagggatcccgattggctggatcttccacctattagtagtcttggaatgggtgtgcattctttaataaatcttcctgccaattcaacaatcaaaaagaaagctgctgttatcatcatggctgtagagaaacaaaccttaatgcaacacattctgcgctgtgactatgaggcctgtcgacaatatctaatgaatcttgagcaagcggttgttttagagcagaatctacagatgctgcagacattcatcagccacagatgtgatggaaatcgaaatattttgcatgcttgtgtatcagtttgctttccaaccagcaataaagaaactaaagaagaagaggaagcggagcgttctgaaagaaatacatttgcagaaaggctttctgctgttgaggccattgcaaatgcaatatcagttgtttcaagtaatggcccaggtaatcgggctggatcatcaagtagccgaagtttgagattacgggaaatgatgagacgttcgttgagagcagctggtttgggtagacatgaagctggagcttcatccagtgaccaccaggatccagtttcaccccccatagctccccctagttgggttcctgaccctcctgcgatggatcctgatggtgacattgattttatcctggcccccgctgtgggatctcttaccacagcagcaaccggtactggtcaaggaccaagcacctccactattccaggtccttccacagagccatctgtagtagaatccaaggatcgaaaggcgaatgctcattttatattgaaattgttatgtgacagtgtggttctccagccctatctacgagaacttctttctgccaaggatgcaagagggatgaccccatttatgtcagctgtaagtggccgagcttatcctgctgcaattaccatcttagaaactgctcagaaaattgcaaaagctgaaatatcctcaagtgaaaaagaggaagatgtattcatgggaatggtttgcccatcaggtaccaaccctgatgactctcctttatatgttttatgttgtaatgacacttgcagttttacatggactggagcagagcacattaaccaggatatttttgagtgtcgaacttgtggcttgctggagtcactgtgttgttgtacggaatgtgcaagggtttgtcataaaggtcatgattgcaaactcaaacggacatcaccaacagcctactgtgattgttgggagaaatgtaaatgtaaaactcttattgctggacagaaatctgctcgtcttgatctactttatcgcctgctcactgctactaatctggttactctgccaaacagcaggggagagcacctcttactattcttagtacagacagtcgcaaggcagacggtggagcattgtcaatacaggccacctcgaatcagggaagatcgtaaccgaaaaacagccagtcctgaagattcagatatgccagatcatgatttagagcctccaagatttgcccagcttgcattggagcgtgttctacaggactggaatgccttgaaatctatgattatgtttgggtcgcaggagaataaagaccctcttagtgccagcagtagaataggccatcttttgccagaagagcaagtatacctcaatcagcaaagtggcacaattcggctggactgtttcactcattgccttatagttaagtgtacagcagatattttgcttttagatactctactaggtacactagtgaaagaactccaaaacaaatatacacctggacgtagagaagaagctattgctgtgacaatgaggtttctacgttcagtggcaagagtttttgttattctgagtgtggaaatggcttcatccaaaaagaaaaacaactttattccacagccaattggaaaatgcaagcgtgtattccaagcattgctaccttacgctgtggaagaattgtgcaacgtagcagagtcactgattgttcctgtcagaatggggattgctcgtccaactgcaccatttaccctggctagtactagcatagatgccatgcagggcagtgaagaattattttcagtggaaccactaccaccacgaccatcatctgatcagtctagcagctccagtcagtctcagtcatcctacatcatcaggaatccacagcagaggcgcatcagccagtcacagcccgttcggggcagagatgaagaacaggatgatattgtttcagcagatgtggaagaggttgaggtggtggagggtgtggctggagaagaggatcatcatgatgaacaggaagaacacggggaagaaaatgctgaggcagagggacaacatgatgagcatgatgaagacgggagtgatatggagctggacttgttagcagcagctgaaacagaaagtgatagtgaaagtaaccacagcaaccaagataatgctagtgggcgcagaagcgttgtcactgcagcaactgctggttcagaagcaggagcaagcagtgttcctgccttcttttctgaagatgattctcaatcgaatgactcaagtgattctgatagcagtagtagtcagagtgacgacatagaacaggagacctttatgcttgatgagccattagaaagaaccacaaatagctcccatgccaatggtgctgcccaagctccccgttcaatgcagtgggctgtccgcaacacccagcatcagcgagcagccagtacagccccttccagtacatctacaccagcagcaagttcagcgggtttgatttatattgatccttcaaacttacgccggagtggtaccatcagtacaagtgctgcagctgcagcagctgctttggaagctagcaacgccagcagttacctaacatctgcaagcagtttagccagggcttacagcattgtcattagacaaatctcggacttgatgggccttattcctaagtataatcacctagtatactctcagattccagcagctgtgaaattgacttaccaagatgcagtaaacttacagaactatgtagaagaaaagcttattcccacttggaactggatggtcagtattatggattctactgaagctcaattacgttatggttctgcattagcatctgctggtgatcctggacatccaaatcatcctcttcacgcttctcagaattcagcgagaagagagaggatgactgcgcgagaagaagctagcttacgaacacttgaaggcagacgacgtgccaccttgcttagcgcccgtcaaggaatgatgtctgcacgaggagacttcctaaattatgctctgtctctaatgcggtctcataatgatgagcattctgatgttcttccagttttggatgtttgctcattgaagcatgtggcatatgtttttcaagcacttatatactggattaaggcaatgaatcagcagacaacattggatacacctcaactagaacgcaaaaggacgcgagaactcttggaactgggtattgataatgaagattcagaacatgaaaatgatgatgacaccaatcaaagtgctactttgaatgataaggatgatgactctcttcctgcagaaactggccaaaaccatccatttttccgacgttcagactccatgacattccttgggtgtatacccccaaatccatttgaagtgcctctggctgaagccatccccttggctgatcagccacatctgttgcagccaaatgctagaaaggaggatctttttggccgtccaagtcagggtctttattcttcatctgccagtagtgggaaatgtttaatggaggttacagtggatagaaactgcctagaggttcttccaacaaaaatgtcttatgctgccaatctgaaaaatgtaatgaacatgcaaaaccggcaaaaaaaagaaggggaagaacagcccgtgctgccagaagaaactgagagttcaaaaccagggccatctgctcatgatcttgctgcacaattaaaaagtagcttactagcagaaataggacttactgaaagtgaagggccacctctcacatctttcaggccacagtgtagctttatgggaatggttatttcccatgatatgctgctaggacgttggcgcctttctttagaactgttcggcagggtattcatggaagatgttggagcagaacctggatcaatcctaactgaattgggtggttttgaggtaaaagaatcaaaattccgcagagaaatggaaaaactgagaaaccagcagtcaagagatttgtcactagaggttgatcgggatcgagatcttctcattcagcagactatgaggcagcttaacaatcactttggtcgaagatgtgctactacaccaatggctgtacacagagtaaaagtcacatttaaggatgagccaggagagggcagtggtgtagcacgaagtttttatacagccattgcacaagcatttttatcaaatgaaaaattgccaaatctagagtgtatccaaaatgccaacaaaggcacccacacaagtttaatgcagagattaaggaaccgaggagagagagaccgggaaagggagagagaaagggaaatgaggaggagtagtggtttgcgagcaggttctcggagggaccgggatagagactttagaagacagctttccatcgacactaggccctttagaccagcctctgaagggaatcctagcgatgatcctgagcctttgccagcacatcggcaggcacttggagagaggctttatcctcgtgtacaagcaatgcaaccagcatttgcaagtaaaatcactggcatgttgttggaattatccccagctcagctgcttctccttctagcaagtgaggattctctgagagcaagagtggatgaggccatggaactcattattgcacatggacgggaaaatggagctgatagtatcctggatcttggattagtagactcctcagaaaaggtacagcaggaaaaccgaaagcgccatggctctagtcgaagtgtagtagatatggatttagatgatacagatgatggtgatgacaatgcccctttgttttaccaacctgggaaaagaggattttatactccaaggcctggcaagaacacagaagcaaggttgaattgtttcagaaacattggcaggattcttggactatgtctgttacagaatgaactctgtcctatcacattgaatagacatgtaattaaagtattgcttggtagaaaagtcaattggcatgattttgctttttttgatcctgtaatgtatgagagtttgcggcaactaatcctcgcgtctcagagttcagatgctgatgctgttttctcagcaatggatttggcatttgcaattgacctgtgtaaagaagaaggtggaggacaggttgaactcattcctaatggtgtaaatataccagtcactccacagaatgtatatgagtatgtgcggaaatacgcagaacacagaatgttggtagttgcagaacagcccttacatgcaatgaggaaaggtctactagatgtgcttccaaaaaattcattagaagatttaacggcagaagattttaggcttttggtaaatggctgcggtgaagtcaatgtgcaaatgctgatcagttttacctctttcaatgatgaatcaggagaaaatgctgagaagcttctgcagttcaagcgttggttctggtcaatagtagagaagatgagcatgacagaacgacaagatcttgtttacttttggacatcaagcccatcactgccagccagtgaagaaggattccagcctatgccctcaatcacaataagaccaccagatgaccaacatcttcctactgcaaatacttgcatttctcgactttacgtcccactctattcctctaaacagattctcaaacagaaattgttactcgccattaagaccaagaattttggttttgtgtagagtataaaaagtgtgtattgctgtgtaatattactagcaaattttgtagatttttttccatttgtctataaaagttaaagtttatggaagttaatgctgtcatacccccctggtggtaccttaaagagataaaatgcagacattccttgctgagtttatagcttaaaggcctaaggagcactagcaacatttggctatattggtttgctagtcaccaacttctgggtctaaccccagccaaagatgacagcagaacaacataatttacactgtgatttatctttttgctgaggggaaaaaaatgtaaatgttctgaaaattcactgctgcctttgtggaaactgtttcagcaaaggttcttgtatagagggaatagggaatttcaaaataaaaaattaagtatgt

SEQ ID NO: 64 - protein, 2799 aa, homo sapiens
MTSIHFVVHPLPGTEDQLNDRLREVSEKLNKYNLNSHPPLNVLEQATIKQCVVGPNHAAFLLEDGRVCRIGFSVQPDRLELGKPDNNDGSKLNSNSGAGRTSRPGRTSDSPWFLSGSETLGRLAGNTLGSRWSSGVGGSGGGSSGRSSAGARDSRRQTRVIRTGRDRGSGLLGSQPQPVIPASVIPEELISQAQVVLQGKSRSVIIRELQRTNLDVNLAVNNLLSRDDEDGDDGDDTASESYLPGEDLMSLLDADIHSAHPSVIIDADAMFSEDISYFGYPSFRRSSLSRLGSSRVLLLPLERDSELLRERESVLRLRERRWLDGASFDNERGSTSKEGEPNLDKKNTPVQSPVSLGEDLQWWPDKDGTKFICIGALYSELLAVSSKGELYQWKWSESEPYRNAQNPSLHHPRATFLGLTNEKIVLLSANSIRATVATENNKVATWVDETLSSVASKLEHTAQTYSELQGERIVSLHCCALYTCAQLENSLYWWGVVPFSQRKKMLEKARAKNKKPKSSAGISSMPNITVGTQVCLRNNPLYHAGAVAFSISAGIPKVGVLMESVWNMNDSCRFQLRSPESLKNMEKASKTTEAKPESKQEPVKTEMGPPPSPASTCSDASSIASSASMPYKRRRSTPAPKEEEKVNEEQWSLREVVFVEDVKNVPVGKVLKVDGAYVAVKFPGTSSNTNCQNSSGPDADPSSLLQDCRLLRIDELQVVKTGGTPKVPDCFQRTPKKLCIPEKTEILAVNVDSKGVHAVLKTGNWVRYCIFDLATGKAEQENNFPTSSIAFLGQNERNVAIFTAGQESPIILRDGNGTIYPMAKDCMGGIRDPDWLDLPPISSLGMGVHSLINLPANSTIKKKAAVIIMAVEKQTLMQHILRCDYEACRQYLMNLEQAVVLEQNLQMLQTFISHRCDGNRNILHACVSVCFPTSNKETKEEEEAERSERNTFAERLSAVEAIANAISVVSSNGPGNRAGSSSSRSLRLREMMRRSLRAAGLGRHEAGASSSDHQDPVSPPIAPPSWVPDPPAMDPDGDIDFILAPAVGSLTTAATGTGQGPSTSTIPGPSTEPSVVESKDRKANAHFILKLLCDSVVLQPYLRELLSAKDARGMTPFMSAVSGRAYPAAITILETAQKIAKAEISSSEKEEDVFMGMVCPSGTNPDDSPLYVLCCNDTCSFTWTGAEHINQDIFECRTCGLLESLCCCTECARVCHKGHDCKLKRTSPTAYCDCWEKCKCKTLIAGQKSARLDLLYRLLTATNLVTLPNSRGEHLLLFLVQTVARQTVEHCQYRPPRIREDRNRKTASPEDSDMPDHDLEPPRFAQLALERVLQDWNALKSMIMFGSQENKDPLSASSRIGHLLPEEQVYLNQQSGTIRLDCFTHCLIVKCTADILLLDTLLGTLVKELQNKYTPGRREEAIAVTMRFLRSVARVFVILSVEMASSKKKNNFIPQPIGKCKRVFQALLPYAVEELCNVAESLIVPVRMGIARPTAPFTLASTSIDAMQGSEELFSVEPLPPRPSSDQSSSSSQSQSSYIIRNPQQRRISQSQPVRGRDEEQDDIVSADVEEVEVVEGVAGEEDHHDEQEEHGEENAEAEGQHDEHDEDGSDMELDLLAAAETESDSESNHSNQDNASGRRSVVTAATAGSEAGASSVPAFFSEDDSQSNDSSDSDSSSSQSDDIEQETFMLDEPLERTTNSSHANGAAQAPRSMQWAVRNTQHQRAASTAPSSTSTPAASSAGLIYIDPSNLRRSGTISTSAAAAAAALEASNASSYLTSASSLARAYSIVIRQISDLMGLIPKYNHLVYSQIPAAVKLTYQDAVNLQNYVEEKLIPTWNWMVSIMDSTEAQLRYGSALASAGDPGHPNHPLHASQNSARRERMTAREEASLRTLEGRRRATLLSARQGMMSARGDFLNYALSLMRSHNDEHSDVLPVLDVCSLKHVAYVFQALIYWIKAMNQQTTLDTPQLERKRTRELLELGIDNEDSEHENDDDTNQSATLNDKDDDSLPAETGQNHPFFRRSDSMTFLGCIPPNPFEVPLAEAIPLADQPHLLQPNARKEDLFGRPSQGLYSSSASSGKCLMEVTVDRNCLEVLPTKMSYAANLKNVMNMQNRQKKEGEEQPVLPEETESSKPGPSAHDLAAQLKSSLLAEIGLTESEGPPLTSFRPQCSFMGMVISHDMLLGRWRLSLELFGRVFMEDVGAEPGSILTELGGFEVKESKFRREMEKLRNQQSRDLSLEVDRDRDLLIQQTMRQLNNHFGRRCATTPMAVHRVKVTFKDEPGEGSGVARSFYTAIAQAFLSNEKLPNLECIQNANKGTHTSLMQRLRNRGERDREREREREMRRSSGLRAGSRRDRDRDFRRQLSIDTRPFRPASEGNPSDDPEPLPAHRQALGERLYPRVQAMQPAFASKITGMLLELSPAQLLLLLASEDSLRARVDEAMELIIAHGRENGADSILDLGLVDSSEKVQQENRKRHGSSRSVVDMDLDDTDDGDDNAPLFYQPGKRGFYTPRPGKNTEARLNCFRNIGRILGLCLLQNELCPITLNRHVIKVLLGRKVNWHDFAFFDPVMYESLRQLILASQSSDADAVFSAMDLAFAIDLCKEEGGGQVELIPNGVNIPVTPQNVYEYVRKYAEHRMLVVAEQPLHAMRKGLLDVLPKNSLEDLTAEDFRLLVNGCGEVNVQMLISFTSFNDESGENAEKLLQFKRWFWSIVEKMSMTERQDLVYFWTSSPSLPASEEGFQPMPSITIRPPDDQHLPTANTCISRLYVPLYSSKQILKQKLLLAIKTKNFGFV

SEQ ID NO: 65 - DNA, 4256 bp, homo sapiens
  CDS (126)..(3359)
tacgagcggctgtgaaggaaactgtttaaccggatcccattgtacccagagtgcagagccgcctttccagcatgcaggggctgctcagcgtttagtcacatcaagaaatagaacagaattcagccatggccccaagaaagagaggtggacgaggtatttcattcatcttttgctgtttccgaaataatgatcacccagaaatcacgtatcggctgcgaaatgatagcaactttgcgcttcagaccatggaaccagcattgcccatgccccctgtggaggagctggatgtcatgttcagtgaactggtggatgaactggacctcacagacaaacacagagaagccatgtttgcacttccagctgagaaaaaatggcaaatatactgtagcaagaaaaaggaccaggaagaaaacaagggagctacaagttggcctgaattctacattgatcagctcaattccatggctgctagaaaatctctgctggctttagagaaggaagaagaagaagaaagaagtaaaactatagagagtttaaagacagcactgaggacaaaaccaatgaggtttgtaaccagattcatcgacttggatggcctatcatgtatcctcaactttctaaagaccatggactacgagacctcagagtctcgaatacatacttctctcattggctgtataaaggcgttaatgaacaactctcaaggccgggctcacgtcctggctcattctgagagtattaatgtaattgctcagagtctgagcacagagaacattaaaacgaaggtggccgtgctggaaatcttgggcgccgtgtgcctggttcccgggggccacaagaaggttctgcaggccatgctgcactaccagaagtatgccagcgaaaggacccgctttcagacattaattaacgacttggataaaagcactgggcggtatcgagatgaagtgagtctcaagactgccatcatgtccttcattaatgcagtgctcagccaaggtgcaggagtggagagtttggactttagacttcatcttcgctatgaatttctgatgttaggaattcaacctgtaatagataaattaagggaacacgaaaattcaacattagataggcatttagacttttttgaaatgctccgaaatgaagatgaactagaatttgccaaaagatttgaactggttcacatagacacaaaaagtgcaactcagatgtttgagctgaccaggaagaggctgacacatagtgaagcttacccgcatttcatgtccatcctgcaccactgcctccaaatgccttacaagaggagtggcaacactgttcagtactggctactactagatagaattatacagcagatagttatccagaatgacaaaggacaggaccctgactccacacctttggaaaactttaatattaagaatgtcgtacgaatgttggttaatgaaaatgaagttaagcagtggaaagaacaagcggaaaaaatgagaaaagagcacaatgagctacaacagaaactggaaaagaaagaacgagaatgtgatgctaagactcaagagaaggaagagatgatgcagaccttaaataaaatgaaagagaaacttgaaaaggagactactgagcataagcaagtcaagcagcaggtggcggacctcacagcacagctccatgagctcagcaggagggccgtctgtgcttcaatcccaggtggaccctcgcctggagcaccaggagggccctttccttcctctgtgcctggatctctccttcctcccccaccacccccacctctaccaggtgggatgcttccccctccaccgcctcccctccctccaggtggccctcctcctcccccagggcctcctcccttaggggcaatcatgccacctcctggtgctccaatgggcctagcactgaagaagaaaagcattcctcagcccacaaatgccctgaaatccttcaactggtctaaactgcccgagaacaaactggaaggaacagtatggaccgaaattgatgatacaaaagtcttcaaaattctagatcttgaagacctggaaagaaccttctctgcctatcaaagacagcaggatttctttgtgaacagtaactccaagcagaaagaagcagatgccattgatgacactctgagttccaaacttaaagttaaagagctttcggtgattgatggtcggagagctcagaattgcaacatccttctatcgaggttgaaattatccaatgacgaaatcaaacgggcaattctaacaatggacgaacaggaagatctgcccaaggacatgttggaacagctcttgaaatttgttcctgaaaaaagtgacattgacctattggaggaacataaacacgaactggatcggatggccaaggctgataggttcctttttgagatgagccgaattaatcactatcagcaaaggttgcaatcgctgtacttcaaaaagaagtttgcagagcgtgtggcagaagtgaaacctaaagtggaagcaattcgttctggctcagaagaggtgtttaggagtggtgccctcaagcagttgctggaggtggttttggcatttggaaattatatgaataaaggtcaaagagggaatgcatatggattcaagatatctagcctaaacaaaattgctgacacaaaatccagcatcgacaaaaacattacccttttgcactatctcatcactattgtggaaaataagtaccccagtgttctcaatctaaatgaagaattgcgagatattcctcaagctgcgaaagtaaacatgactgagctggacaaagaaataagtaccttgagaagtggcttgaaagcagtagagacagagctggaatatcagaagtctcagcccccacagcccggagataagtttgtgtctgttgtcagccagttcatcacagtagccagcttcagcttctctgatgttgaagaccttctagcagaagctaaagacctgtttactaaagcagtgaagcactttggggaagaggctggcaaaatacaaccagatgagttctttggcatttttgatcaatttcttcaagctgtgtcagaagccaaacaagaaaacgaaaatatgagaaagaaaaaggaggaagaagaacgtcgagctcgcatggaagctcagctcaaagaacaacgtgaaagggaacgtaaaatgagaaaagctaaagagaatagtgaagaaagcggagagtttgatgaccttgtttcagctttacgctcaggagaagtgtttgacaaagacctttctaaattgaaacggaatcgcaaacgtattaccaaccagatgactgacagcagcagagagagaccaatcacaaaacttaatttctaattttccatgaatacttttttttagaaagctcattagcagccctctaaagtgactagaacgtttcattacactgccttgcaatccaaacagtggcaattttttccttcatctgtgagtgaatgtgtgaacgtgtgtatgtaaatgtatgtgtgtatatattaaaaaatgtatatagatgtctgagtgttgtctggagacctatacgtatggttaaaaagatttatgttaatgtatgtgctccaaaacctttcgtgtatgcattcacattgagtgtggctcattttctttccccgaacgccatgactgttcagaagcacaatactatctcctgaaagagataagagacattccctagattcaaaggcaaaacagaagaaacaaacaaacaaacaaacaaagcttgcaaaatattttatggtttccaagcttgatatcctttaaaattattttcattgatggaactggagttgttggaaaaacatagatttaaaatgatttttgatagctgacattgtgatgttgatgtatcacatcagtaataggaccagctttgaatttctgacattggtgtggggatacagtctgtaaatgtttattgagaacatcttgcacacaatttgaattatgtagaatgtcaatcaagtttttgtatatttaaaagttggacatcaattttttcccctgatttcatcaagttatctctgccaagtgctcttgataatttcttcagatttttggaaaaaaacactatataaatgcaatccatgctttttttaaagaacaacattgccagagtatgcttgttctaacaatatagatatataaaccttaaaaataataaaatatctcacccaagacttaaaggaagaattctctgaagggataaagattact

SEQ ID NO: 66 - protein, 1078 aa, homo sapiens
MAPRKRGGRGISFIFCCFRNNDHPEITYRLRNDSNFALQTMEPALPMPPVEELDVMFSELVDELDLTDKHREAMFALPAEKKWQIYCSKKKDQEENKGATSWPEFYIDQLNSMAARKSLLALEKEEEEERSKTIESLKTALRTKPMRFVTRFIDLDGLSCILNFLKTMDYETSESRIHTSLIGCIKALMNNSQGRAHVLAHSESINVIAQSLSTENIKTKVAVLEILGAVCLVPGGHKKVLQAMLHYQKYASERTRFQTLINDLDKSTGRYRDEVSLKTAIMSFINAVLSQGAGVESLDFRLHLRYEFLMLGIQPVIDKLREHENSTLDRHLDFFEMLRNEDELEFAKRFELVHIDTKSATQMFELTRKRLTHSEAYPHFMSILHHCLQMPYKRSGNTVQYWLLLDRIIQQIVIQNDKGQDPDSTPLENFNIKNVVRMLVNENEVKQWKEQAEKMRKEHNELQQKLEKKERECDAKTQEKEEMMQTLNKMKEKLEKETTEHKQVKQQVADLTAQLHELSRRAVCASIPGGPSPGAPGGPFPSSVPGSLLPPPPPPPLPGGMLPPPPPPLPPGGPPPPPGPPPLGAIMPPPGAPMGLALKKKSIPQPTNALKSFNWSKLPENKLEGTVWTEIDDTKVFKILDLEDLERTFSAYQRQQDFFVNSNSKQKEADAIDDTLSSKLKVKELSVIDGRRAQNCNILLSRLKLSNDEIKRAILTMDEQEDLPKDMLEQLLKFVPEKSDIDLLEEHKHELDRMAKADRFLFEMSRINHYQQRLQSLYFKKKFAERVAEVKPKVEAIRSGSEEVFRSGALKQLLEVVLAFGNYMNKGQRGNAYGFKISSLNKIADTKSSIDKNITLLHYLITIVENKYPSVLNLNEELRDIPQAAKVNMTELDKEISTLRSGLKAVETELEYQKSQPPQPGDKFVSVVSQFITVASFSFSDVEDLLAEAKDLFTKAVKHFGEEAGKIQPDEFFGIFDQFLQAVSEAKQENENMRKKKEEEERRARMEAQLKEQRERERKMRKAKENSEESGEFDDLVSALRSGEVFDKDLSKLKRNRKRITNQMTDSSRERPITKLNF

SEQ ID NO: 67 - DNA, 1096 bp, homo sapiens
  CDS (1)..(765)
atgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtgaatcccatcctggaagggaaggtgcatcgccatctacaggagcagaagaatggacttgctaaatgacctagcactattctctggcccgatttatcatatcccttttctcctccaaatatttctcctctcaccttttctctgggacttaagctgctatatcccctcagagctcacaaatgcctttacattctttccctgacctcctgattttttttttcttttctcaaatgttacctacaatacatgcctggggtaagccacccggctacctaattcctcagtaacctccatctaaaatctccaaggaagcaataaattccttttatgag

SEQ ID NO: 68 - protein, 255 aa, homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 69 - DNA, 2820 bp, homo sapiens
  CDS (238)..(1275)
ggcgggttcgcgccccgaaggctgagagctggcgctgctcgtgccctgtgtgccagacggcggagctccgcggccggaccccgcggccccgctttgctgccgactggagtttgggggaagaaactctcctgcgccccagaagatttcttcctcggcgaagggacagcgaaagatgagggtggcaggaagagaaggcgctttctgtctgccggggtcgcagcgcgagagggcagtgccatgttcctctccatcctagtggcgctgtgcctgtggctgcacctggcgctgggcgtgcgcggcgcgccctgcgaggcggtgcgcatccctatgtgccggcacatgccctggaacatcacgcggatgcccaaccacctgcaccacagcacgcaggagaacgccatcctggccatcgagcagtacgaggagctggtggacgtgaactgcagcgccgtgctgcgcttcttcttctgtgccatgtacgcgcccatttgcaccctggagttcctgcacgaccctatcaagccgtgcaagtcggtgtgccaacgcgcgcgcgacgactgcgagcccctcatgaagatgtacaaccacagctggcccgaaagcctggcctgcgacgagctgcctgtctatgaccgtggcgtgtgcatttcgcctgaagccatcgtcacggacctcccggaggatgttaagtggatagacatcacaccagacatgatggtacaggaaaggcctcttgatgttgactgtaaacgcctaagccccgatcggtgcaagtgtaaaaaggtgaagccaactttggcaacgtatctcagcaaaaactacagctatgttattcatgccaaaataaaagctgtgcagaggagtggctgcaatgaggtcacaacggtggtggatgtaaaagagatcttcaagtcctcatcacccatccctcgaactcaagtcccgctcattacaaattcttcttgccagtgtccacacatcctgccccatcaagatgttctcatcatgtgttacgagtggcgttcaaggatgatgcttcttgaaaattgcttagttgaaaaatggagagatcagcttagtaaaagatccatacagtgggaagagaggctgcaggaacagcggagaacagttcaggacaagaagaaaacagccgggcgcaccagtcgtagtaatccccccaaaccaaagggaaagcctcctgctcccaaaccagccagtcccaagaagaacattaaaactaggagtgcccagaagagaacaaacccgaaaagagtgtgagctaactagtttccaaagcggagacttccgacttccttacaggatgaggctgggcattgcctgggacagcctatgtaaggccatgtgccccttgccctaacaactcactgcagtgctcttcatagacacatcttgcagcatttttcttaaggctatgcttcagtttttctttgtaagccatcacaagccatagtggtaggtttgccctttggtacagaaggtgagttaaagctggtggaaaaggcttattgcattgcattcagagtaacctgtgtgcatactctagaagagtagggaaaataatgcttgttacaattcgacctaatatgtgcattgtaaaataaatgccatatttcaaacaaaacacgtaatttttttacagtatgttttattaccttttgatatctgttgttgcaatgttagtgatgttttaaaatgtgatgaaaatataatgtttttaagaaggaacagtagtggaatgaatgttaaaagatctttatgtgtttatggtctgcagaaggatttttgtgatgaaaggggattttttgaaaaattagagaagtagcatatggaaaattataatgtgtttttttaccaatgacttcagtttctgtttttagctagaaacttaaaaacaaaaataataataaagaaaaataaataaaaaggagaggcagacaatgtctggattcctgttttttggttacctgatttccatgatcatgatgcttcttgtcaacaccctcttaagcagcaccagaaacagtgagtttgtctgtaccattaggagttaggtactaattagttggctaatgctcaagtattttatacccacaagagaggtatgtcactcatcttacttcccaggacatccaccctgagaataatttgacaagcttaaaaatggccttcatgtgagtgccaaattttgtttttcttcatttaaatattttctttgcctaaatacatgtgagaggagttaaatataaatgtacagagaggaaagttgagttccacctctgaaatgagaattacttgacagttgggatactttaatcagaaaaaaagaacttatttgcagcattttatcaacaaatttcataattgtggacaattggaggcatttattttaaaaaacaattttattggccttttgctaacacagtaagcatgtattttataaggcattcaataaatgcacaacgcccaaaggaaataaaatcctatctaatcctactctccactacacagaggtaatcactattagtattttggcatattattctccaggtgtttgcttatgcacttataaaatgatttgaacaaataaaactaggaacctgtatacatgtgtttcataacctgcctcctttgcttggccctttattgagataagttttcctgtcaagaaagcagaaaccatctcatttctaacagctgtgttatattccatagtatgcattactcaacaaactgttgtgctattggatacttaggtggtttcttcactgacaatactgaataaacatctcaccggaattc

SEQ ID NO: 70 - protein, 346 aa, homo sapiens
MFLSILVALCLWLHLALGVRGAPCEAVRIPMCRHMPWNITRMPNHLHHSTQENAILAIEQYEELVDVNCSAVLRFFFCAMYAPICTLEFLHDPIKPCKSVCQRARDDCEPLMKMYNHSWPESLACDELPVYDRGVCISPEAIVTDLPEDVKWIDITPDMMVQERPLDVDCKRLSPDRCKCKKVKPTLATYLSKNYSYVIHAKIKAVQRSGCNEVTTVVDVKEIFKSSSPIPRTQVPLITNSSCQCPHILPHQDVLIMCYEWRSRMMLLENCLVEKWRDQLSKRSIQWEERLQEQRRTVQDKKKTAGRTSRSNPPKPKGKPPAPKPASPKKNIKTRSAQKRTNPKRV

SEQ ID NO: 71 - DNA, 1362 bp, homo sapiens
  CDS (50)..(1066)
catttggggacgctctcagctctcggcgcacggcccagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtacttt

SEQ ID NO: 72 - protein, 339 aa, homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 73 - DNA, 850 bp, homo sapiens
  CDS (107)..(781)
gcgccttcctctccgcagccccccgggatgcggtagcggccgctgtgcggaggccgcgaagcagctgcagccgccgccgcgcagatccacgctggctccgtgcgccatggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgtaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaaggggtaaagggcgcaaagggcatgggtcgggagaggggacgcaggcccctctcctccgtggcacat

SEQ ID NO: 74 - protein, 225 aa, homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVYHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 75 - DNA, 369 bp, homo sapiens
  CDS (1)..(366)
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 76 - protein, 122 aa, homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 77 - DNA, 895 bp, homo sapiens
  CDS (147)..(497)
gcggccgcgtcgaccggcgcggctggagcgcagcgccgaagggactggcagggctgaagtgtgcgggacagcaagcccccgaatagccccggctgccacctcgcaggacccaaggccacgcgcgccgggcccagctgagccgcctcatgaagccgcccgcggaggacctgtcggacgcgctgtgcgagtttgacgcggtgctggccgacttcgcgtcgcccttccacgagcgccacttccactacgaggagcacctggagcgcatgaagcggcgcagcagcgccagtgtcagcgacagcagcggcttcagcgactcggagagtgcagattcactttataggaacagcttcagcttcagtgatgaaaaactgaattctccaacagactctaccccagctcttctctctgccactgtcactcctcagaaagctaaattaggagacacaaaagagctagaagccttcattgctgatcttgacaaaactttagcaagtatgtgaaacaagaagttctgggtcctttcatcataagggagaagcttcagaaagttccgaggacctgctaaaatcagctactagaatctgctgccagaggggacaaagacgtgcactcaaccttctaccaggccactctcaggctcaccttaaaatcagcccttgatcccatttctgggcaatttagacagtgaaactgactttgtttacctgcttgcagcatattagaacagacgatccatgctaatattgtattttctcttaaaacatagctttcctgtaatttaaagtgcttttatgaaaatatttgtaattaattatatatagttggaaatagcagtaagctttcccattataatatatttttgtatacaaataaaatttgaactgaacctcgtgcc

SEQ ID NO: 78 - protein, 117 aa, homo sapiens
MKPPAEDLSDALCEFDAVLADFASPFHERHFHYEEHLERMKRRSSASVSDSSGFSDSESADSLYRNSFSFSDEKLNSPTDSTPALLSATVTPQKAKLGDTKELEAFIADLDKTLASM

SEQ ID NO: 79 - DNA, 1564 bp, homo sapiens
  CDS (314)..(1138)
gaagcacatctggacagctgtgcggcctccttgcgggccgacgtcagccgagcacgtcccccacgtcctctccttctcgccacttattatttattcgttttcccaaagaagcgactagggacccaagtttaaaaattcctccccccactcaatgcgagacgtggccagatcccatccaacacacggtttaattttcatggggctctgggatcaaaagaacagaaacagcaacaacaaaagcccagccgctgtctgattttaagctggcaaagtgggaaaaataaagtgttgagtaaacagaccaagttggatcatggggaatttcagaggtcatgccctccctggaaccttcttttttattattggtctttggtggtgtacaaagagtattctgaagtatatctgcaaaaagcaaaagcgaacctgctatcttggttccaaaacattattctatcgattggaaattttggagggaattacaatagttggcatggctttaactggcatggctggggagcagtttattcctggagggccccatctgatgttatatgactataaacaaggtcactggaatcaactcctgggctggcatcatttcaccatgtatttcttctttgggctgttgggtgtggcagatatcttatgtttcaccatcagttcacttcctgtgtccttaaccaagttaatgttgtcaaatgccttatttgtggaggcctttatcttctacaaccacactcatggccgggaaatgctggacatctttgtgcaccagctgctggttttggtcgtctttctgacaggcctcgttgccttcctagagttccttgttcggaacaatgtacttctggagctattgcggtcaagtctcattctgcttcaggggagctggttctttcagattggatttgtcctgtatccccccagtggaggtcctgcatgggatctgatggatcatgaaaatattttgtttctcaccatatgcttttgttggcattatgcagtaaccattgtcatcgttggaatgaattatgctttcattacctggttggttaaatctagacttaagaggctctgctcctcagaagttggacttctgaaaaatgctgaacgagaacaagaatcagaagaagaaatgtgactttgatgagcttccagtttttctagataaaccttttcttttttacattgttcttggttttgtttctcgatcttttgtttggagaacagctggctaaggatgactctaagtgtactgtttgcatttccaatttggttaaagtatttgaatttaaatattttctttttagctttgaaaatattttgggtgatactttcattttgcacatcatgcacatcatggtattcaggggctagagtgatttttttccagattatctaaagttggatgcccacactatgaaagaaatatttgttttatttgccttatagatatgctcaaggttactgggcttgctactatttgtaactccttgaccatggaattatacttgtttatcttgttgctgcaatgagaaataaatgaatgtatgtattttggtgc

SEQ ID NO: 80 - protein, 275 aa, homo sapiens
MGNFRGHALPGTFFFIIGLWWCTKSILKYICKKQKRTCYLGSKTLFYRLEILEGITIVGMALTGMAGEQFIPGGPHLMLYDYKQGHWNQLLGWHHFTMYFFFGLLGVADILCFTISSLPVSLTKLMLSNALFVEAFIFYNHTHGREMLDIFVHQLLVLVVFLTGLVAFLEFLVRNNVLLELLRSSLILLQGSWFFQIGFVLYPPSGGPAWDLMDHENILFLTICFCWHYAVTIVIVGMNYAFITWLVKSRLKRLCSSEVGLLKNAEREQESEEEM

SEQ ID NO: 81 - DNA, 2311 bp, homo sapiens
  CDS (199)..(876)
gatttaatcctatgacaaactaagttggttctgtcttcacctgttttggtgaggttgtgtaagagttggtgtttgctcaggaagagatttaagcatgcttgcttacccagactcagagaagtctccctgttctgtcctagctatgttcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccagcccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgccagcatttcccaacacaaagattctgaccttaaatgcaaccatttgaaacccctgtaggcctcaggtgaaactccagatgccacaatgagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagagaggctgtctgttgtattcattgtggtcatagcacctaacaacattgtagcctcaatcgagtgagacagactagaagttcctagttggcttatgatagcaaatggcctcatgtcaaatattagatgtaattttgtgtaagaaatacagactggatgtaccaccaactactacctgtaatgacaggcctgtccaacacatctcccttttccatgctgtggtagccagcatcggaaagaacgctgatttaaagaggtgagcttgggaattttattgacacagtaccatttaatggggagacaaaaatgggggccaggggagggagaagtttctgtcgttaaaaacgagtttggaaagactggactctaaattctgttgattaaagatgagctttgtctaccttcaaaagtttgtttggcttacccccttcagcctccaattttttaagtgaaaatataactaataacatgtgaaaagaatagaagctaaggtttagataaatattgagcagatctataggaagattgaacctgaatattgccattatgcttgacatggtttccaaaaaatggtactccacatacttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataat

SEQ ID NO: 82 - protein, 226 aa, homo sapiens
MDWGTLQTILGGVNKHSTSIGKIWLTVLFIFRIMILVVAAKEVWGDEQADFVCNTLQPGCKNVCYDHYFPISHIRLWALQLIFVSSPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 83 - DNA, 2389 bp, homo sapiens
  CDS (73)..(1143)
gggaaggcgagcagtgccaatctacagcgaagaaagtctcgtttggtaaaagcgagaggggaaagcctgagcatgcagagtgtgcagagcacgagcttttgtctccgaaagcagtgcctttgcctgaccttcctgcttctccatctcctgggacaggtcgctgcgactcagcgctgccctccccagtgcccgggccggtgccctgcgacgccgccgacctgcgcccccggggtgcgcgcggtgctggacggctgctcatgctgtctggtgtgtgcccgccagcgtggcgagagctgctcagatctggagccatgcgacgagagcagtggcctctactgtgatcgcagcgcggaccccagcaaccagactggcatctgcacggcggtagagggagataactgtgtgttcgatggggtcatctaccgcagtggagagaaatttcagccaagctgcaaattccagtgcacctgcagagatgggcagattggctgtgtgccccgctgtcagctggatgtgctactgcctgagcctaactgcccagctccaagaaaagttgaggtgcctggagagtgctgtgaaaagtggatctgtggcccagatgaggaggattcactgggaggccttacccttgcagcttacaggccagaagccaccctaggagtagaagtctctgactcaagtgtcaactgcattgaacagaccacagagtggacagcatgctccaagagctgtggtatggggttctccacccgggtcaccaataggaaccgtcaatgtgagatgctgaaacagactcggctctgcatggtgcggccctgtgaacaagagccagagcagccaacagataagaaaggaaaaaagtgtctccgcaccaagaagtcactcaaagccatccacctgcagttcaagaactgcaccagcctgcacacctacaagcccaggttctgtggggtctgcagtgatggccgctgctgcactccccacaataccaaaaccatccaggcagagtttcagtgctccccagggcaaatagtcaagaagccagtgatggtcattgggacctgcacctgtcacaccaactgtcctaagaacaatgaggccttcctccaggagctggagctgaagactaccagagggaaaatgtaacctatcactcaagaagcacacctacagagcacctgtagctgctgcgccacccaccatcaaaggaatataagaaaagtaatgaagaatcacgatttcatccttgaatcctatgtattttcctaatgtgatcatatgaggacctttcatatctgtcttttatttaacaaaaaatgtaattaactgtaaacttggaatcaaggtaagctcaggatatggcttaggaatgacttactttcctgtggttttattacaaatgcaaatttctataaatttaagaaaacaagtatataatttactttgtagactgtttcacattgcactcatcatattttgttgtgcactagtgcaattccaagaaaatatcactgtaatgagtcagtgaagtctagaatcatacttaacatttcattgtacaagtattacaaccatatattgaggttcattgggaagattctctattggctccctttttgggtaaaccagctctgaacttccaagctccaaatccaaggaaacatgcagctcttcaacatgacatccagagatgactattacttttctgtttagttttacactaggaaacgtgttgtatctacagtaatgaaatgtttactaagtggactggtgtcataaactttctccatttaagacacattgactcctttccaatagaaagaaactaaacagaaaactcccaatacaaagatgactggtccctcatagccctcagacatttatatattggaagctgctgaggcccccaagttttttaattaagcagaaacagcatattagcagggattctctcatctaactgatgagtaaactgaggcccaaagcacttgcttacatcctctgatagctgtttcaaatgtgcattttgtggaattttgagaaaaatagagcaaaatcaacatgactggtggtgagagaccacacattttatgagagtttggaattattgtagacatgcccaaaacttatccttgggccataattatgaaaactcatgatcaagatatatgtgtatacatacatgtatctggtttgtcaggctacaaggtaggctgcaaaattaaatctagacattcttttaatgccaccacacgtgttccgcttctctcttttaaagtatttataaaaatataaattgtacattttgtaaaatattatgtttgatttctctacttgtcatatcactaaataaacacgattttattgctgaaaaaaaaaaaaaaaaaaa

SEQ ID NO: 84 - protein, 357 aa, homo sapiens
MQSVQSTSFCLRKQCLCLTFLLLHLLGQVAATQRCPPQCPGRCPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM